(19) United States
(12) Patent Application Publication
(10) Pub. No.: US 2024/0335405 A1
(43) Pub. Date: Oct. 10, 2024

(54) VIDOFLUDIMUS AND RELATED STRUCTURES ACTING AS NURR1 AGONISTS

(71) Applicant: IMMUNIC AG, Gräfelfing (DE)

(72) Inventors: Christian GEGE, Ehingen (DE); Hella KOHLHOF, Munich (DE); Evelyn PEELEN, Munich (DE); Andreas MÜHLER, Munich (DE); Daniel VITT, Germering (DE)

(21) Appl. No.: 18/621,364

(22) Filed: Mar. 29, 2024

Related U.S. Application Data
(60) Provisional application No. 63/492,961, filed on Mar. 29, 2023.

(51) Int. Cl. A61K 31/196; A61P 25/28

(52) U.S. Cl. CPC A61K 31/196; A61P 25/28

(57) ABSTRACT

The present invention relates to methods of treating or ameliorating a neurodegenerative disease, such as Parkinson's disease or multiple sclerosis, with a compound according to Formula (I) to (V), or with a compound according to Formula (VI), or a pharmaceutically acceptable salt or solvate thereof, for example, vidofludimus, which acts as a Nurr1 agonist.

### FIELD OF THE DISCLOSURE

Described herein are methods of treating or ameliorating a neurological disease, e.g., a neurodegenerative disease such as Parkinson's disease (PD) or multiple sclerosis (MS), with a compound according to Formula (I) to (V), or with a compound according to Formula (VI), or a pharmaceutically acceptable salt or solvate thereof, especially vidofludimus, which surprisingly act also as a nuclear receptor related 1 (Nurr1) agonist beside the known mode of action as dihydroorotate dehydrogenase (DHODH) inhibitors. This dual mode of action provides significant benefits: a) inhibition of DHODH is beneficial against inflammation and b) activation of Nurr1 is known to protecting neurons from damage and death. Compounds formerly described as DHODH inhibitors, as well as new compounds, are shown to bind directly to Nurr1, activate expression of Nurr1, and mediate activity through Nurr1 in cells. Accordingly, the compounds may be used to inhibit DHODH and/or activate Nurr1, providing benefits for treating neurological diseases, such as inflammatory neurological diseases like PD, MS, and others.

### BACKGROUND

Neurodegenerative diseases are characterized by the progressive loss of structure and function of neurons. In subjects suffering from these diseases, neurodegeneration usually worsens over time, resulting in impairment or loss of nervous system function. To date, there are no known cures for neurodegenerative diseases.

The rising prevalence of neurodegenerative diseases has spurred research into improved methods for diagnosing affected subjects, as well as treatments that can slow or reduce progression of disease and/or improve disease symptoms. For some diseases, biomarkers associated with neurodegeneration have provided valuable information on molecular mechanisms whose absence or dysfunction may contribute to disease. Exemplary biomarkers include, but are not limited to, Nurr1, NFL, GFAP, GDNF, BDNF, VMAT2, TH, L-DOPA, CXCL13, LTA, FCN2, ICAM3, LY9, SLAMF7, TYMP, CHI3L1, FYB1, TNFRSF1B, and combinations thereof.

Nurr1 is the second member of the nerve growth factor-induced b subfamily of orphan nuclear receptors (NR4A2). Nurr1 is a neuroprotective transcription factor mainly found in the central nervous system with high expression in neurons. Nurr 1 has neuroprotective and antineuroinflammatory activity and emerges as an attractive target to treat neurodegenerative pathologies including Alzheimer's disease (AD), Parkinson's disease (PD), and multiple sclerosis (MS). The expression level of miR-132 is negatively correlated with its downstream molecule Nurr1, which is one of the key factors for the maintenance of dopaminergic function and is downregulated in PD.

Neurofilament light chain (NFL) may be measured in the cerebrospinal fluid (CSF), and is useful for the diagnosis and the prediction of progression of several neurodegenerative diseases (Delaby et al. (Sci. Rep. 2020; 10:9161) and Gaetani et al. (J. Neurol. Neurosurg. Psychiatry 2019; 90:870).

GFAP may be well suited for detecting the relapse independent neurodegeneration in contrast to relapse related disability worsening in MS (Meier et al. JAMA Neurol. 2023; 80:287).

Other representative examples of biomarkers whose gene/protein induction or activity have been associated with neurodegenerative diseases are GDNF, BDNF, VMAT2, TH, L-DOPA or GFAP in serum, plasma or CSF. Additional biomarkers such as CXCL13, LTA, FCN2, ICAM3, LY9, SLAMF7, TYMP, CHI3L1, FYB1, TNFRSF1B, NFL, and combinations of these have been used to predict the severity of disability worsening in MS in a study of age-related MS severity (Nat. Commun. 2023; 14:6903).

These studies indicate that the exemplary biomarkers play a role in development and/or progression of multiple sclerosis. MS is a neurodegenerative disease characterized by an autoimmune attack on the myelin sheath in neurons of the brain and spinal cord. The symptoms of MS include physical disorders such as fatigue, a loss or change in sensation and coordination, vision impairment, muscle weakness and/or spasms, as well as mental disorders such as cognitive impairment, unstable moods, depression, and anxiety. There is no known cure for MS, and the disease symptoms are particularly challenging to treat due to the combined effects of inflammation and neurodegeneration across multiple disease pathways. In addition, MS manifests in different forms, e.g., relapsing forms (relapsing MS, or RMS) in which disease symptoms occur in isolated attacks, and progressive forms (progressive MS, or PMS) in which disease symptoms build up over time. Patients show extensive heterogeneity in presentation, duration, progression of symptoms, and response to treatments. Thus, while current disease-modifying therapies (DMTs) for MS may delay disability accrual in some patients, the majority of DMTs address only relapse-associated disability worsening and show the most benefit in patients with relapse-associated worsening (RAW) of disability at the earliest stages of MS. To date, most DMTs are approved for the treatment of relapses associated with RMS or PMS, while other forms of MS cannot be adequately treated. There is an unmet need for treatments that are suitable for different forms of MS, as well as treatments for both early and late stages of MS.

Recent preclinical and clinical studies with vidofludimus suggest that this potent DHODH inhibitor is effective in treating immune-related disorders, including MS. In an animal model of relapsing-remitting MS (RRMS), treatment with vidofludimus calcium led to a greater reduction in brain lesions than a control treatment (Muehler et al., Mult. Scler. Relat. Disord. 2020; 43:102129). Similar results were demonstrated in the EMPhaSIS clinical trial (NCT03846219), where RRMS patients treated with vidofludimus calcium for 12 weeks or 24 weeks had significantly fewer unique active brain lesions and a lower rate of confirmed disability worsening over time when compared to patients treated with a placebo (U.S. patent Ser. No. 11/877,994B2). The positive effects for patients with relapsing-remitting multiple sclerosis may be attributed to the anti-inflammatory properties of vidofludimus, e.g., mediated by DHODH inhibition.

In other studies, vidofludimus has been shown to be efficacious and well-tolerated in human patients. In the COMPONENT trial (NCT01010581), vidofludimus safety data was established in patients with rheumatoid arthritis (Muehler et al., Drugs R D 2019; 19:351). Safety, tolerability, and pharmokinetics was also determined in healthy subjects (Muehler et al., Eur. J. Drug Metab. Pharmacokinet. 2020; 45:557). In addition, anti-viral effects of vidofludimus were demonstrated in studies of mammarenaviruses (Kim et al., Viruses 2020; 12:821) and SARS-CoV-2 (Hahn et al., Viruses 2020; 12:1394; Stegmann et al., iScience 2022; 25:104293; and Vehreschild et al., Infect. Dis. Ther. 2022; 11:2159). Finally, vidofludimus has been studied as a farnesoid X receptor (FXR) modulator (Zhu et al., Front. Pharmacol. 2020; 11:590 and Heering et al. ACS Chem. Biol. 2022; 17:3159), a New Delhi metallo-beta-lactamase 1 (NDM-1) inhibitor, an antibiotic protective agent (Chinese Patent Application No. CN113842380), and a potential treatment of inflammatory diseases including Alzheimer's disease (Patent Application No. WO 2018/177151).

Taken together, these studies suggest the vidofludimus is a promising candidate for treatment of many disorders, including MS. However, there remains a need in the art for applying knowledge of immune diseases, neurodegenerative diseases, and biomarkers to new methods for diagnosing and/or treating neurodegenerative diseases.

### BRIEF DESCRIPTION OF THE DRAWINGS

FIG. 1 depicts the binding of Example 1 (vidofludimus) and Example 3 to transcription factor Nurr1 (NR4A2) in the isothermal titration calorimetry (ITC) assay according to Example 1. The data shows a K_{d} of 0.7 μM and 0.3 μM, respectively.

FIG. 2 depicts as representative graphs the activation of the transcription factor Nurr1 (NR4A2) in the Gal4 assay according to Example 101-A (left) and in the full-length assay according to Example 101-B (right) with compound Example 1. The data shows an EC₅₀ (Gal4-Nurr1) of 0.40±0.20 PM.

FIG. 3 shows the gene expression on tyrosine hydroxylase (TH) with compound Example 1 (as its calcium salt IMU-838) and Example 3. The dose-dependent activation of the target gene vesicular amino acid transporter 2 (VMAT2) is depicted with compound Example 3.

FIG. 4 shows the gene expression in PBMCs on Nurr1 target gene brain-derived neurotrophic factor (BDNF) when treated with IMU-838 (3 μM) while comparative DHODH inhibitor teriflunomide has no effect at the same concentration.

FIG. 5 shows reduced apoptosis in neuronal-like cell lines when treated with Example 3.

FIG. 6 shows the change from baseline in serum neurofilament by number of relapses during main treatment period. Full analysis set of Cohort 1 and Cohort 2 for patients with no relapse up to week 24.

FIG. 7 depicts a powder X-Ray diffraction pattern of Polymorph A of IMU-838.

FIG. 8 shows a plot of disability worsening vs time for RRMS, active SPMS, non-active SPMS and PPMS.

FIG. 9 shows the serum neurofilament data from biomarker interim analysis CALLIPER (Phase 2 PMS) for IMU-838: Change to week 24 as compared to placebo in % of baseline.

FIG. 10 shows a comparison of CALLIPER interim neurofilament data to historical studies in PPMS and SPMS. NFL reduction compares favourably with other PMS available therapies.

### SUMMARY

One aspect of the present disclosure relates to a compound of Formula (V) or a pharmaceutically acceptable salt or solvate thereof for use in a method of treating a neurogenerative condition and/or a method of neuroprotection in a subject having a neurogenerative disease, wherein the compound is of Formula (V):

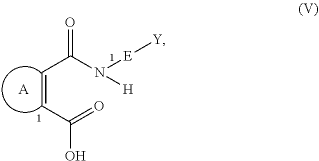

wherein:
   Ring A is

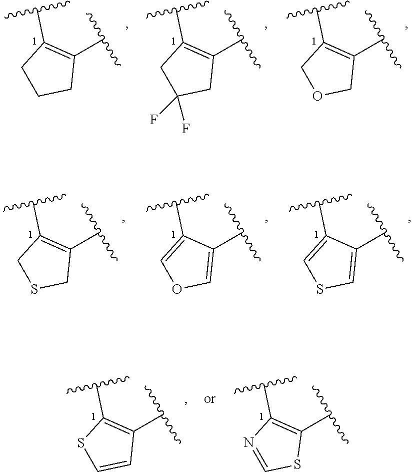

E is

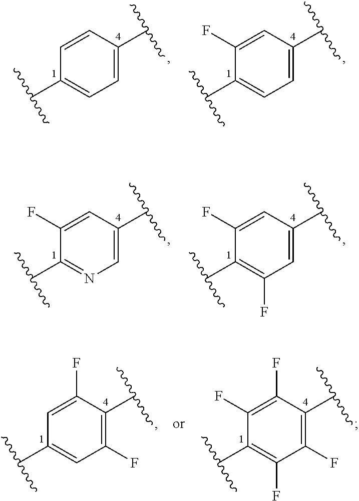 [SMILES: *c1ccc(*)cc1 ; *c1ccc(*)c(F)c1 ; *c1cnc(*)c(F)c1 ; *c1cc(F)c(*)c(F)c1 ; *c1cc(F)c(*)c(F)c1 ; *c1c(F)c(F)c(*)c(F)c1F]

and
Y is

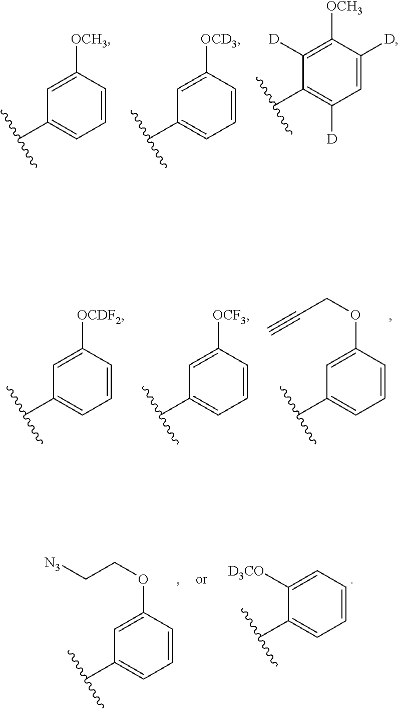

In some embodiments, the subject exhibits an aberrant level of a biomarker associated with a neurodegenerative condition prior to the administering.

In some embodiments, the compound is:

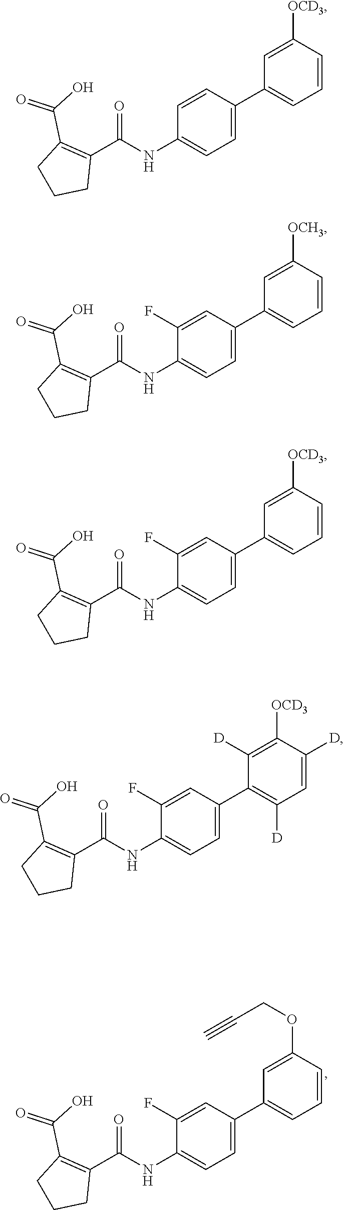

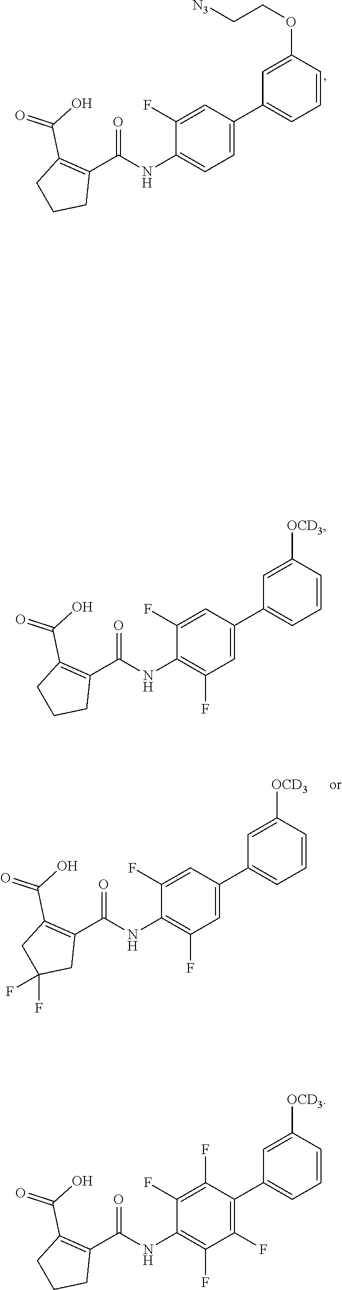

In some embodiments, the compound is

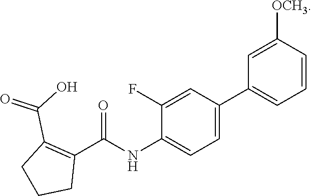 [SMILES: COc1cccc(-c2ccc(NC(=O)C3=C(C(=O)O)CCC3)c(F)c2)c1]

In some embodiments, the compound is

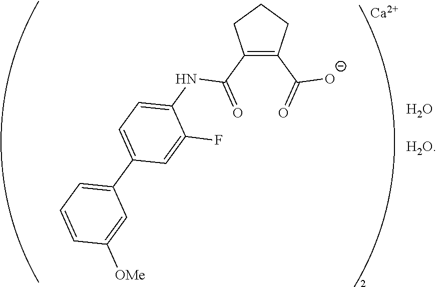

In some embodiments, the biomarker is Nurr1.

In some embodiments, an aberrant level of the biomarker associated with the neurodegenerative condition is downregulated Nurr1.

In some embodiments, the neurodegenerative condition is multiple sclerosis.

In some embodiments, the neurodegenerative condition is multiple sclerosis, wherein the disability is acquired through progression independent of relapse activity (PIRA).

In some embodiments, the neurodegenerative condition is brain atrophy induced by multiple sclerosis.

In some embodiments, the neurodegenerative condition is non-inflammatory multiple sclerosis worsening.

In some embodiments, the neuroprotection is slowing or preventing loss of neurons induced by multiple sclerosis.

In some embodiments, the neuroprotection is slowing or preventing loss of dopaminergic neurons induced by multiple sclerosis.

In some embodiments, the neurodegenerative condition is Parkinson's Disease.

In some embodiments, the method comprises:
determining a level or activity of Nurr1 in the ex vivo biological sample of the subject with an assay selected from (a) a real-time PCR assay of Nurr1 gene expression against relevant housekeeping genes/internal controls (e.g. GAPDH), (b) an immunoassay (such as ELISA) with the suitable antibodies for Nurr1 protein, or (c) a Western blot for Nurr1 protein from the biological sample selected from peripheral blood, peripheral blood lymphocytes, serum, plasma, CSF or peripheral blood mononuclear cells; and
if the test sample from the patient comprises a level of Nurr1 of no greater than about 90% of the level in a healthy subject of same age, gender and/or BMI, administering to the patient an effective amount of Formula (V) or a pharmaceutically acceptable salt or a solvate thereof.

In some embodiments, the method comprises:
a) obtaining a level of a protein in a subject, wherein the protein is downstream of Nurr1 in a biological pathway in the subject; and
b) based on the level of the protein in the subject, determining whether to administer to the subject a Nurr1 agonist.

In some embodiments, the protein is selected from the group consisting of BDNF, GDNF, C-RET, GFAP, DAT, Pitx3, TH, VMAT2, SOD1, AADC, TNFα, iNOS and IL-1β.

An aspect of the present disclosure relates to a compound of Formula (V) or a pharmaceutically acceptable salt or solvate thereof for use in a method of treating a neurogenerative condition and/or a method of neuroprotection in a subject having a neurogenerative disease, wherein the compound is

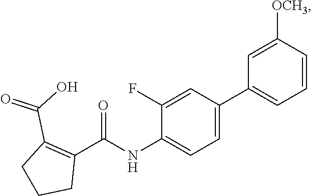 [SMILES: COc1cccc(-c2ccc(NC(=O)C3=C(C(=O)O)CCC3)c(F)c2)c1]

and wherein said condition is PIRA.

In some embodiments, the compound is

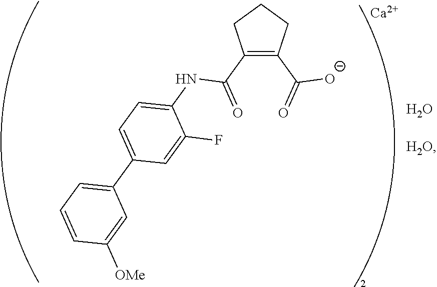

and wherein said condition is PIRA.

In some embodiments, the neurodegenerative condition is multiple sclerosis, wherein a disability is acquired through progression independent of relapse activity (PIRA).

In some embodiments, the therapeutically-effective amount is about 5 mg to about 100 mg.

In some embodiments, the administering is oral by a solid dosage form.

In some embodiments, the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 30 mg to about 50 mg.

In some embodiments, the method aims to prevent or slow disease progression and secondary injuries by halting or at least slowing (mitigate) the loss of neurons.

A further aspect of the present disclosure relates to a method of treating multiple sclerosis (MS) in a patient diagnosed with progression independent of relapse (PIRA), comprising administering the subject a compound of Formula (V), wherein the compound is:

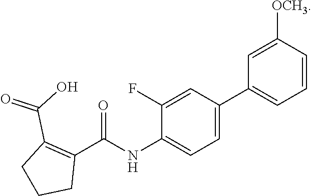 [SMILES: COc1cccc(-c2ccc(NC(=O)C3=C(C(=O)O)CCC3)c(F)c2)c1]

Another aspect of the present disclosure relates to a method of treating multiple sclerosis (MS) in a subject, comprising administering to the subject a compound of Formula (V), wherein the compound is

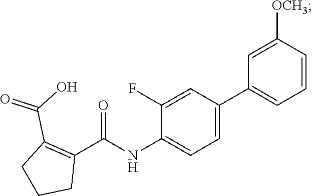 [SMILES: COc1cccc(-c2ccc(NC(=O)C3=C(C(=O)O)CCC3)c(F)c2)c1]

the MS is characterized by progression independent of relapse (PIRA).

Another aspect of the present disclosure relates to a method of treating progression independent of relapse (PIRA) in a subject suffering from multiple sclerosis (MS), comprising administering to the subject a compound of Formula (V), wherein the compound is

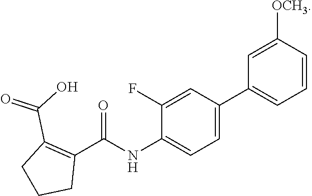 [SMILES: COc1cccc(-c2ccc(NC(=O)C3=C(C(=O)O)CCC3)c(F)c2)c1]

Another aspect of the present disclosure relates to a method of treating multiple sclerosis (MS) in a subject, comprising administering to the subject a compound of Formula (V), wherein:
the compound is

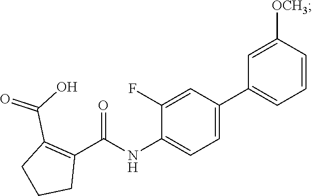 [SMILES: COc1cccc(-c2ccc(NC(=O)C3=C(C(=O)O)CCC3)c(F)c2)c1]

and
the subject has MS characterized by progression independent of relapse (PIRA).

In some embodiments, the patient has no evidence of relapse for 24 months.

In some embodiments, the MS is progressive MS (PMS).

In some embodiments, the PMS is characterized by few or no active lesions.

In some embodiments, the MS is primary progressive MS (PPMS).

In some embodiments, the MS is non-active secondary progressive MS (n-aSPMS).

In some embodiments, the n-aSPMS is characterized by no lesions for 12 months.

In some embodiments, the MS is active secondary progressive MS (a-SPMS).

In some embodiments, the method reduces serum NFL levels in the subject, as compared to a control.

In some embodiments, the method reduces serum GFAP levels in the subject, as compared to a control.

In some embodiments, the method reduces the rate of percent brain volume change (PBVC) in the subject, as compared to a control.

In some embodiments, the method reduces the rate of change in brain parenchymal fraction (BPF) in the subject, as compared to a control.

In some embodiments, the method increases the time to confirmed disability worsening, e.g, based on an expanded disability status scale (EDSS), in the subject as compared to a control.

In some embodiments, the method prevents and/or slows disease progression and secondary injuries by halting or at least slowing (mitigate) the loss of neurons.

In some embodiments, the compound is administered in a therapeutically-effective amount of about 5 mg to about 100 mg.

In some embodiments, the compound is administered orally by a solid dosage form.

In some embodiments, the compound is administered over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 30 mg to about 50 mg.

### DETAILED DESCRIPTION

### Overview

Described herein, inter alia, are methods of treating or ameliorating a disease in a subject having, suspected of having, or at risk of developing the disease, comprising administering to the subject a therapeutically effective amount of a compound of Formulas (I) to (V), or a compound of Formula (VI), or a pharmaceutically acceptable salt or solvate thereof, especially vidofludimus. Compounds (I), (II), (III), (IV), (V), (VI), and salts and solvates are described in detail herein.

In some embodiments, the disease is a neurological disease, e.g., a neurodegenerative disease.

In some embodiments, the disclosure provides a method of treating a condition in a subject, the method comprising administering to the subject a therapeutically-effective amount of a compound of Formula (I),

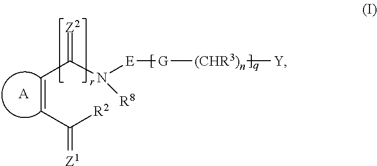

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof, wherein said condition is a neurogenerative condition.

In one embodiment the disclosure provides a method of neuroprotection in a subject having a neurogenerative disease the method comprising administering to the subject a therapeutically-effective amount of a compound of Formula (I) as above outlined. In one embodiment the subject exhibits an aberrant level of a biomarker associated with a neurodegenerative condition prior to the administering.

In one embodiment, a method of providing neuroprotection in a subject having a neurogenerative disease comprises administering to the subject a therapeutically-effective amount of a compound of Formula (I) as above outlined. In one embodiment the subject exhibits an aberrant level of a biomarker associated with a neurodegenerative condition prior to the administering, in particular an aberrant level of Nurr1 in the subject.

In one embodiment vidofludimus or structures according to Formula (I) to (V), or with a compound according to Formula (VI), modulate Nurr1 and therefore Parkinson's disease (PD), amyotrophic lateral sclerosis (ALS), schizophrenia, PIRA in MS patients and drug addiction can be treated with structures according to Formula (I) to (V), or with a compound according to Formula (VI).

Vidofludimus or structures according to Formula (I) to (V), or with a compound according to Formula (VI), that are in the scope of the present invention are characterized by their binding directly to the Nurr1 and their acting as Nurr1 agonists in cells with a different structure-activity-relationship compared to DHODH (see Example 102).

The neurogenerative disease or neurogenerative condition that maybe treated according to the present invention may be selected from the group comprising multiple sclerosis (MS), Parkinson's disease.

In some embodiments, the condition is Parkinson's disease (PD)

In some embodiments, the disclosure provides a method of treating Parkinson's disease in a subject, the method comprising administering to the subject a therapeutically-effective amount of a compound of Formula (I),

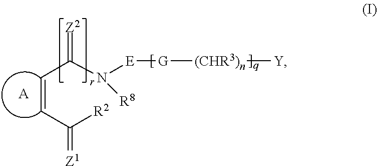

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

In one embodiment the subject exhibits an aberrant level of a biomarker associated with Parkinson's Disease prior to the administering.

In some embodiments, the condition is drug addiction.

In some embodiments, the disclosure provides a method of treating drug addiction in a subject, the method comprising administering to the subject a therapeutically-effective amount of a compound of Formula (I),

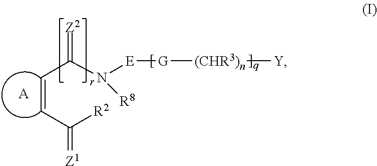

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof. In one embodiment the subject exhibits a level of a biomarker associated with drug addiction prior to the administering.

In some embodiments, the disease is multiple sclerosis (MS).

In some embodiments, the disclosure provides a method of treating multiple sclerosis in a subject, the method comprising administering to the subject a therapeutically-effective amount of a compound of Formula (I),

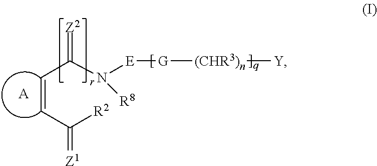

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

In one embodiment the subject exhibits an aberrant level of a biomarker associated with multiple sclerosis prior to the administering.

In some embodiments, the disclosure provides a method comprising: a) obtaining a level of Nurr1 in a subject; and b) based on the level of Nurr1 in the subject, determining whether to administer to the subject a Nurr1 agonist.

In some embodiments, the disclosure provides a method comprising: a) obtaining a level of Nurr1 in a subject who is undergoing a therapy for a neurodegenerative condition; and b) based on the level of Nurr1 in the subject, determining whether to continue the therapy for the neurodegenerative condition, wherein the therapy is Nurr1 agonism.

In some embodiments, the disclosure provides a method comprising: a) obtaining a level of a protein in a subject, wherein the protein is downstream of Nurr1 in a biological pathway in the subject; and b) based on the level of the protein in the subject, determining whether to administer to the subject a Nurr1 agonist.

In some embodiments, the disclosure provides a method comprising: a) obtaining a level of a protein in a subject who is undergoing a therapy for a neurodegenerative condition, wherein the protein is downstream of Nurr1 in a biological pathway in the subject, wherein the therapy is Nurr1 agonism; and b) based on the level of the protein in the subject, determining whether to continue the therapy for the neurodegenerative condition.

In some embodiments, the disclosure provides a method comprising: a) obtaining a level of activity of a gene in a subject, wherein the gene is downstream of Nurr1 in a biological pathway in the subject; and b) based on the level of activity of the gene in the subject, determining whether to administer to the subject a Nurr1 agonist for a neurodegenerative condition.

In some embodiments, the disclosure provides a method comprising: a) obtaining a level of activity of a gene in a subject who is undergoing a therapy for a neurodegenerative condition, wherein the gene is downstream of Nurr1 in a biological pathway in the subject, wherein the therapy is Nurr1 agonism; and b) based on the level of activity of the gene in the subject, determining whether to continue the therapy for the neurodegenerative condition.

In some embodiments, the disclosure provides a method comprising: a) determining that a subject exhibits downregulated Nurr1; b) determining that the subject exhibits upregulated miR-132; and c) based on the determining that the subject exhibits downregulated Nurr1 and the determining that the subject exhibits upregulated miR-132, identifying the subject as being at risk for a condition.

In some embodiments, the disclosure provides a method of treating a neurogenerative condition and/or a method of neuroprotection wherein the method comprises: a) determining a level or activity of Nurr1 in an ex vivo biological sample of a subject with an assay selected from (a) a real-time PCR assay of Nurr1 gene expression against relevant housekeeping genes/internal controls (e.g. GAPDH), (b) an immunoassay (such as ELISA) with the suitable antibodies for Nurr1 protein, and (c) a Western blot for Nurr1 protein from the biological sample selected from peripheral blood, peripheral blood lymphocytes, serum, plasma, CSF and, peripheral blood mononuclear cells; and b) if the level or activity of Nurr1 is no greater than about 90% of the level in a healthy subject of same age, gender and/or BMI, administering to the patient a therapeutically-effective amount of a compound herein.

### I. Neurodegenerative Diseases and Biomarkers

One aspect of the present disclosure relates to methods of treating or ameliorating a disease, e.g., a neurological disease such as a neurodegenerative disease, in a subject, comprising administering to the subject a compound according to Formula (I) to (V), or with a compound according to Formula (VI). In some embodiments, the compound is vidofludimus. In some embodiments, vidofludimus may act as a Nurr1 agonist.

In some embodiments, the neurological disease is Alexander's disease, Alper's disease, Alzheimer's disease, amyotrophic lateral sclerosis (ALS), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, corticobasal syndrome (CBS), Creutzfeldt-Jakob disease, Down syndrome (DS), frontotemporal dementia, Gerstmann-Straussler-Scheinker syndrome, Huntington's disease, HIV-associated dementia (FTD), Kennedy's disease, Krabbe's disease, kuru, dementia with Lewy bodies (DLB), Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, progressive supranuclear palsy (PSP), Refsum's disease, Sandhoffs disease, Schilder's disease, subacute combined degeneration of spinal cord secondary to pernicious anemia, schizophrenia, spinocerebellar ataxia (multiple types with varying characteristics), spinal muscular atrophy, Steele-Richardson-Olszewski disease, or tabes dorsalis.

In some embodiments, the disease is Parkinson's disease, Alzheimer's disease, multiple sclerosis, amyotrophic lateral sclerosis, schizophrenia, or drug addiction. In some embodiments, the disease is Parkinson's disease. In some embodiments, the disease is Alzheimer's disease.

In some embodiments, the disease is multiple sclerosis (MS). In some embodiments, the MS is a relapsing form of multiple sclerosis (RMS), such as relapsing-remitting multiple sclerosis (RRMS) or active secondary progressive multiple sclerosis (active SPMS). In some embodiments, the MS is a progressive form of multiple sclerosis, such as primary progressive multiple sclerosis (PPMS) or non-active secondary progressive multiple sclerosis (non-active SPMS).

Neurodegenerative diseases may be characterized by a change in a measurement of a biomarker in a subject that has, is suspected of having, or is at risk for developing a neurodgenerative disease, as compared to a reference measurement. In some embodiments, the reference measurement is a measurement of the biomarker obtained from a healthy control subject. In some embodiments, the measurement of the biomarker is an increase or a decrease in expression and/or activity of the biomarker, and/or the rate of change of the expression and/or activity of the biomarker. For example, low levels of Nurr1 in the central nervous system of a subject when compared to a reference may indicate neurodegenerative disease in the subject.

In some embodiments, biomarkers associated with neurodegenerative diseases include but are not limited to Nurr1, GFAP, miR132, neurofilament light chain (NFL), BDNF, GDNF, C-RET, YKL-40 (CHI3L1), DAT, pituitary homeobox 3 (Pitx3), tyrosine hydroxylase (TH), vesciular monoamine transporter 2 (VMAT2), superoxide dismutase (SOD), and aromatic amino acid decarboxylase (AADC). In some embodiments, a change in a measurement of one or more of these biomarkers indicates neurodegenerative disease.

Nurr1 or NR4A2 is the protein that in humans is encoded by the NR4A2 gene. Nurr1 is a nuclear receptor and plays a key role in the maintenance of the dopaminergic system of the brain. The term “Nurr1” may refer to the nucleotide sequence or protein sequence of human NR4A2 (e.g., Entrez 4929, Uniprot P43354, RefSeq NM_006186.3, or RefSeq NP_006177.1).

Nurr1 patients, for example, often exhibit lower Nurr1 expression or have lower levels of Nurr1 protein than healthy controls have. Expression can be determined by measuring Nurr1 RNA levels or Nurr1 protein level in blood derived cells. The activity of Nurr1 can also be determined by measuring the amount of target genes or the related protein levels which are regulated in brain cells, blood derived cells, CSF derived cells, plasma, serum or CSF (see e.g. Front. Immunol. 2021; 12:676644 or Sci. Rep. 2020; 10:10755). The activity of Nurr1 can also be determined by measuring a corresponding cellular phenotype. For example, one can measure the regulation of apoptosis/survival in neurons (this can for example be determined by measurement of NFL in serum or plasma or CSF of patients). Nurr1 activation induces survival factors for dopaminergic neurons. Loss of this survival signal leads to an increased number of dying neurons. It is known from literature, that NFL is a marker for axonal damage of dying neurons. Therefore, Nurr1 activation by compound according to Formula (I) to (V), or with a compound according to Formula (VI), may reduce the levels of NFL in serum, plasma, or CSF.

Glial fibrillary acidic protein (GFAP) is a type III intermediate filament (IF) protein and is a marker for brain injury. GFAP is expressed in cells of the central nervous system including astrocytes. Levels of GFAP in patients' serum correlate with neurodegeneration aspects mediated by astrocytes. Astrocytes that become activated increase their production of GFAP. GFAP can be degraded into GFAP-BDP. Both products can be detected in the CSF, serum, or plasma due to astrocyte injury (see e.g. Trends Neurosci. 2015; 38:364). In some embodiments, Nurr1 activity reduces astrogliosis and thereby reduce astrocyte injury which results in reduced GFAP/BDP levels, when activated by a compound according to Formula (I) to (V), or with a compound according to Formula (VI).

The relative expression of the Nurr1 protein or target gene in healthy controls and patients has been reviewed in Int. J. Mol. Sci. 2019; 20:4858. The relative expression of Nurr1 in patients or healthy controls can be obtained from peripheral blood via peripheral blood lymphocytes separation, total RNA extraction and then real-time PCR assay of Nurr1 gene expression against internal control GAPDH (see e.g., J. Neurol. Sci. 2008; 273:29).

The term “NR4A3” refers to the nuclear receptor 4A3 (nuclear receptor subfamily 4, group A, member 3; NR4A3) also known as neuron-derived orphan receptor 1 (NOR1), which is a protein that in humans is encoded by the NR4A3 gene. NR4A3 is a member of the nuclear receptor family of intracellular transcription factors.

miR-132 is a short, non-coding RNA molecule. This microRNA regulates the expression levels of other genes by several mechanisms, generally reducing protein levels through the cleavage of mRNAs or the repression of their translation. miR-132 expression in serum, plasma or CSF can be used to determine Nurr1 brain expression levels and is in some embodiments a marker that can be used to distinguish patients who might benefit more from treatment with a compound according to Formula (I) to (V), or with a compound according to Formula (VI).

Neurofilament light chain (NFL) is a marker for axonal damage and neuron destruction in general and specifically in MS. Low levels of NFL correlate with protection/survival of neurons. In PD, where Nurr1 shows lower expression levels compared to healthy volunteers and low levels of Nurr1 are significantly associated with dopaminergic neuron loss, NFL is a potential biomarker for motor decline. The cardinal motor symptoms of PD are caused by the death of dopaminergic neurons in the substantia nigra pars compacta. Nurr1 activation leads to survival signals within neurons and therefore lower levels of NFL might be an outcome of Nurr1 activation (e.g., caused by compound according to Formula (I) to (V), or with a compound according to Formula (VI)) and beneficial for patients with neurodegenerative diseases.

Quantification of NFL in blood and CSF can be assessed by a highly sensitive electrochemiluminescence (ECL) based immunoassay (e.g. PLOS One 2013; 8: e75091). For blood samples, in which NFL levels are lower compared to CSF, the single-molecule or “Simoa” assay (Quanterix Corp.) is currently the most used method, because it is very sensitive with a lower limit of quantitation of 0.1 μg/mL (eBioMedicine 2024; 101:104970). Quanterix received FDA breakthrough device designation for the method in 2022 to test for NFL in MS patients.

In some embodiments, NFL level in blood and CSF is measured by an electrochemiluminescence (ECL) based immunoassay. In some embodiments, NFL level in blood is measured by a single-molecule or “Simoa” assay (Quanterix Corp.).

The same applies for the Roche Elecsys platform, which might become also one of the standards to be used.

In some embodiments, patients with a neurological disease have higher NFL levels in serum (sNFL), e.g., AD (30.8 μg/mL), GBS (79.4 μg/mL) or ALS (95.4 μg/mL) than do neurological patients without evidence of structural central nervous system (CNS) damage and healthy controls. Similar differences were seen in corresponding CSF samples (PLOS One 2013; 8: e75091). The NFL levels increase during normal aging, e.g., from mean sNFL of 20.4 μg/mL at age<50 years to mean sNFL of 45.9 μg/mL at age>70 years (Nat. Commun. 2020; 11:812), indicating an acceleration of neuronal injury at higher age, which may be driven by subclinical comorbid pathologies. This must be considered when defining the level of NFL for the healthy control or the success of treatment with a compound according to Formula (I) to (V), or with a compound according to Formula (VI). An overview of neurofilaments as biomarkers in neurological disorders is given in Nat. Rev. Neurol. 2018; 14:577. In addition to NFL, Yuang and Nixon describe other neurofilament proteins, which can be quantified and monitored during the treatment of a neurological disease (Front. Neurosci. 2021; 15:689938).

Brain-derived neurotrophic factor (BDNF) is a protein of which expression levels are controlled by Nurr1. Nurr1 activation leads to enhanced BDNF expression. Measurement of the mature form of the BDNF protein in plasma, serum or CSF can be used as a marker for Nurr1 activity and to assess the treatment effect with a compound according to Formula (I) to (V), or with a compound according to Formula (VI). Increased mature BDNF level correlate to higher neuron survival. Various BDNF isoform protein levels in CSF, blood and plasma samples can be determined with commercially available immunoassay kits (see e.g., Int. J. Neuropsychopharmacol. 2011; 14:347 or Nat. Rev. Neurosci. 2005; 6:603).

Glial cell line-derived neurotrophic factor (GDNF) is also a target gene of Nurr1. Nurr1 activation leads to enhanced GDNF expression. Measurement of GDNF expression in plasma, serum or CSF can be used as a marker for Nurr1 activity and to assess the treatment effect with a compound according to Formula (I) to (V), or with a compound according to Formula (VI). Increased GDNF levels correlate to higher neuron survival.

C-RET is the RET proto-oncogene and encodes a receptor tyrosine kinase for members of the GDNF family of extracellular signalling molecules and promotes survival of the dopaminergic neuron. Nurr1 activity induces C-RET expression in dopamine neurons. C-RET expression can be measured on RNA or protein level from brain biopsies taken from patients with neurodegenerative diseases. In some embodiments, lower expression C-RET in the brain of patients with neurodegenerative diseases compared to controls correlates to a greater benefit for patients treated with a compound according to Formula (I) to (V), or with a compound according to Formula (VI).

Glial fibrillary acidic protein (GFAP) is a type III intermediate filament (IF) protein and is a marker for brain injury. GFAP is expressed in cells of the central nervous system including astrocytes. Levels of GFAP in patients' serum correlate with neurodegeneration aspects mediated by astrocytes. Astrocytes that become activated increase their production of GFAP. GFAP can be degraded into GFAP-BDP. Both products can be detected in the CSF, serum, or plasma due to astrocyte injury (see e.g. Trends Neurosci. 2015; 38:364). In some embodiments, Nurr1 activity reduces astrogliosis and thereby reduce astrocyte injury which results in reduced GFAP/BDP levels, when activated by a compound according to Formula (I) to (V), or with a compound according to Formula (VI).

YKL-40 (CHI3L1) is a glycoprotein predominantly produced by reactive astrocytes and microglia in chronic active MS lesions (J. Neuroimmunol. 2016; 292:52). YKL-40 levels are increased in the serum of RRMS patients compared to controls and is therefore proposed to be a useful marker for the inflammatory process of MS (Arq. Neuropsiquiatr. 2021; 79:795). Within the CNS, CHI3L1 is linked to neuroinflammatory processes and reactive gliosis (Neurol. Neuroimmunol. Neuroinflamm. 2022; 9: e1164), which can be reduced by Nurr1 activity (see above).

Dopamine active transporter (DAT; also SLC6A3) is a membrane-spanning protein responsible for the reuptake of dopamine from the synapse back into the cytosol of the dopaminergic neurons. DAT is a target gene of Nurr1 (Development 2009; 136:2363). DAT may be reduced by 50-70% in PD patients. DAT imaging with single-photon emission computed tomography (SPECT) can be used to confirm or exclude a diagnosis of dopamine deficient parkinsonism. Patients with low levels of Nurr1 expression and activity have lower expression levels and activity of dopamine transporter and therefore benefit from Nurr1 activation by a compound according to Formula (I) to (V), or with a compound according to Formula (VI).

Pituitary homeobox 3 (Pitx3) is the gene that encodes a member of the RIEG/PITX homeobox family, which is in the bicoid class of homeodomain proteins and act as transcription factors. Pitx3 is involved in the maintenance of dopaminergic neurons. The term “Pitx3” may refer to the nucleotide sequence or protein sequence of human Pitx3.

Tyrosine hydroxylase (TH) is an enzyme responsible for catalyzing the conversion of the amino acid L-tyrosine to L-3,4-dihydroxyphenylalanine (L-DOPA). In humans, tyrosine hydroxylase is encoded by the TH gene. In some embodiments, Nurr1 activation in dopaminergic neurons leads to upregulation of tyrosine hydroxylase expression. Tyrosine hydroxylase activity converts tyrosine to L-DOPA. L-DOPA converts into dopamine, which is a neurotransmitter that provides signals for executive functions, motor control, motivation, arousal, reinforcement and reward. Lower-level functions are lactation, sexual gratification, and nausea.

Vesicular monoamine transporter 2 (VMAT2; also solute carrier family 18 member 2 or SLC18A2) is an integral membrane protein that transports neurotransmitters such as dopamine, norepinephrine, serotonin and histamine from cellular cytosol into synaptic vesicles. VMAT2 is a direct target gene of Nurr1 and is upregulated by Nurr1 activation. VMAT2 expression can be determined on RNA and protein level in neurons from brain biopsies. In addition, VMAT2 activity can be determined by DTBZ PET. [¹¹C]dihydrotetrabenazine (DTBZ) can be used as a radioligand for VMAT2 and can be visualized by PET imaging. VMAT2 is involved in packaging of dopamine. In nigrostriatal pathway and mesolimbic pathway dopamine-releasing neurons, SLC18A2 function is also necessary for the vesicular release of the neurotransmitter GABA. In some embodiments, Nurr1 activation by a compound according to Formula (I) to (V), or with a compound according to Formula (VI), induces higher VMAT2 expression and therefore is beneficial for patients lacking proper VMAT2 activity due to mutations, and low Nurr1 activation status or cocaine abuse. Increasing VMAT2 expression by two single-nucleotide polymorphisms (SNPs) in the promotor region reduces PD risk. In some embodiments, induction of higher expression and activity levels of VMAT2 is beneficial in neurogenerative diseases.

Superoxide dismutase (SOD) is an enzyme that alternately catalyzes the dismutation (or partitioning) of the superoxide (O₂⁻) radical into ordinary molecular oxygen and hydrogen peroxide. SOD is upregulated by Nurr1 activation. SOD is an enzyme reducing oxidative stress in neurons and therefore reduces apoptotic signals in neurons. SOD expression can be measured on RNA or protein level from brain biopsies, CSF, or blood samples. Patients exhibit lower levels of SOD compared to control can benefit from treatment with a compound according to Formula (I) to (V), or with a compound according to Formula (VI), due to enhanced Nurr1 activation.

Aromatic amino acid decarboxylase (AADC; also known as DOPA decarboxylase (DDC)), is a lyase enzyme. AADC is a direct target gene of Nurr1 and is upregulated by Nurr1 activation. AADC mediates the final step in the synthesis for the neurotransmitters dopamine and serotonin. Lower levels of AADC or dysfunction of AADC in neurons lead to accumulation of intracellular L-DOPA, which is then chemically transformed into 5-hydroxytryptophan and 3-O-methyldopa. 5-hydroxytryptophan (5-HTP) and 3-O-methyldopa (3-OMD) can be determined in blood by using UPLC-MS/MS (described e.g. J. Chromatogr. B Biomed. Appl. 2021; 1185:122999). In some embodiments, patients with lower levels or lower functionality of AADC determined by UPLC-MS/MS compared to controls can benefit from a treatment with a compound according to Formula (I) to (V), or with a compound according to Formula (VI), by activation of Nurr1 and subsequent upregulation of AADC.

### II. Multiple Sclerosis

A further aspect of the present disclosure relates to methods of treating or ameliorating multiple sclerosis (MS) in a subject who has MS, comprising administering to the subject a compound according to Formula (I) to (V), or with a compound according to Formula (VI), for example, vidofludimus.

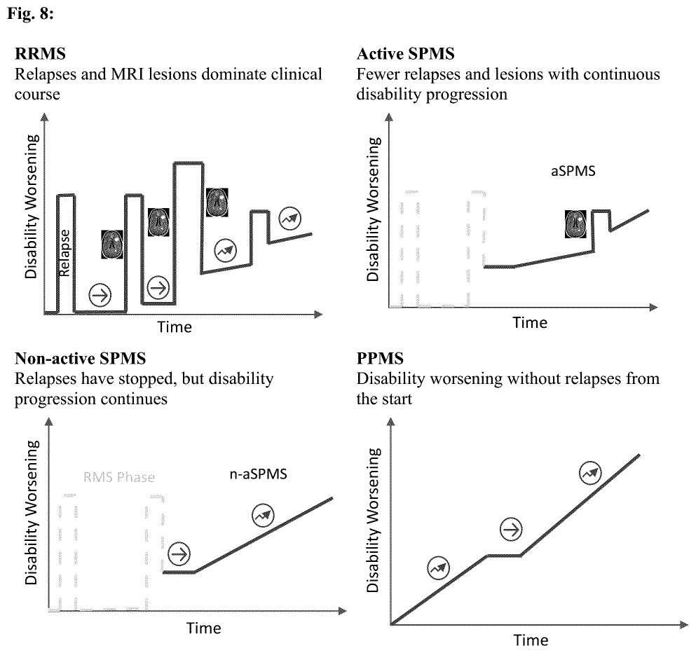

Multiple sclerosis (MS) can be divided to relapsing forms of MS (RMS) and progressive forms of MS (PMS). Relapsing-remitting MS (RRMS) and active secondary progressive MS (active SPMS) are primarily driven by focal inflammatory disease and characterized by the presence of magnetic resonance imaging (MRI) lesions and relapses. It is however known from a recent meta-analysis of a large patient database that disability worsening in these two subtypes are driven by relapse-associated worsening (RAW) and by progression independent of relapse activity (PIRA). RRMS is characterized by the domination of relapses and MRI lesions over the clinical course. Active SPMS (aSPMS) is characterized by fewer relapses and lesions with continuous disability progression (see FIG. 8).

Primary progressive MS (PPMS) and non-active secondary progressive MS (non-active SPMS or n-aSPMS) are the two predominantly progressive forms of MS, which are characterized by an ongoing disability worsening without or almost without any MRI lesions or relapses being present. It is known from a recent meta-analysis of a large patient database that disability worsening in these two subtypes are almost exclusively driven by PIRA. Non-active SPMS is characterized by continuous disability progression while relapses have stopped. PPMS is characterized by disability worsening from the start (see FIG. 8).

Definitions and subcategorizations of PPMS and SPMS may vary. In one exemplary definition, all SPMS may be considered to belong to the category of PMS, so that all SPMS patients considered to be PMS patients, with no differentiation between aSPMS and n-aSPMS. In another exemplary definition, active-SPMS may be considered to belong to the category of RMS, while non-active-SPMS may be considered to belong to the category of PMS. Thus, the terms “n-aSPMS” and “SPMS” are used synonymously in the present invention.

The separation of PIRA compared to RAW can be as follows (JAMA Neurol. 2023; 80:151): A PIRA event can be defined as experiencing confirmed disability worsening (CDW) in the EDSS scale at 6 months during a period free of relapses (PFRs). A PFR is the time between two consecutive relapses, starting 3 months after a relapse (or 6 months after the first demyelinating event). The first EDSS score obtained at least 6 months after the first attack or 3 months after any other attack was referred to as the baseline EDSS score and rebaseline EDSS score, respectively. It was set that no rebaseline EDSS score could be lower than the first recorded (baseline) EDSS score. Confirmed disability accumulation (CDA) was defined as an increase in the EDSS score of 1.5, 1.0, or 0.5 if the baseline/rebaseline EDSS score was, respectively, 0, 1.0 to 5.0, or greater than 5.0. The date of PIRA was the date of the confirmation of the CDA. Any other episodes of CDA that did not qualify for PIRA (i.e., which occurred outside the PFR) were considered to be RAW events. Those patients with at least 1 CDA but who did not present with any PIRA event were considered patients with RAW.

In the clinical study of Kopp et al. (Mult. Scler. Relat. Disord. 2021; 56:103319) the following inclusion criteria were applied on a MS population with a diagnosis of clinical SPMS assigned by an MS-neurologist and RRMS patients fulfilling the MSBase diagnostic definition for conversion to SPMS. This m-EXPAND criteria identify patients with recent worsening on the EDSS score likely not explained by a recent relapse:
(a) An EDSS from 3.0 to 6.5 (both inclusive) (at index date+/−6 months); and
(b) EDSS progression within the last 2 years before data extraction, defined as EDSS progression of 1 point or more in patients with an EDSS score of less than 6.0 or ≥0.5 point in patients with EDSS score≥6.0, in the absence of relapses 6 months prior to progression and EDSS≥3.0 at time of progression; and
(c) Disability progression.

Currently, there is no FDA-approved treatment option for SPMS other than mitoxantrone. However, for treatment with mitoxantrone, no distinction regarding relapse activity (active SPMS vs non-active SPMS) is made. Notably, treatment of mitoxantrone is associated with significant toxicities including cardiotoxicity and secondary malignancy, which limits the length of treatment to −3 years (lifetime dose of 140 mg/m²). In addition, patient populations in this trial were predominately with relapsing type of SPMS, and the benefit in non-active SPMS is unclear. Siponimod, natalizumab, and interferon beta-1b are approved for active SPMS but have not shown benefit in non-active SPMS. For PPMS patients, ocrelizumab has been approved, however due to the strong attenuation of the immune response it contains a black box warning towards progressive multifocal leukoencephalopathy and immune-mediated colitis. Other treatment options currently being tested in clinical trials (e.g. fenebrutinib with risk of liver damages) also do not offer the favorable safety profile of vidofludimus. Therefore, there is an urgent unmet need given the lack of effective therapies for PIRA in its entirety.

MS usually begins with a clinically isolated syndrome (CIS). This is the first episode of symptoms caused by inflammation and damage to the myelin covering on nerves in the brain or spinal cord. In CIS, a person has an attack suggestive of demyelination, but does not fulfill the criteria for MS. 30 to 70% of persons experiencing CIS later develop MS.

There exists an additional subgroup of MS patients, i.e. patients with MS transitioning between relapsing MS and PIRA. For this disease the term “transitioning MS” is used herein. Transitioning MS patients can be identified by one or more of the following test methods:
(a) The symbol digit modalities test (SDMT),
(b) Multiple Sclerosis Functional Composite (MSFC),
(c) EDSS,
(d) timed 25-foot walk (T25FW),
(e) 9-hole peg test (9HPT)
(f) The MSProDiscuss™ clinical tool
(g) Composite scores integrating several tests like e.g. EDSS, timed 25-foot walk (T25FW), SDMT and 9-hole peg test (9HPT), MSProDiscuss™ clinical tool.

### III. Progression Independent of Relapse (PIRA)

Another aspect of the present disclosure relates to methods of treating progression of MS, e.g., progression independent of relapse (PIRA) in a subject, comprising administering to the subject a compound according to Formula (I) to (V), or with a compound according to Formula (VI). In some embodiments, the compound is vidofludimus.

PIRA is a contributing factor in all forms of multiple sclerosis (MS). PIRA plays a significant role in disease worsening in RRMS and is the principal way by which patients acquire disability in progressive multiple sclerosis (PMS), e.g., forms of PMS in which there are few or no relapses. PIRA can occur even early in the disease, as shown in a study in patients with successful suppression of inflammation with efficacious DMTs, providing evidence for an ongoing treatment-resistant pathology from the start.

This elevates the importance of any new drug that reduces or is slowing down PIRA (measured as confirmed disability worsening but also by assessment of brain atrophy). It can also be of importance to provide a new drug that: (a) reduces relapse activity AND (b) reduces or is slowing down PIRA (measured as confirmed disability worsening but also by assessment of brain atrophy).

Thus, the present invention aims for treating the relapse-independent accumulation of neurological deficits worsening in one embodiment.

Thus, the present invention aims for treating progression independent of relapse activity (PIRA).

Relapse-independent accumulation of neurological deficits worsening or progression independent of relapse activity has been defined by https://doi.org/10.1093/brain/awac016 (Brain 2022; 145:3147) by Fred Lublin wherein detection of neurological deficits worsening or progression independent of relapse activity is detected as described in https://doi.org/10.1093/brain/awac016 (Brain 2022; 145:3147) by Fred Lublin and there are included herein by reference as one embodiment.

There are two main mechanisms by which patients with multiple sclerosis acquire disability: (i) step-wise accrual of impairment due to incomplete recovery from a relapse [i.e. relapse-associated worsening (RAW)]; and (ii) progression independent of relapse activity (PIRA). While the former is considered to be the main source of permanent disability in relapsing multiple sclerosis, the latter is thought to drive the insidious progression typical in primary and secondary progressive multiple sclerosis (PPMS and SPMS). One example of detection of progression independent of relapse activity is detected and measured according to Kurtzke, J F “Rating neurologic impairment in multiple sclerosis: An expanded disability status scale (EDSS)”, Neurology. 1983; 33:1444-1452.

No satisfactory medication is currently available to treat the major smoldering portion of disability worsening, which is independent of relapses. Surprisingly, the present inventions show that a compound according to Formula (I) to (V), or with a compound according to Formula (VI), in particular vidofludimus, which acts as a dual DHODH inhibitor and Nurr1 agonist, can function as a neuroprotective drug in addition to their established activity as anti-inflammatory drug.

In particular, patients with low levels of Nurr1 will benefit from a treatment with a compound according to Formula (I) to (V), or with a compound according to Formula (VI). Thus, for the first time there is evidence that patients with a reduced Nurr1 activity benefit more from a treatment with vidofludimus calcium (IMU-838) than patients with a non-reduced range of Nurr1 activity: the DHODH inhibition is good against inflammation and Nurr1 activation is new and is directed to protecting neurons from death. Diseases and conditions exhibiting lower Nurr1 expression or activity share reduced survival or functioning of dopaminergic neurons, resulting in neurodegeneration.

Patients with diseases comprising brain and spinal cord disorders or neurodegeneration can be beneficially treated with a compound according to Formula (I) to (V), or with a compound according to Formula (VI). These conditions comprise diseases with symptoms like motor deficits, cognitive deficits, fatigue, depression, or drug addiction.

When focused on the disease MS, the positive effects for patients with RRMS can be attributed to the anti-inflammatory properties of the compounds of Formula (I) to (V), or with a compound according to Formula (VI), preferably vidofludimus, or an isotopic variant, a pharmaceutically acceptable salt or solvate thereof.

In some embodiments, MS patients can be classified in two groups: (a) the one with relapses, i.e. relapse-associated worsening (RAW) patients and (b) the others with progredient (smoldering) disease worsening, i.e. patients with progression independent of relapse activity (PIRA), which need fundamentally different treatments. In some embodiments, Group (a) needs an anti-inflammatory treatment, while group (b) needs a neuroprotective treatment to attenuate the disease worsening. For group (b) nearly no treatment options exist, particularly since a pure DHODH inhibitor would not be expected to lead to beneficial treatment. In one embodiment, a compound according to the present invention may act as a nuclear receptor related 1 (Nurr1).

Thus, vidofludimus has so far not been tested in a MS patient group, which has been without any relapses or which had a free-relapse period of at least 12 or 24 months. Surprisingly we found that the compounds according to Formula (I) to (V), or with a compound according to Formula (VI), preferably vidofludimus, or an isotopic variant, a pharmaceutically acceptable salt or solvate thereof, has not only beneficial effects for group (a) but also beneficial effects for group (b), which seem even superior to the other currently tested treatment options for this group. This can be attributed to the neuroprotective Nurr1 agonism, which is caused by compounds of Formula (I) to (V), or with a compound according to Formula (VI), preferably vidofludimus. This new finding opens the use of compounds of Formula (I) to (V), or with a compound according to Formula (VI), preferably vidofludimus, for all diseases with an abberant level of Nurr1.

In one embodiment the patient treated according to the present invention is a patient with MS that has been without any relapses or that has a free-relapse period of at least 12 months. In another embodiment the patient treated according to the present invention is a patient with MS that has been without any relapses or that has a free-relapse period of at least 24 months.

In one embodiment the patient treated according to the present invention is a patient with PIRA that has been without any relapses or that has a free-relapse period of at least 12 or 24 months.

In one embodiment of the invention PIRA was defined as a 3- or 6 or preferably, 12 or 24-month confirmed disability worsening (CDW) event with either no prior relapse or an onset more than 90 days after the start date of the last investigator-reported relapse (irrespective of the EDSS confirmation).

In addition, to qualify as a PIRA event, no relapse must occur within 30 days before or after the EDSS confirmation. If a relapse with incomplete recovery occurred, the baseline (i.e. the reference EDSS value) maybe reset>90 days after the relapse onset to identify the next PIRA event. In an individual patient, the baseline could be reset multiple times (i.e. after each relapse) until either a PIRA event was discovered, or until the individual EDSS profile ended.

Sustained PIRA may be a 3- or 6-month PIRA event in which the EDSS-worsening was sustained in all following assessments, i.e. the patient never recovered in the available longitudinal data.

Clinical evidence for disability may come from surrogate endpoints, e.g.,
annualized rate of percent brain volume change up to 120 weeks
annualized rate of change in whole brain atrophy.

Clinical evidence may also come from biomarker response by
slowing down or reducing increase of serum NfL levels by at least 5% (or defining Hazard ration of 0.9)
reducing the level of GFAP in CSF, serum, or plasma by at least 3% for a period of 48 weeks.

Disability progression may evaluated using the Expanded Disability Status Scale (EDSS), the 9-Hole Peg Test (9-HPT), or the Timed 25-Foot Walk Test (T25FWT), or any combinations thereof.

Methods for evaluating disability progression may comprise evaluating the onset of composite 12-week confirmed disability progression (cCDP12), wherein onset of the cCDP12 comprises at least one progression event selected from the group consisting of:
a. an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
b. increase from baseline of at least 20% in time to complete the 9-HPT; and
c. increase from baseline of at least 20% in T25FWT, and wherein the progression event is confirmed at least 12 weeks after the initial progression.

Observational and controlled clinical trials provide evidence that PIRA is likely the most frequent manifestation of disability accumulation across the full spectrum of traditional MS phenotypes, including CIS and early RRMS, thus providing another perspective of the conceptual distinction between relapsing and progressive disease courses or stages. In some embodiments, as an additional set of determinants, the following determinants may be used for diagnosing PIRA both in RRMS and progressive MS:

A. Baseline/reference score: A roving baseline may be applied, that sets a new reference score every time the EDSS or individual measure of the composite is lower than the previous measure and confirmed at the following visit. The reference score may also be reset if a relapse causes residual disability.

B. Event score: An increase of EDSS or composite measure may be considered for classification to PIRA, if it is not determined within 30 days before and 90 days after the onset of an investigator reported relapse. Besides EDSS (increase of 1.5 points if baseline is 0, 1.0 point between 1 and 5.5, 0.5 points>5.5), a composite measure may be recommended and should include upper limb function (9HPT, threshold: >20% decline), walking speed (T25FWT, threshold: >20% decline) and cognitive testing (SDMT, threshold: 4 points or >10% decline).

C. Confirmation score: The confirmation visit may take place no earlier than 3 months, preferably 6, or 12 months after the initial disability increase and should not happen 30 days before and 90 days after the onset of an investigator-reported relapse.

D. Sustained score: The EDSS score defining PIRA may not improve until the end of follow up, sensibly at least 12 or even 24 months apart from start of PIRA.

In one embodiment the patient treated according to the present invention is a patient with PIRA that has been without any relapses or that has a free-relapse period of at least 12 or 24 months and the medicament is vidofludimus in a daily dose of 30 mg to 45 mg daily doses of vidofludimus calcium.

In one embodiment the patient treated according to the present invention is a patient with PIRA that has been without any relapses or that has a free-relapse period of at least 12 or months and the medicament is vidofludimus in a daily dose of 30 mg daily doses of vidofludimus calcium.

In one embodiment the patient treated according to the present invention is a patient with PIRA that has been without any relapses or that has a free-relapse period of at least 12 or 24 months and the medicament is vidofludimus in a daily dose of 45 mg daily doses of vidofludimus calcium.

It has been discovered that patients with MS acquire disability not only by relapse-associated worsening (RAW) but to a large extend by progression independent of relapse activity (PIRA).

PIRA maybe quantified with help of confirmed disability worsening (CDW) events. PIRA may start early in the disease process, may occur in all phenotypes, and may become the principal driver of disability accumulation in the progressive phase of the disease.

PIRA is the principal driver of disability accumulation during the progressive phase of the disease. PIRA may be associated with worsening unknown “hidden symptoms” such as fatigue, sphincter, and cognitive symptoms. Patients may have MS transitioning between relapsing and progressive disease. There is an underlying progressive course in all MS patients independently of the disease classification. In one embodiment PIRA patients maybe SPMS patients without a recent relapse and no MRI activity suggestive of active inflammation, and with evidence of recent progression independent of relapses.

PIRA and RAW can be subdivided in smaller groups like “early PIRA”, “late PIRA”, “active PIRA” or “nonactive PIRA” (JAMA Neurol. 2023; 80:151).

Patients with early PIRA show significantly steeper Expanded Disability Status Scale (EDSS) increase rates than those with late PIRA.

Furthermore, patients with early PIRA had a greater risk of reaching EDSS 6.0 at faster rates than those with late PIRA.

PIRA may be a nonreversible phenomenon associated with unfavorable long-term disability outcomes, especially if such PIRA events occur early in the disease course.

Identifying all who will develop PIRA as soon as possible after the first demyelinating event, especially early PIRA, may lead to better treatment choices, and subsequently, better long-term outcomes.

Provided herein are methods of treating PIRA in a subject in need thereof, by administering to the subject a therapeutically effective amount of vidofludimus, or an isotopic variant, pharmaceutically acceptable salt or solvate thereof, preferably vidofludimus calcium salt dihydrate as “Polymorph A”. Further provided are methods of treating PIRA in a subject in need thereof, by administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Further provided is a compound for use in a method of treating PIRA in a subject in need thereof, wherein the compound is vidofludimus of a once daily dose in the maintenance phase of about 30 to 45 mg, or an equivalent amount of an isotopic variant, pharmaceutically acceptable salt or solvate thereof, preferably vidofludimus calcium salt dihydrate as “Polymorph A”. In further embodiments, provided herein is a compound tor use in the manufacture of a medicament for the treatment of PIRA in a subject in need thereof, wherein the compound is vidofludimus of a once daily dose in the maintenance phase of about 30 to 45 mg, or an equivalent amount of an isotopic variant, pharmaceutically acceptable salt or solvate thereof, preferably vidofludimus calcium salt dihydrate as “Polymorph A”. In some embodiments, the treatment of PIRA is evaluated using the Expanded Disability Status Scale (EDSS), the 9-Hole Peg Test (9-HPT), or the Timed 25-Foot Walk Test (T25FWT), or any combinations thereof. In some embodiments, the treatment of PIRA is evaluated based the time to onset of confirmed disability progression (e.g., 12-week or 24-week CDP), or based on the time to onset of a composite confirmed disability progression (e.g., 12-week or 24-week cCDP). For example, in some embodiments, treating a subject with PIRA by administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”, results in a delay in worsening of the EDSS (e.g., increase of 0.5, 1.0, 1.5, or more points compared to baseline), a delay in the worsening of the 9-HPT time (e.g., by over 20% compared to baseline), a delay in the worsening of the T25FWT time (e.g., by over 20% compared to baseline), delaying to onset of CDP12, delaying to onset of CDP24, delaying to the onset of cCDP12, delaying the onset of cCDP24, delaying the onset of at least one progression event, reducing the risk of having at least one progression event, or decreasing disability in a subject with PIRA. In other embodiments, the treatment of PIRA is evaluated based on MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level. In other embodiments, the treatment of PIRA is evaluated based on BDNF, GDNF, C-RET, GFAP, DAT, Pitx3, TH, VMAT2, SOD1, AADC, TNFα, iNOS, YKL-40 or IL-1β. For example, in some embodiments, treating a subject with PIRA comprises delaying the progression of PIRA, wherein the progression is evaluated based on MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level; or the onset of at least one progression event, which may be described by CDP12, cCDP12, CDP24, or cCDP24. In some embodiments, treating PIRA comprises delaying progression of PIRA. In certain embodiments, treating PIRA comprises delaying the onset of at least one progression event in the subject. In some embodiments, treating PIRA comprises reducing the risk of the subject experiencing at least one progression event. In certain embodiments, treating PIRA comprises delaying progression, or delaying the onset of at least one progression event, by at least 5%, at least 10%, at least 15%, at least 20%, at least 25%, at least 30%, or at least 35% (e.g., as evaluated using T25FWT time, or 9-HPT time, or EDSS score, or CDP12, or cCDP12, or CDP24, or cCDP24 etc.). In certain embodiments, treating PIRA comprises delaying progression, or delaying the onset of at least one progression event, by at least 5%, at least 10%, at least 15%, at least 20%, at least 25%, at least 30%, or at least 35% as compared to another subject with PIRA (e.g., a comparative subject), wherein the other subject is not administered vidofludimus, or an isotopic variant, pharmaceutically acceptable salt or solvate thereof. In some embodiments, the delay is at least 5%. In some embodiments, the delay is at least 10%. In some embodiments, the delay is at least 15%. In some embodiments, the delay is at least 20%. In some embodiments, the delay is at least 25%. In some embodiments, the delay is at least 30%. In some embodiments, the delay is at least 35% In some embodiments, the other subject is administered an anti-CD20 antibody (such as a CD20-directed cytolytic antibody). In still further embodiments, treating PIRA comprises reducing the risk the subject has at least one progression event by at least 5%, at least 10%, at least 15%, at least 20%, at least 25%, at least 30%, or at least 35%. In certain embodiments, the risk is reduced over a period of time, for example reducing the risk of having at least one progression event over 12 weeks, 18 weeks, 24 weeks, 36 weeks, 48 weeks, 60 weeks, 72 weeks, 84 weeks, 96 weeks, 108 weeks, or 120 weeks. In some embodiments, the risk is reduced as compared to another subject with PIRA (e.g., a comparative subject) who is not administered vidofludimus, or an isotopic variant, pharmaceutically acceptable salt or solvate thereof, and who is optionally administered an anti-CD20 antibody. In some embodiments, the other subject is administered an anti-CD20 antibody (such as a CD20-directed cytolytic antibody). In some embodiments, the risk is reduced by at least 5%. In some embodiments, the risk is reduced by at least 10%. In some embodiments, the risk is reduced by at least 15%. In some embodiments, the risk is reduced by at least 20%. In some embodiments, the risk is reduced by at least 25% In some embodiments, the risk is reduced by at least 25%. In some embodiments, the risk is reduced by at least 30%. In some embodiments, the risk is reduced by at least 35%. In certain embodiments, treating PIRA comprises an improvement of at least 5%, at least 10%, at least 15%, at least 20%, at least 25%, or at least 30% in a metric of PIRA (e.g., in T25FWT time, or 9-HPT time, or EDSS score, etc.), as compared to the same metric evaluated in the same subject prior to beginning administration of vidofludimus, or an equivalent amount of an isotopic variant, pharmaceutically acceptable salt or solvate thereof, preferably vidofludimus calcium salt dihydrate as “Polymorph A”. In some embodiments, the improvement is compared to the same metric evaluated in the same subject within 1 week, or within 0 to 28 days, or within 6 weeks prior to beginning administration of vidofludimus, or an equivalent amount of an isotopic variant, pharmaceutically acceptable salt or solvate thereof. In other embodiments, treating PIRA comprises an improvement of at least 5%, at least 10%, at least 15%, at least 20%, at least 25%, at least 30%, or at least 40% in a metric of PIRA (e.g, in T25FWT time, or 9-HPT time, or EDSS score, etc.), as compared to the same metric evaluated in another subject with PIRA, wherein the other subject is not administered vidofludimus, or an isotopic variant, pharmaceutically acceptable salt or solvate thereof. In some embodiments, the improvement is at least 5%. In some embodiments, the improvement is at least 10%. In some embodiments, the improvement is at least 15%. In some embodiments, the improvement is at least 20%. In some embodiments, the improvement is at least 25%. In some embodiments, the improvement is at least 30%, In some embodiments, the improvement is at least 35%. In some embodiments, the other subject is administered an anti-CD20 antibody (such as a CD20-directed cytolytic antibody). In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

Further provided is a method of treating (e.g., slowing) progression of PIRA in a subject in need thereof, by administering to the subject a therapeutically effective amount of vidofludimus, or an isotopic variant, pharmaceutically acceptable salt or solvate thereof, preferably vidofludimus calcium salt dihydrate as “Polymorph A”. Further provided is administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Thus, provided herein are methods of treating (e.g. slowing) the progression of PIRA in a subject in need thereof, by administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Further provided is a compound for use in a method of slowing the progression of PIRA in a subject in need thereof, wherein the compound is vidofludimus, or an isotopic variant, pharmaceutically acceptable salt or solvate thereof, and wherein the method comprises administering to the subject a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In further embodiments, provided herein is a compound for use in the manufacture of a medicament for use in a method of treating (e.g., slowing) the progression of PIRA in a subject in need thereof, wherein the compound is vidofludimus, or an isotopic variant, pharmaceutically acceptable salt or solvate thereof, and wherein the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In some embodiments, the progression of PIRA is evaluated using the Expanded Disability Status Scale (EDSS), the 9-Hole Peg Test (9-HPT), or the Timed 25-Foot Walk Test (T25FWT), or any combinations thereof. In some embodiments, the progression of PIRA is evaluated based the time to onset of confirmed disability progression (e.g., 12-week or 24-week CDP), or based on the time to onset of a composite confirmed disability progression (e.g., 12-week or 24-week cCDP). In certain embodiments, the progression of PIRA is slowed at least 5%, at least 10%, at least 15%, at least 20%, at least 25%, at least 30%, or at least 35%. In some embodiments, the progression is slowed at least 5%. In some embodiments, the progression is slowed at least 10%. In some embodiments, the progression is slowed at least 15%. In some embodiments, the progression is slowed at least 20%. In some embodiments, the progression is slowed at least 25%. In some embodiments, the progression is slowed at least 30%. In some embodiments, the progression is slowed at least 35%. In some embodiments, progression is slowed as measured by the onset of cCDP12 (e.g., by increasing the time to onset of cCDP12) or by the risk of cCDP12 (e.g., reducing the risk of experiencing cCDP12 during a period of time). In some embodiments, the progression of PIRA is slowed relative to another subject with PIRA (e.g., a comparator subject), wherein the other subject is not administered vidofludimus, or an isotopic variant, pharmaceutically acceptable salt or solvate thereof. In some embodiments, the other subject is administered an anti-CD20 antibody (such as a CD20-directed cytolytic antibody) and is not administered a DHODH inhibitor (such as vidofludimus, or an isotopic variant, pharmaceutically acceptable salt or solvate thereof). In certain embodiments, the total evaluation time period is 12 weeks, 18 weeks, 24 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g, PIRA progression is slowed by at least 5%, at least 10%, at least 15%, at least 20%, at least 25%, at least 30%, or at least 35%, as evaluated over 120 weeks, when compared to another subject with PIRA who is not administered vidofludimus, or an isotopic variant, pharmaceutically acceptable salt or solvate thereof and is optionally administered a CD20-directed cytolytic antibody. In some embodiments, the other subject is administered an anti-CD20 antibody (such as a CD20-directed cytolytic antibody). In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

In still further embodiments, provided herein is a method of decreasing disability in a subject with PIRA, comprising administering to the subject a therapeutically effective amount of vidofludimus, or an isotopic variant, pharmaceutically acceptable salt or solvate thereof, preferably vidofludimus calcium salt dihydrate as “Polymorph A”. In further embodiments provided herein is a method administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In still further embodiments, provided herein is a method of decreasing disability in a subject with PIRA, comprising administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In some embodiments, provided is a compound for use in a method of decreasing disability in a subject with PIRA, wherein the compound is vidofludimus, or an isotopic variant, pharmaceutically acceptable salt or solvate thereof, and the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In further embodiments, provided herein is a compound for use in the manufacture of a medicament for use in a method of decreasing disability in a subject with PIRA, wherein the compound is vidofludimus, or an isotopic variant, pharmaceutically acceptable salt or solvate thereof, and wherein the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Decreasing disability may comprise reducing the psychological impact of MS; increasing upper limb function; increasing walking ability; decreasing fatigue; improving work status; or decreasing global impression of MS severity; or any combinations thereof. Decreasing disability may further include decreasing one or more symptoms of PIRA, or decreasing one or more physical impacts of PIRA on the subject. The decrease in disability (including, for example, one or more symptoms or physical impacts, or other aspects as described herein) may be evaluated as described herein, such as using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level. In some embodiments, one or more of 9-HPT, T25FWT, or EDSS is used. In some embodiments, decreasing disability comprises a subject that can complete the T25FWT anchor 9-HPT more quickly, or a decrease in the EDSS score (e.g., closer to “normal”). In certain embodiments, a decrease in disability comprises an improvement in one or more metrics of PIRA, such as one evaluated using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level. In certain embodiments, the improvement is at least 5%, at least 10%, at least 15%, at least 20%, at least 25%, at least 30%, or at least 35% in at least one metric of PIRA (e.g., in T25FWT time, or 9-HPT time, or EDSS score), as compared to the same metric evaluated in the same subject prior to beginning administration of vidofludimus, or an isotopic variant, pharmaceutically acceptable salt or solvate thereof, preferably vidofludimus calcium salt dihydrate as “Polymorph A”. In some embodiments, two, three, four, five or more metrics are improved, wherein each improvement level is independent (e.g., one metric improves by at least 10%, another metric improves by at least 20%). In some embodiments, the improvement is at least 5%. In some embodiments, the improvement is at least 10%. In some embodiments, the improvement is at least 15%. In some embodiments, the improvement is at least 20%. In some embodiments, the improvement is at least 25%. In some embodiments, the improvement is at least 30%. In some embodiments, the improvement is at least 35% In some embodiments, the improvement is compared to the same metric evaluated in the same subject within 1 week, or within 0 to 28 days, or within 6 weeks prior to beginning administration of vidofludimus, or an isotopic variant, pharmaceutically acceptable salt or solvate thereof. In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

In still further embodiments, provided is a method of delaying the onset of at least one progression event in a subject with PIRA, the method comprising administering to the subject a therapeutically effective amount of vidofludimus, or an isotopic variant, pharmaceutically acceptable salt or solvate thereof, preferably vidofludimus calcium salt dihydrate as “Polymorph A”. In further embodiments, provided is a method of delaying the onset of at least one progression event in a subject with PIRA, the method comprising administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Further provided is a compound for use in a method of delaying the onset of at least one progression event in a subject with PIRA, wherein the compound is vidofludimus, or an isotopic variant, pharmaceutically acceptable salt or solvate thereof, and wherein the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In yet further embodiments, provided is a compound for use in manufacture of a medicament for a method of delaying the onset of at least one progression event in a subject with PIRA, wherein the compound is vidofludimus, or an isotopic variant, pharmaceutically acceptable salt or solvate thereof, and wherein the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. A progression event may include, for example, an increase from baseline in the time needed to complete the 9-HPT, or an increase from baseline in the time needed to complete the T25FWT, or an increase from baseline of the EDSS score. In some embodiments, the increase from baseline in time needed to complete the 9-HPT is an increase of at least 20% (e g., may be 20%, 25%, 30%, 35% etc.). In some embodiments, the increase from baseline in the time needed to complete the T25FWT is an increase of at least 20% (e.g., may be 20%, 25%, 30%, 35% etc.). In still further embodiments, the increase from baseline of the EDSS score is an increase of at least 1.0 wherein the baseline is less than or equal to 5.5 points; or an increase of at least 0.5 point, in a subject with a baseline score of greater than 5.5 points. In certain embodiments, the progression event is confirmed a certain time period after the initial progression, such as at least 12 weeks, or at least 24 weeks. In certain embodiments, the baseline used in determining a progression event is the same metric (e.g., T25FWT, 9-HPT, EDSS, or combinations thereof) evaluated in the same subject within 1 week, or within 0 to 28 days, or within 6 weeks prior to beginning administration of vidofludimus, or an isotopic variant, pharmaceutically acceptable salt or solvate thereof. In certain embodiments, the methods, compounds for use, or use of a compound in the manufacture of a medicament, delays the onset of at least one progression event by at least 5%, at least 10%, at least 15%, at least 20%, at least 25%, at least 30%, or at least 35%. In some embodiments, the delay is at least 5%. In some embodiments, the delay is at least 10%. In some embodiments, the delay is at least 15%. In some embodiments, the delay is at least 20%. In some embodiments, the delay is at least 25%. In some embodiments, the delay is at least 25%. In some embodiments, the delay is at least 30%. In some embodiments, the delay is at least 35%. In some embodiments, the time to onset is delayed relative to another subject with PIRA (e.g., a comparator subject), wherein the other subject is not administered a DHODH inhibitor (such as vidofludimus, or an isotopic variant, pharmaceutically acceptable salt or solvate thereof). In some embodiments, the other subject is administered an anti-CD20 antibody (such as a CD20-directed cytolytic antibody), and is not administered a DHODH inhibitor (such as vidofludimus, or an isotopic variant, pharmaceutically acceptable salt or solvate thereof). In certain embodiments, the total evaluation time period is 12 weeks, 18 weeks, 2.4 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g., the time period until onset of at least one progression event is increased by at least 5%, at least 10%, at least 15%, at least 20%, at least 25%, at least 30%, or at least 35%, as evaluated over 120 weeks, when compared to another subject with PIRA who is not administered a DHODH inhibitor (such as vidofludimus, or an isotopic variant, pharmaceutically acceptable salt or solvate thereof) and is optionally administered an anti-CD20 antibody (such as a CD20-directed cytolytic antibody). In some embodiments, calculating the delay in onset of at least one progression event may comprise, for example, calculating the additional time until the onset of a progression event in subject administered vidofludimus, or an isotopic variant, pharmaceutically acceptable salt or solvate thereof, as compared to a subject not administered vidofludimus, or an isotopic variant, pharmaceutically acceptable salt or solvate thereof (and optionally administered an anti-CD20 antibody). In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

Further provided herein is a method of reducing the risk of a subject with PIRA having at least one progression event, the method comprising administering to the subject a therapeutically effective amount of vidofludimus, or an isotopic variant, pharmaceutically acceptable salt or solvate thereof, preferably vidofludimus calcium salt dihydrate as “Polymorph A”. Also provided herein is a method of reducing the risk of a subject with PIRA having at least one progression event, the method comprising administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Further provided is a compound for use in a method of reducing the risk of a subject with PIRA having at least one progression event, wherein the compound is vidofludimus, or an isotopic variant, pharmaceutically acceptable salt or solvate thereof, and wherein the method comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. In yet further embodiments, provided is a compound for use in manufacture of a medicament for reducing the risk of a subject with PIRA having at least one progression event, wherein the compound is vidofludimus, or an isotopic variant, pharmaceutically acceptable salt or solvate thereof, and wherein the subject is administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. A progression event may include, for example, an increase from baseline in the time needed to complete the 9-HPT, or an increase from baseline in the time needed to complete the T25FWT, or an increase from baseline of the EDSS score. In some embodiments, the increase from baseline in time needed to complete the 9-HPT is an increase of at least 20% (e.g., may be 20%, 25%, 30%, etc.). In some embodiments, the increase from baseline in the time needed to complete the T25FWT is an increase of at least 20% (e.g., may be 20%, 25%, 30%, etc.). In still further embodiments, the increase from baseline of the EDSS score is an increase of at least 1.0 (e.g, may be 1.0, 1.5, 2.0, etc.) wherein the baseline is less than or equal to 5.5 points; or an increase of at least 0.5 point (e.g., may be 0.5, 1.0, 1.5, etc.) in a subject with a baseline score of greater than 5.5 points. In certain embodiments, the progression event is continued a certain time period after the initial progression, such as at least 12 weeks, or at least 24 weeks (e.g., as CDP12, cCDP12, CDP24, or cCDP24). In certain embodiments, the baseline used in determining a progression event is the same metric (e.g., T25FWT, 9-HPT, EDSS, or combinations thereof) evaluated in the same subject within 1 week, or within 0 to 28 days, or within 6 weeks prior to beginning administration of vidofludimus, or an isotopic variant, pharmaceutically acceptable salt or solvate thereof. In certain embodiments, the methods, compounds for use, or use of a compound in the manufacture of a medicament, reduces the risk of the subject with PIRA having at least one progression event by at least 5%, at least 10%, at least 15%, at least 20%, at least 25%, at least 30%, or at least 35%. In some embodiments, the risk is reduced at least 5%. In some embodiments, the risk is reduced at least 10%. In some embodiments, the risk is reduced at least 15%. In some embodiments, the risk is reduced at least 20%. In some embodiments, the risk is reduced at least 25%. In some embodiments, the risk is reduced at least 30%. In some embodiments, the risk is reduced at least 35%. In some embodiments, the risk of having at least one progression event comprises reducing the risk of experiencing cCDP12, or reducing the risk of worsening according to EDSS. In some embodiments, the reduced risk of having at least one progression event is reduced relative to another subject with PIRA, wherein the other subject is not administered a DHODH inhibitor (such as vidofludimus, or an isotopic variant, pharmaceutically acceptable salt or solvate thereof). In some embodiments, the other subject is administered an anti-CD20 antibody (such as a CD20-directed cytolytic antibody), and is not administered a DHODH inhibitor (such as vidofludimus, or an isotopic variant, pharmaceutically acceptable salt or solvate thereof). In certain embodiments, the total evaluation time period is 12 weeks, 18 weeks, 24 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g., a subject with PIRA administered vidofludimus, or an isotopic variant, pharmaceutically acceptable salt or solvate thereof has at least 5%, at least 10%, at least 15%, at least 20%, at least 25%, at least 30%, or at least 35% lower risk of having at least one progression event over 120 weeks, when compared to another subject with PIRA who is not administered vidofludimus, or an isotopic variant, pharmaceutically acceptable salt or solvate thereof and is optionally administered a CD20-directed cytolytic antibody. Such reduced nsk of having at least one progression event may be calculated, for example, by calculating the rate of progression events in one or more subject with PIRA administered vidofludimus, or an isotopic variant, pharmaceutically acceptable salt or solvate thereof (e.g., over 60 weeks, or over 120 weeks) and comparing that rate to the rate of progression events in one or more subject with PIRA not administered vidofludimus, or an isotopic variant, pharmaceutically acceptable salt or solvate thereof over and optionally administered an anti-CD20 antibody (such as a CD20-directed cytolytic antibody). In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

Further provided herein are methods of increasing mobility in a subject in need thereof, wherein the subject has PIRA, comprising administering to the subject a therapeutically effective amount of vidofludimus, or an isotopic variant, pharmaceutically acceptable salt or solvate thereof, preferably vidofludimus calcium salt dihydrate as “Polymorph A”. Also provided herein are methods of increasing mobility in a subject in need thereof, wherein the subject has PIRA, comprising administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Thus, provided herein are methods of increasing mobility in a subject in need thereof, wherein the subject has PIRA, by administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”. Increased mobility in subject may include, for example, increased ability to walk, increased ability to run, increased ability to climb up and/or downstairs. Increased ability to stand, improved balance when walking or standing, increased distance a subject is able to walk, decreased effort needed to walk, decreased reliance on supports when walking indoors and/or outdoors (e g., walking stick, leaning on furniture, walking frame, etc.), decreased amount of concentration required to walk, or increase in the evenness/smoothness of walking, or any combination of the foregoing. In some embodiments, one, two, or more of these aspects of mobility is improved, while one or more is not improved. For example, increasing mobility in a subject may comprise increased ability to walk, while one or more other components of mobility is not improved. Such increased mobility may, for example, be assessed using a subject questionnaire. In some embodiments, increased mobility, or one or more components of increased mobility as described herein, may be evaluated using the MSWS-12. In some embodiments, an increase in mobility is evaluated compared to the mobility (e.g., as evaluated using MSWS-12) of the subject prior to beginning administration of vidofludimus, or an isotopic variant, pharmaceutically acceptable salt or solvate thereof. In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

In some embodiments as provided herein, the progression of PIRA may be evaluated by one or more clinical or laboratory endpoints selected from the group consisting of MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level. Thus, for example, in some embodiments the progression of PIRA is evaluated by one or more of EDSS, T25FWT, or 9-HPT. Further, in some embodiments the progression of PIRA is evaluated by a sustained increase in one or more PIRA symptoms or signs, such as an increase that is sustained over at least 12 weeks (e.g., confirmed to still be increased at least 12 weeks after the initial increase observed), or at least 24 weeks (e.g., confirmed to still be increased at least 24 weeks after the initial increase observed). In certain embodiments, the progression of PIRA is evaluated by a cCDP or CDP, such as cCDP-12, CDP-12, cCDP24, or CDP24, or any combinations thereof. In some embodiments, progression of PIRA is evaluated by cCDP12. In certain embodiments, progression of PIRA is evaluated by EDSS. In certain embodiments, the risk of experiencing cCDP12 is decreased, or time to onset of cCDP12 is increased, in combination with reducing the risk of experiencing CDP12, or in combination with increasing the time to onset of CDP12. In certain embodiments, the risk of experiencing cCDP12 is decreased, or time to onset of cCDP12 is increased, in combination with reducing the risk of experiencing cCDP24, or in combination with increasing the time to onset of cCDP24. In still further embodiments, the risk of experiencing cCDP12 is decreased, in combination with both: reducing the risk of experiencing CDP12, and reducing the risk of experiencing cCDP24. In still further embodiments, time to onset of cCDP12 is increased in combination with both: increasing the time to onset of CDP 12, and increasing the time to onset of cCDP24. In some embodiments, the PIRA subgroup is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof. In some embodiments, the PIRA subgroup is aSPMS. In some embodiments, the PIRA subgroup is n-aSPMS. In some embodiments, the PIRA subgroup is SPMS. In some embodiments, the PIRA subgroup is PPMS. In some embodiments, the PIRA subgroup is PMS. In some embodiments, the subject has POMS. In some embodiments, the subject has LOMS. In some embodiments, the subject has AOMS.

In some embodiments described herein, the response of a subject administered vidofludimus, or an isotopic variant, pharmaceutically acceptable salt or solvate thereof may be compared to another subject who is administered an antibody to CD20 (e.g., an anti-CD20 antibody). As used herein an anti-CD20 antibody may include antibodies which bind to CD20, a cell surface antigen present on pre-B and mature B lymphocytes. In some embodiments, the antibody is a humanized monoclonal antibody directed against CD20-expressing B-cells. In certain embodiments, binding of the anti-CD20 antibody to the cell surface of B lymphocytes may result in antibody-dependent cellular cytolysis, and complement mediated lysis. In certain embodiments, the anti-CD20 antibody is a CD20-directed cytolytic antibody. Examples of such antibodies may include, for example, ocrelizumab. Ocrelizumab is a recombinant humanized, glycosylated, monoclonal IgG1 antibody that selectively targets and depletes CD20-expressing B cells.

In some embodiments of the methods, compounds for use, or use of a compound as described herein, a therapeutically effective amount of vidofludimus, or an isotopic variant, pharmaceutically acceptable salt or solvate thereof, preferably vidofludimus calcium salt dihydrate as “Polymorph A”, is administered to a subject with PIRA, wherein the subject with PIRA has had progressive disease from the onset, and a progressive stage for at least 12 months prior to beginning administration of vidofludimus, or an isotopic variant, pharmaceutically acceptable salt or solvate thereof. In certain embodiments of the methods, compounds for use, or use of a compound as described herein, in the maintenance phase about 30 to 45 mg of vidofludimus, or an equivalent amount of an isotopic variant, pharmaceutically acceptable salt or solvate thereof, preferably vidofludimus calcium salt dihydrate as “Polymorph A”, is administered once daily to a subject with PIRA, wherein the subject with PIRA has had progressive disease from the onset, and a progressive stage for at least 12 months prior to beginning administration of vidofludimus, or an isotopic variant, pharmaceutically acceptable salt or solvate thereof. In some embodiments, the subject with PIRA has one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. In certain embodiments, the subject with PIRA has at least two of: one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. In still further embodiments, the subject with PIRA has had progressive disease from the onset, and a progressive stage for at least 12 months prior to beginning administration of vidofludimus, or an isotopic variant, pharmaceutically acceptable salt or solvate thereof; and has at least two of: one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. T2-hyperintense lesions may be evaluated, for example, by MRI. The presence of cerebrospinal fluid-specific oligoclonal bands may be evaluated, for example, by lumbar puncture. In further embodiments, the subject with PIRA may have an EDSS score from between 3.0 to 6.5 prior to beginning administration of vidofludimus, or an isotopic variant, pharmaceutically acceptable salt or solvate thereof. In some embodiments, the subject with PIRA is neurologically stable for at least 30 days prior to beginning administration of vidofludimus, or an isotopic variant, pharmaceutically acceptable salt or solvate thereof. In some such embodiments, the method, compound for use, or use of a compound, is for treating PIRA; treating (e.g. slowing) progression of PIRA; decreasing disability; delaying the onset of at least one progression event; reducing the risk of having at least one progression event; increasing mobility; or increasing time to onset of cCDP12, in a subject with PIRA in need thereof; and comprises administering to the subject in the maintenance phase a once daily dose of about 30 to 45 mg of vidofludimus, or an equivalent amount of an isotopic variant, pharmaceutically acceptable salt or solvate thereof, preferably vidofludimus calcium salt dihydrate as “Polymorph A”.

Also provided herein are pharmaceutical compositions and formulations comprising vidofludimus, or an isotopic variant, pharmaceutically acceptable salt or solvate thereof, for use in the methods of treatment described herein (e.g., treating PIRA, delaying the progression of PIRA, etc.). In some embodiments, the pharmaceutical compositions and formulations further comprise one or more pharmaceutically acceptable carriers.

Vidofludimus, or an isotopic variant, pharmaceutically acceptable salt or solvate thereof, preferably vidofludimus calcium salt dihydrate as “Polymorph A”, can be administered by any suitable means, including oral, parenteral, intrapulmonary, and intranasal, and, if desired for local treatment, intralesional administration. In certain embodiments, oral administration is used.

Pharmaceutically acceptable salts of a compound according to Formula (I) to (V), or with a compound according to Formula (VI), preferably vidofludimus or an isotopic variant thereof, may be used in the methods herein. As used herein, the term “pharmaceutically acceptable salt” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanol amine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

In some of the embodiments provided herein, an oral dose of a compound according to Formula (I) to (V), or with a compound according to Formula (VI), preferably vidofludimus, or an isotopic variant, pharmaceutically acceptable salt or solvate thereof, is administered as one or more tablets or capsules. For example, in some embodiments, the maintenance phase about 30 to 45 mg vidofludimus calcium salt dihydrate as “Polymorph A”, is administered once daily as one tablet.

In further embodiments as provided herein, an article of manufacture or a kit is provided comprising a compound according to Formula (I) to (V), or with a compound according to Formula (VI), preferably vidofludimus, or an isotopic variant, pharmaceutically acceptable salt or solvate thereof, and a container. In certain embodiments, further include is a package insert comprising instructions for using a compound according to Formula (I) to (V), or with a compound according to Formula (VI), preferably vidofludimus, or an isotopic variant, pharmaceutically acceptable salt or solvate thereof. Suitable containers for kits include, for example, a bottle, a box, a blister pack, or a combination thereof (e.g., a blister pack in a box). In some embodiments, the container holds the formulation and the label on, or associated with, the container may indicate directions for use. The article of manufacture or kit may further include other materials desirable from a commercial and user standpoint, including package inserts with instructions for use.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### IV. Medical Uses of Compounds

One aspect of the present disclosure relates to a compound according to Formula (I) to (V), or with a compound according to Formula (VI), for use in treating a disease caused by lower levels of Nurr1 in the central nervous system of a subject in need thereof.

In some embodiments, the disease caused by lower levels of Nurr1 is Alexander's disease, Alper's disease, Alzheimer's disease, amyotrophic lateral sclerosis (ALS), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, corticobasal syndrome (CBS), Creutzfeldt-Jakob disease, Down syndrome (DS), frontotemporal dementia, Gerstmann-Straussler-Scheinker syndrome, Huntington's disease, HIV-associated dementia (FTD), Kennedy's disease, Krabbe's disease, kuru, dementia with Lewy bodies (DLB), Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, progressive supranuclear palsy (PSP), Refsum's disease, Sandhoffs disease, Schilder's disease, subacute combined degeneration of spinal cord secondary to pernicious anemia, schizophrenia, spinocerebellar ataxia (multiple types with varying characteristics), spinal muscular atrophy, Steele-Richardson-Olszewski disease, or tabes dorsalis.

In some embodiments, the disease caused by lower levels of Nurr1 is Parkinson's disease, Alzheimer's disease, multiple sclerosis, amyotrophic lateral sclerosis, schizophrenia, or drug addiction.

In some embodiments, the disease is Parkinson's disease.

In some embodiments, the disease is Alzheimer's disease.

In some embodiments, the disease is multiple sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

In some embodiments the subject in need of the above-mentioned method for treating MS is human, in some embodiments particularily female humans.

In some embodiments the subject in need of the above-mentioned method for treating MS belongs to the patient group with a paediatric-onset MS (POMS), which means an onset until the age of 18.

In some embodiments the subject in need of the above-mentioned method for treating MS belongs to the patient group with a late-onset MS (LOMS), which means an onset in the time period from 19-50 years.

In some embodiments the subject in need of the above-mentioned method for treating MS belongs to the patient group with an adult-onset MS (AOMS), which means an onset >50 years.

The age of a patient at onset MS seems to be e.g. a PIRA-associated factor: In one study AOMS compared with POMS has a hazard ratio of 1.42; LOMS compared with POMS has a hazard ratio of 2.98 (JAMA Neurol. 2024; 81:50).

Other PIRA-associated factors seem to be the length of time of the disease (the higher the disease terms the higher the PIRA risk) and the length of time of the treatment with a DMT (the shorter the term of treatment with DMT the higher the PIRA risk).

In some embodiments the subject in need of the above-mentioned method for treating MS belongs to the patient group with clinically isolated syndrome (CIS).

In some embodiments the subject in need of the above-mentioned method for treating MS belongs to the patient group characterized as transitioning MS patient.

In some embodiments the subject in need of a method for preventing and or treating MS belongs to the patient group before or around onset MS or while not yet diagnosed MS if there are one or more indications e.g. by biomarkers and or genetic dispositions and or symptomes like MS prodrom which increase the risk score. Generally, onset MS means that there has been taken place a first demyelinating event. As there are many cases, e.g. patients with the the so called benign MS, that remain undetected over many years it could be of advantage to include them into a treatment according to the invention even during the term before the detection of the MS disease, if they have a correspondent increased risk score, which can be determined by suitable biomarkers and genetic disposition.

In some embodiments, the disease is progressive multiple sclerosis.

In some embodiments, the disease is primary progressive multiple sclerosis.

In some embodiments, the disease is non-active secondary progressive multiple sclerosis (non-active SPMS).

In some embodiments, the disease is active secondary progressive multiple sclerosis (active SPMS).

In some embodiments, the disease is relapsing remitting multiple sclerosis.

In some embodiments, the disease is multiple sclerosis, wherein the disability is acquired through relapse-associated worsening (RAW).

In some embodiments, the disease is multiple sclerosis, wherein the disability is acquired through progression independent of relapse activity (PIRA).

In some embodiments, the disease is relapsing-remitting multiple sclerosis (RRMS), wherein the disability is acquired through relapse-associated worsening (RAW).

In some embodiments, the disease is relapsing-remitting multiple sclerosis (RRMS), wherein the disability is acquired through progression independent of relapse activity (PIRA).

In some embodiments, the disease is active secondary progressive multiple sclerosis (active SPMS), wherein the disability is acquired through relapse-associated worsening (RAW).

In some embodiments, the disease is active secondary progressive multiple sclerosis (active SPMS), wherein the disability is acquired through progression independent of relapse activity (PIRA).

In some embodiments, the disease is primary progressive multiple sclerosis (PPMS), wherein the disability is acquired through progression independent of relapse activity (PIRA).

In some embodiments, the disease is non-active secondary progressive multiple sclerosis (non-active SPMS), wherein the disability is acquired through progression independent of relapse activity (PIRA).

In some embodiments, the disease is clinically isolated syndrome (CIS).

In some embodiments, the disease is transitioning MS.

In some embodiments, the condition PIRA.

In some embodiments, the disease is amyotrophic lateral sclerosis.

In some embodiments, the disease is schizophrenia.

In some embodiments, the disease is drug addiction.

In some embodiments, the disease caused by lower levels of Nurr1 is a cancer.

In some embodiments, the compound is according to Formula (I),

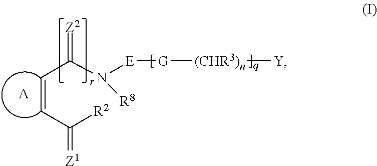

wherein:
   A is a ring that is unsubstituted or substituted;
   Z¹ and Z² are each independently O, S, or NR⁹;
   E is a linker or is absent;
   G is a linker or is absent;
   Y is a ring;
   R² is H, OR¹¹, NR¹¹OR¹¹, NR¹¹SO₂R¹¹, or NR¹¹R¹²
   R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted;
   each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted;
   n is 0, 1, 2, 3, 4, or 5;
   q is 0, 1, 2, 3, 4, 5, 6, 7, 8, 9, or 10; and
   r is 0 or 1,
having one or more hydrogen atoms optionally replaced by deuterium, or a pharmaceutically acceptable salt or solvate thereof. In some embodiments, the compound is a calcium salt, a hemi-calcium salt, a solvate, a disolvate, a hydrate, a dihydrate, a calcium salt hydrate, or a hemi-calcium salt dihydrate.

In some embodiments, the compound is according to Formula (I),

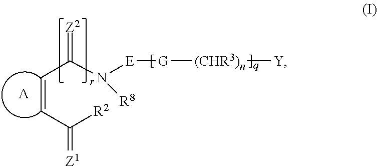

wherein:
   Ring A is a 5- or 6-membered carbocyclic or heterocyclic ring, which is optionally substituted with 1 to 4 residues R¹;
   Z¹ and Z² are each independently O, S, or NR⁹;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted;
   R² is H, OR¹¹, NR¹¹OR¹¹, NR¹¹SO₂R¹¹, or NR¹¹R¹²;
   R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted;
   n is 0 or 1;
   q is 0, 1, 2, 3, 4, 5, 6, 7, 8, 9, or 10; and
   r is 0 or 1,
having one or more hydrogen atoms optionally replaced by deuterium, or a pharmaceutically acceptable salt or solvate thereof.

In some embodiments, the compound is according to Formula (II), or a pharmaceutically acceptable salt or solvate thereof:

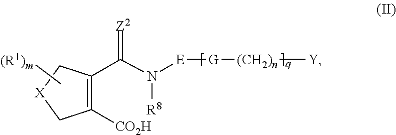

wherein:
   X is O, S, NR⁹, SO, or SO₂;
   Z² is O, S, or NR¹²;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted;
   m is 0, 1, 2, 3, or 4;
   n is 0 or 1; and
   q is 0, 1, 2, 3, 4, 5, 6, 7, 8, 9, or 10.

In some embodiments, the compound is according to Formula (III), or a pharmaceutically acceptable salt or solvate thereof:

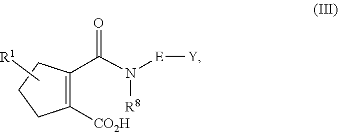

wherein:
   E is alkylene, alkenylene, alkynylene, arylene, heteroarylene, or cycloalkylene;
   Y is aryl, heteroaryl, heterocyclyl, or cycloalkyl;
   R¹ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; and
   each R⁸, R¹³, and R¹⁴ is independently H, alkyl, alkenyl, alkynyl, aryl, cycloalkyl, heteroaryl, or heterocyclyl, each of which is independently substituted or unsubstituted.

In some embodiments, E is alkylene or cycloalkylene, each of which is optionally substituted with cyclopropyl, cyclobutyl, cyclopentyl, cyclohexyl, cycloheptyl, cyclooctyl, phenyl, 1-naphthyl, 2-naphthyl, 2-naphthyl, anthracenyl, N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 4-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl or 5-oxazolyl. In some embodiments, E is a fused polycyclic arylene or a fused polycyclic heteroarylene. In some embodiments, E is 9H-thioxanthene-10,10-dioxide.

In some embodiments, ring A is a 5- or 6-membered carbocyclic or heterocyclic ring. In some embodiments, Ring A is partially unsaturated. In some embodiments, Ring A is an aromatic ring. In some embodiments, Ring A is a cyclopentenyl group with the double bond positioned as depicted in Formula (I). In some embodiments, Ring A is 2,3-dihydrofuranyl, 2,5-dihydrofuranyl, 2,5-dihydrothiophenyl, thiophene or 2,5-dihydro-1H-pyrrole.

In some embodiments, the compound is according to Formula (IV), or a pharmaceutically acceptable salt or solvate thereof:

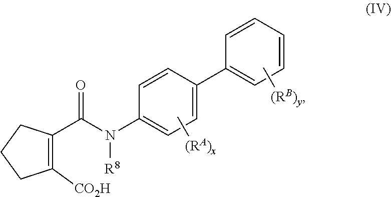

wherein:
   each R^{A} and R^{B} is independently H, D, halo, —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted;
   each R⁸, R¹³, and R¹⁴ is independently H, D, alkyl, alkenyl, alkynyl, aryl, cycloalkyl, heteroaryl, or heterocyclyl, each of which is independently substituted or unsubstituted;
   x is 0, 1, 2, 3, or 4; and
   y is 0, 1, 2, 3, 4, or 5.

In some embodiments, the compound is according to Formula (V), or a pharmaceutically acceptable salt or solvate thereof:
wherein:

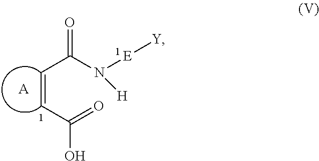

Ring A is

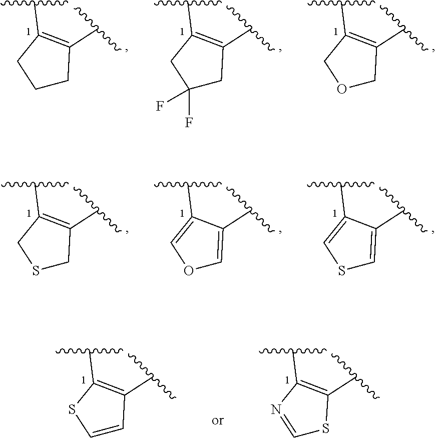

E is

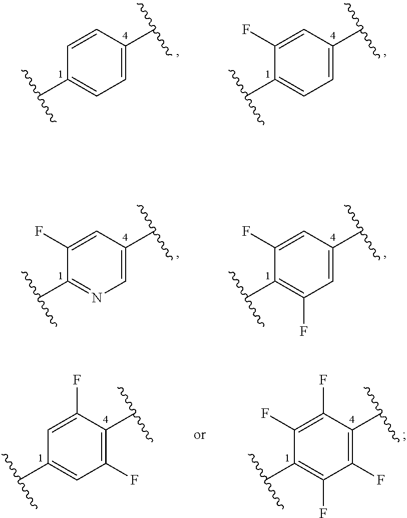 [SMILES: *c1ccc(*)cc1 ; *c1ccc(*)c(F)c1 ; *c1cnc(*)c(F)c1 ; *c1cc(F)c(*)c(F)c1 ; *c1cc(F)c(*)c(F)c1 ; *c1c(F)c(F)c(*)c(F)c1F]

and
Y is

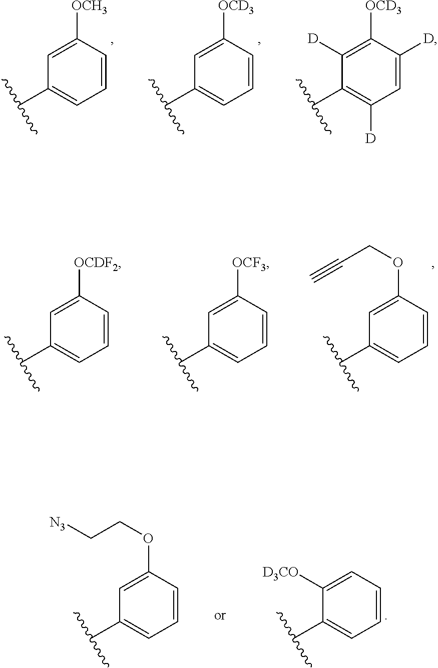

In some embodiments, the compound is

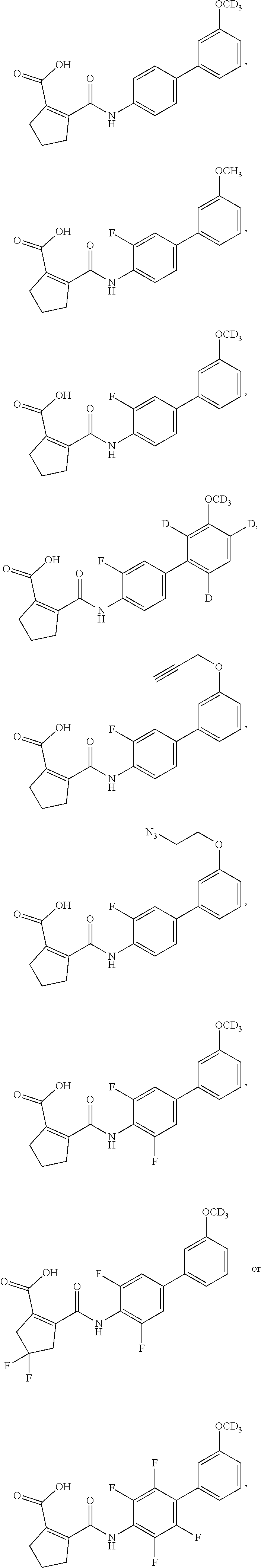

or a pharmaceutically acceptable salt or solvate thereof.

In some embodiments, the compound is

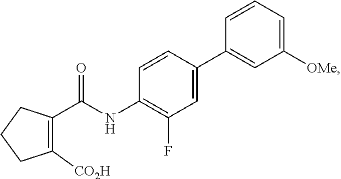 [SMILES: COc1cccc(-c2ccc(NC(=O)C3=C(C(=O)O)CCC3)c(F)c2)c1]

or an isotopic variant or a pharmaceutically acceptable salt or solvate thereof.

In some embodiments, the compound is

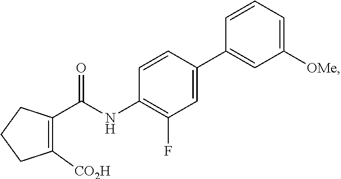 [SMILES: COc1cccc(-c2ccc(NC(=O)C3=C(C(=O)O)CCC3)c(F)c2)c1]

or a pharmaceutically acceptable salt or solvate thereof.

In some embodiments, the compound is

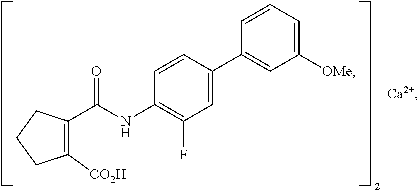

or a solvate thereof.

In some embodiments, the compound is

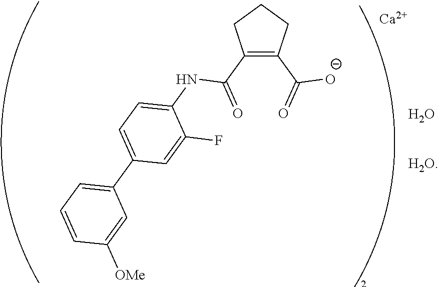

In some embodiments, the compound is according to Formula (VI), or a pharmaceutically acceptable salt or solvate thereof:

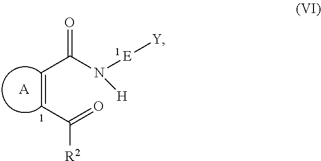

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

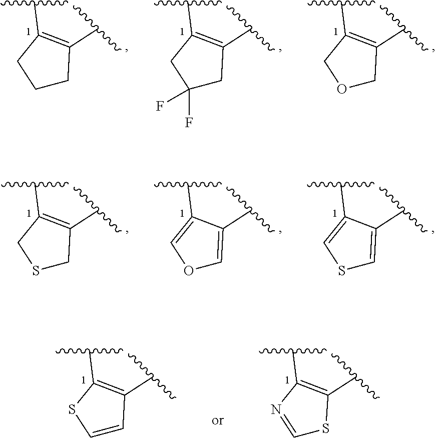

E is

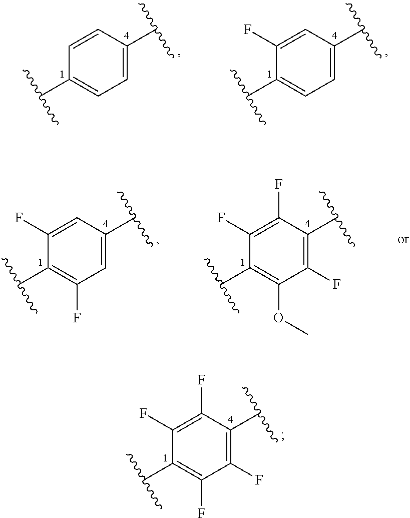 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

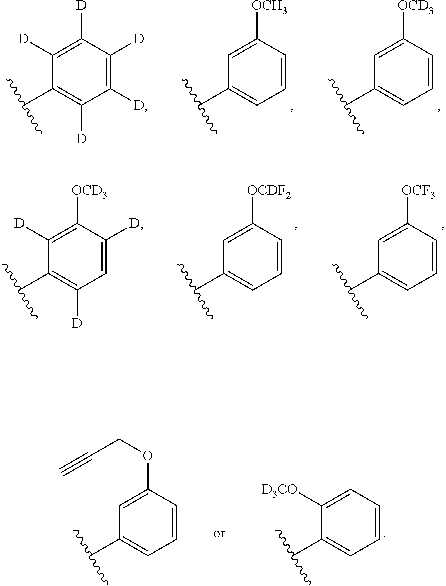

In some embodiments, the compound is according to Formula (VI), or a pharmaceutically acceptable salt or solvate thereof:

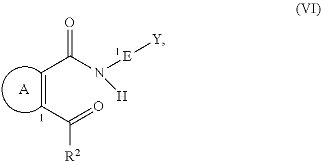

wherein:
   R² is NHR¹¹;
   R¹¹ is selected from H or alkyl, having one or more hydrogen atoms in the alkyl optionally replaced by deuterium;
   Ring A is

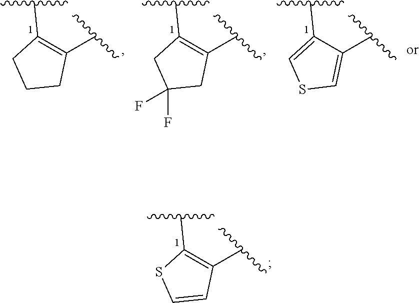 [SMILES: *C1=C(*)CCC1 ; *C1=C(*)CC(F)(F)C1 ; *c1cscc1* ; *c1ccsc1*]

E is

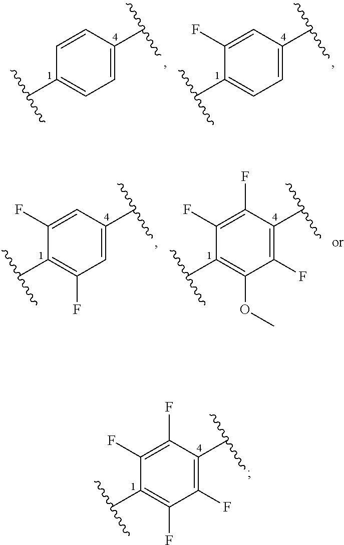 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

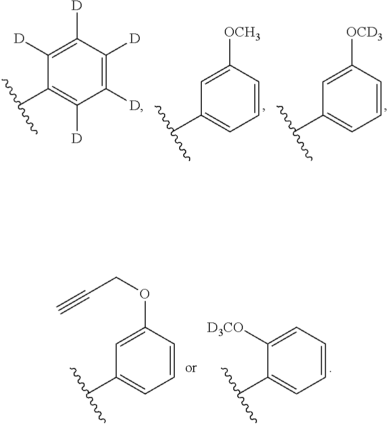

In some embodiments, the compound is

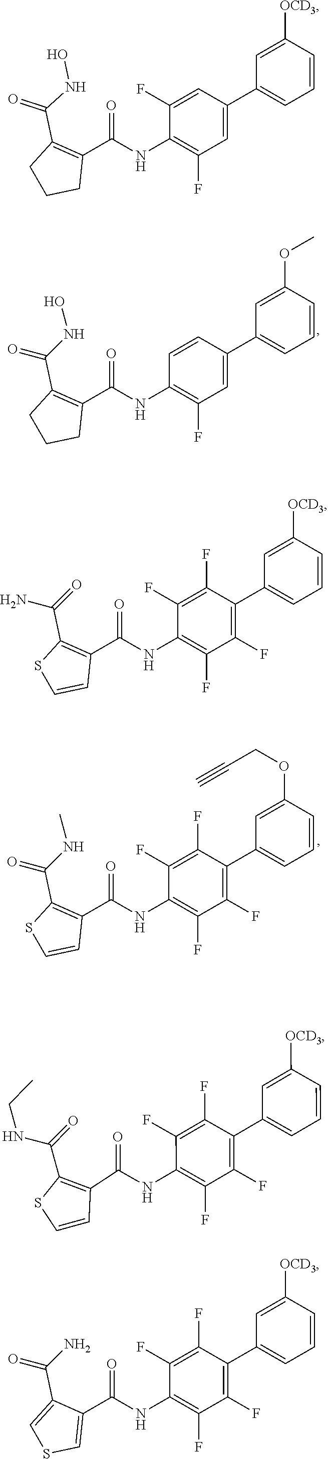

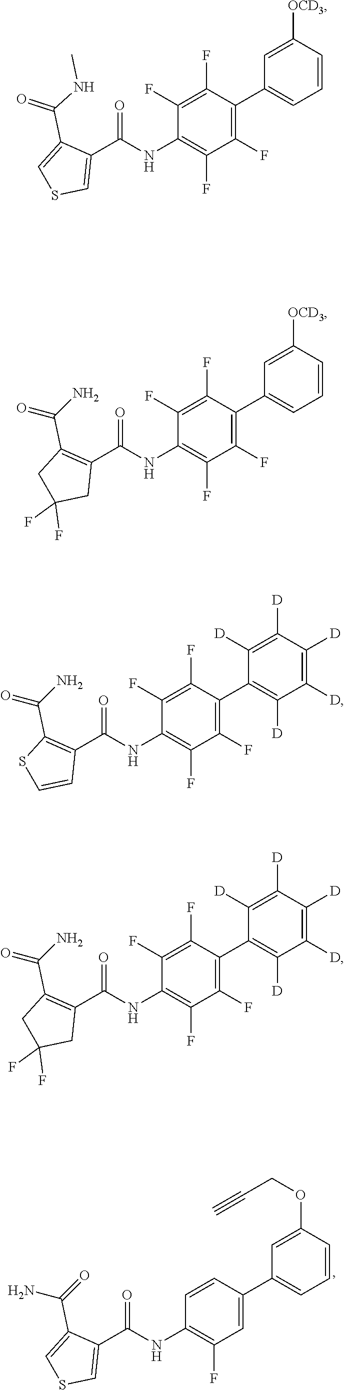

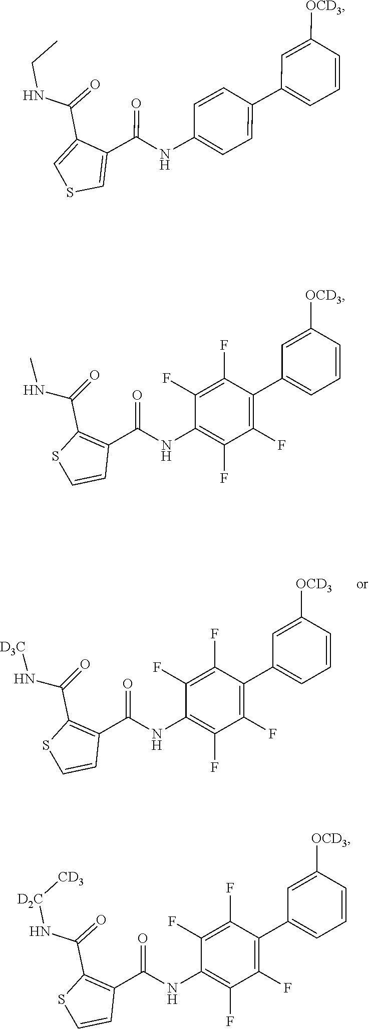

or a pharmaceutically acceptable salt or solvate thereof.

In some embodiments, each alkyl, alkenyl, alkynyl, alkoxy, aryl, cycloalkyl, heteroaryl, and heterocyclyl is optionally substituted by one or more R′, wherein each R′ is independently H, D, —CO₂R″, —CONHR″, —CR″O, —SO₂N(R″)₂, —NR″—CO-haloalkyl, —NR″—CO-alkyl, —NO₂, —N₃, —NR″—SO₂-haloalkyl, —NR″—SO₂-alkyl, —SO₂-alkyl, —CN, alkyl, cycloalkyl, aminoalkyl, alkylamino, alkoxy, —OH, oxo, —SH, alkylthio, hydroxyalkyl, hydroxyalkylamino, halogen, haloalkyl, haloalkyloxy, aryl, arylalkyl or heteroaryl, wherein each R″ is independently H, D, haloalkyl, hydroxyalkyl, alkyl, cycloalkyl, aryl, heteroaryl or aminoalkyl.

### IV. Methods of Treatment

In an aspect is provided a method for treating a disease caused by lower levels of Nurr1 in the central nervous system of a subject in need thereof, the method including administering to the subject in need thereof a therapeutically effective amount of a compound according to Formula (I) to (V), or with a compound according to Formula (VI), described herein.

In an aspect is provided a method for treating a disease caused by lower levels of Nurr1 in the central nervous system of a subject in need thereof, the method including administering to the subject in need thereof a therapeutically effective amount of vidofludimus or a solvate or pharmaceutically acceptable salt thereof.

In an aspect is provided a method for treating a disease caused by lower levels of Nurr1 in the central nervous system of a subject in need thereof, the method including administering to the subject in need thereof a therapeutically effective amount of IMU-838, for example, as Polymorph A.

In some embodiments, the disease caused by lower levels of Nurr1 is Alexander's disease, Alper's disease, Alzheimer's disease, amyotrophic lateral sclerosis (ALS), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, corticobasal syndrome (CBS), Creutzfeldt-Jakob disease, Down syndrome (DS), frontotemporal dementia, Gerstmann-Straussler-Scheinker syndrome, Huntington's disease, HIV-associated dementia (FTD), Kennedy's disease, Krabbe's disease, kuru, dementia with Lewy bodies (DLB), Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, progressive supranuclear palsy (PSP), Refsum's disease, Sandhoffs disease, Schilder's disease, subacute combined degeneration of spinal cord secondary to pernicious anemia, schizophrenia, spinocerebellar ataxia (multiple types with varying characteristics), spinal muscular atrophy, Steele-Richardson-Olszewski disease, or tabes dorsalis.

In some embodiments, the disease caused by lower levels of Nurr1 is Parkinson's disease, Alzheimer's disease, multiple sclerosis, amyotrophic lateral sclerosis, schizophrenia, or drug addiction.

In some embodiments, the disease is Parkinson's disease.

In some embodiments, the disease is Alzheimer's disease.

In some embodiments, the disease is multiple sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

In some embodiments the subject in need of the above-mentioned method for treating MS is human, in some embodiments particularly female humans.

In some embodiments the subject in need of the above-mentioned method for treating MS belongs to the patient group with a paediatric-onset MS (POMS), which means an onset until the age of 18.

In some embodiments the subject in need of the above-mentioned method for treating MS belongs to the patient group with a late-onset MS (LOMS), which means an onset in the time period from 19-50 years.

In some embodiments the subject in need of the above-mentioned method for treating MS belongs to the patient group with an adult-onset MS (AOMS), which means an onset >50 years.

The age of a patient at onset MS seems to be e.g. a PIRA-associated factor: In one study AOMS compared with POMS has a hazard ratio of 1.42; LOMS compared with POMS has a hazard ratio of 2.98 (JAMA Neurol. 2024; 81:50).

Other PIRA-associated factors seem to be the length of time of the disease (the higher the disease terms the higher the PIRA risk) and the length of time of the treatment with a DMT (the shorter the term of treatment with DMT the higher the PIRA risk).

In some embodiments the subject in need of the above-mentioned method for treating MS belongs to the patient group with clinically isolated syndrome (CIS).

In some embodiments the subject in need of the above-mentioned method for treating MS belongs to the patient group characterized as transitioning MS patient.

In some embodiments the subject in need of a method for preventing and or treating MS belongs to the patient group before or around onset MS or while not yet diagnosed MS if there are one or more indications e.g. by biomarkers and or genetic dispositions and or symptomes like MS prodrom which increase the risk score. Generally, onset MS means that there has been taken place a first demyelinating event. As there are many cases, e.g. patients with the the so called benign MS, that remain undetected over many years it could be of advantage to include them into a treatment according to the invention even during the term before the detection of the MS disease, if they have a correspondent increased risk score, which can be determined by suitable biomarkers and genetic disposition.

In some embodiments, the disease is progressive multiple sclerosis.

In some embodiments, the disease is primary progressive multiple sclerosis.

In some embodiments, the disease is non-active secondary progressive multiple sclerosis (non-active SPMS).

In some embodiments, the disease is active secondary progressive multiple sclerosis (active SPMS).

In some embodiments, the disease is relapsing remitting multiple sclerosis.

In some embodiments, the disease is multiple sclerosis, wherein the disability is acquired through relapse-associated worsening (RAW).

In some embodiments, the disease is multiple sclerosis, wherein the disability is acquired through progression independent of relapse activity (PIRA).

In some embodiments, the disease is relapsing-remitting multiple sclerosis (RRMS), wherein the disability is acquired through relapse-associated worsening (RAW).

In some embodiments, the disease is relapsing-remitting multiple sclerosis (RRMS), wherein the disability is acquired through progression independent of relapse activity (PIRA).

In some embodiments, the disease is active secondary progressive multiple sclerosis (active SPMS), wherein the disability is acquired through relapse-associated worsening (RAW).

In some embodiments, the disease is active secondary progressive multiple sclerosis (active SPMS), wherein the disability is acquired through progression independent of relapse activity (PIRA).

In some embodiments, the disease is primary progressive multiple sclerosis (PPMS), wherein the disability is acquired through progression independent of relapse activity (PIRA).

In some embodiments, the disease is non-active secondary progressive multiple sclerosis (non-active SPMS), wherein the disability is acquired through progression independent of relapse activity (PIRA).

In some embodiments, the disease is clinically isolated syndrome (CIS).

In some embodiments, the disease is transitioning MS.

In some embodiments, the condition PIRA.

In some embodiments, the disease is amyotrophic lateral sclerosis.

In some embodiments, the disease is schizophrenia.

In some embodiments, the disease is drug addiction.

In some embodiments, the disease caused by lower levels of Nurr1 is a cancer.

In an aspect is provided a method for treating a disease in a subject in need thereof, the method including administering to the subject in need thereof a therapeutically effective amount of a compound according to Formula (I) to (V), or with a compound according to Formula (VI), described herein.

In some embodiments, the change of a target gene (e.g., VMAT2), a target protein (e.g., Nurr1) or a peptide (e.g., NFL) is assessed after a treatment period of about 6 weeks, about 12 weeks, about 24 weeks, about 1 month, about 3 months, about 6 months, about 12 months in relation to the initial value at the start of treatment.

In some embodiments, the change of a target gene (e.g., VMAT2), a target protein (e.g., Nurr1) or a peptide (e.g., NFL) is assessed at beginning of treatment in relation to a healthy person of same age, gender and/or BMI.

In an aspect is provided a method of modulating the level or activity of Nurr1 in a subject in need thereof, the method including administering to the subject in need thereof a therapeutically effective amount of a compound described herein. In some embodiments, the level or activity of Nurr1 in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of Nurr1 in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of decreasing the level of NFL in plasma in a subject in need thereof, the method including administering to the subject in need thereof a therapeutically effective amount of a compound according to Formula (I) to (V), or with a compound according to Formula (VI), described herein. In embodiments, the level of NFL in plasma in the subject is decreased by about 3%, 4%, 5%, 7%, 10%, 12%, 15%, 20%, 30%, 40%, 50%, or more compared to the level at beginning of treatment. In embodiments, the level of NFL in plasma in the subject is decreased by at least 3%, 4%, 5%, 7%, 10%, 12%, 15%, 20%, 30%, 40%, 50%, or more compared to the level at beginning of treatment.

In an aspect is provided a method of decreasing the level of NFL in serum in a subject in need thereof, the method including administering to the subject in need thereof a therapeutically effective amount of a compound according to Formula (I) to (V), or with a compound according to Formula (VI), described herein. In embodiments, the level of NFL in serum in the subject is decreased by about 3%, 4%, 5%, 7%, 10%, 12%, 15%, 20%, 30%, 40%, 50%, or more compared to the level at beginning of treatment. In embodiments, the level of NFL in serum in the subject is decreased by at least 3%, 4%, 5%, 7%, 10%, 12%, 15%, 20%, 30%, 40%, 50%, or more compared to the level at beginning of treatment.

In an aspect is provided a method of decreasing the level of NFL in CSF in a subject in need thereof, the method including administering to the subject in need thereof a therapeutically effective amount of a compound according to Formula (I) to (V), or with a compound according to Formula (VI), described herein. In embodiments, the level of NFL in CSF in the subject is decreased by about 3%, 4%, 5%, 7%, 10%, 12%, 15%, 20%, 30%, 40%, 50%, or more compared to the level at beginning of treatment. In embodiments, the level of NFL in CSF in the subject is decreased by at least 3%, 4%, 5%, 7%, 10%, 12%, 15%, 20%, 30%, 40%, 50%, or more compared to the level at beginning of treatment.

In an aspect is provided a method of decreasing the increased level of NFL in plasma in a subject in need thereof, the method including administering to the subject in need thereof a therapeutically effective amount of a compound according to Formula (I) to (V), or with a compound according to Formula (VI), described herein. In embodiments, the level of NFL in plasma in the subject is decreased by about 3%, 4%, 5%, 7%, 10%, 12%, 15%, 20%, 30%, 40%, 50%, or more towards the level of a healthy person with the same age, gender and/or BMI. In embodiments, the level of NFL in plasma in the subject is decreased by at least 3%, 4%, 5%, 7%, 10%, 12%, 15%, 20%, 30%, 40%, 50%, or more towards the level of a healthy person with the same age, gender and/or BMI.

In an aspect is provided a method of decreasing the increased level of NFL in serum in a subject in need thereof, the method including administering to the subject in need thereof a therapeutically effective amount of a compound according to Formula (I) to (V), or with a compound according to Formula (VI), described herein. In embodiments, the level of NFL in serum in the subject is decreased by about 3%, 4%, 5%, 7%, 10%, 12%, 15%, 20%, 30%, 40%, 50%, or more towards the level of a healthy person with the same age, gender and/or BMI. In embodiments, the level of NFL in serum in the subject is decreased by at least 3%, 4%, 5%, 7%, 10%, 12%, 15%, 20%, 30%, 40%, 50%, or more towards the level of a healthy person with the same age, gender and/or BMI.

In an aspect is provided a method of decreasing the increased level of NFL in CSFIn an aspect is provided a method of decreasing the level of NFL in plasma in a subject in need thereof, the method including administering to the subject in need thereof a therapeutically effective amount of a compound described herein. In some embodiments, the level of NFL in plasma in the subject is decreased by about 3%, about 4%, about 5%, about 7%, about 10%, about 12%, about 15%, about 20%, about 30%, about 40%, about 50%, or more compared to the level at beginning of treatment. In some embodiments, the level of NFL in plasma in the subject is decreased by at least 3%, at least 4%, at least 5%, at least 7%, at least 10%, at least 12%, at least 15%, at least 20%, at least 30%, at least 40%, at least 50%, or more compared to the level at beginning of treatment.

In an aspect is provided a method of decreasing the level of NFL in serum in a subject in need thereof, the method including administering to the subject in need thereof a therapeutically effective amount of a compound described herein. In some embodiments, the level of NFL in serum in the subject is decreased by about 3%, about 4%, about 5%, about 7%, about 10%, about 12%, about 15%, about 20%, about 30%, about 40%, about 50%, or more compared to the level at beginning of treatment. In some embodiments, the level of NFL in serum in the subject is decreased by at least 3%, at least 4%, at least 5%, at least 7%, at least 10%, at least 12%, at least 15%, at least 20%, at least 30%, at least 40%, at least 50%, or more compared to the level at beginning of treatment.

In an aspect is provided a method of decreasing the level of NFL in CSF in a subject in need thereof, the method including administering to the subject in need thereof a therapeutically effective amount of a compound described herein. In some embodiments, the level of NFL in CSF in the subject is decreased by about 3%, about 4%, about 5%, about 7%, about 10%, about 12%, about 15%, about 20%, about 30%, about 40%, about 50%, or more compared to the level at beginning of treatment. In some embodiments, the level of NFL in CSF in the subject is decreased by at least 3%, at least 4%, at least 5%, at least 7%, at least 10%, at least 12%, at least 15%, at least 20%, at least 30%, at least 40%, at least 50%, or more compared to the level at beginning of treatment.

In an aspect is provided a method of decreasing the increased level of NFL in plasma in a subject in need thereof, the method including administering to the subject in need thereof a therapeutically effective amount of a compound described herein. In some embodiments, the level of NFL in plasma in the subject is decreased by about 3%, about 4%, about 5%, about 7%, about 10%, about 12%, about 15%, about 20%, about 30%, about 40%, about 50%, or more towards the level of a healthy person with the same age, biological sex and/or BMI. In some embodiments, the level of NFL in plasma in the subject is decreased by at least 3%, at least 4%, at least 5%, at least 7%, at least 10%, at least 12%, at least 15%, at least 20%, at least 30%, at least 40%, at least 50%, or more towards the level of a healthy person with the same age, gender and/or BMI.

In an aspect is provided a method of decreasing the increased level of NFL in serum in a subject in need thereof, the method including administering to the subject in need thereof a therapeutically effective amount of a compound described herein. In some embodiments, the level of NFL in serum in the subject is decreased by about 3%, about 4%, about 5%, about 7%, about 10%, about 12%, about 15%, about 20%, about 30%, about 40%, about 50%, or more towards the level of a healthy person with the same age, biological sex and/or BMI. In some embodiments, the level of NFL in serum in the subject is decreased by at least 3%, at least 4%, at least 5%, at least 7%, at least 10%, at least 12%, at least 15%, at least 20%, at least 30%, at least 40%, at least 50%, or more towards the level of a healthy person with the same age, gender and/or BMI.

In an aspect is provided a method of decreasing the increased level of NFL in CSF in a subject in need thereof, the method including administering to the subject in need thereof a therapeutically effective amount of a compound described herein. In some embodiments, the level of NFL in CSF in the subject is decreased by about 3%, about 4%, about 5%, about 7%, about 10%, about 12%, about 15%, about 20%, about 30%, about 40%, about 50%, or more towards the level of a healthy person with the same age, gender and/or BMI. In some embodiments, the level of NFL in CSF in the subject is decreased by at least 3%, at least 4%, at least 5%, at least 7%, at least 10%, at least 12%, at least 15%, at least 20%, at least 30%, at least 40%, at least 50%, or more towards the level of a healthy person with the same age, gender and/or BMI.

In an aspect is provided a method of increasing the level or activity of Pitx3 in a subject in need thereof, the method including administering to the subject in need thereof a therapeutically effective amount of a compound described herein. In some embodiments, the level or activity of Pitx3 in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of Pitx3 in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of VMAT2 in a subject in need thereof, the method including administering to the subject in need thereof a therapeutically effective amount of a compound described herein. In some embodiments, the level or activity of VMAT2 in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of VMAT2 in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of AADC in a subject in need thereof, the method including administering to the subject in need thereof a therapeutically effective amount of a compound described herein. In some embodiments, the level or activity of AADC in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of AADC in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of DAT in a subject in need thereof, the method including administering to the subject in need thereof a therapeutically effective amount of a compound described herein. In some embodiments, the level or activity of DAT in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of DAT in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-, or more.

In an aspect is provided a method of increasing the level or activity of BDNF in a subject in need thereof, the method including administering to the subject in need thereof a therapeutically effective amount of a compound described herein. In some embodiments, the level or activity of BDNF in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of BDNF in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of GDNF in a subject in need thereof, the method including administering to the subject in need thereof a therapeutically effective amount of a compound described herein. In some embodiments, the level or activity of GDNF in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of GDNF in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of GDNF receptor C-RET in a subject in need thereof, the method including administering to the subject in need thereof a therapeutically effective amount of a compound described herein. In some embodiments, the level or activity of GDNF receptor C-RET in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of GDNF receptor C-RET in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of GFAP in a subject in need thereof, the method including administering to the subject in need thereof a therapeutically effective amount of a compound described herein. In some embodiments, the level or activity of GFAP in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of GFAP in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of tyrosine hydroxylase (TH) in a subject in need thereof, the method including administering to the subject in need thereof a therapeutically effective amount of a compound described herein. In some embodiments, the level or activity of TH in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of TH in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of increasing the level or activity of SOD in a subject in need thereof, the method including administering to the subject in need thereof a therapeutically effective amount of a compound described herein. In some embodiments, the level or activity of SOD in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of SOD in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of reducing the level or activity of TNFα in a subject in need thereof, the method including administering to the subject in need thereof a therapeutically effective amount of a compound described herein. In some embodiments, the level or activity of TNFα in the subject is reduced by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of TNFα in the subject is reduced by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of reducing the level or activity of iNOS in a subject in need thereof, the method including administering to the subject in need thereof a therapeutically effective amount of a compound described herein. In some embodiments, the level or activity of iNOS in the subject is reduced by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of iNOS in the subject is reduced by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In an aspect is provided a method of reducing the level or activity of IL-1β in a subject in need thereof, the method including administering to the subject in need thereof a therapeutically effective amount of a compound described herein. In some embodiments, the level or activity of IL-1β in the subject is reduced by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of IL-1β in the subject is reduced by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In some embodiments, the method includes increasing the level of dopamine in a subject in need thereof, the method including administering to the subject in need thereof a therapeutically effective amount of a compound described herein. In some embodiments, the level of dopamine in the subject is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level or activity of dopamine in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In some embodiments, the method includes increasing development of dopaminergic neurons with a compound described herein as compared to a control (e.g., absence of the compound). In some embodiments, the level of development of dopaminergic neurons is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level of development of dopaminergic neurons in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In some embodiments, the method includes increasing maintenance of dopaminergic neurons with a compound described herein as compared to a control (e.g., absence of the compound). In some embodiments, the level of maintenance of dopaminergic neurons is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level of maintenance of dopaminergic neurons in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In some embodiments, the method includes increasing survival of dopaminergic neurons with a compound described herein as compared to a control (e.g., absence of the compound). In some embodiments, the level of survival of dopaminergic neurons is increased by about 1.2-, about 1.3-, about 1.4-, about 1.5-, about 2-, about 3-, about 4-, about 5-, about 6-, about 7-, about 8-, about 9-, about 10-, about 15-, about 20-, about 25-, about 30-, about 35-, about 40-, about 45-, about 50-, about 60-, about 70-, about 80-, about 90-, about 100-fold, or more. In some embodiments, the level of survival of dopaminergic neurons in the subject is increased by at least 1.2-, at least 1.3-, at least 1.4-, at least 1.5-, at least 2-, at least 3-, at least 4-, at least 5-, at least 6-, at least 7-, at least 8-, at least 9-, at least 10-, at least at least 15-, at least 20-, at least 25-, at least 30-, at least 35-, at least 40-, at least 45-, at least 50-, at least 60-, at least 70-, at least 80-, at least 90-, at least 100-fold, or more.

In embodiments, the method includes stabilizing a Nurr1 monomer with a compound according to Formula (I) to (V), or with a compound according to Formula (VI), described herein. In embodiments, the method includes stabilizing a Nurr1 homodimer with a compound according to Formula (I) to (V), or with a compound according to Formula (VI), described herein. In embodiments, the method includes stabilizing a head-to-tail Nurr1 homodimer with a compound according to Formula (I) to (V), or with a compound according to Formula (VI), described herein. In embodiments, the method includes stabilizing a Nurr1 heterodimer with a compound according to Formula (I) to (V), or with a compound according to Formula (VI), described herein. In embodiments, the Nurr1 heterodimer is a heterodimer with RXRα.

In embodiments, the method includes contacting a Nurr1 monomer with a compound according to Formula (I) to (V), or with a compound according to Formula (VI), described herein. In embodiments, the method includes contacting a Nurr1 homodimer with a compound according to Formula (I) to (V), or with a compound according to Formula (VI), described herein. In embodiments, the method includes contacting a head-to-tail Nurr1 homodimer with a compound according to Formula (I) to (V), or with a compound according to Formula (VI), described herein. In embodiments, the method includes contacting a Nurr1 heterodimer with a compound according to Formula (I) to (V), or with a compound according to Formula (VI), described herein. In embodiments, the Nurr1 heterodimer is a heterodimer with RXRα.

In embodiments, the method includes binding a Nurr1 monomer with a compound according to Formula (I) to (V), or with a compound according to Formula (VI), described herein. In embodiments, the method includes binding a Nurr1 homodimer with a compound according to Formula (I) to (V), or with a compound according to Formula (VI), described herein. In embodiments, the method includes binding a head-to-tail Nurr1 homodimer with a compound according to Formula (I) to (V), or with a compound according to Formula (VI), described herein. In embodiments, the method includes binding a Nurr1 heterodimer with a compound according to Formula (I) to (V), or with a compound according to Formula (VI), described herein. In embodiments, the Nurr1 heterodimer is a heterodimer with RXRα.

In embodiments, the method includes precluding the formation of Nurr1:RXR heterodimers with a compound according to Formula (I) to (V), or with a compound according to Formula (VI), described herein.

In embodiments, the method includes binding a Nurr1 and inducing Nurr1 binding to a NBRE, a NuRE, or a DR-5 response element. In embodiments, the method includes binding a Nurr1 and inducing Nurr1 binding to a NBRE. In embodiments, the method includes binding a Nurr1 and inducing Nurr1 binding to a NuRE. In embodiments, the method includes binding a Nurr1 and inducing Nurr1 binding to a DR-5 response element.

In embodiments, the compound according to Formula (I) to (V), or with a compound according to Formula (VI), stabilizes a Nurr1 monomer. In embodiments, the compound according to Formula (I) to (V), or with a compound according to Formula (VI), stabilizes a Nurr1 homodimer. In embodiments, the compound according to Formula (I) to (V), or with a compound according to Formula (VI), stabilizes a head-to-tail Nurr1 homodimer. In embodiments, the compound according to Formula (I) to (V), or with a compound according to Formula (VI), stabilizes a Nurr1 heterodimer. In embodiments, the Nurr1 heterodimer is a heterodimer with RXRα.

In embodiments, the compound according to Formula (I) to (V), or with a compound according to Formula (VI), stabilizes a Nurr1 monomer relative to a control (e.g., absence of the compound). In embodiments, the compound according to Formula (I) to (V), or with a compound according to Formula (VI), stabilizes a Nurr1 homodimer relative to a control (e.g., absence of the compound). In embodiments, the compound according to Formula (I) to (V), or with a compound according to Formula (VI), stabilizes a head-to-tail Nurr1 homodimer relative to a control (e.g., absence of the compound). In embodiments, the compound according to Formula (I) to (V), or with a compound according to Formula (VI), stabilizes a Nurr1 heterodimer relative to a control (e.g., absence of the compound). In embodiments, the Nurr1 heterodimer is a heterodimer with RXRα.

In embodiments, the compound according to Formula (I) to (V), or with a compound according to Formula (VI), contacts a Nurr1 monomer. In embodiments, the compound according to Formula (I) to (V), or with a compound according to Formula (VI), contacts a Nurr1 homodimer. In embodiments, the compound according to Formula (I) to (V), or with a compound according to Formula (VI), contacts a head-to-tail Nurr1 homodimer. In embodiments, the compound according to Formula (I) to (V), or with a compound according to Formula (VI), contacts a Nurr1 heterodimer. In embodiments, the Nurr1 heterodimer is a heterodimer with RXRα.

In embodiments, the compound according to Formula (I) to (V), or with a compound according to Formula (VI), binds a Nurr1 monomer. In embodiments, the compound according to Formula (I) to (V), or with a compound according to Formula (VI), binds a Nurr1 homodimer. In embodiments, the compound according to Formula (I) to (V), or with a compound according to Formula (VI), binds a head-to-tail Nurr1 homodimer. In embodiments, the compound according to Formula (I) to (V), or with a compound according to Formula (VI), binds a Nurr1 heterodimer. In embodiments, the Nurr1 heterodimer is a heterodimer with RXRα.

In embodiments, the compound according to Formula (I) to (V), or with a compound according to Formula (VI), precludes the formation of Nurr1:RXR heterodimers. In embodiments, the compound according to Formula (I) to (V), or with a compound according to Formula (VI), inhibits the formation of Nurr1:RXR heterodimers. In embodiments, compound according to Formula (I) to (V), or with a compound according to Formula (VI), binding to Nurr1 inhibits the resulting compound:Nurr1 complex from binding to RXR.

In embodiments, the compound according to Formula (I) to (V), or with a compound according to Formula (VI), acts as an agonist to a Nurr1 monomer. In embodiments, the compound according to Formula (I) to (V), or with a compound according to Formula (VI), acts as an agonist to a Nurr1 homodimer. In embodiments, the compound according to Formula (I) to (V), or with a compound according to Formula (VI), acts as an agonist to a head-to-tail Nurr1 homodimer. In embodiments, the compound according to Formula (I) to (V), or with a compound according to Formula (VI), acts as an agonist to a Nurr1 heterodimer. In embodiments, the Nurr1 heterodimer is a heterodimer with RXRα.

In embodiments, the compound according to Formula (I) to (V), or with a compound according to Formula (VI), binds Nurr1 and induces Nurr1 binding to a NBRE, a NuRE, or a DR-5 response element. In embodiments, the compound according to Formula (I) to (V), or with a compound according to Formula (VI), binds Nurr1 and induces Nurr1 binding to a NBRE. In embodiments, the compound according to Formula (I) to (V), or with a compound according to Formula (VI), binds Nurr1 and induces Nurr1 binding to a NuRE. In embodiments, the compound according to Formula (I) to (V), or with a compound according to Formula (VI), binds Nurr1 and induces Nurr1 binding to a DR-5 response element.

In embodiments, the lower levels of Nurr1 results in an impaired function or activity of Nurr1. In embodiments, the lower levels of Nurr1 can be restored by applying a compound according to Formula (I) to (V), or with a compound according to Formula (VI). In embodiments, the compounds according to Formula (I) to (V), or with a compound according to Formula (VI), act as Nurr1 agonists.

### V. Pharmaceutical Compositions

In some embodiments is provided a pharmaceutical composition including a compound according to Formula (I) to (V), or with a compound according to Formula (VI), described herein and a pharmaceutically acceptable excipient.

In some embodiments, the pharmaceutical composition includes a therapeutically effective amount of the compound according to Formula (I) to (V), or with a compound according to Formula (VI).

In some embodiments, the pharmaceutical composition includes a therapeutically-effective amount of a second agent, wherein the second agent is an agent for treating a neurodegenerative disease. In some embodiments, the neurodegenerative disease is Parkinson's disease. In some embodiments, the second agent is a Parkinson's disease drug, for example, levodopa, carbidopa, selegiline, amantadine, donepezil, galantamine, rivastigmine, tacrine, bromocriptine, pergolide, pramipexole, ropinirole, trihexyphenidyl, benztropine, biperiden, procyclidine, tolcapone or entacapone. In some embodiments, the pharmaceutical composition includes a therapeutically effective amount of the second agent.

In some embodiments, the pharmaceutical composition includes a therapeutically-effective amount of a second agent, wherein the second agent is an agent for treating an inflammatory disease, for example, acetaminophen, duloxetine, aspirin, ibuprofen, naproxen, diclofenac, prednisone, beta-methasone, cortisone, dexamethasone, hydrocortisone, methylprednisolone, prednisolone, codeine, fentanyl, hydrocodone, hydromorphone, morphine, meperidine, or oxycodone. In embodiments, the pharmaceutical composition includes a therapeutically effective amount of the second agent.

In some embodiments, the pharmaceutical composition includes a therapeutically-effective amount of a second agent, wherein the second agent is an anti-cancer agent.

### VI. Dosing and Duration of Treatment

In some embodiments, the present invention provides a method of treating a subject, for example, a human patient in need thereof, the method comprising administering to the subject, in particular orally administering to the subject, a compound herein, in particular a compound according Formula (I) to (V), or with a compound according to Formula (VI), and/or a pharmaceutically acceptable salt and/or a solvate, in particular a hydrate, thereof and/or a solvate, in particular a hydrate, of a pharmaceutically acceptable salt thereof at a daily dose of about 12 to 120 μmol, for example, about 12.3 μmol to about 38.2 μmol, about 76.5 μmol or about 115 μmol, for example the daily dose can be about 12.3 μmol to about 38.2 μmol, about 25.5 μmol, or about 26.5 μmol.

In some embodiments, the present invention provides a method of treating a human patient in need the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 12 to 120 μmol vidofludimus or a pharmaceutically acceptable salt or a solvate, in particular a hydrate, thereof or a solvate, in particular a hydrate, of a pharmaceutically acceptable salt thereof so as to thereby treat the human patient, preferably the daily dose is about 12.3 μmol to about 38.2 μmol, about 76.5 μmol or about 115 μmol, more preferably the daily dose is about 12.3 μmol to about 38.2 μmol, and most preferably the daily dose is about 26.5 μmol.

In some embodiments, the present invention provides a method of treating a human patient in need the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 115 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient in need the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 76.5 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient in need the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 12 to 38 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient in need the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 25.5 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In the following table μmol of the active moiety of vidofludimus is converted into mg of the free acid of vidofludimus (i.e., active moiety) and the calcium salt dihydrate of vidofludimus:

| mg Ca | mg | μmol |
|---|---|---|
| (vidofludimus)₂ × 2 H₂O | (vidofludimus) | (vidofludimus) |
| 5.52 | 5 | 14.1 |
| 11.0 | 10 | 28.1 |
| 16.6 | 15 | 42.2 |
| 22.1 | 20 | 56.3 |
| 24.9 | 22.5 | 63.3 |
| 33.1 | 30 | 84.4 |
| 49.7 | 45 | 127 |
| 55.2 | 50 | 141 |
| 77.3 | 70 | 197 |

The daily dose in the following embodiments is based on the active moiety, i.e., the free acid of vidofludimus.

In some embodiments, the present invention provides a method of treating a subject, for example, a human patient in need thereof, the method comprising orally administering to the subject vidofludimus calcium salt dihydrate with the following structure:

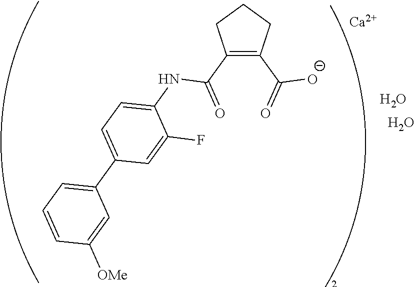

as Polymorph A at a dose of about 5 mg to about 45 mg, for example, the dose is about 5 mg to about 15 mg, about 30 mg or about 45 mg, about 5 mg to about 15 mg, about 45 mg, or about 10 mg. In some embodiments, the administering is daily.

In some embodiments, the present invention provides a method of treating a subject, for example, a human patient in need thereof, the method comprising orally administering to the human patient vidofludimus calcium salt dihydrate with the following structure:

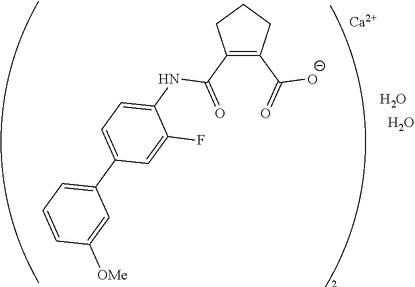

as “Polymorph A” at a daily dose of about 10 mg.

In some embodiments, the present invention provides a method of treating a human patient in need the method comprising orally administering to the human patient vidofludimus calcium salt dihydrate with the following structure:

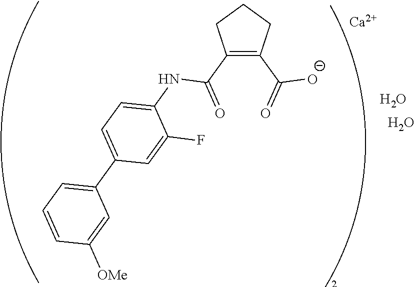

as “Polymorph A” at a daily dose of about 45 mg in the form of a tablet or a capsule.

In some embodiments, the present invention provides a method of treating a human patient in need the method comprising orally administering to the human patient vidofludimus calcium salt dihydrate with the following structure:

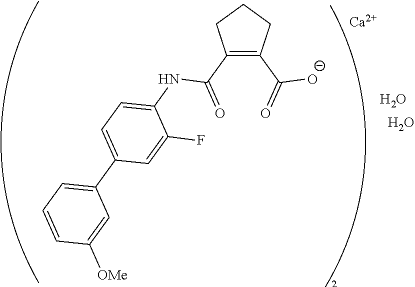

as “Polymorph A” at a daily dose of about 30 mg in the form of a tablet or a capsule.

In some embodiments, the administering is daily.

In some embodiments, the present invention provides a method of treating a human patient in need the method comprising orally administering to the human patient vidofludimus calcium salt dihydrate with the following structure:

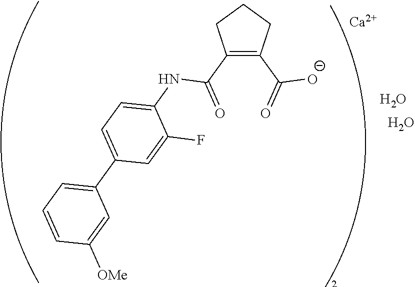

as “Polymorph A” at a daily dose of about 15 mg in the form of a tablet or a capsule during the first week of treatment.

In some embodiments, the present invention provides a method of treating a human patient in need the method comprising orally administering to the human patient vidofludimus calcium salt dihydrate with the following structure:

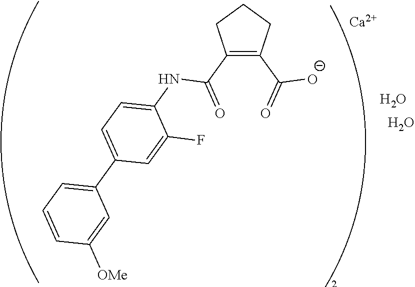

as “Polymorph A” at a daily dose of about 22.5 mg in the form of a tablet or a capsule during the first week of treatment.

### VIII. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Unless defined otherwise, all technical and scientific terms used herein have the same meaning as commonly understood by one of ordinary skill in the art to which the invention pertains.

The term “level or activity” refers to the expression level of either mRNA or protein of the respective target or the activity, which is due to the expression level either of mRNA or protein regulated by the respective target. The activity can also be measured for certain target genes and proteins by means of PET scan or SPECT. In case of dopamine or its metabolites, “activity” means an increase or decrease of the respective molecule.

The term “effective amount” or “therapeutically effective amount” refers to that amount of a compound or pharmaceutical composition described herein that is sufficient to affect the intended application including, but not limited to, disease treatment or biomarker/target gene outcome, as illustrated below. The therapeutically effective amount can vary depending upon the intended application, or the subject and disease condition being treated, e.g., the weight (e.g. as assessed by the body mass index (BMI)), age and/or gender, the severity of the disease condition, the manner of administration and the like, the response towards a biomarker or Nurr1 target gene and the like, which can readily be determined by one of ordinary skill in the art. The specific dose will vary depending on, for example, the particular compounds chosen, the dosing regimen to be followed, whether it is administered in combination with other agents, timing of administration, duration of treatment, the tissue to which it is administered, and the physical delivery system in which it is carried.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically engineered animal, or a clone.

In some embodiments, a subject can be any member of the animal kingdom. Subjects can be, for example, humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, and infants. In some embodiments, a subject is a patient.

Subjects can be of a certain age, such as, for example, about 1 to about 5 years old, about 5 to about 10 years old, about 10 to about 15 years old, about 15 to about 20 years old, about 20 to about 25 years old, about 25 to about 30 years old, about 30 to about 35 years old, about 35 to about 40 years old, about 40 to about 45 years old, about 45 to about 50 years old, about 50 to about 55 years old, about 55 to about 60 years old, about 60 to about 65 years old, about 65 to about 70 years old, about 70 to about 75 years old, about 75 to about 80 years old, about 80 to about 85 years old, about 85 to about 90 years old, about 90 to about 95 years old, about 95 to about 100 years old, about 1 to 8 years old, about 1 to 10 years old, about 1 to 12 years old, about 1 to 14 years old, about 1 to 16 years old, about 1 to 18 years old, about 1 to 20 years old, about 20 to 40 years old, about 20 to 60 years old, about 20 to 80 years old, about 20 to 100 years old, about 40 to 80 years old, or about 60 to 80 years old.

In some embodiments, a subject is at least about 1 year old, at least about 5 years old, at least about 10 years old, at least about 12 years old, at least about 14 years old, at least about 16 years old, at least about 18 years old, at least about 20 years old, at least about 25 years old, at least about 30 years old, at least about 40 years old, at least about 50 years old, at least about 60 years old, at least about 70 years old, or at least about 80 years old. In some embodiments, a subject is no more than about 1 year old, no more than about 5 years old, no more than about 10 years old, no more than about 12 years old, no more than about 14 years old, no more than about 16 years old, no more than about 18 years old, no more than about 20 years old, no more than about 25 years old, no more than about 30 years old, no more than about 40 years old, no more than about 50 years old, no more than about 60 years old, no more than about 70 years old, or no more than about 80 years old.

In some embodiments the subject in need of the above-mentioned method for treating MS belongs to the patient group with a paediatric-onset MS (POMS), which means an onset until the age of 18.

In some embodiments the subject in need of the above-mentioned method for treating MS belongs to the patient group with a late-onset MS (LOMS), which means an onset in the time period from 19-50 years.

In some embodiments the subject in need of the above-mentioned method for treating MS belongs to the patient group with an adult-onset MS (AOMS), which means an onset >50 years.

Subjects can have a certain body mass index (BMI), such as, for example, about 15 kg/m² to about 18 kg/m², about 15 kg/m² to about 18.5 kg/m², about 18.5 kg/m² to about 24.9 kg/m², about 25 kg/m² to about 29.9 kg/m², or about 30 kg/m² to about 40 kg/m². In some embodiments, a subject has a BMI of at least about 15 kg/m², at least about 18.5 kg/m², or at least about 25 kg/m². In some embodiments, a subject has a BMI of no more than about 25 kg/m², no more than about 30 kg/m², or no more than about 40 kg/m².

The terms “agonist”, “activator”, “upregulator” etc. refer to a substance capable of detectably increasing the expression or activity of a given gene or protein. The agonist can increase expression or activity by at least 5%, 10%, 20%, 30%, 40%, 50%, 60%, 70%, 80%, 90%, 95%, 96%, 97%, 98%, or 99% in comparison to a control in the absence of the agonist. In certain instances, expression or activity is 1.2-fold, 1.3-fold, 1.4-fold, 1.5-fold, 2-fold, 3-fold, 4-fold, 5-fold, 10-fold or higher than the expression or activity in the absence of the agonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. For example, as applied to the effects of a modulator on a target gene, to modulate means to change by increasing or decreasing expression of the gene or the activity of the gene.

The term “aberrant” as used herein refers to different from normal. When used to describe enzymatic activity, aberrant refers to activity that is greater or less than a normal control or the average of normal non-diseased control samples. In one embodiment the aberrant level is at least 10%, preferably at least 20%, more preferably at least 30%, even more preferably at least 40%, yet more preferably at least 50% different from the level of a healthy subject. In particular, the upregulated level is at least 10%, preferably at least 20%, more preferably at least 30%, even more preferably at least 40%, yet more preferably at least 50% higher from the level of a healthy subject. In particular, the downregulated level is at least 10%, preferably at least 20%, more preferably at least 30%, even more preferably at least 40%, yet more preferably at least 50% lower from the level of a healthy subject. When used to describe a target gene level, aberrant refers to a gene expression that is greater or less than a normal control or the average of normal non-diseased control samples. Aberrant activity may refer to an amount of activity that results in a disease, wherein returning the aberrant activity to a normal or non-disease-associated amount (e.g., by using a method as described herein), results in reduction of the disease or one or more disease symptoms.

A “week” preferably refers to a period of or about 5, about 6 or about 7 days. It may be about 5-8 days. Most preferred week is a period of 7 days.

A “month” preferably refers to a period of or about 28, about 29, about 30 or about 31 days. It may be about 26-33 days. Most preferred month is a period of 30 days.

As used herein, the term “evening” refers to the period of time between late afternoon and early nightfall, preferably from 16:00 to 23:00 local time.

As used herein, the term “morning” refers to the period of time from the early hours of dawn until approximately midday, preferably from 5:00 to 12:00 local time.

The term “about” as used herein with respect to numbers, figures, ranges and/or amounts is preferably meant to mean “circa” and/or “approximately”. The meaning of those terms is well known in the art and preferably includes a variance, deviation and/or variability of the respective number, figure, range and/or amount of plus/minus 15% and especially of plus/minus 10%.

“Treatment” as used herein preferably comprises the sequential succession of an “induction treatment” and then the “maintenance treatment”. For example, a treatment according to the invention comprises an induction treatment for about one week in which the half daily dose is administered, followed by maintenance treatment in which the full daily dose is administered (see e.g. WO 2019/101888).

“Daily dose” preferably refers to the total dose of a compound according to Formula (I) to (V), or with a compound according to Formula (VI), preferably vidofludimus, or a pharmaceutically acceptable salt or solvate thereof, orally administered to the patient each day of administration. The daily dose can be reached through a single or several administrations per day, such as for example once a day, twice a day or three times a day. Preferably, it is reached or achieved by single administration per day, preferably consisting of one or more tablets or capsules, preferably tablets or capsules as described herein.

“Relapses” preferably involve neurologic problems that occur over a short period, typically days but sometimes as short as hours or even minutes. These attacks most often involve motor, sensory, visual or coordination problems early in the disease. Later, bladder, bowel, sexual and cognitive problems may be shown. Sometimes the attack onset occurs over several weeks. Typical MS relapse involves a period of worsening, with development of neurological deficits, then a plateau, in which the patient is not getting any better but also not getting any worse followed by a recovery period. Recovery usually begins within a few weeks.

The “annualized relapse rate” is the average number of relapses a group of patients in a clinical study have in one year. See e.g. Multiple Sclerosis Coalition. The Use Of Disease—Modifying Therapies In Multiple Sclerosis: Principles and Current Evidence Summary. Available at http://www.nationalmssociety.org/getmedia/1e64b96c-9e55-400e-9a64-Ocdf5e2d60fe/summaryDMTpaper-final.

The “hazard ratio” is measure of how often a particular event happens in one group compared to how often it happens in another group over time. A hazard ratio of exactly 1.0 means that the study drug provides zero risk reduction, compared to the control treatment.

The “Expanded Disability Status Scale” (EDSS) is a clinician-reported outcome measure for quantifying changes in the disability level of a subject with MS overtime. The EDSS is based on a standard neurological examination, incorporating functional systems (visual, brainstem, pyramidal, cerebellar, sensory, bowel and bladder, and cerebral [or mental]) that are rated and then scored as a functional system score (FSS), and ambulation, which is scored as ambulation score. Each FSS is an ordinal clinical rating scale ranging from 0 to 5 or 6, and an ambulation score that is rated from 0 to 12. These ratings may then be used in conjunction with observations, as well as information, concerning ambulation and use of assistive devices to determine the total EDSS score. The EDSS is a disability scale that ranges in 0.5-point steps from 0 (normal) to 10.0 (death) (Kurtzke, Neurology 1983; 33:1444). In some embodiments of the methods provided herein, the item sexual dysfunction and fatigue are not included in the EDSS score. Typically, a decrease in EDSS score corresponds to an improvement in the disease and conversely, an increase in EDSS score corresponds to a worsening of the disease.

The “9-Hole Peg Test” (9-HPT) is a quantitative measure of upper extremity (arm and hand) function (Arch. Phys. Med. Rehabil. 1988; 69:850). The test device consists of a container with nine pegs and a block containing nine empty holes. The subject is to pick up each of the nine pegs one at a time and as quickly as possible place them in the nine holes. Once all the pegs are in the holes, the subject is to remove them again one at a time as quickly as possible and replace them into the container. The total time to complete the task is recorded. Both the dominant and non-dominant hands are tested twice (two successfully completed trials of the dominant hand, followed immediately by two successfully completed trials of the non-dominant hand). The two trials for each hand are averaged, converted to the reciprocals of the mean times for each hand, and the two reciprocals are averaged. The 9-HPT may be administered, for example, as described in the Multiple Sclerosis Functional Composite (MSFC) Administration and Scoring Manual (National Multiple Sclerosis Society, 2001). A meaningful change in upper extremity function may, for example, be indicated by a 20% worsening from baseline of the averaged 9-HPT times.

The “Timed 25-Foot Walk Test” (T25FWT) is a quantitative measure of mobility and leg function, based on a timed 25-foot walk. The subject is directed to start at one end of a clearly marked 25-foot course and is instructed to walk 25 feet as quickly and safely as possible, and how long it takes the subject to go from start of the walk to the end of the 25 feet is timed. In some embodiments, the task is administered immediately again by having the subject walk back the same distance, and the time for both completed trials averaged to produce the score for the T25FWT. Subjects may use assistive devices (e.g., cane or wheelchair) when performing the task. The T25FWT may be administered, for example, as described in the MSFC Administration and Scoring Manual. A clinically meaningful change in mobility and leg function may, for example, be indicated by a 20% worsening from baseline of the averaged T25FWT time.

The “Symbol Digit Modalities Test” (SDMT) is a test used to evaluate the presence of cognitive impairment and/or changes in cognitive functioning over time and in response to treatment. The SDMT may be particularly sensitive to slowed processing of information that is commonly seen in MS (Mult. Scler. 2017; 23:721). The SDMT comprises a substitution task. Using a reference key, the subject has 90 seconds to pair specific numbers with given geometric figures. Responses may be collected orally, and the number of correct responses is considered the SDMT score. A clinically meaningful change in cognitive processing may, for example, be indicated by a decrease by 4 points on the SDMT score from baseline.

The “Columbia-Suicide Severity Rating Scale” (C-SSRS) is a tool used to assess the lifetime suicidality of a subject and may be used to track suicidal events through treatment or a portion thereof. The structured interview prompts recollection of suicidal ideation, including the intensity of the ideation, behavior, and attempts with actual/potential lethality. A “baseline” C-SSRS may include, for example, C-SSRS collected prior to beginning administration of a compound of Formula (I) to (V), or with a compound according to Formula (VI), or an isotopic variant, a pharmaceutically acceptable salt or solvate thereof, especially vidofludimus. Such score may be compared, for example, to subsequent C-SSRS collected after beginning administration of a compound of Formula (I) to (V), or with a compound according to Formula (VI), or an isotopic variant, a pharmaceutically acceptable salt or solvate thereof, especially vidofludimus. Comparisons between different e valuation periods (which may, for example, occur during visits with a clinician) may be described, in some embodiments, as “since last visit” C-SSRS.

The “EQ-5D-5L” is a validated self-reported health status questionnaire that can used to calculate a health status utility score for use in health economic analyses (Qual. Life Res. 2011; 20:1727; Qual. Life Res. 2013; 22:1717). There are two components to the EQ-5D-5L: a five-item health state profile that assesses mobility, self-care, usual activities, pain/discomfort, and anxiety/depression, as well as a visual analog scale (VAS) that measures health state. The EQ-5D-5L is designed to capture a subjects current health status. Published weighting systems may allow for creation of a single composite score of the subject's health status. The “Multiple Sclerosis Impact Scale-29 Version 2” (MSIS-29, Version 2) is a 29-item subject-reported measure of the physical and psychological impacts of MS (Brain 2001; 124:962). Subjects are asked to rate how much their functioning and well-being has been impacted over the past 14 days on a 4-point scale, from “Not at all” (1) to “Extremely” (4). The physical score is the sum of items 1 to 20, which is then transformed to a 0-100 scale. The psychological score is the sum of items 21-29, transformed to a 0-100 scale. Higher scores may indicate a greater impact of MS. A clinically meaningful impact is indicated by a change of at least 7.5 points on the physical scale in Version 1 of the MSIS-29. In Version 2 of the MSIS-29, this level of change may also indicate a meaningful impact.

The “Multiple Sclerosis Walking Scale, 12-Item” (MSWS-12) is a 12-item self-report measure of the impact of MS on the individual's ability to walk during the past 2 weeks. Each item is scored on a 5-point Likert scale, and total scores are converted to a 0-100 scale with higher scores indicating greater impact of MS on walking ability.

The “Quality of Life in Neurological Disorders, Upper Extremity” (fine motor skills and activities of daily living; Neuro-QoL, Upper Extremity) is a 20-item questionnaire used to assess upper limb function, which involves subjects with MS through each stage of its development (Qual. Life Res. 2012; 21:475). Items include assessments of dressing, cooking, eating, cleaning, and writing from which the subject uses a 5-point Likert scale to rate his or her performance ranging from “without any difficulty” (5) to “unable to do” (1). Item scores are summed, multiplied by 20 and divided by 20 minus the number of any unanswered items. Scores range from 20-100, where a higher score indicates better upper limb function. In accordance with the NINDS User Manual (2015), scores can be calculated as long as at least 50% of the items have been answered.

The “PROMIS-FatigueMS” is an 8-item scale developed as a measure of fatigue for subjects with MS (Qual. Life Res. 2012; 21:1021) with a recall period of the previous 7 days. It comprises a 5-point Likert-type scale that produces a score between 1 and 5 for each scored question. The total raw score is the sum of the values of each scored question. The total raw score ranges from 8-40. Scores can also be transformed to a PROMIS T-score where the mean is 50 and a standard deviation of 10. T-scores range from 34.7-81.3. A higher score is associated with worse fatigue.

The “Patient Global Impression of Change” (PGI-C) is a single-item assessment of a subject's impression of his or her change in MS symptoms compared with a point 6 months previous. Subjects respond on a 7-point Likert scale from “very much better” (i) to “very much worse” (7). The PGI-C is used as an anchor for determining what is a clinically meaningful change in the MSIS-29.

The “Patient Global Impression of Severity” (PGI-S) is a single-item assessment of a subject's impression of the severity of his or her MS symptoms from the past 7 days. A subject respond on a 5-point Likert scale from “none” (1) to “very severe” (5). The PGI-S is used as an anchor for determining what is a clinically meaningful change in the MSIS-29.

The “Work Productivity and Activity Impairment: Multiple Sclerosis” (WPAI:MS) is a 6-item scale. A subject estimates the amount of time that their work and daily activities were affected by their MS over the previous 7 days (Pharmacoeconomics 1993; 4:353). The WPAI:MS assesses absenteeism as well as “presenteeism,” which accounts for the time when a subject was present for work or activities, but believed their health had a negative effect on their ability to perform at the usual level. A higher score represents a greater impairment in productivity.

“Confirmed Disability Progression” (CDP) refers to an increase in the subject's EDSS score that is sustained over a particular time period. This may be evaluated, for example, by calculating the subject's EDSS score, determining that the score is increased over a previous score (such as a baseline score, which may be a score taken before the subject began administration of a compound of Formula (I) to (V), or with a compound according to Formula (VI), or an isotopic variant, a pharmaceutically acceptable salt or solvate thereof, especially vidofludimus), and then confirming the score is still increased after a specified period of time has elapsed from the initial increase (e.g., by reevaluating the subject and recalculating it again). For example, a 12-week confirmed disability progression (CDP12) refers to an EDSS score that remains increased at least 12 weeks after the initial increase (e.g., as confirmed by recalculating the EDSS score at least 12 weeks after the initial increase). A 24-week confirmed disability progression (CDP24) refers to an EDSS score remains increased at least 24 weeks after the initial increase (e.g., as confirmed by recalculating the EDSS score at least 24 weeks after the initial increase). The initial increase may be compared to a baseline EDSS score (such as prior to beginning administration of a compound of Formula (I) to (V), or with a compound according to Formula (VI), or an isotopic variant, a pharmaceutically acceptable salt or solvate thereof, especially vidofludimus), or may be compared to a prior EDSS score that had remained stable over time, such as over 12, 24, 36, 48, or 60 weeks. In some embodiments, a CDP refers to an increase of ≥1.0 point from the baseline EDSS score in a subject with a baseline EDSS score of ≤5.5 points, or an increase of ≥0.5 point from the baseline EDSS score in a subject with a baseline EDSS score of ≥5.5 points. Time to onset of a CDP (e.g., time to onset of CDP 12 or CDP24) refers to the time period from when the prior EDSS score was established (for example, a baseline EDSS score from before beginning administration of vidofludimus, or an isotopic variant, pharmaceutically acceptable salt or solvate thereof) until the sustained increase of EDSS score is observed.

“Composite Confirmed Disability Progression” (cCDP) is a composite measure of disability progression using a combination of EDSS, 9-HPT and T25FWT. It evaluates the progression of subject's disability over a particular time period as determined by the first occurrence of a progression event. A progression event may include any one of the following: a CDP (e.g., increase of ≥1.0 point from the baseline EDSS score in a subject with a baseline EDSS score of ≤5.5 points, or an increase of ≥0.5 point from the baseline EDSS score in a subject with a baseline EDSS score of ≥5.5 points); an increase of ≥20% from baseline in time to complete the 9-Hole Peg Test (9-HPT); or an increase of ≥20% from baseline in the Timed 25-Foot Walk Test (T25FWT); wherein the occurrence of the progression event is confirmed at after a specified period of time has elapsed from the initial occurrence. For example, a composite 12-week continued disability progression (cCDP12) refers to the occurrence of at least one progression event at an initial time point, and the same progression event is confirmed at least 12 weeks later (e.g., by re-evaluating the subject using the same test). A composite 24-week confirmed disability progression (cCDP12) refers to the occurrence of at least one progression event at an initial time period, and same progression event is confirmed at least 24 weeks later. Time to onset of a cCDP (e.g., time to onset of cCDP12 or cCDP24) refers to the time period from when the prior evaluation scores were established (for example, baseline scores before beginning administration of vidofludimus, or an isotopic variant, pharmaceutically acceptable salt or solvate thereof) until the initial progression event is observed. Without wishing to be bound by theory, compared with endpoints based exclusively on the Expanded Disability Status Scale (EDSS), which emphasizes lower limb function, the cCDP12 requires at least one of the following: 1) an increase in EDSS score of ≥1.0 point from a baseline (BL) score of ≤5.5 points, or ≥0.5 point increase from a BL score of ≥5.5 points (Confirmed Disability Progression); 2) a 20% increase from BL in time to complete the 9-Hole Peg Test; 3) a 20% increase from BL in the Timed 25-Foot Walk Test. Thus, the cCDP 12 is a more sensitive assessment of disability, especially at early disease stages. The use of the cCDP12 as a primary outcome may provide a clearer, more complete picture of disability progression or improvement than the EDSS alone.

The “Confirmed Disease Worsening” (CDW), measures the increase in a patient's EDSS score that is sustained over a pre-determined time period, which means a patient's physical disability has increased as described in e.g. Drugs 2015; 75:947.

In some embodiments the confirmed disease progression is measured by Kurtzke Expanded Disability Status Scale (EDSS) score in a subject having MS in need of.

In some embodiments the confirmed disease progression is at least a 1 point increase of the EDSS score.

In some embodiments, the MS patient had confirmed disease progression of at least a 0.5 point increase of the EDSS score.

In some embodiments, the hazard ratio for no confirmed disability worsening is decreased by 20-60%.

In some embodiments, the hazard ratio for no confirmed disability worsening is decreased by 30-50%.

In some embodiments, the hazard ratio for no confirmed disability worsening is decreased by at least 30%.

In some embodiments, the hazard ratio for no confirmed disability worsening is decreased by at least 40%.

In some embodiments, the hazard ratio for no confirmed disability worsening is decreased by at least 50%.

The term “brain atrophy” describes one of the most destructive consequences of MS. Brain atrophy can be seen in the earliest stages of MS and may lead to irreversible neurological and cognitive impairments. Progressive loss of brain tissue bulk can be detected in vivo in a sensitive and reproducible manner by MRI. See e.g. Lancet Neurol. 2006; 5:158.

“Efficacy” of a treatment according to the invention can be preferably measured based on changes in the course of disease in response to a use according to the invention. For example, treatment of MS efficacy can be measured by the frequency of relapses in RRMS and the presence or absence of new lesions in the CNS as detected using methods such as the MRI technique (Neurology 1996; 47(Suppl 4): S217; Ann. Neurology 1997; 41:125).

Preferably, the observation of the reduction and/or suppression of MRI T1 gadolinium-enhanced lesions (thought to represent areas of active inflammation) gives a primary efficacy variable. Shows active lesions that appear bright white on an MRI scan after administration of an intravenous imaging contrast agent (gadolinium). Secondary efficacy variables preferably include MRI T1 enhanced brain lesion volume, MRI T1 enhanced lesion number, MRI T2 lesion volume (thought to represent total disease burden, i.e. demyelination, gliosis, inflammation and axon loss), MRI T1 enhanced hypointense lesion volume (thought to represent primarily demyelination and axon loss), time-to-progression of MS, frequency and severity of exacerbations and time-to-exacerbation, Expanded Disability Status Scale score and Scripps Neurologic Rating Scale (SNRS) score (Neurology 1984; 34:1368). Methods of early and accurate diagnosis of multiple sclerosis and of following the disease progression are described in Mattson, Expert Rev. Neurother. 2002; 2:319.

White matter lesions on brain MRI in MS may contribute to misdiagnosis, especially when trying to assign a patient to a subgroup. A subset of MS lesions shows paramagnetic rims on susceptibility-weighted MRI sequences, reflecting iron accumulation in microglia. These para-magnetic rim lesions have been proposed as a marker of compartmentalized smoldering disease (AJR 2022; 219:120). Paramagnetic rim lesion detection occurs rarely in other neurological conditions (52% of MS vs 7% of non-MS cases) and yielded high specificity (93%) in differentiating MS from non-MS (Ann Neurol. 2020; 88:1034). Paramagnetic rim lesions may be an emerging marker of chronic neuroinflammation in MS and could help to assign a MS patient to a subgroup, e.g. PPMS.

As used herein, the term “effective amount” includes a dosage sufficient to produce a desired result with respect to the indicated disorder, condition, or mental state. The desired result may comprise a subjective or objective improvement in the recipient of the dosage.

As used herein, the term “administering” includes activities associated with providing a patient an amount of a compound according to Formula (I) to (V), or with a compound according to Formula (VI), preferably vidofludimus, or a pharmaceutically acceptable salt or solvate thereof. Administering includes providing unit dosages of compositions set forth herein to a patient in need thereof. Administering includes providing effective amounts of compounds, e.g. vidofludimus and/or a pharmaceutically acceptable salt and/or a solvate, in particular a hydrate, thereof and/or a solvate, in particular a hydrate, of a pharmaceutically acceptable salt thereof, for a specified period of time, e.g. for about 6, 9, 12, 15 or more months, or about 1, 2, 3, 4, 5 or more years.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is an inflammatory disease. In embodiments, the disease is a cancer.

In some embodiments, a level of Nurr1, a level of activity of a gene downstream of Nurr1, or a level of a protein downstream of Nurr1 (e.g., NCAM, CFB, LTA, A2M, HSD11B1, BHLHE41, MARCO, BDNF, GDNF, C-RET, GFAP, DAT, Pitx3, TH, VMAT2, SOD1, AADC, TNFα, iNOS, or IL-10) is measured in a subject, and then compared to the analogous value obtained from a healthy control subject that has the same biological sex, a similar or identical body mass index (BMI), or a similar or identical age relative to the subject. For example, the healthy control subject can have an age that is within ±1 year, ±2 years, ±3 years, ±4 years, ±5 years, ±10 years, ±15 years, ±20 years, or ±30 years of the age of the subject. In some embodiments, the healthy control subject has a BMI that is within ±1 kg/m², ±2 kg/m², ±3 kg/m², ±4 kg/m², ±5 kg/m², ±10 kg/m², ±15 kg/m², or ±20 kg/m² of the BMI of the subject. Age, BMI, and biological sex can be independently assessed in the subject or the healthy control subject when a level of a protein (eg., Nurr1) or gene is measured.

In some embodiments, a level of Nurr1, a level of activity of a gene downstream of Nurr1, or a level of a protein downstream of Nurr1 in a sample (e.g., NCAM, CFB, LTA, A2M, HSD11B1, BHLHE41, MARCO, BDNF, GDNF, C-RET, GFAP, DAT, Pitx3, TH, VMAT2, SOD1, AADC, TNFα, iNOS, or IL-1β) is measured in a subject, and then compared to a level of Nurr1, a level of activity of a gene downstream of Nurr1, or a level of a protein downstream of Nurr1 in another sample of the subject to see decline or decrease of said level.

In some embodiments, a level of Nurr1, a level of activity of a gene downstream of Nurr1, or a level of a protein downstream of Nurr1 in a sample (e.g., GDNF, GFAP, or VMAT2) is measured in a subject more than once and then compared to a level of Nurr1, a level of activity of a gene downstream of Nurr1, or a level of a protein downstream of Nurr1 in another sample of the subject to monitor the decline or decrease of said level over time.

Non-limiting examples of a mental condition include anxiety, depression, bipolar disorder, dementia, schizophrenia, and other psychoses.

Non-limiting examples of a central nervous system condition or disease include neurological diseases or disorders that affect the structure or function of the brain or spinal cord, which collectively form the central nervous system (CNS), for example addiction, encephalitis, Parkinson's and multiple sclerosis.

Non-limiting examples of a neurodegenerative disease or condition include a disease or condition in which the function of a subject's nervous system becomes impaired, for example, Alper's disease, Alzheimer's disease, amyotrophic lateral sclerosis (ALS), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, corticobasal syndrome (CBS), Creutzfeldt-Jakob disease, Down syndrome (DS), frontotemporal dementia, Gerstmann-Straussler-Scheinker syndrome, Huntington's disease, HIV-associated dementia (FTD), Kennedy's disease, Krabbe's disease, kuru, dementia with Lewy bodies (DLB), Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, progressive supranuclear palsy (PSP), Refsum's disease, Sandhoffs disease, Schilder's disease, subacute combined degeneration of spinal cord secondary to pernicious anemia, schizophrenia, spinocerebellar ataxia (multiple types with varying characteristics), spinal muscular atrophy, Steele-Richardson-Olszewski disease, and tabes dorsalis.

Multiple sclerosis (MS) can be divided to relapsing forms of MS (RMS) and progressive forms of MS (PMS). Relapsing-remitting MS (RRMS) and active secondary progressive MS (active SPMS) are primarily driven by focal inflammatory disease and characterized by the presence of magnetic resonance imaging (MRI) lesions and relapses. It is however known from a recent meta-analysis of a large patient database that disability worsening in these two subtypes are driven by relapse-associated worsening (RAW) and by progression independent of relapse activity (PIRA). RRMS is characterized by the domination of relapses and MRI lesions over the clinical course. Active SPMS (aSPMS) is characterized by fewer relapses and lesions with continuous disability progression (see FIG. 8).

Primary progressive MS (PPMS) and non-active secondary progressive MS (non-active SPMS or n-aSPMS) are the two predominantly progressive forms of MS, which are characterized by an ongoing disability worsening without or almost without any MRI lesions or relapses being present. It is known from a recent meta-analysis of a large patient database that disability worsening in these two subtypes are almost exclusively driven by PIRA. Non-active SPMS is characterized by continuous disability progression while relapses have stopped. PPMS is characterized by disability worsening from the start (see FIG. 8).

Definitions and subcategorizations of PPMS and SPMS may vary. In one exemplary definition, all SPMS may be considered to belong to the category of PMS, so that all SPMS patients considered to be PMS patients, with no differentiation between aSPMS and n-aSPMS. In another exemplary definition, active-SPMS may be considered to belong to the category of RMS, while non-active-SPMS may be considered to belong to the category of PMS. Thus, the terms “n-aSPMS” and “SPMS” are used synonymously in the present invention.

The separation of PIRA compared to RAW can be as follows (JAMA Neurol. 2023; 80:151): A PIRA event can be defined as experiencing confirmed disability worsening (CDW) in the EDSS scale at 6 months during a period free of relapses (PFRs). A PFR is the time between two consecutive relapses, starting 3 months after a relapse (or 6 months after the first demyelinating event). The first EDSS score obtained at least 6 months after the first attack or 3 months after any other attack was referred to as the baseline EDSS score and rebaseline EDSS score, respectively. It was set that no rebaseline EDSS score could be lower than the first recorded (baseline) EDSS score. Confirmed disability accumulation (CDA) was defined as an increase in the EDSS score of 1.5, 1.0, or 0.5 if the baseline/rebaseline EDSS score was, respectively, 0, 1.0 to 5.0, or greater than 5.0. The date of PIRA was the date of the confirmation of the CDA. Any other episodes of CDA that did not qualify for PIRA (i.e., which occurred outside the PFR) were considered to be RAW events. Those patients with at least 1 CDA but who did not present with any PIRA event were considered patients with RAW.

In the clinical study of Kopp et al. (Mult. Scler. Relat. Disord. 2021; 56:103319) the following inclusion criteria were applied on a MS population with a diagnosis of clinical SPMS assigned by an MS-neurologist and RRMS patients fulfilling the MSBase diagnostic definition for conversion to SPMS. This m-EXPAND criteria identify patients with recent worsening on the EDSS score likely not explained by a recent relapse:
(a) An EDSS from 3.0 to 6.5 (both inclusive) (at index date+/−6 months); and
(b) EDSS progression within the last 2 years before data extraction, defined as EDSS progression of 1 point or more in patients with an EDSS score of less than 6.0 or ≥0.5 point in patients with EDSS score≥6.0, in the absence of relapses 6 months prior to progression and EDSS ≥3.0 at time of progression; and
(c) Disability progression.

Currently, there is no FDA-approved treatment option for SPMS other than mitoxantrone. However, for treatment with mitoxantrone, no distinction regarding relapse activity (active SPMS vs non-active SPMS) is made. Notably, treatment of mitoxantrone is associated with significant toxicities including cardiotoxicity and secondary malignancy, which limits the length of treatment to ˜3 years (lifetime dose of 140 mg/m²). In addition, patient populations in this trial were predominately with relapsing type of SPMS, and the benefit in non-active SPMS is unclear. Siponimod, natalizumab, and interferon beta-1b are approved for active SPMS but have not shown benefit in non-active SPMS. For PPMS patients, ocrelizumab has been approved, however due to the strong attenuation of the immune response it contains a black box warning towards progressive multifocal leukoencephalopathy and immune-mediated colitis. Other treatment options currently being tested in clinical trials (e.g. fenebrutinib with risk of liver damages) also do not offer the favorable safety profile of vidofludimus. Therefore, there is an urgent unmet need given the lack of effective therapies for PIRA in its entirety.

MS usually begins with a clinically isolated syndrome (CIS). This is the first episode of symptoms caused by inflammation and damage to the myelin covering on nerves in the brain or spinal cord. In CIS, a person has an attack suggestive of demyelination, but does not fulfill the criteria for MS. 30 to 70% of persons experiencing CIS later develop MS.

There exists an additional subgroup of MS patients, i.e. patients with MS transitioning between relapsing MS and PIRA. For this disease the term “transitioning MS” is used herein. Transitioning MS patients can be identified by one or more of the following test methods:
(a) The symbol digit modalities test (SDMT),
(b) Multiple Sclerosis Functional Composite (MSFC),
(c) EDSS,
(d) timed 25-foot walk (T25FW),
(e) 9-hole peg test (9HPT)
(f) The MSProDiscuss™ clinical tool
(g) Composite scores integrating several tests like e.g. EDSS, timed 25-foot walk (T25FW), SDMT and 9-hole peg test (9HPT), MSProDiscuss™ clinical tool.

As used herein, the term “neuroprotection” refers to the relative preservation of neuronal structure and/or function, i.e. to a therapeutic strategy for slowing or preventing the otherwise irreversible loss of neurons over time. In the case of an ongoing insult (a neurodegenerative insult) the relative preservation of neuronal integrity implies a reduction in the rate of neuronal loss over time. It is a widely explored treatment option for many CNS disorders including neurodegenerative diseases, stroke, traumatic brain injury, spinal cord injury, and acute management of neurotoxin consumption (i.e. methamphetamine overdoses). Neuroprotection aims to prevent or slow disease progression and secondary injuries by halting or at least slowing (mitigate) the loss of neurons. Despite differences in symptoms or injuries associated with CNS disorders, many of the mechanisms behind neurodegeneration are the same. Common mechanisms of neuronal injury include decreased delivery of oxygen and glucose to the brain, energy failure, increased levels in oxidative stress, mitochondrial dysfunction, excitotoxicity, inflammatory changes, iron accumulation, and protein aggregation.

As used herein, the term “inflammatory disease” or “inflammatory condition” refers to an inflammatory disease or condition can be, for example, a disease or condition characterized by aberrant inflammation (e.g., an increased level of inflammation compared to a control such as a healthy person not suffering from a disease). Non-limiting examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

Non-limiting examples of cancer include neoplasm, malignant tumors, leukemia, lymphoma, carcinomas, sarcomas, cancer of the thyroid, endocrine system, brain, breast, cervix, colon, head and neck, liver, kidney, lung, non-small cell lung, ovary, sarcoma, stomach, or uterus, melanoma, mesothelioma, medulloblastoma, colorectal cancer, pancreatic cancer, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

Non-limiting examples of leukemia include progressive, malignant diseases of the blood-forming organs characterized by a distorted proliferation and development of leukocytes and precursors in the blood and bone marrow, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Non-limiting examples of lymphoma include cancers affecting hematopoietic and lymphoid tissues, non-Hodgkin lymphoma (NHL), Hodgkin's disease, high grade NHL, low grade NHL, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, precursor B-lymphoblastic lymphoma, cutaneous T-cell lymphoma, peripheral T-cell lymphoma, anaplastic large cell lymphoma, mycosis fungoides, and precursor T-lymphoblastic lymphoma.

Non-limiting examples of sarcoma includes tumors made of a substance like the embryonic connective tissue and are generally composed of closely packed cells embedded in a fibrillar or homogeneous substance. Sarcomas that may be treated with a compound or method provided herein include a, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of melanomas include tumors arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, or superficial spreading melanoma.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In some embodiments, the present disclosure provides a composition comprising a population of molecules, wherein each molecule of the population is independently a compound of Formula (I), (II), (III), (IV), (V) or (VI), wherein each molecule in a proportion of the population independently comprises a deuterium atom. The level of deuterium incorporation can be any value, such as a natural abundance or a level that is greater than or lesser than a natural abundance. The level of protium in a sample can be any value, such as a natural abundance or a level that is greater than or lesser than a natural abundance. The level of tritium can be any value, such as a natural abundance or a level that is greater than or lesser than a natural abundance.

It will be recognized that some variation of natural isotopic abundance occurs in a synthesized compound depending upon the origin of chemical materials used in the synthesis. Thus, a preparation of compounds according to Formula (I) to (V), or with a compound according to Formula (VI), will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Comp. Biochem. Physiol. 1998; 119A: 725.

When a chemical name or structure is silent as to whether a particular position in a compound normally occupied by hydrogen is isotopically enriched, it is intended that the particular position is occupied by hydrogen at its natural abundance. By way of example, the term “phenyl” or

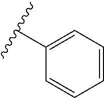 [SMILES: *c1ccccc1]

without any further designation as to isotopic enrichment indicates that all hydrogen atoms are present at natural abundance.

In some embodiments, each molecule in the proportion of the population is substituted with a deuterium atom at the same position. The proportion of molecules of the population that comprise a deuterium atom at a specific atomic position can be represented by an isotopic enrichment factor ε_{p/s}, where ε_{p/s}=(Rₚ/Rₛ−1)×1000, where Rₚ is the abundance of deuterium at that position within the population of molecules, and Rₛ is the natural abundance of deuterium at the position. The proportion of molecules of the population that comprise a deuterium atom at a specific atomic position can alternatively be represented by the molar percent of the population that is substituted with deuterium at that atomic position (i.e., % deuterium incorporation).

Non-limiting examples of an isotopic enrichment factor include at least 835 (12.5% deuterium incorporation), at least 1670 (25% deuterium incorporation), at least 3500 (52.5% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Non-limiting examples of an abundance of deuterium in a sample of a compound herein include a natural abundance, and abundance that is at least 3340 times of the natural abundance of deuterium, which is 0.015% (i.e., at least 50.1% incorporation of deuterium), at least 3500 times of the natural abundance of deuterium (52.5% deuterium incorporation), at least 4500 times of the natural abundance of deuterium (67.5% deuterium incorporation), at least 5000 (75% deuterium), at least 5500 times of the natural abundance of deuterium (82.5% deuterium incorporation), at least 6000 times of the natural abundance of deuterium (90% deuterium incorporation), at least 6333.3 times of the natural abundance of deuterium (95% deuterium incorporation), at least 6466.7 times of the natural abundance of deuterium (97% deuterium incorporation), at least 6600 times of the natural abundance of deuterium (99% deuterium incorporation), at least 6633.3 times of the natural abundance of deuterium (99.5% deuterium incorporation), or any other abundance.

When a particular position in a compound of the invention (e.g., a compound represented by Formula (I) to (V), or with a compound according to Formula (VI), or a pharmaceutically acceptable salt and/or solvate thereof) is designated specifically by name or structure as “H” or “hydrogen”, the position is understood to have hydrogen at its natural abundance isotopic composition.

The percentage of deuterium incorporation can be obtained by quantitative analysis using, for example, mass spectroscopy (peak area) or by quantifying the remaining residual ¹H-NMR signals of the specific deuteration site compared to signals from internal standards or other, non-deuterated ¹H signals in the compound.

The term “compound,” when referring to any compound of this disclosure, including a compound represented by Formula (I) to (V), or with a compound according to Formula (VI), or a pharmaceutically acceptable salt and/or solvate thereof, refers to a collection of molecules having an identical chemical structure, except that there may be isotopic variation among the constituent hydrogen atoms of the molecules. The relative amount of isotopic variation in a compound of this invention will depend upon a number of factors including the isotopic purity of deuterated reagents used to make the compound and the efficiency of incorporation of deuterium in the various synthesis steps used to prepare the compound.

“D” and “d” both refer to deuterium. “H” refers to hydrogen.

“Substituted with deuterium” refers to the replacement of one or more hydrogen atoms with a corresponding number of deuterium atoms.

An “isotopic variant” refers within the scope of the invention to deuterated variations of compounds of Formula (I) to (V), or with a compound according to Formula (VI), preferably vidofludimus, or a pharmaceutically acceptable salt or solvate thereof.

Any formula or structure given herein, is also intended to represent deuterated compounds comprising in addition further isotopically labelled atoms. Examples of additional isotopes that can be incorporated into compounds of the disclosure include further isotopes of hydrogen (i.e., tritium or 3H), as well as isotopes of carbon, nitrogen, oxygen, phosphorous, fluorine and chlorine, such as, but not limited to ¹¹C, ¹³C, ¹⁴C, ¹⁵N, ¹⁸F, ³¹P, ³²P, ³⁵S, ³⁶Cl and ¹²⁵I. The disclosure further comprises various isotopically labelled compounds into which radioactive isotopes such as ³H, ¹³C and ¹⁴C are incorporated. Such isotopically labelled compounds may be useful in metabolic studies, reaction kinetic studies, detection, or imaging techniques, such as positron emission tomography (PET) or single-photon emission computed tomography (SPECT) including drug or substrate tissue distribution assays or radioactive treatment of patients.

Halogen can be, for example, fluorine, chlorine, bromine, and iodine, in some embodiment fluorine or chlorine and in some embodiment fluorine.

An alkyl group, if not stated otherwise, is preferably a linear or branched chain of 1 to 10 carbon atoms (C₁₋₁₀-alkyl), preferably 1 to 8 carbon atoms (C₁₋₈-alkyl), more preferably 1 to 6 carbon atoms (C₁₋₆-alkyl), in particular 1 to 4 carbon atoms (C₁₋₄-alkyl). Non-limiting examples of straight alkyl groups include methyl, ethyl, propyl, butyl, pentyl, hexyl, heptyl, octyl, nonyl, and decyl. Branched alkyl groups include any straight alkyl group substituted with any number of alkyl groups. Non-limiting examples of branched alkyl groups include isopropyl, isobutyl, sec-butyl, and t-butyl. Preferably alkyl is selected from the group consisting of methyl, ethyl, propyl, isopropyl, butyl, t-butyl, isobutyl, pentyl or hexyl group. More preferably alkyl is selected from the group consisting of methyl, ethyl, isopropyl or t-butyl group. The term “alkyl”, unless otherwise noted, is also meant to include those derivatives of alkyl defined in more detail below as “unsaturated alkyl”. An unsaturated alkyl group is one having one or more double bonds (i.e. “alkenyl”) or triple bonds (i.e. “alkynyl”), preferably vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The alkyl group in the compounds of Formula (I) to (IV) can optionally be substituted by one or more substituents R′, in some embodiments by fluorine.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkyl can be C₁₋₄-alkyl.

The terms “alkylene”, “alkenylene”, “alkynylene”, “arylene”, “heteroarylene” or “cycloalkylene” means that the respective group is divalent and connects the attached residue with the remaining part of the molecule. Moreover, in the context of the present invention, C₁-alkylene means a methylene linker, C₂-alkylene means an ethylene linker or a methyl-substituted methylene linker and so on. In the context of the present invention, an alkylene preferably represents a methylene, an ethylene group or a propylene group. Similarly, the term “arylene” is a divalent aryl group, which can be substituted as defined for aryl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl. Non-limiting examples of cyclic alkyl groups include cyclopropyl, cyclobutyl, cyclopentyl, cyclohexyl, cycloheptlyl, and cyclooctyl groups. Cyclic alkyl (i.e. “cycloalkyl”) groups include saturated or partially unsaturated mono-, bi-, fused-, bridged-, and spiro-bicycles and higher fused-, bridged-, and spiro-systems, preferably comprising in the ring system 3 to 10 carbon atoms, more preferably 3 to 8 carbon atoms, even more preferably 3 to 6 carbon atoms, wherein each of the atoms forming the ring system (i.e., skeletal atoms) is a carbon atom. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl. A cycloalkyl group can optionally be substituted by one or more substituents R′, where R′ is as defined below.

R′ is independently H, D, —CO₂R″, —CONHR″, —CR″O, —SO₂N(R″)₂, —NR″—CO-haloalkyl, —NR″—CO-alkyl, —NO₂, —N₃, —NR″—SO₂-haloalkyl, —NR″—SO₂-alkyl, —SO₂-alkyl, —CN, alkyl, cycloalkyl, aminoalkyl, alkylamino, alkoxy, —OH, oxo, —SH, alkylthio, hydroxyalkyl, hydroxyalkylamino, halogen, haloalkyl, haloalkyloxy, aryl, arylalkyl or heteroaryl.

R″ is independently H, D, haloalkyl, hydroxyalkyl, alkyl, cycloalkyl, aryl, heteroaryl or aminoalkyl.

Non-limiting examples of deuterated alkyl includes mono-deuterated, di-deuterated, tri-deuterated through per-deuterated alkyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. Non-limiting examples of halo alkyl can be C₁₋₄-haloalkyl. A halo-alkenyl group can be any alkenyl group substituted with any number of halogen atoms. A halo-alkynyl group can be any alkynyl group substituted with any number of halogen atoms. In one embodiment haloalkyl is CF₃.

An alkoxy group can be, for example, an oxygen atom substituted with any alkyl, alkenyl, or alkynyl group. Non-limiting examples of alkoxy can be C₁₋₄-alkoxy. An ether or an ether group comprises an alkoxy group. Non-limiting examples of alkoxy groups include methoxy, ethoxy, propoxy, isopropoxy, and isobutoxy. Non-limiting examples of deuterated alkyl includes monodeuterated, dideuterated, tri deuterated through perdeuterated alkyl.

An alkylthio group denotes an S-alkyl group, the alkyl group being as defined above.

A hydroxyalkyl group denotes an HO-alkyl group, the alkyl group being as defined above.

An haloalkyloxy group denotes an alkoxy group which is substituted by one to five preferably three halogen atoms, the alkoxy group being as defined above; a OCF₃ being preferred.

A hydroxyalkylamino group denotes an (HO-alkyl)₂-N-group or HO-alkyl-NH-group, the alkyl group being as defined above.

An alkylamino group denotes an —HN-alkyl or —N-dialkyl group, the alkyl group being as defined above.

An aminoalkyl group denotes an H₂N-alkyl-, monoalkylaminoalkyl, or dialkylaminoalkyl group, the alkyl group being as defined above.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. This aryl group can optionally be substituted by one or more substituents R′, where R′ is as defined above. Preferred R′ residues are fluoro, OCH₃ and OCD₃. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl. Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

An arylalkyl group denotes an alky group which is substituted by one to three preferably one aryl groups, the alkyl and aryl group being as defined above.

A heterocyclyl group can be any ring containing a ring atom that is not carbon, for example, N, O, S, P, Si, B, or any other heteroatom. A heterocyclyl group can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran. In one embodiment a heterocyclyl group comprises 1, 2, 3 or 4 heteroatoms, respectively, wherein the heteroatoms are independently selected from N, O or S. Sulfur atoms in heterocyclic rings can be oxidized to S═O or SO₂. Carbon atoms heterocyclic rings can be oxidized to C═O.

Non-limiting examples of heterocyclyl groups include: heterocyclic units having a single ring containing one or more heteroatoms, non-limiting examples of which include, diazirinyl, aziridinyl, azetidinyl, oxetanyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl. This heterocyclyl group can optionally be substituted by one or more substituents R′, where R′ is as defined below. The heterocyclyl group can be connected with the remaining part of the molecule via a carbon, nitrogen (e.g., in morpholine or piperidine) or sulfur atom. An example for a S-linked heterocycloalkyl is the cyclic sulfonimidamide

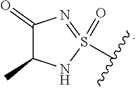 [SMILES: *S1(=O)=NC(=O)[C@H](C)N1]

Heteroaryl groups can include aromatic groups that contain at least one heteroatom. In one embodiment a heteroaryl group comprises 1, 2, 3 or 4 heteroatoms, respectively, wherein the heteroatoms are independently selected from N, O or S. Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Non-limiting examples of groups with one or more hydrogen atoms optionally replaced by deuterium include, when applied to an alkyl group containing 1 to 4 carbon atoms, the following residues: —CD₃, —CH₂D, —CHD₂, CD₃CH₂(CH₂)ₙ—, CD₃CH₂(CHD)ₙ—, CD₃CH₂(CD₂)ₙ—, CH₂DCH₂(CH₂)ₙ—, CH₂DCH₂(CHD)ₙ—, CH₂DCH₂(CD₂)ₙ—, CHD₂CH₂(CH₂)ₙ—, CHD₂CH₂(CHD)ₙ—, CHD₂CH₂(CD₂)ₙ—, CD₃CHD(CH₂)ₙ—, CD₃CHD(CHD)ₙ—, CD₃CHD(CD₂)ₙ—, CH₂DCHD(CH₂)ₙ—, CH₂DCHD(CHD)ₙ—, CH₂DCHD(CD₂)ₙ—, CHD₂CHD(CH₂)ₙ—, CHD₂CHD(CHD)ₙ—, CHD₂CHD(CD₂)ₙ—, CH₃CHD(CH₂)ₙ—, CH₃CHD(CHD)ₙ—, CH₃CHD(CD₂)ₙ—, CD₃CD₂(CH₂)ₙ—, CD₃CD₂(CHD)ₙ—, CD₃CD₂(CD₂)ₙ—, CH₂DCD₂(CH₂)ₙ—, CH₂DCD₂(CHD)ₙ—, CH₂DCD₂(CD₂)ₙ—, CHD₂CD₂(CH₂)ₙ—, CHD₂CD₂(CHD)ₙ—, CHD₂CD₂(CD₂)ₙ—, CH₃CD₂(CH₂)ₙ—, CH₃CD₂(CHD)ₙ—, CH₃CD₂(CD₂)ₙ—, wherein n is an integer from 0 to 2, and CH₃CH₂(CHD)ₘ-, CH₃CH₂(CD₂)ₘ-, wherein m is an integer from 1 to 2, as well as —CD(CD₃)₂, —CH(CD₃)₂ and —C(CD₃)₃.

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

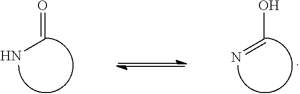

In some embodiments, a compound presented herein exists as tautomers. In circumstances where tautomerization is possible, a chemical equilibrium of the tautomers can exist. The exact ratio of the tautomers depends on several factors, including physical state, temperature, solvent, and pH. Non-limiting examples of tautomeric equilibrium include:

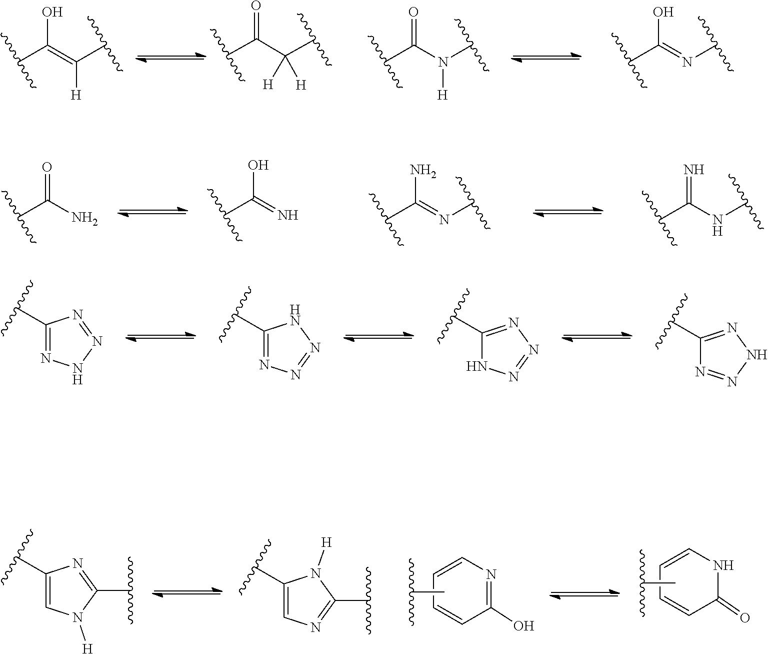

When ring A is a partially saturated cycle, the double bond in ring A is located in the depicted position:

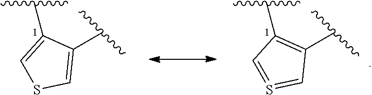 [SMILES: *c1cscc1* ; *C1=C(*)C=S=C1]

In case ring A is a 5-membered heteroaryl ring, then the double bond is within a delocated π-system and can exist in mesomeric forms. An example are the following thiophene mesomeric forms:

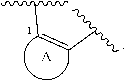

A cycloalkyl or heterocyclyl group can be connected straight or spirocyclic, e.g., when cyclohexane is substituted with the heterocyclyl group oxetane, the following structures are possible:

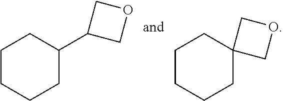 [SMILES: C1CCC(C2COC2)CC1 ; C1CCC2(CC1)COC2]

The present disclosure provides the use of pharmaceutically acceptable salts of any therapeutic compound described herein. Pharmaceutically acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically acceptable salt is a metal salt. In some embodiments, a pharmaceutically acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pipyrrazole, imidazole, pyrazine, or piperazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pipyrrazole salt, an imidazole salt, a pyrazine salt, or a piperazine salt.

Acid addition salts can arise from the addition of an acid to a compound of the disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

In some embodiments, the compounds of the present disclosure are present in the form of solvates, such as those which include as solvate water (hydrate), or pharmaceutically acceptable solvates, such as alcohols, for example, ethanol. A stoichiometric, sub-stochiometric, super-stochiometric, or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, hydrate, or solvate thereof, in a particular crystal packing arrangement. All polymorphs have the same elemental composition. The term “crystalline” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility.

The term “effective amount” is meant to include the amount of a compound that, when administered, is sufficient to prevent development of, or alleviate to some extent, one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

“IMU-838” (also termed “vidofludimus calcium”) is a calcium salt of vidofludimus, including in some embodiments pharmaceutically acceptable solvates, hydrates, crystals and polymorphs.

An illustrative structure for IMU-838 is the dihydrate of 1-cyclopentene-1-carboxylic acid, 2-(((3-fluoro-3′-methoxy(1,1′-biphenyl)-4-yl)amino)carbonyl)-, calcium salt (2:1) with the structure as follows:

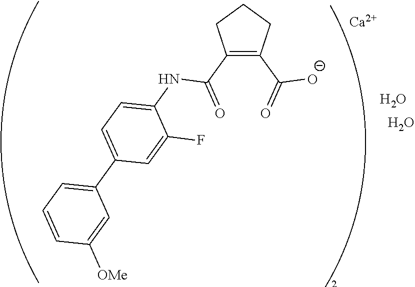

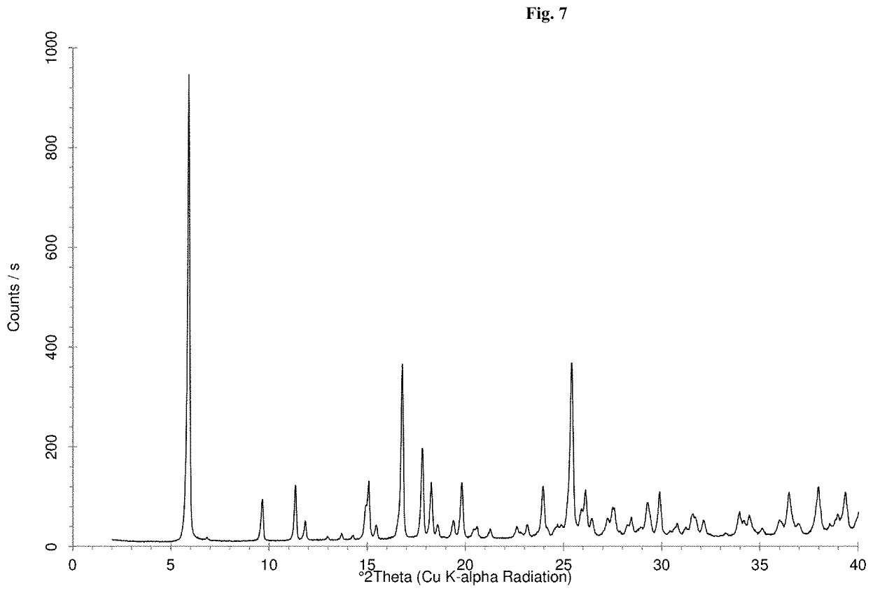

The Polymorph A of IMU-838 is a crystalline material with the structure described above. In some embodiments, Polymorph A of IMU-838 is characterized by an X-ray powder diffraction pattern having characteristic peaks at 2 theta (±0.2°) of 5.91°, 9.64°, 16.780, 17.81°, 19.81°, and 25.41°. In some embodiments, Polymorph A of IMU-838 is characterized by the X-ray diffraction pattern as outlined in FIG. 7.

### ENUMERATED EMBODIMENTS

With the above context, the following consecutively numbered embodiments provide further specific aspects of the invention:

Embodiment 1: A compound of Formula (I),

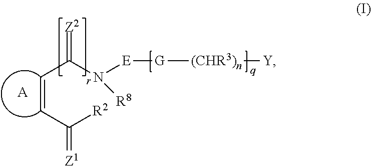

wherein:
   Ring A is a 5- or 6-membered carbocyclic or heterocyclic ring, which is optionally substituted with 1 to 4 residues R¹;
   Z¹ and Z² are each independently O, S, or NR⁹;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl;
   R² is H, OR¹¹, NR¹¹OR¹¹, NR¹¹SO₂R¹¹, or NR¹¹R¹²
   R³ is H, halo, —OR¹³, —SR¹³, —NR¹³R¹⁴, alkyl, alkenyl, alkynyl, alkoxy, aryl, cycloalkyl, heteroaryl, or heterocyclyl;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl;
   n is 0 or 1;
   q is 0, 1, 2, 3, 4,5,6, 7, 8, 9, or 10; and
   r is 0 or 1,
having one or more hydrogen atoms optionally replaced by deuterium, or a pharmaceutically acceptable salt or solvate thereof for use in a method of treating a neurogenerative condition and/or a method of neuroprotection in a subject having a neurogenerative disease.

Embodiment 2: The compound of Formula (I) or a pharmaceutically acceptable salt or solvate thereof for use in a method of treating a neurogenerative condition and/or a method of neuroprotection according to embodiment 1, wherein the subject exhibits an aberrant level of a biomarker associated with a neurodegenerative condition prior to the administering.

Embodiment 3: The compound of Formula (I) or a pharmaceutically acceptable salt or solvate thereof for use in a method of treating a neurogenerative condition and/or a method of neuroprotection according to embodiment 1 or 2, wherein the compound is of Formula (II):

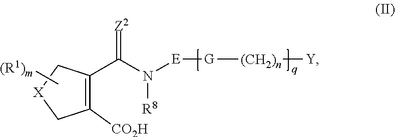

wherein:
   X is O, S, NR⁹, SO, or SO₂;
   Z² is O, S, or NR¹²;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR³, —SR¹³, —NR¹³R¹⁴, alkyl, alkenyl, alkynyl, alkoxy, aryl, cycloalkyl, heteroaryl, or heterocyclyl;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl;
   m is 0, 1, 2, 3, or 4;
   n is 0 or 1; and
   q is 0, 1, 2, 3, 4, 5, 6, 7, 8, 9, or 10.

Embodiment 4: The compound of Formula (I) or a pharmaceutically acceptable salt or solvate thereof for use in a method of treating a neurogenerative condition and/or a method of neuroprotection according to any of embodiments 1 to 3, wherein the compound is of Formula (III):

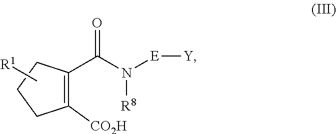

wherein:
   E is alkylene, alkenylene, alkynylene, arylene, heteroarylene, or cycloalkylene;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl; and
   each R⁸, R¹³, and R¹⁴ is independently H, alkyl, alkenyl, alkynyl, aryl, cycloalkyl, heteroaryl, or heterocyclyl.

Embodiment 5: The compound of Formula (I) or a pharmaceutically acceptable salt or solvate thereof for use in a method of treating a neurogenerative condition and/or a method of neuroprotection according to any of embodiments 1 to 4, wherein the compound is of Formula (IV):

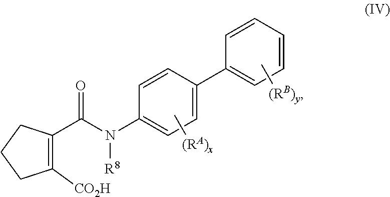

wherein:
   each R^{A} and R^{B} is independently H, D, halo, —OR¹³, —SR¹³, —NR¹³R¹⁴, alkyl, alkenyl, alkynyl, alkoxy, aryl, cycloalkyl, heteroaryl, or heterocyclyl;
   each R⁸, R¹³, and R¹⁴ is independently H, D, alkyl, alkenyl, alkynyl, aryl, cycloalkyl, heteroaryl, or heterocyclyl;
   x is 0, 1, 2, 3, or 4; and
   y is 0, 1, 2, 3, 4, or 5.

Embodiment 6: The compound of Formula (I) or a pharmaceutically acceptable salt or solvate thereof for use in a method of treating a neurogenerative condition and/or a method of neuroprotection according to any of embodiments 1 to 5, wherein the compound is of Formula (V):

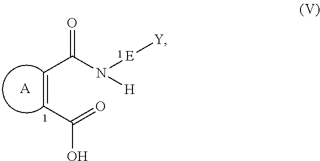

wherein:
   Ring A is

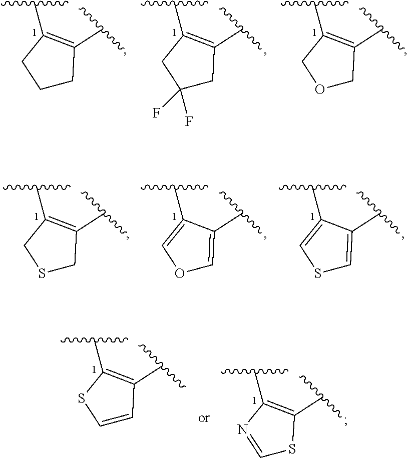

E is

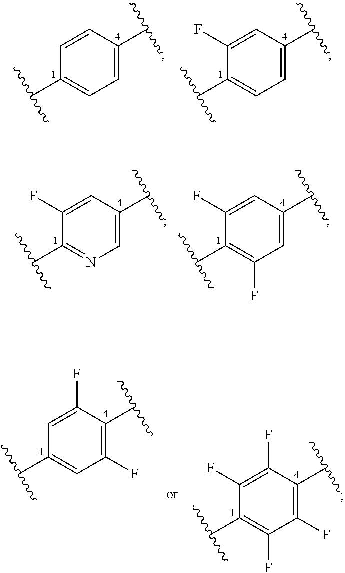

and
Y is

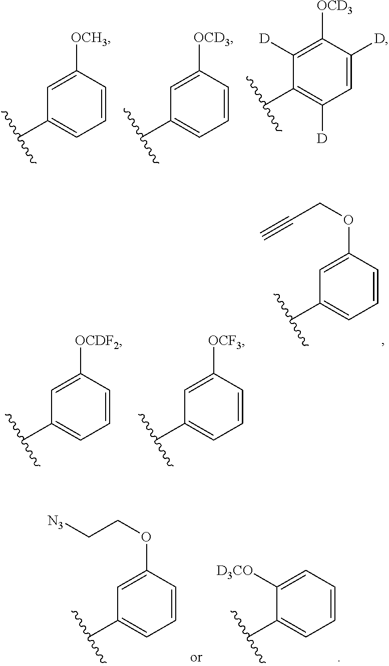

Embodiment 7: The compound of Formula (I) or a pharmaceutically acceptable salt or solvate thereof for use in a method of treating a neurogenerative condition and/or a method of neuroprotection according to any of embodiments 1 to 6, wherein the compound is

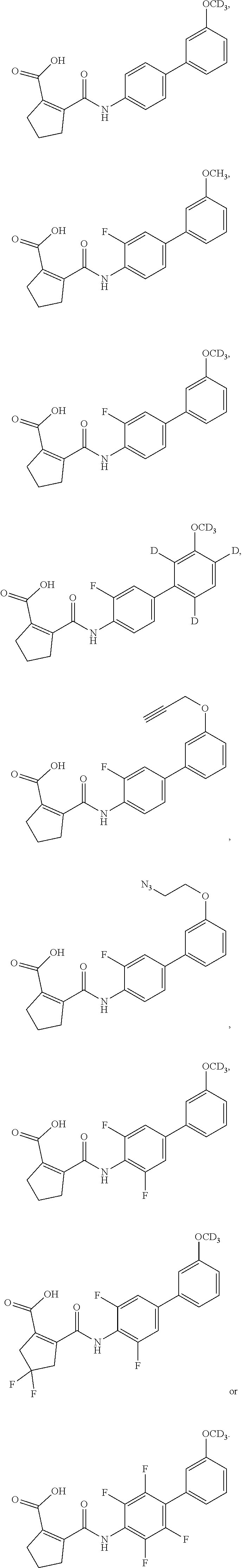

Embodiment 7a: The compound of Formula (I) or a pharmaceutically acceptable salt or solvate thereof for use in a method of treating a neurogenerative condition and/or a method of neuroprotection according to embodiment 1, wherein the compound is of Formula (VI):

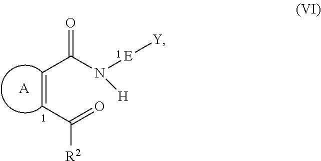

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

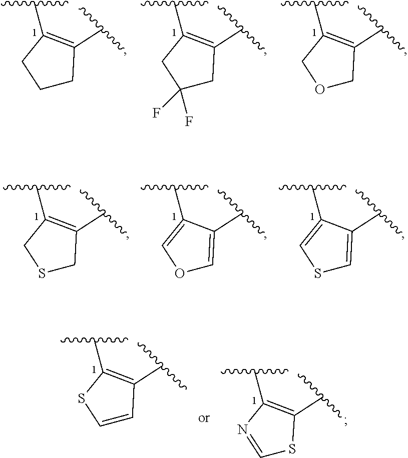

E is

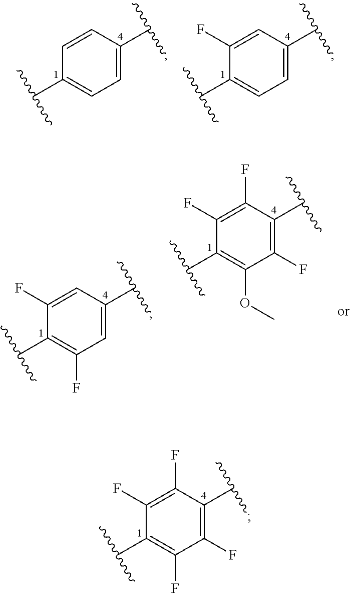

and
Y is

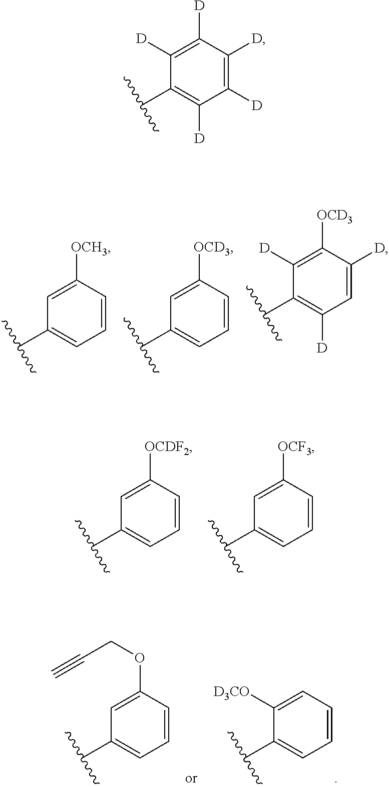

Embodiment 7b: The compound of Formula (I) or a pharmaceutically acceptable salt or solvate thereof for use in a method of treating a neurogenerative condition and/or a method of neuroprotection according to embodiment 1 or 7a, wherein the compound is of Formula (VI):

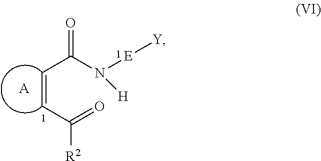

wherein:
   R² is NHR¹¹;
   R¹¹ is selected from H or alkyl, having one or more hydrogen atoms in the alkyl optionally replaced by deuterium;
   Ring A is

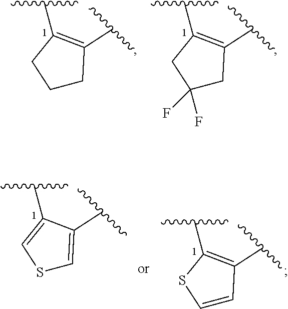

E is

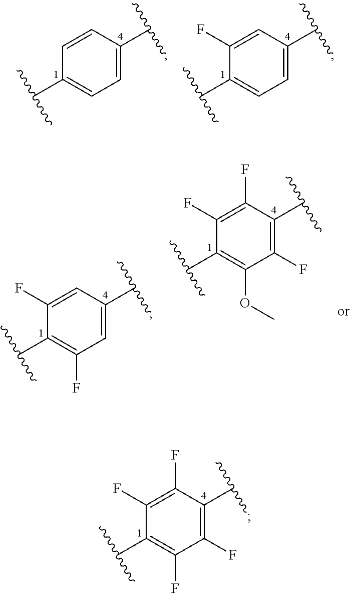

and
Y is

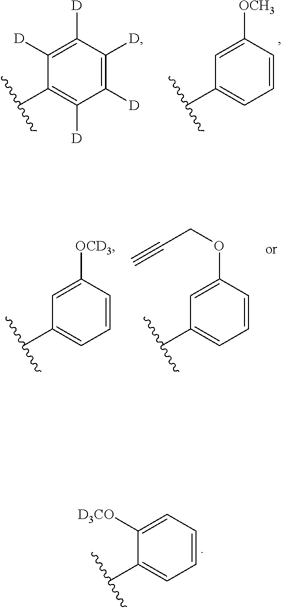

Embodiment 7c: The compound of Formula (I) or a pharmaceutically acceptable salt or solvate thereof for use in a method of treating a neurogenerative condition and/or a method of neuroprotection according to any of embodiments 1, 7a or 7b, wherein the compound is

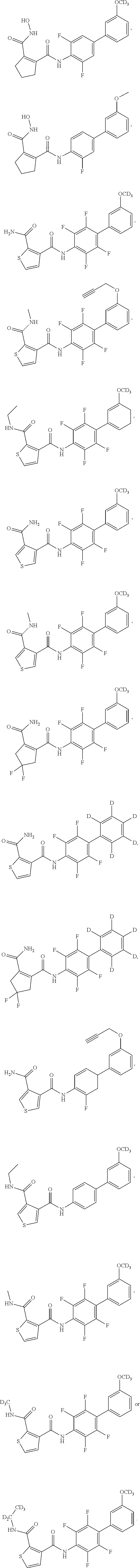

Embodiment 8: The compound of Formula (I) or a pharmaceutically acceptable salt or solvate thereof for use in a method of treating a neurogenerative condition and/or a method of neuroprotection according to any of embodiments 1 to 7c, wherein the compound is

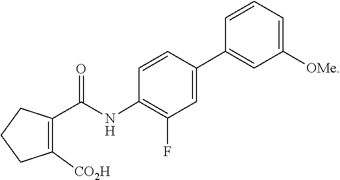 [SMILES: COc1cccc(-c2ccc(NC(=O)C3=C(C(=O)O)CCC3)c(F)c2)c1]

Embodiment 9: The compound of Formula (I) or a solvate thereof for use in a method of treating a neurogenerative condition and/or a method of neuroprotection according to any of embodiments 1 to 8, wherein the compound is

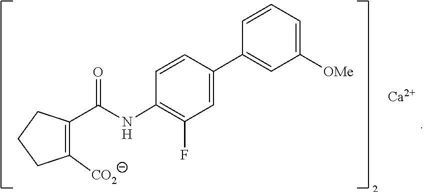

Embodiment 10: The compound of Formula (I) or a solvate thereof for use in a method of treating a neurogenerative condition and/or a method of neuroprotection according to embodiment 9, wherein the compound is the solvate, wherein the solvate is a dihydrate.

Embodiment 11: The compound of Formula (I) or a pharmaceutically acceptable salt or solvate thereof for use in a method of treating a neurogenerative condition and/or a method of neuroprotection according to any of embodiment 1 to 10, wherein the neurodegenerative condition is selected from the group consisting of an inflammatory condition, a non-inflammatory disease worsening loss of neurons, a mental condition, a neurological disorder, a central nervous system condition, Parkinson's Disease, Alzheimer's Disease, Multiple Sclerosis, PIRA, amyotrophic lateral sclerosis, schizophrenia, brain atrophy, and drug addiction, and wherein the neuroprotection is slowing or preventing loss of dopaminergic neurons.

Embodiment 12: The compound of Formula (I) or a pharmaceutically acceptable salt or solvate thereof for use in a method of treating a neurogenerative condition and/or a method of neuroprotection according to any of embodiments 1 to 11, wherein the therapeutically effective amount is about 5 mg to about 100 mg.

Embodiment 13: The compound of Formula (I) or a pharmaceutically acceptable salt or solvate thereof for use in a method of treating a neurogenerative condition and/or a method of neuroprotection according to any of embodiments 1 to 12, wherein the administering is oral.

Embodiment 14: The compound of Formula (I) or a pharmaceutically acceptable salt or solvate thereof for use in a method of treating a neurogenerative condition and/or a method of neuroprotection according to any of embodiments 1 to 13, wherein the administering is oral by a solid dosage form.

Embodiment 15: The compound of Formula (I) or a pharmaceutically acceptable salt or solvate thereof for use in a method of treating a neurogenerative condition and/or a method of neuroprotection according to any of embodiments 1 to 14, wherein the administering is over a first period and a second period, wherein:
a) the first period comprises at least five consecutive days of once-daily dosing of a first period amount of the compound, wherein the first period amount of the compound is about 5 mg to about 50 mg;
b) the second period follows the first period; and
c) the second period comprises once-daily dosing of a second period amount of the compound, wherein the second period amount is greater than the first period amount is.

Embodiment 16: The compound of Formula (I) or a pharmaceutically acceptable salt or solvate thereof for use in a method of treating a neurogenerative condition and/or a method of neuroprotection according to any of embodiments 1 to 15, wherein:
a) the first period is five to ten consecutive days of once-daily dosing of a first period amount of the compound, wherein the first period amount of the compound is about 15 mg to about 25 mg;
b) the second period follows the first period; and
c) the second period comprises once-daily dosing of a second period amount of the compound, wherein the second period amount is about 40 mg to about 50 mg, and wherein the subject is human.

Embodiment 17: The compound of Formula (I) or a pharmaceutically acceptable salt or solvate thereof for use in a method of treating a neurogenerative condition and/or a method of neuroprotection according to any of embodiments 2 to 16, wherein the biomarker is Nurr1.

Embodiment 18: The compound of Formula (I) or a pharmaceutically acceptable salt or solvate thereof for use in a method of treating a neurogenerative condition and/or a method of neuroprotection according to any of embodiments 2 to 16, wherein the biomarker is miR-132.

Embodiment 19: The compound of Formula (I) or a pharmaceutically acceptable salt or solvate thereof for use in a method of treating a neurogenerative condition and/or a method of neuroprotection according to any of embodiments 2 to 16, wherein the aberrant level of the biomarker associated with the neurodegenerative condition is downregulated Nurr1.

Embodiment 20: The compound of Formula (I) or a pharmaceutically acceptable salt or solvate thereof for use in a method of treating a neurogenerative condition and/or a method of neuroprotection according to any of embodiments 2 to 16, wherein the aberrant level of the biomarker associated with the neurodegenerative condition is upregulated miR-132.

Embodiment 21: The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is Parkinson's Disease.

Embodiment 22: The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is multiple sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment 22a. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is active SPMS.

Embodiment 22b. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is PPMS.

Embodiment 22c. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is non-active SPMS.

Embodiment A12d. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is PIRA.

Embodiment 22e. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is clinically isolated syndrome (CIS).

Embodiment 22f. The compound of Formula (I) or a pharmaceutically acceptable salt or solvate thereof for use in a method of treating a neurogenerative condition and/or a method of neuroprotection according to any of embodiments 1 to 20, wherein the neurodegenerative condition is transitioning MS.

Embodiment 23: The compound of Formula (I) or a pharmaceutically acceptable salt or solvate thereof for use in a method of treating a neurogenerative condition and/or a method of neuroprotection according to any of embodiments 1 to 16, wherein the neurodegenerative condition is drug addiction.

Embodiment 24: The compound of Formula (I) or a pharmaceutically acceptable salt or solvate thereof for use in a method of treating a neurogenerative condition and/or a method of neuroprotection according to embodiment 23, wherein the subject exhibits an aberrant level of a biomarker associated with the drug addiction, wherein the biomarker is dopamine.

Embodiment 25: The compound of Formula (I) or a pharmaceutically acceptable salt or solvate thereof for use in a method of treating a neurogenerative condition and/or a method of neuroprotection according to embodiment 23 or 24, wherein the drug addiction is cocaine addiction.

Embodiment 26: The compound of Formula (I) or a pharmaceutically acceptable salt or solvate thereof for use in a method of treating a neurogenerative condition and/or a method of neuroprotection according to embodiment 23 or 24, wherein the drug addiction is amphetamine addiction.

Embodiment 27: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of Nurr1 in a subject; and
b) based on the level of Nurr1 in the subject, determining whether to administer to the subject a Nurr1 agonist.

Embodiment 28: The compound of Formula (I) or a pharmaceutically acceptable salt or solvate thereof for use in a method of treating a neurogenerative condition and/or a method of neuroprotection according to embodiment 27, wherein the level of Nurr1 in the subject is no greater than about 90%, preferably about 80%, more preferably about 70%, even more preferably about 60%, yet more preferably about 50% of the level in a healthy subject of same age, gender and/or BMI, and the determining is to administer to the subject the Nurr1 agonist.

Embodiment 29: The compound of Formula (I) or a pharmaceutically acceptable salt or solvate thereof for use in a method of treating a neurogenerative condition and/or a method of neuroprotection according to embodiment 27, wherein the level of Nurr1 in the subject is at least 98%, preferably at least 95% of the level in a healthy subject of same age, gender and/or BMI, and the determining is not to administer to the subject the Nurr1 agonist.

Embodiment 30: The compound of Formula (I) or a pharmaceutically acceptable salt or solvate thereof thereof for use in a method of treating a neurogenerative condition and/or a method of neuroprotection according to embodiment 27, wherein the level of Nurr1 in the subject is no greater than about 90%, preferably about 80%, more preferably about 70%, even more preferably about 60%, yet more preferably about 50% of the level in a healthy subject of same age, gender and/or BMI, and further comprising identifying that the subject has the neurodegenerative condition.

Embodiment 31: The compound of Formula (I) or a pharmaceutically acceptable salt or solvate thereof for use in a method of treating a neurogenerative condition and/or a method of neuroprotection according to embodiment 27, wherein the level of Nurr1 in the subject is at most 95%, preferably at least 90%, more preferably at least 80%, even more preferably at least 70%, yet more preferably at least 60%, yet more preferably at least 50% of the level in a healthy subject of same age, gender and/or BMI, and further comprising identifying that the subject has the neurodegenerative condition.

Embodiment 32: The compound of Formula (I) or a pharmaceutically acceptable salt or solvate thereof for use in a method of treating a neurogenerative condition and/or a method of neuroprotection according to any of embodiments 27 to 31, wherein the method further comprises, based on the level of Nurr1 in the subject, administering to the subject a therapeutically effective amount of the Nurr1 agonist.

Embodiment 33: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of Nurr1 in a subject who is undergoing a therapy for a neurodegenerative condition; and
b) based on the level of Nurr1 in the subject, determining whether to start or continue the therapy for the neurodegenerative condition, wherein the therapy is Nurr1 agonism.

Embodiment 34: The compound of Formula (I) or a pharmaceutically acceptable salt or solvate thereof for use in a method of treating a neurogenerative condition and/or a method of neuroprotection according to embodiment 33, wherein the level of Nurr1 in the subject is no greater than about 90%, preferably about 80%, more preferably about 70%, even more preferably about 60%, yet more preferably about 50% of the level in a healthy subject of same age, gender and/or BMI, and the determining is to start or continue the therapy.

Embodiment 35: The compound of Formula (I) or a pharmaceutically acceptable salt or solvate thereof for use in a method of treating a neurogenerative condition and/or a method of neuroprotection according to embodiment 33, wherein the level of Nurr1 in the subject is at least 98%, preferably at least 95% of the level in a healthy subject of same age, gender and/or BMI, and the determining is not to start the therapy.

Embodiment 36: The compound of Formula (I) or a pharmaceutically acceptable salt or solvate thereof for use in a method of treating a neurogenerative condition and/or a method of neuroprotection according to embodiment 33, wherein the level of Nurr1 in the subject is no greater than about 90%, preferably about 80%, more preferably about 70%, even more preferably about 60%, yet more preferably about 50% of the level in a healthy subject of same age, gender and/or BMI, and further comprising identifying that the subject needs start of or further treatment for the neurodegenerative condition.

Embodiment 37: The compound of Formula (I) or a pharmaceutically acceptable salt or solvate thereof for use in a method of treating a neurogenerative condition and/or a method of neuroprotection according to embodiment 33, wherein the level of Nurr1 in the subject is at most 95%, preferably at least 90%, more preferably at least 80%, even more preferably at least 70%, yet more preferably at least 60%, yet more preferably at least 50% of the level in a healthy subject of same age, gender and/or BMI, and further comprising identifying that the subject needs start of or further treatment for the neurodegenerative condition.

Embodiment 38: The compound of Formula (I) or a pharmaceutically acceptable salt or solvate thereof for use in a method of treating a neurogenerative condition and/or a method of neuroprotection according to embodiment 33, wherein the therapy for the neurodegenerative condition comprises administering to the subject a therapeutically effective amount of a Nurr1 agonist for the neurodegenerative condition.

Embodiment 39: The compound of Formula (I) or a pharmaceutically acceptable salt or solvate thereof for use in a method of treating a neurogenerative condition and/or a method of neuroprotection according to any of embodiments 27 to 38, wherein the obtaining the level of Nurr1 in the subject comprises obtaining a result of an assay of an ex vivo biological sample of the subject, wherein the assay determines a level of Nurr1 in the ex vivo biological sample of the subject.

Embodiment 40: The compound of Formula (I) or a pharmaceutically acceptable salt or solvate thereof for use in a method of treating a neurogenerative condition and/or a method of neuroprotection according to any of embodiments 27 to 38, wherein the obtaining the level of Nurr1 in the subject comprises performing an assay of an ex vivo biological sample of the subject, wherein the assay determines a level of Nurr1 in the ex vivo biological sample of the subject.

Embodiment 41: The compound of Formula (I) or a pharmaceutically acceptable salt or solvate thereof for use in a method of treating a neurogenerative condition and/or a method of neuroprotection according to embodiment 39 or 40, wherein the assay determines the level of Nurr1 in the ex vivo biological sample of the subject by determining a concentration of Nurr1 in the ex vivo biological sample of the subject.

Embodiment 42: The compound of Formula (I) or a pharmaceutically acceptable salt or solvate thereof for use in a method of treating a neurogenerative condition and/or a method of neuroprotection according to any of embodiments 39 or 40, wherein the assay determines the level of Nurr1 in the ex vivo biological sample of the subject by determining a level of activity of Nurr1 in the ex vivo biological sample of the subject.

Embodiment 43: The compound of Formula (I) or a pharmaceutically acceptable salt or solvate thereof for use in a method of treating a neurogenerative condition and/or a method of neuroprotection according to any of embodiments 39 to 40, wherein the assay is (a) a real-time PCR assay of Nurr1 gene expression against relevant housekeeping genes/internal controls (e.g. GAPDH), (b) an immunoassay (such as ELISA) with the suitable antibodies for Nurr1 protein, or (c) a Western blot for Nurr1 protein from the tissue selected from peripheral blood, peripheral blood lymphocytes, serum, plasma, CSF or peripheral blood mononuclear cells.

Embodiment 44: The compound of Formula (I) or a pharmaceutically acceptable salt or solvate thereof for use in a method of treating a neurogenerative condition and/or a method of neuroprotection according to any of embodiments 39 to 43, wherein the ex vivo biological sample is a blood sample.

Embodiment 45: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of a protein in a subject, wherein the protein is downstream of Nurr1 in a biological pathway in the subject; and
b) based on the level of the protein in the subject, determining whether to administer to the subject a Nurr1 agonist.

Embodiment 46: The compound of Formula (I) or a pharmaceutically acceptable salt or solvate thereof for use in a method of treating a neurogenerative condition and/or a method of neuroprotection according to embodiment 45, wherein the level of the protein in the subject indicates that the protein is upregulated.

Embodiment 47: The compound of Formula (I) or a pharmaceutically acceptable salt or solvate thereof for use in a method of treating a neurogenerative condition and/or a method of neuroprotection according to embodiment 45, wherein the level of the protein in the subject indicates that the protein is downregulated.

Embodiment 48: The compound of Formula (I) or a pharmaceutically acceptable salt or solvate thereof for use in a method of treating a neurogenerative condition and/or a method of neuroprotection according to any of embodiments 45 to 47, wherein the protein is selected from the group consisting of BDNF, GDNF, C-RET, GFAP, DAT, Pitx3, TH, VMAT2, SOD1, AADC, TNFα, iNOS and IL-1β.

Embodiment 49: The compound of Formula (I) or a pharmaceutically acceptable salt or solvate thereof for use in a method of treating a neurogenerative condition and/or a method of neuroprotection according to any of embodiments 45 to 48, wherein the method further comprises, based on the level of the protein in the subject, administering to the subject a therapeutically effective amount of the Nurr1 agonist.

Embodiment 50: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of a protein in a subject who is undergoing a therapy for a neurodegenerative condition, wherein the protein is downstream of Nurr1 in a biological pathway in the subject, wherein the therapy is Nurr1 agonism; and
b) based on the level of the protein in the subject, determining whether to continue the therapy for the neurodegenerative condition.

Embodiment 51: The compound of Formula (I) or a pharmaceutically acceptable salt or solvate thereof for use in a method of treating a neurogenerative condition and/or a method of neuroprotection according to embodiment 50, wherein the level of the protein in the subject is higher than the level was prior to the therapy.

Embodiment 52: The compound of Formula (I) or a pharmaceutically acceptable salt or solvate thereof for use in a method of treating a neurogenerative condition and/or a method of neuroprotection according to embodiment 50, wherein the level of the protein in the subject is lower than the level was prior to the therapy.

Embodiment 53: The compound of Formula (I) or a pharmaceutically acceptable salt or solvate thereof for use in a method of treating a neurogenerative condition and/or a method of neuroprotection according to any of embodiments 50 to 52, wherein the protein is selected from the group consisting of BDNF, GDNF, C-RET, GFAP, DAT, Pitx3, TH, VMAT2, SOD1, AADC, TNFα, iNOS and IL-1β.

Embodiment 54: The compound of Formula (I) or a pharmaceutically acceptable salt or solvate thereof for use in a method of treating a neurogenerative condition and/or a method of neuroprotection according to embodiment 53, wherein the method further comprises determining that the level of the protein in the subject is higher than the level was prior to the therapy.

Embodiment 55: The compound of Formula (I) or a pharmaceutically acceptable salt or solvate thereof for use in a method of treating a neurogenerative condition and/or a method of neuroprotection according to any of embodiments 45 to 54, wherein the obtaining the level of the protein in the subject comprises obtaining a result of an assay of an ex vivo biological sample of the subject, wherein the assay determines a level of the protein in the ex vivo biological sample of the subject.

Embodiment 56: The compound of Formula (I) or a pharmaceutically acceptable salt or solvate thereof for use in a method of treating a neurogenerative condition and/or a method of neuroprotection according to any of embodiments 45 to 54, wherein the obtaining the level of the protein in the subject comprises performing an assay of an ex vivo biological sample of the subject, wherein the assay determines a level of the protein in the ex vivo biological sample of the subject.

Embodiment 57: The compound of Formula (I) or a pharmaceutically acceptable salt or solvate thereof for use in a method of treating a neurogenerative condition and/or a method of neuroprotection according to embodiment 55 or 56, wherein the assay determines the level of the protein in the ex vivo biological sample of the subject by determining a concentration of the protein in the ex vivo biological sample of the subject.

Embodiment 58: The compound of Formula (I) or a pharmaceutically acceptable salt or solvate thereof for use in a method of treating a neurogenerative condition and/or a method of neuroprotection according to embodiment 55 or 56, wherein the assay determines the level of the protein in the ex vivo biological sample of the subject by determining a level of activity of the protein in the ex vivo biological sample of the subject.

Embodiment 59: The compound of Formula (I) or a pharmaceutically acceptable salt or solvate thereof for use in a method of treating a neurogenerative condition and/or a method of neuroprotection according to any of embodiments 55 to 58, wherein the assay is (a) an immunoassay (such as ELISA) with the suitable antibodies for the target protein, or (b) a Western blot for the target protein from the tissue selected from peripheral blood, peripheral blood lymphocytes, serum, plasma, CSF or peripheral blood mononuclear cells.

Embodiment 60: The compound of Formula (I) or a pharmaceutically acceptable salt or solvate thereof for use in a method of treating a neurogenerative condition and/or a method of neuroprotection according to any of embodiments 55 to 59, wherein the ex vivo biological sample is a blood sample.

Embodiment 61: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of activity of a gene in a subject, wherein the gene is downstream of Nurr1 in a biological pathway in the subject; and
b) based on the level of activity of the gene in the subject, determining whether to administer to the subject a Nurr1 agonist for a neurodegenerative condition.

Embodiment 62: The compound of Formula (I) or a pharmaceutically acceptable salt or solvate thereof for use in a method of treating a neurogenerative condition and/or a method of neuroprotection according to embodiment 61, wherein the level of activity of the gene in the subject indicates that the gene is upregulated.

Embodiment 63: The compound of Formula (I) or a pharmaceutically acceptable salt or solvate thereof for use in a method of treating a neurogenerative condition and/or a method of neuroprotection according to embodiment 61 or 62, wherein the method further comprises, based on the level of activity of the gene in the subject, administering to the subject a therapeutically effective amount of the Nurr1 agonist.

Embodiment 64: The compound of Formula (I) or a pharmaceutically acceptable salt or solvate thereof for use in a method of treating a neurogenerative condition and/or a method of neuroprotection according to any of embodiments 1 to 17, wherein the method comprises:
a) obtaining a level of activity of a gene in a subject who is undergoing a therapy for a neurodegenerative condition, wherein the gene is downstream of Nurr1 in a biological pathway in the subject, wherein the therapy is Nurr1 agonism; and
b) based on the level of activity of the gene in the subject, determining whether to continue the therapy for the neurodegenerative condition.

Embodiment 65: The compound of Formula (I) or a pharmaceutically acceptable salt or solvate thereof for use in a method of treating a neurogenerative condition and/or a method of neuroprotection according to embodiment 64, wherein the level of activity of the gene in the subject is higher than the level was prior to the therapy.

Embodiment 66: The compound of Formula (I) or a pharmaceutically acceptable salt or solvate thereof for use in a method of treating a neurogenerative condition and/or a method of neuroprotection according to any of embodiments 61 to 65, wherein the obtaining the level of activity of the gene in the subject comprises obtaining a result of an assay of an ex vivo biological sample of the subject, wherein the assay determines a level of activity of the gene in the ex vivo biological sample of the subject.

Embodiment 67: The compound of Formula (I) or a pharmaceutically acceptable salt or solvate thereof for use in a method of treating a neurogenerative condition and/or a method of neuroprotection according to any of embodiments 61 to 65, wherein the obtaining the level of activity of the gene in the subject comprises performing an assay of an ex vivo biological sample of the subject, wherein the assay determines a level of activity of the gene in the ex vivo biological sample of the subject.

Embodiment 68: The compound of Formula (I) or a pharmaceutically acceptable salt or solvate thereof for use in a method of treating a neurogenerative condition and/or a method of neuroprotection according to embodiment 66 or 67, wherein the assay is a real-time PCR assay of Nurr1 gene expression against relevant housekeeping genes/internal controls (e.g. GAPDH) from the tissue selected from peripheral blood, peripheral blood lymphocytes, serum, plasma, CSF or peripheral blood mononuclear cells.

Embodiment 69: The compound of Formula (I) or a pharmaceutically acceptable salt or solvate thereof for use in a method of treating a neurogenerative condition and/or a method of neuroprotection according to any of embodiments 66 to 68, wherein the ex vivo biological sample is a blood sample.

Embodiment 70: The compound of Formula (I) or a pharmaceutically acceptable salt or solvate thereof for use in a method of treating a neurogenerative condition and/or a method of neuroprotection according to any of embodiments 61 to 69, wherein the gene is selected from the group consisting of NCAM, CFB, LTA, A2M, HSD11B1, BHLHE41, MARCO, BDNF, GDNF, C-RET, GFAP, DAT, Pitx3, TH, VMAT2, SOD1, AADC, TNFα, iNOS and IL-1β.

Embodiment 71: A method comprising:
a) determining in a sample obtained from a subject that the subject exhibits downregulated Nurr1;
b) determining in a sample obtained from the subject that the subject exhibits upregulated miR-132; and
c) based on the determining that the subject exhibits downregulated Nurr1 and the determining that the subject exhibits upregulated miR-132, identifying the subject as being at risk for a condition.

Embodiment 72: The method of embodiment 71, wherein the determining that the subject exhibits downregulated Nurr1 comprises obtaining a result of a first assay of an ex vivo biological sample of the subject, wherein the first assay determines a level of Nurr1 in the ex vivo biological sample of the subject; and the determining that the subject exhibits upregulated miR-132 comprises obtaining a result of a second assay of the ex vivo biological sample of the subject, wherein the second assay determines a level of miR-132 in the ex vivo biological sample of the subject.

Embodiment 73: The method of embodiment 71, wherein the determining that the subject exhibits downregulated Nurr1 comprises performing a first assay of an ex vivo biological sample of the subject, wherein the first assay determines a level of Nurr1 in the ex vivo biological sample of the subject; and the determining that the subject exhibits upregulated miR-132 comprises performing a second assay of the ex vivo biological sample of the subject, wherein the second assay determines a level of miR-132 in the ex vivo biological sample of the subject.

Embodiment 74: The method of embodiment 72 or 73, wherein the first assay determines the level of Nurr1 in the ex vivo biological sample of the subject by determining a concentration of Nurr1 in the ex vivo biological sample of the subject.

Embodiment 75: The method of embodiment 72 or 73, wherein the first assay determines the level of Nurr1 in the ex vivo biological sample of the subject by determining a level of activity of Nurr1 in the ex vivo biological sample of the subject.

Embodiment 76: The method of any of embodiments 72 to 75, wherein the first assay is a reverse transcription real-time quantitative PCR (RT-qPCR) assay from peripheral blood lymphocytes.

Embodiment 77: The method of any of embodiments 72 to 76, wherein the second assay determines the level of miR-132 in the ex vivo biological sample of the subject by determining a concentration of miR-132 in the ex vivo biological sample of the subject.

Embodiment 78: The method of any of embodiments 72 to 76, wherein the second assay determines the level of miR-132 in the ex vivo biological sample of the subject by determining a level of activity of miR-132 in the ex vivo biological sample of the subject.

Embodiment 79: The method of any of embodiments 72 to 78, wherein the second assay is a reverse transcription real-time quantitative PCR (RT-qPCR) assay from plasma.

Embodiment 80: The method of any of embodiments 71 to 79, wherein the ex vivo biological sample is a blood sample.

Embodiment 81: The method of any of embodiments 71 to 80, wherein the level of Nurr1 in the ex vivo biological sample of the subject is no greater than about 90% of the level in a healthy subject of same age, gender and/or BMI.

Embodiment 82: The method of any of embodiments 71 to 81, wherein the level of miR-132 in the ex vivo biological sample of the subject is at least 110% of the level in a healthy subject of same age, gender and/or BMI.

Embodiment 83: The method of any of embodiments 71 to 82, wherein the condition is selected from the group consisting of a neurodegenerative condition, an inflammatory condition, a mental condition, a central nervous system condition, Parkinson's Disease, Alzheimer's Disease, Multiple Sclerosis, PIRA, amyotrophic lateral sclerosis, schizophrenia and drug addiction.

Embodiment 84: The method of any of embodiments 71 to 83, further comprising, based on the determining that the subject exhibits downregulated Nurr1 and the determining that the subject exhibits upregulated miR-132, identifying the subject as a candidate for therapy for a condition.

Embodiment 85: The method of any of embodiments 71 to 83, further comprising, based on the determining that the subject exhibits downregulated Nurr1 and the determining that the subject exhibits upregulated miR-132, administering to the subject a therapeutically effective amount of a compound of Formula (I) or a pharmaceutically acceptable salt or solvate thereof that modulates Nurr1.

Embodiment 86: The method of embodiment 85, wherein the compound that modulates Nurr1 agonizes Nurr1.

Embodiment 87: The method of embodiment 85 or 86, wherein the therapeutically effective amount is about 5 mg to about 100 mg.

Embodiment 88: The method of any of embodiments 85 to 87, wherein the administering is oral.

Embodiment 89: The method of any of embodiments 85 to 88, wherein the administering is oral by a solid dosage form.

Embodiment 90: The method of any of embodiments 85 to 89, wherein the administering is over a first period and a second period, wherein:
a) the first period comprises at least five consecutive days of once-daily dosing of a first period amount of the compound, wherein the first period amount of the compound is about 5 mg to about 50 mg;
b) the second period follows the first period; and
c) the second period comprises once-daily dosing of a second period amount of the compound, wherein the second period amount is greater than the first period amount is.

Embodiment 91: The method of any of embodiments 85 to 90, wherein the administering is over a first period and a second period, wherein:
a) the first period is five to ten consecutive days of once-daily dosing of a first period amount of the compound, wherein the first period amount of the compound is about 15 mg to about 25 mg;
b) the second period follows the first period; and
c) the second period comprises once-daily dosing of a second period amount of the compound, wherein the second period amount is about 40 mg to about 50 mg.
wherein the subject is human.

Embodiment 92: The method of any of embodiments 85 to 91, wherein the compound of Formula (I),

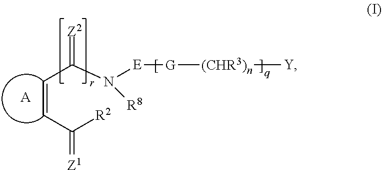

is characterized in that
   Ring A is a 5- or 6-membered carbocyclic or heterocyclic ring, which is optionally substituted with 1 to 4 residues R′;
   Z¹ and Z² are each independently O, S, or NR⁹;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl;
   R² is H, OR¹¹, NR¹¹OR¹¹, NR¹¹SO₂R¹¹, or NR¹¹R¹²
   R³ is H, halo, —OR¹³, —SR¹³, —NR¹³R¹⁴, alkyl, alkenyl, alkynyl, alkoxy, aryl, cycloalkyl, heteroaryl, or heterocyclyl;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl;
   n is 0 or 1;
   q is 0, 1, 2, 3, 4, 5, 6, 7, 8, 9, or 10; and
   r is 0 or 1,
having one or more hydrogen atoms optionally replaced by deuterium, or a pharmaceutically acceptable salt or solvate thereof.

Embodiment 93: The method of embodiment 92, wherein the compound of Formula (I) is the compound of Formula (II) or a pharmaceutically acceptable salt or solvate thereof:

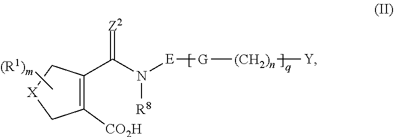

wherein:
   X is O, S, NR⁹, SO, or SO₂;
   Z² is O, S, or NR¹²;
   E is alkylene, alkenylene, alkynylene, arylene, heteroarylene, or cycloalkylene;
   G is O, S, SO₂, NR¹⁰, or CH₂;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl;
   each R⁸, R⁹, R¹⁰, R¹¹, R¹², R¹³, and R¹⁴ is independently H, alkyl, alkenyl, alkynyl, aryl, cycloalkyl, heteroaryl, or heterocyclyl;
   m is 0, 1, 2, 3, or 4;
   n is 0 or 1; and
   q is 0, 1, 2, 3, 4, 5, 6, 7, 8, 9, or 10.

Embodiment 94: The method of embodiment 92 or 93, wherein the compound of Formula (I) is the compound of Formula (III) or a pharmaceutically acceptable salt or solvate thereof:

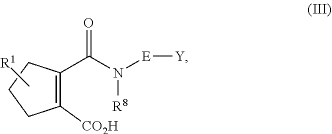

wherein:
   E is alkylene, alkenylene, alkynylene, arylene, heteroarylene, or cycloalkylene;
   Y is aryl, heteroaryl, heterocyclyl, or cycloalkyl;
   R¹ is H, halo, —OR¹¹, —SR¹³, —NR¹³R¹⁴, alkyl, alkenyl, alkynyl, alkoxy, aryl, cycloalkyl, heteroaryl, or heterocyclyl; and
   each R⁸, R¹³, and R¹⁴ is independently H, alkyl, alkenyl, alkynyl, aryl, cycloalkyl, heteroaryl, or heterocyclyl.

Embodiment 95: The method of any of embodiments 92 to 94, wherein the compound of Formula (I) is the compound of Formula (IV) or a pharmaceutically acceptable salt or solvate thereof:

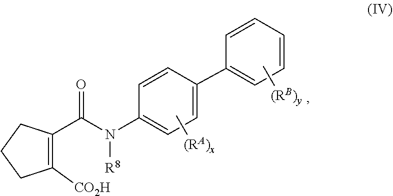

wherein:
   each R^{A} and R^{B} is independently H, D, halo, —OR¹³, —SR¹³, —NR¹³R¹⁴, alkyl, alkenyl, alkynyl, alkoxy, aryl, cycloalkyl, heteroaryl, or heterocyclyl;
   each R⁸, R¹³, and R¹⁴ is independently H, D, alkyl, alkenyl, alkynyl, aryl, cycloalkyl, heteroaryl, or heterocyclyl;
   x is 0, 1, 2, 3, or 4; and
   y is 0, 1, 2, 3, 4, or 5.

Embodiment 96: The method of any of embodiments 92 to 95, wherein the compound of Formula (I) is the compound of Formula (V) or a pharmaceutically acceptable salt or solvate thereof:

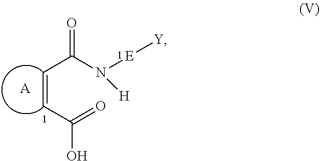

wherein:
   Ring A is

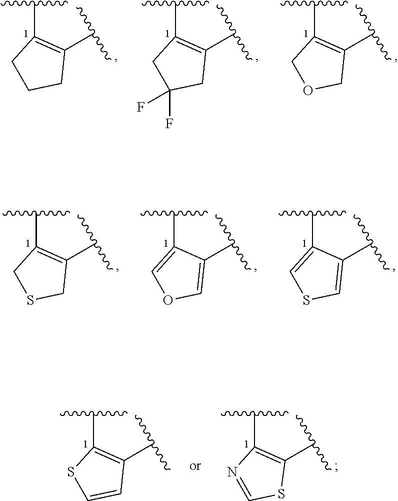

E is

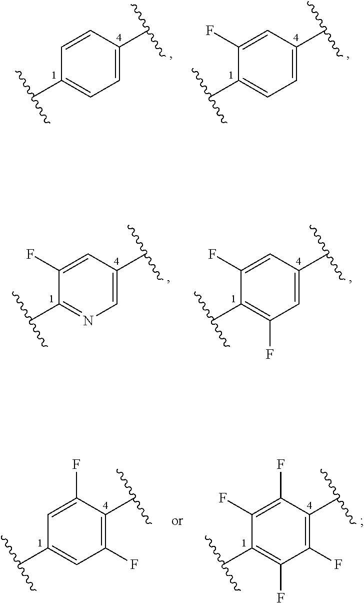 [SMILES: *c1ccc(*)cc1 ; *c1ccc(*)c(F)c1 ; *c1cnc(*)c(F)c1 ; *c1cc(F)c(*)c(F)c1 ; *c1cc(F)c(*)c(F)c1 ; *c1c(F)c(F)c(*)c(F)c1F]

and
Y is

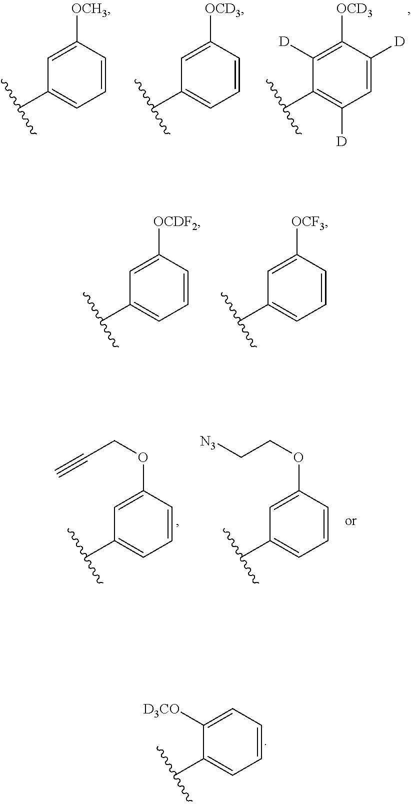

Embodiment 97: The method of any of embodiments 92 to 96, wherein the compound of Formula (I) or a pharmaceutically acceptable salt or solvate thereof is

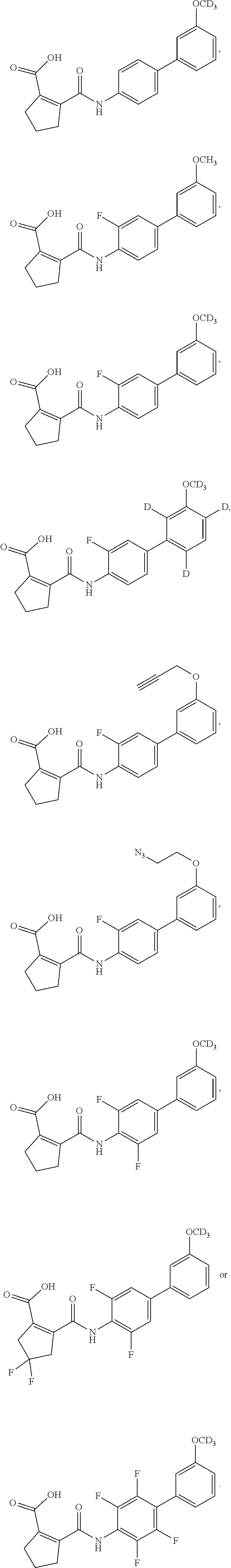

Embodiment 98: The method of any of embodiments 92 to 97, wherein the compound of Formula (I) is

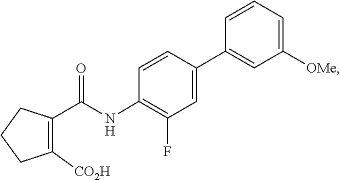 [SMILES: COc1cccc(-c2ccc(NC(=O)C3=C(C(=O)O)CCC3)c(F)c2)c1]

or a pharmaceutically acceptable salt or solvate thereof.

Embodiment 99: The method of any of embodiments 92 to 98, wherein the compound of Formula (I) is

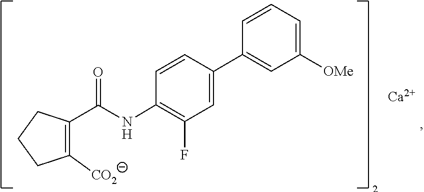

or a solvate thereof.

Embodiment 100: The method of embodiment 99, wherein the compound of Formula (I) is the solvate, wherein the solvate is a dihydrate.

Embodiment 101. The method of embodiment 92, wherein the compound of Formula (I) is the compound of Formula (VI) or a pharmaceutically acceptable salt or solvate thereof:

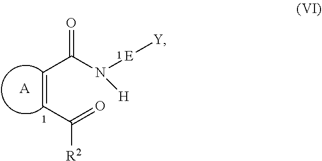

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

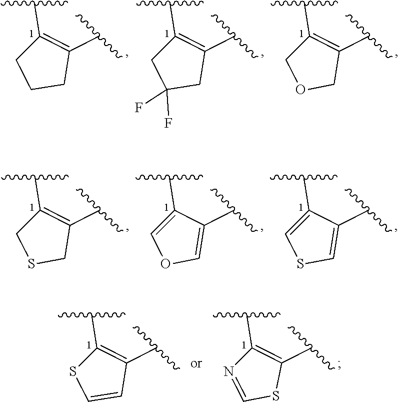

E is

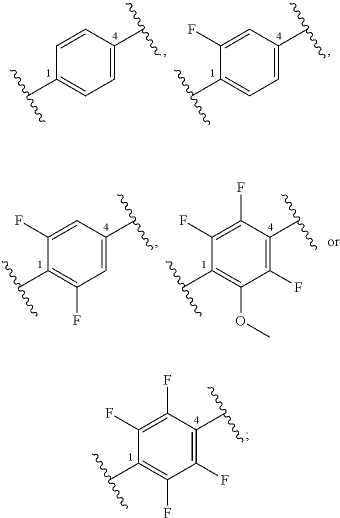 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

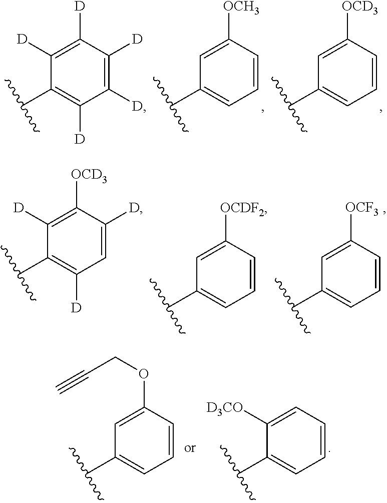

Embodiment 102. The method of embodiment 101, wherein the compound of Formula (I) is the compound of Formula (VI) or a pharmaceutically acceptable salt or solvate thereof:

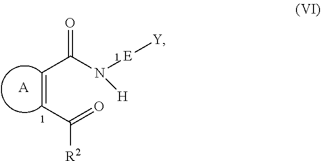

wherein:
   R² is NHR¹¹;
   R¹¹ is selected from H or alkyl, having one or more hydrogen atoms in the alkyl optionally replaced by deuterium;
   Ring A is

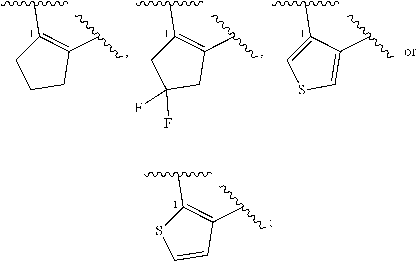 [SMILES: *C1=C(*)CCC1 ; *C1=C(*)CC(F)(F)C1 ; *c1cscc1* ; *c1ccsc1*]

E is

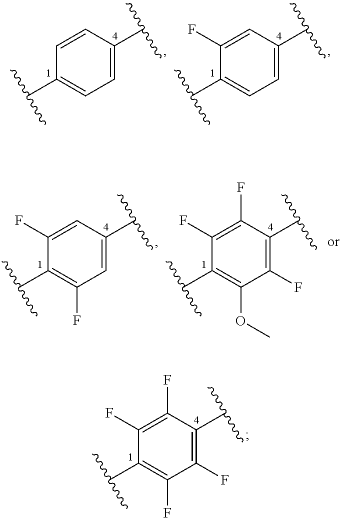 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

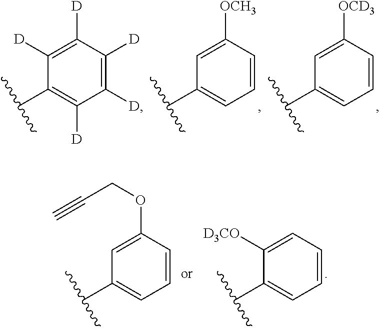

Embodiment 103. The method of any of embodiments 101 to 102, wherein the compound of Formula (I) or a pharmaceutically acceptable salt or solvate thereof is

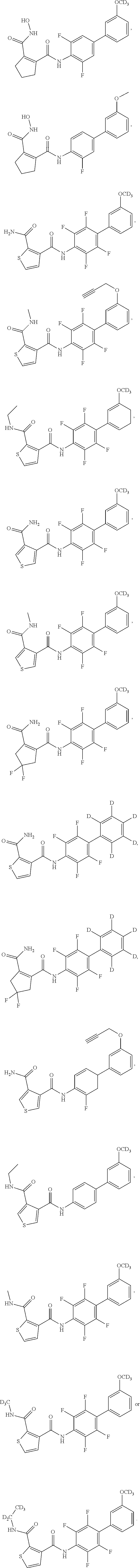

Embodiment 104: The compound of Formula (I) or a pharmaceutically acceptable salt or a solvate thereof for use in a method of treating a neurogenerative condition and/or a method of neuroprotection wherein the method comprises:
determining a level or activity of Nurr1 in the ex vivo biological sample of the subject with an assay selected from (a) a real-time PCR assay of Nurr1 gene expression against relevant housekeeping genes/internal controls (e.g. GAPDH), (b) an immunoassay (such as ELISA) with the suitable antibodies for Nurr1 protein, or (c) a Western blot for Nurr1 protein from the biological sample selected from peripheral blood, peripheral blood lymphocytes, serum, plasma, CSF or peripheral blood mononuclear cells; and
if the test sample from the patient comprises a level of Nurr1 of no greater than about 90% of the level in a healthy subject of same age, gender and/or BMI, administering to the patient an effective amount of Formula (I) or a pharmaceutically acceptable salt or a solvate thereof.

Embodiment 105: The compound of Formula (I) or a pharmaceutically acceptable salt or solvate thereof for use in a method of treating a neurogenerative condition and/or a method of neuroprotection according to embodiment 22, wherein the neurodegenerative condition is primary progressive multiple sclerosis.

Embodiment 106: The compound of Formula (I) or a pharmaceutically acceptable salt or solvate thereof for use in a method of treating a neurogenerative condition and/or a method of neuroprotection according to embodiment 22, wherein the neurodegenerative condition is relapsing remitting multiple sclerosis.

Embodiment 107: The compound of Formula (I) or a pharmaceutically acceptable salt or solvate thereof for use in a method of treating a neurogenerative condition and/or a method of neuroprotection according to embodiment 22, wherein the neurodegenerative condition is multiple sclerosis, wherein the disability is acquired through relapse-associated worsening (RAW).

Embodiment 108: The compound of Formula (I) or a pharmaceutically acceptable salt or solvate thereof for use in a method of treating a neurogenerative condition and/or a method of neuroprotection according to embodiment 22, wherein the neurodegenerative condition is multiple sclerosis, wherein the disability is acquired through progression independent of relapse activity (PIRA).

Embodiment 109: The compound of Formula (I) or a pharmaceutically acceptable salt or solvate thereof for use in a method of treating a neurogenerative condition and/or a method of neuroprotection according to any of embodiments 1 to 20 and 22, wherein the neurodegenerative condition is brain atrophy induced by multiple sclerosis.

Embodiment 110: The compound of Formula (I) or a pharmaceutically acceptable salt or solvate thereof for use in a method of treating a neurogenerative condition and/or a method of neuroprotection according to any of embodiments 1 to 20 and 22, wherein the neurodegenerative condition is non-inflammatory multiple sclerosis worsening.

Embodiment 111: The compound of Formula (I) or a pharmaceutically acceptable salt or solvate thereof for use in a method of treating a neurogenerative condition and/or a method of neuroprotection according to any of embodiments 1 to 20 and 22, wherein the neuroprotection is slowing or preventing loss of neurons induced by multiple sclerosis.

Embodiment 112: The compound of Formula (I) or a pharmaceutically acceptable salt or solvate thereof for use in a method of treating a neurogenerative condition and/or a method of neuroprotection according to any of embodiments 1 to 20 and 22, wherein the neuroprotection is slowing or preventing loss of dopaminergic neurons induced by multiple sclerosis.

Embodiment 113: The compound of Formula (I) or a pharmaceutically acceptable salt or solvate thereof for use in a method of treating a neurogenerative condition and/or a method of neuroprotection according to any of embodiments 1 to 20 and 22, wherein the neurodegenerative condition is PIRA.

Embodiment A1. A method of treating a condition in a subject, the method comprising administering to the subject a therapeutically-effective amount of a compound of Formula (I),

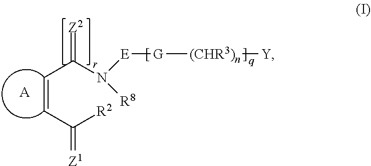

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof, wherein the subject exhibits an aberrant level of a biomarker associated with a neurodegenerative condition prior to the administering.

Embodiment A2. The method of Embodiment A1, wherein the biomarker is Nurr1.

Embodiment A3. The method of Embodiment A1, wherein the biomarker is miR-132.

Embodiment A4. The method of Embodiment A1, wherein the aberrant level of the biomarker associated with the neurodegenerative condition is downregulated Nurr1.

Embodiment A5. The method of Embodiment A1, wherein the aberrant level of the biomarker associated with the neurodegenerative condition is upregulated miR-132.

Embodiment A6. The method of Embodiment A1, wherein the condition is a neurodegenerative condition.

Embodiment A7. The method of Embodiment A1, wherein the condition is an inflammatory condition.

Embodiment A8. The method of Embodiment A1, wherein the condition is a mental condition.

Embodiment A9. The method of Embodiment A1, wherein the condition is a central nervous system condition.

Embodiment A10. The method of Embodiment A1, wherein the condition is Parkinson's Disease.

Embodiment A11. The method of Embodiment A1, wherein the condition is Alzheimer's Disease.

Embodiment A12. The method of Embodiment A1, wherein the condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment A12a. The method of Embodiment A1, wherein the neurodegenerative condition is active SPMS.

Embodiment A12b. The method of Embodiment A1, wherein the neurodegenerative condition is PPMS.

Embodiment A12c. The method of Embodiment A1, wherein the neurodegenerative condition is non-active SPMS.

Embodiment A12d. The method of Embodiment A1, wherein the neurodegenerative condition is PIRA.

Embodiment A12e. The method of Embodiment A1, wherein the neurodegenerative condition is clinically isolated syndrome (CIS).

Embodiment A12f. The method of Embodiment A1, wherein the neurodegenerative condition is transitioning MS.

Embodiment A13. The method of Embodiment A1, wherein the condition is amyotrophic lateral sclerosis.

Embodiment A14. The method of Embodiment A1, wherein the condition is schizophrenia.

Embodiment A15. The method of Embodiment A1, wherein the condition is drug addiction.

Embodiment A16. The method of Embodiment A1, wherein the therapeutically-effective amount is about 5 mg to about 100 mg.

Embodiment A17. The method of Embodiment A1, wherein the administering is oral.

Embodiment A18. The method of Embodiment A1, wherein the administering is oral by a solid dosage form.

Embodiment A19. The method of Embodiment A1, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment A20. The method of Embodiment A1, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg, wherein the subject is human.

Embodiment A21. The method of Embodiment A1, wherein Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR³, —SR³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment A22. The method of Embodiment A1, wherein the compound is of Formula (II):

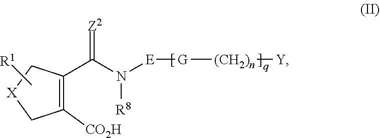

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹, —SR³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment A23. The method of Embodiment A1, wherein the compound is of Formula (III):

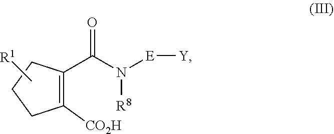

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment A24. The method of Embodiment A1, wherein the compound is of Formula (IV):

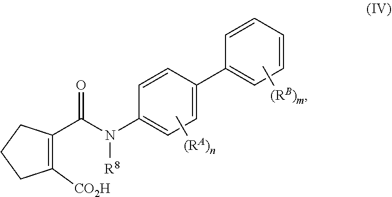

wherein: each R^{A} and R^{B} is independently —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment A25. The method of Embodiment A1, wherein the compound is

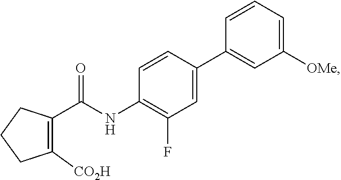 [SMILES: COc1cccc(-c2ccc(NC(=O)C3=C(C(=O)O)CCC3)c(F)c2)c1]

or a pharmaceutically-acceptable salt or solvate thereof.

Embodiment A26. The method of Embodiment A1, wherein the compound is

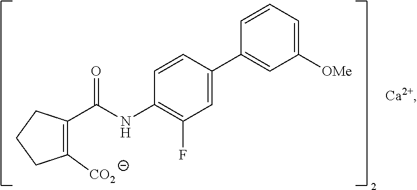

or a solvate thereof.

Embodiment A27. The method of Embodiment A26, wherein the compound is the solvate, wherein the solvate is a dihydrate.

Embodiment A28. The method of Embodiment A1, wherein the compound of Formula (I) is the compound of Formula (VI) or a pharmaceutically acceptable salt or solvate thereof:

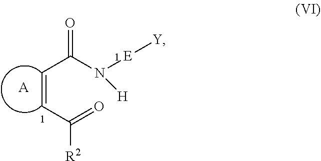

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

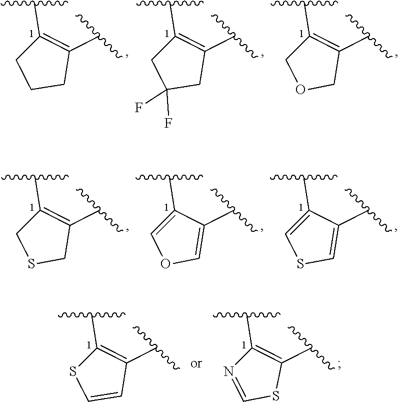

E is

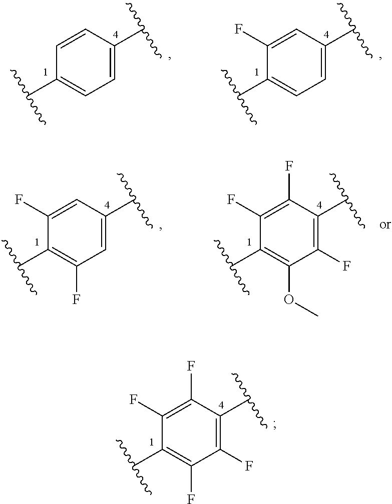 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

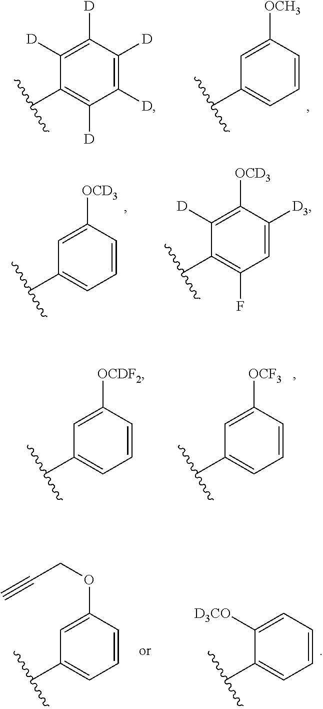

Embodiment B1. A method of treating Parkinson's Disease in a subject, the method comprising administering to the subject a therapeutically-effective amount of a compound of Formula (I),

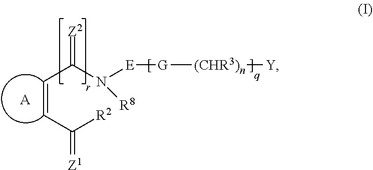

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof, wherein the subject exhibits an aberrant level of a biomarker associated with Parkinson's Disease prior to the administering.

Embodiment B2. The method of Embodiment B1, wherein the biomarker is Nurr1.

Embodiment B3. The method of Embodiment B1, wherein the biomarker is miR-132.

Embodiment B4. The method of Embodiment B1, wherein the aberrant level of the biomarker associated with the neurodegenerative condition is downregulated Nurr1.

Embodiment B5. The method of Embodiment B1, wherein the aberrant level of the biomarker associated with the neurodegenerative condition is upregulated miR-132.

Embodiment B6. The method of Embodiment B1, wherein the therapeutically-effective amount is about 5 mg to about 100 mg.

Embodiment B7. The method of Embodiment B1, wherein the administering is oral.

Embodiment B8. The method of Embodiment B1, wherein the administering is oral by a solid dosage form.

Embodiment B9. The method of Embodiment B1, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment B10. The method of Embodiment B1, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment B11. The method of Embodiment B1, wherein the subject is human.

Embodiment B12. The method of Embodiment B1, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment B13. The method of Embodiment B1, wherein the compound is of Formula (II):

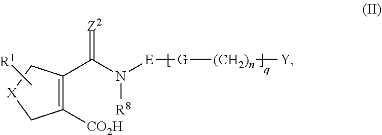

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment B14. The method of Embodiment B1, wherein the compound is of Formula (III):

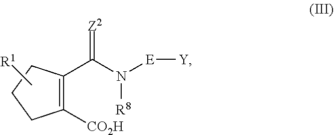

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment B15. The method of Embodiment B1, wherein the compound is of Formula (IV):

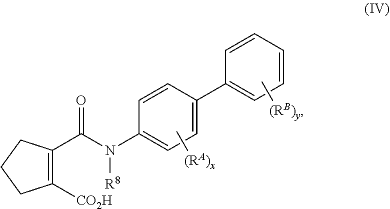

wherein: each R^{A} and R^{B} is independently —OR, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment B16. The method of Embodiment B1, wherein the compound is

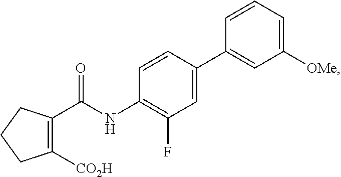 [SMILES: COc1cccc(-c2ccc(NC(=O)C3=C(C(=O)O)CCC3)c(F)c2)c1]

or a pharmaceutically-acceptable salt or solvate thereof.

Embodiment B17. The method of Embodiment B1, wherein the compound is

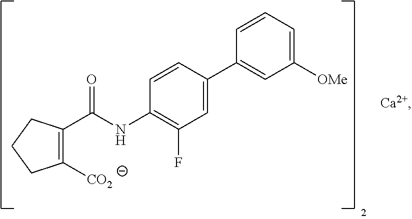

or a solvate thereof.

Embodiment B18. The method of Embodiment B17, wherein the compound is the solvate, wherein the solvate is a dihydrate.

Embodiment B19. The method of Embodiment B1, wherein the compound of Formula (I) is the compound of Formula (VI) or a pharmaceutically acceptable salt or solvate thereof:

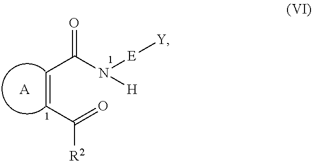

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

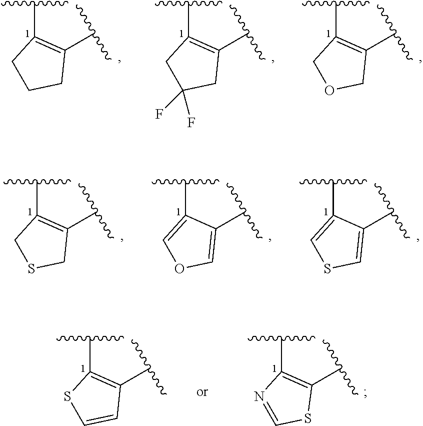

E is

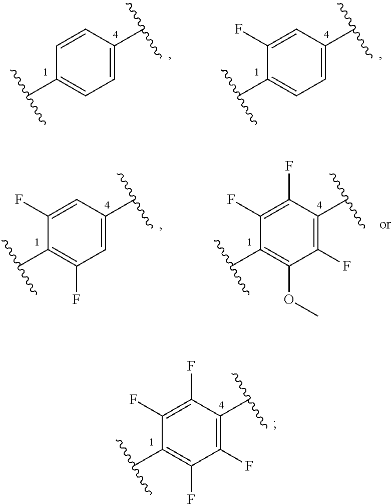 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

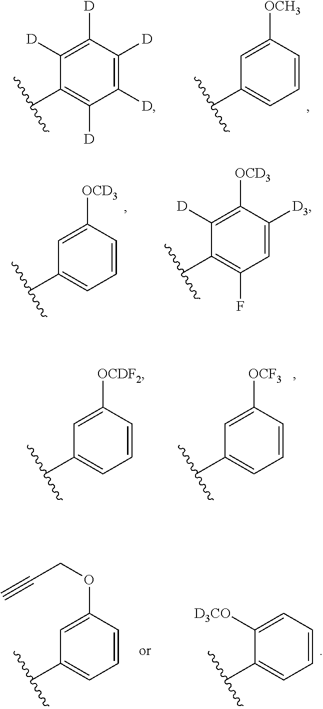

Embodiment C1. A method of treating drug addiction in a subject, the method comprising administering to the subject a therapeutically-effective amount of a compound of Formula (I),

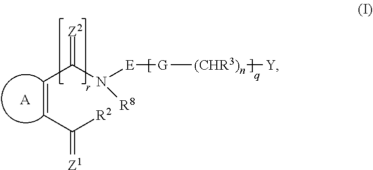

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R¹, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof, wherein the subject exhibits a level of a biomarker associated with drug addiction prior to the administering.

Embodiment C2. The method of Embodiment C1, wherein the biomarker is dopamine.

Embodiment C3. The method of Embodiment C1, wherein the drug addiction is cocaine addiction

Embodiment C4. The method of Embodiment C1, wherein the drug addiction is amphetamine addiction.

Embodiment C5. The method of Embodiment C1, wherein the therapeutically-effective amount is about 5 mg to about 100 mg.

Embodiment C6. The method of Embodiment C1, wherein the administering is oral.

Embodiment C7. The method of Embodiment C1, wherein the administering is oral by a solid dosage form.

Embodiment C8. The method of Embodiment C1, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment C9. The method of Embodiment C1, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment C10. The method of Embodiment C1, wherein the subject is human.

Embodiment C11. The method of Embodiment C1, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹, —SR¹¹, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment C12. The method of Embodiment C1, wherein the compound is of Formula (II):

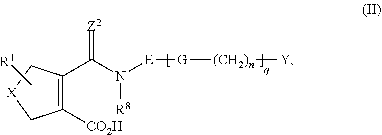

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹, —SR¹, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment C13. The method of Embodiment C1, wherein the compound is of Formula (III):

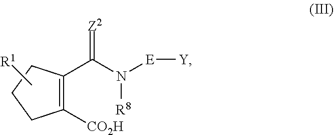

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment C14. The method of Embodiment C1, wherein the compound is of Formula (IV):

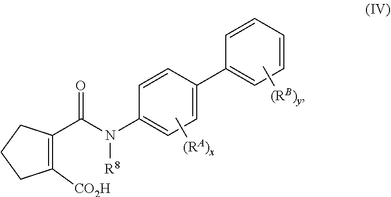

wherein: each R^{A} and R^{B} is independently —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment C15. The method of Embodiment C1, wherein the compound is

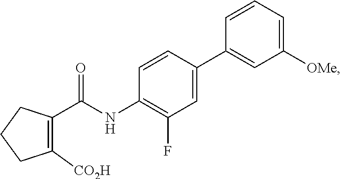 [SMILES: COc1cccc(-c2ccc(NC(=O)C3=C(C(=O)O)CCC3)c(F)c2)c1]

or a pharmaceutically-acceptable salt or solvate thereof.

Embodiment C16. The method of Embodiment C1, wherein the compound is

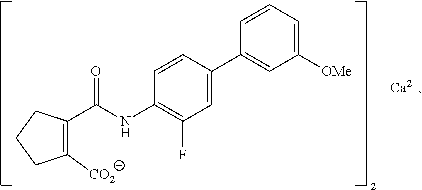

or a solvate thereof.

Embodiment C17. The method of Embodiment C16, wherein the compound is the solvate, wherein the solvate is a dihydrate.

Embodiment C18. The method of Embodiment C1, wherein the compound of Formula (I) is the compound of Formula (VI) or a pharmaceutically acceptable salt or solvate thereof:

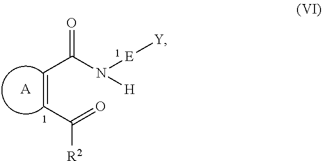

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

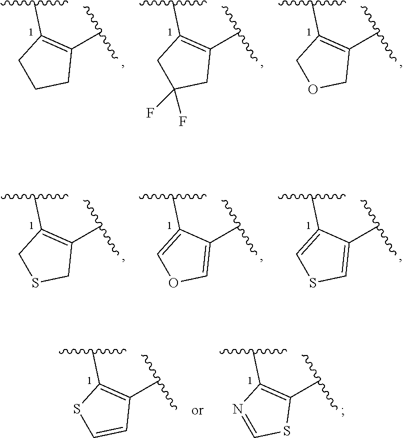

E is

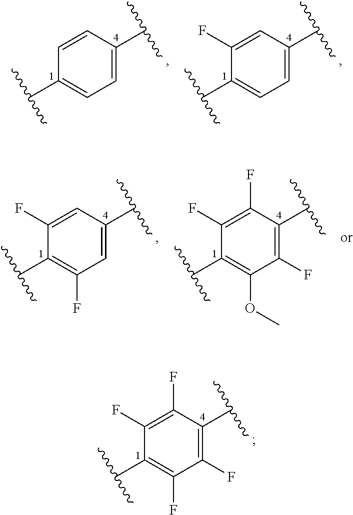 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

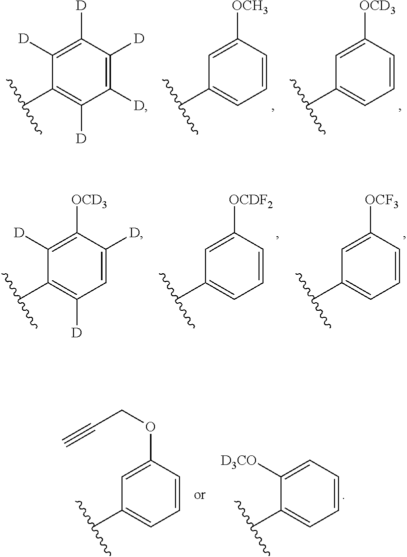

Embodiment D1. A method comprising: a) obtaining a level of Nurr1 in a subject; and b) based on the level of Nurr1 in the subject, determining whether to administer to the subject a Nurr1 agonist.

Embodiment D2. The method of Embodiment D1, wherein the obtaining the level of Nurr1 in the subject comprises obtaining a result of an assay of an ex vivo biological sample of the subject, wherein the assay determines a level of Nurr1 in the ex vivo biological sample of the subject.

Embodiment D3. The method of Embodiment D1, wherein the obtaining the level of Nurr1 in the subject comprises performing an assay of an ex vivo biological sample of the subject, wherein the assay determines a level of Nurr1 in the ex vivo biological sample of the subject.

Embodiment D4. The method of Embodiment D3, wherein the assay determines the level of Nurr1 in the ex vivo biological sample of the subject by determining a concentration of Nurr1 in the ex vivo biological sample of the subject.

Embodiment D5. The method of Embodiment D3, wherein the assay determines the level of Nurr1 in the ex vivo biological sample of the subject by determining a level of activity of Nurr1 in the ex vivo biological sample of the subject.

Embodiment D6. The method of Embodiment D3, wherein the assay is an ELISA assay, multiplex nucleic acid hybridization assay, a quantitative real time (qRT)-PCR assay, or a RNA sequencing (RNAseq) assay.

Embodiment D7. The method of Embodiment D3, wherein the ex vivo biological sample is a blood sample.

Embodiment D8. The method of Embodiment D1, wherein the neurodegenerative condition is an inflammatory condition.

Embodiment D9. The method of Embodiment D1, wherein the neurodegenerative condition is a mental condition.

Embodiment D10. The method of Embodiment D1, wherein the neurodegenerative condition is a central nervous system condition.

Embodiment D11. The method of Embodiment D1, wherein the neurodegenerative condition is Parkinson's Disease.

Embodiment D12. The method of Embodiment D1, wherein the neurodegenerative condition is Alzheimer's Disease.

Embodiment D13. The method of Embodiment D1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment D13a. The method of Embodiment D1, wherein the neurodegenerative condition is active SPMS.

Embodiment D13b. The method of Embodiment D1, wherein the neurodegenerative condition is PPMS.

Embodiment D13c. The method of Embodiment D1, wherein the neurodegenerative condition is non-active SPMS.

Embodiment D13d. The method of Embodiment D1, wherein the neurodegenerative condition is PIRA.

Embodiment D13e. The method of Embodiment D1, wherein the neurodegenerative condition is clinically isolated syndrome (CIS).

Embodiment D13f. The method of Embodiment D1, wherein the neurodegenerative condition is transitioning MS.

Embodiment D14. The method of Embodiment D1, wherein the neurodegenerative condition is amyotrophic lateral sclerosis.

Embodiment D15. The method of Embodiment D1, wherein the neurodegenerative condition is schizophrenia.

Embodiment D16. The method of Embodiment D1, further comprising, based on the level of Nurr1 in the subject, administering to the subject a therapeutically-effective amount of the Nurr1 agonist.

Embodiment D17. The method of Embodiment D16, wherein the therapeutically-effective amount is about 5 mg to about 100 mg.

Embodiment D18. The method of Embodiment D16, wherein the administering is oral.

Embodiment D19. The method of Embodiment D16, wherein the administering is oral by a solid dosage form.

Embodiment D20. The method of Embodiment D16, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment D21. The method of Embodiment D16, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment D22. The method of Embodiment D1 wherein the subject is human.

Embodiment D23. The method of Embodiment D1, wherein the Nurr1 agonist is a compound of Formula (I),

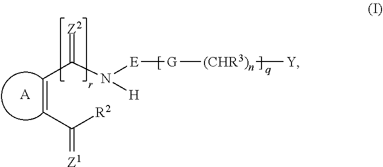

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹¹, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment D24. The method of Embodiment D23, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR, —SR¹³, —NR¹¹R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment D25. The method of Embodiment D24, wherein the compound is of Formula (II):

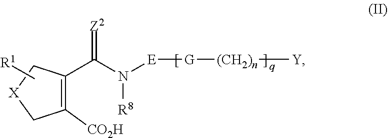

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR, —SR¹³, —NR¹¹R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment D26. The method of Embodiment D24, wherein the compound is of Formula (III):

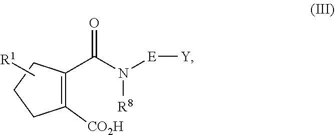

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment D27. The method of Embodiment D24, wherein the compound is of Formula (IV):

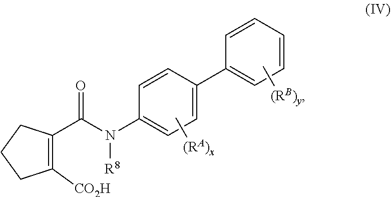

wherein: each R^{A} and R^{B} is independently —OR, —SR¹³, —NR¹¹R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment D28. The method of Embodiment D24, wherein the compound is

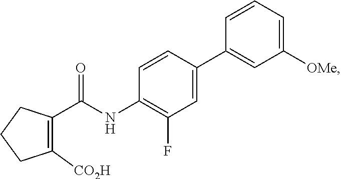 [SMILES: COc1cccc(-c2ccc(NC(=O)C3=C(C(=O)O)CCC3)c(F)c2)c1]

or a pharmaceutically-acceptable salt or solvate thereof.

Embodiment D29. The method of Embodiment D24, wherein the compound is

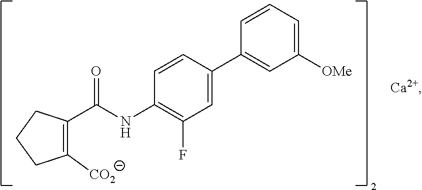

or a solvate thereof.

Embodiment D30. The method of Embodiment D29, wherein the compound is the solvate, wherein the solvate is a dihydrate.

Embodiment D31. The method of Embodiment D24, wherein the compound of Formula (I) is the compound of Formula (VI) or a pharmaceutically acceptable salt or solvate thereof:

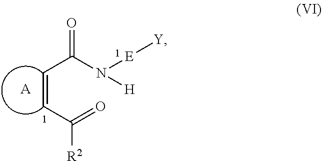

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

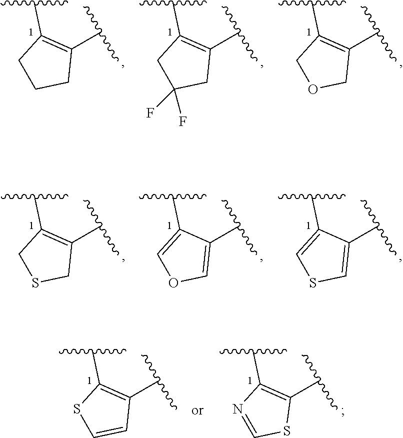

E is

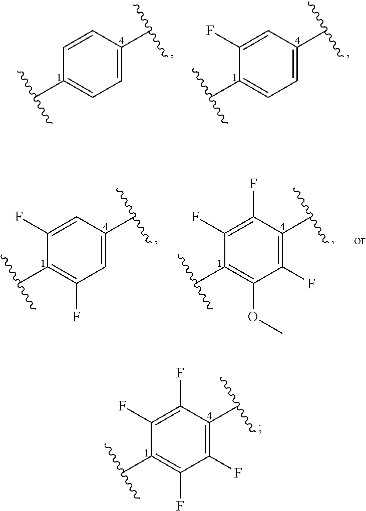 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

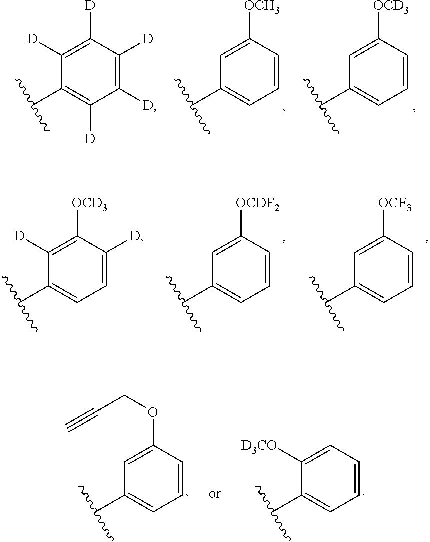

Embodiment E1. A method comprising: a) obtaining a level of Nurr1 in a subject who is undergoing a therapy for a neurodegenerative condition; and b) based on the level of Nurr1 in the subject, determining whether to continue the therapy for the neurodegenerative condition, wherein the therapy is Nurr1 agonism.

Embodiment E2. The method of Embodiment E1, wherein the obtaining the level of Nurr1 in the subject comprises obtaining a result of an assay of an ex vivo biological sample of the subject, wherein the assay determines a level of Nurr1 in the ex vivo biological sample of the subject.

Embodiment E3. The method of Embodiment E1, wherein the obtaining the level of Nurr1 in the subject comprises performing an assay of an ex vivo biological sample of the subject, wherein the assay determines a level of Nurr1 in the ex vivo biological sample of the subject.

Embodiment E4. The method of Embodiment E3, wherein the assay determines the level of Nurr1 in the ex vivo biological sample of the subject by determining a concentration of Nurr1 in the ex vivo biological sample of the subject.

Embodiment E5. The method of Embodiment E3, wherein the assay determines the level of Nurr1 in the ex vivo biological sample of the subject by determining a level of activity of Nurr1 in the ex vivo biological sample of the subject.

Embodiment E6. The method of Embodiment E3, wherein the assay is an ELISA assay, a multiplex nucleic acid hybridization assay, a quantitative real time (qRT)-PCR assay, or a RNA sequencing (RNAseq) assay.

Embodiment E7. The method of Embodiment E3, wherein the ex vivo biological sample is a blood sample.

Embodiment E8. The method of Embodiment E1, wherein the neurodegenerative condition is an inflammatory condition.

Embodiment E9. The method of Embodiment E1, wherein the neurodegenerative condition is a mental condition.

Embodiment E10. The method of Embodiment E1, wherein the neurodegenerative condition is a central nervous system condition.

Embodiment E11. The method of Embodiment E1, wherein the neurodegenerative condition is Parkinson's Disease.

Embodiment E12. The method of Embodiment E1, wherein the neurodegenerative condition is Alzheimer's Disease.

Embodiment E13. The method of Embodiment E1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment E13a. The method of Embodiment E1, wherein the neurodegenerative condition is active SPMS.

Embodiment E13b. The method of Embodiment E1, wherein the neurodegenerative condition is PPMS.

Embodiment E13c. The method of Embodiment E1, wherein the neurodegenerative condition is non-active SPMS.

Embodiment E13d. The method of Embodiment E1, wherein the neurodegenerative condition is PIRA.

Embodiment E13e. The method of Embodiment E1, wherein the neurodegenerative condition is clinically isolated syndrome (CIS).

Embodiment E13f. The method of Embodiment E1, wherein the neurodegenerative condition is transitioning MS.

Embodiment E14. The method of Embodiment E1, wherein the neurodegenerative condition is amyotrophic lateral sclerosis.

Embodiment E15. The method of Embodiment E1, wherein the neurodegenerative condition is schizophrenia.

Embodiment E16. The method of Embodiment E1, wherein the therapy for the neurodegenerative condition comprises administering to the subject a therapeutically-effective amount of a Nurr1 agonist for the neurodegenerative condition.

Embodiment E17. The method of Embodiment E16, wherein the therapeutically-effective amount is about 5 mg to about 100 mg.

Embodiment E18. The method of Embodiment E16, wherein the administering is oral.

Embodiment E19. The method of Embodiment E16, wherein the administering is oral by a solid dosage form.

Embodiment E20. The method of Embodiment E16, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment E21. The method of Embodiment E16, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment E22. The method of Embodiment E16, wherein the subject is human.

Embodiment E23. The method of Embodiment E16, wherein the Nurr1 agonist is a compound of Formula (I),

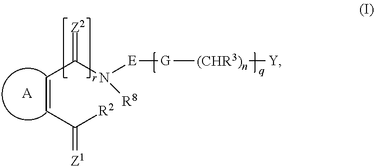

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment E24. The method of Embodiment E23, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment E25. The method of Embodiment E23, wherein the compound is of Formula (II):

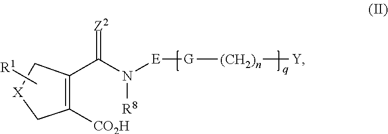

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment E26. The method of Embodiment E23, wherein the compound is of Formula (III):

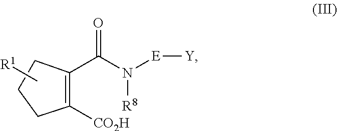

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment E27. The method of Embodiment E23, wherein the compound is of Formula (IV):

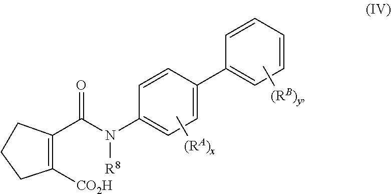

wherein: each R^{A} and R^{B} is independently —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment E28. The method of Embodiment E23, wherein the compound is

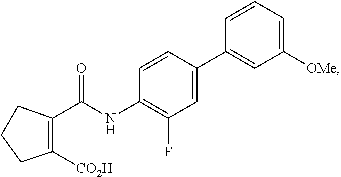 [SMILES: COc1cccc(-c2ccc(NC(=O)C3=C(C(=O)O)CCC3)c(F)c2)c1]

or a pharmaceutically-acceptable salt or solvate thereof.

Embodiment E29. The method of Embodiment E23, wherein the compound is

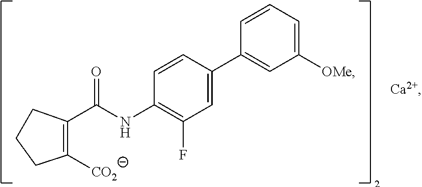

or a solvate thereof.

Embodiment E30. The method of Embodiment E29, wherein the compound is the solvate, wherein the solvate is a dihydrate.

Embodiment E31. The method of Embodiment E23, wherein the compound of Formula (I) is the compound of Formula (VI) or a pharmaceutically acceptable salt or solvate thereof:

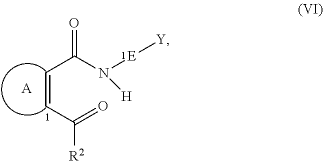

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

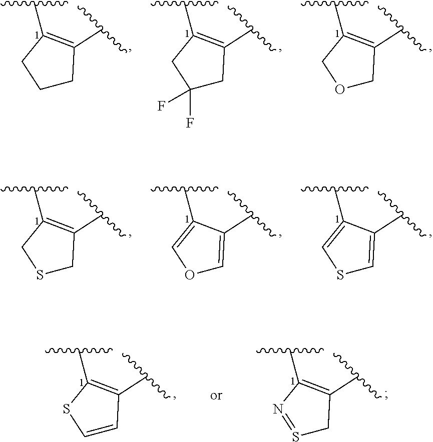

E is

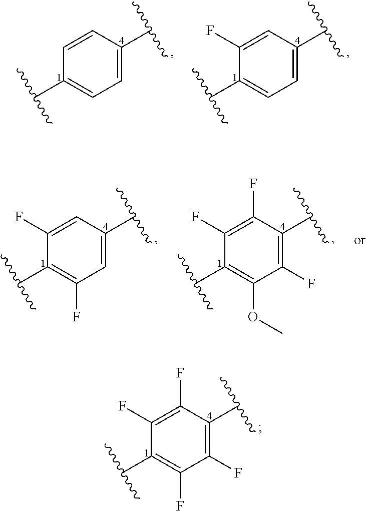 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

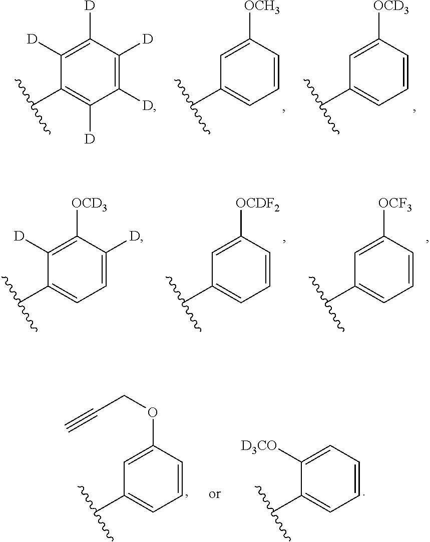

Embodiment F1. A method comprising: a) obtaining a level of a protein in a subject, wherein the protein is downstream of Nurr1 in a biological pathway in the subject; and b) based on the level of the protein in the subject, determining whether to administer to the subject a Nurr1 agonist.

Embodiment F2. The method of Embodiment F1, wherein the obtaining the level of the protein in the subject comprises obtaining a result of an assay of an ex vivo biological sample of the subject, wherein the assay determines a level of the protein in the ex vivo biological sample of the subject.

Embodiment F3. The method of Embodiment F1, wherein the obtaining the level of the protein in the subject comprises performing an assay of an ex vivo biological sample of the subject, wherein the assay determines a level of the protein in the ex vivo biological sample of the subject.

Embodiment F4. The method of Embodiment F3, wherein the assay determines the level of the protein in the ex vivo biological sample of the subject by determining a concentration of the protein in the ex vivo biological sample of the subject.

Embodiment F5. The method of Embodiment F3, wherein the assay determines the level of the protein in the ex vivo biological sample of the subject by determining a level of activity of the protein in the ex vivo biological sample of the subject.

Embodiment F6. The method of Embodiment F3, wherein the assay is an ELISA assay, a multiplex nucleic acid hybridization assay, a quantitative real time (qRT)-PCR assay, or a RNA sequencing (RNAseq) assay.

Embodiment F7. The method of Embodiment F1, wherein the level of the protein in the subject indicates that the protein is upregulated.

Embodiment F8. The method of Embodiment F1, wherein the level of the protein in the subject indicates that the protein is downregulated.

Embodiment F9. The method of Embodiment F3, wherein the ex vivo biological sample is a blood sample.

Embodiment F10. The method of Embodiment F1, wherein the protein is Pitx3.

Embodiment F11. The method of Embodiment F1, wherein the protein is TH.

Embodiment F12. The method of Embodiment F1, wherein the protein is VMAT2.

Embodiment F13. The method of Embodiment F1, wherein the protein is dopa decarboxylase (DDC).

Embodiment F14. The method of Embodiment F1, wherein the protein is dopamine transporter (DAT).

Embodiment F15. The method of Embodiment F1, wherein the protein is BDNF.

Embodiment F16. The method of Embodiment F1, wherein the protein is NGF.

Embodiment F17. The method of Embodiment F1, wherein the protein is GDNF receptor c-Ret.

Embodiment F18. The method of Embodiment F1, wherein the protein is GFAP.

Embodiment F19. The method of Embodiment F1, wherein the protein is SOD1.

Embodiment F20. The method of Embodiment F1, wherein the protein is TNFα.

Embodiment F21. The method of Embodiment F1, wherein the protein is iNOS.

Embodiment F22. The method of Embodiment F1, wherein the protein is IL-1β.

Embodiment F23. The method of Embodiment F1, wherein the neurodegenerative condition is an inflammatory condition.

Embodiment F24. The method of Embodiment F1, wherein the neurodegenerative condition is a mental condition.

Embodiment F25. The method of Embodiment F1, wherein the neurodegenerative condition is a central nervous system condition.

Embodiment F26. The method of Embodiment F1, wherein the neurodegenerative condition is Parkinson's Disease.

Embodiment F27. The method of Embodiment F1, wherein the neurodegenerative condition is Alzheimer's Disease.

Embodiment F28. The method of Embodiment F1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment F28a. The method of Embodiment F1, wherein the neurodegenerative condition is active SPMS.

Embodiment F28b. The method of Embodiment F1, wherein the neurodegenerative condition is PPMS.

Embodiment F28c. The method of Embodiment F1, wherein the neurodegenerative condition is non-active SPMS.

Embodiment F28d. The method of Embodiment F1, wherein the neurodegenerative condition is PIRA.

Embodiment F28e. The method of Embodiment F1, wherein the neurodegenerative condition is clinically isolated syndrome (CIS).

Embodiment F28f. The method of Embodiment F1, wherein the neurodegenerative condition is transitioning MS.

Embodiment F29. The method of Embodiment F1, wherein the neurodegenerative condition is amyotrophic lateral sclerosis.

Embodiment F30. The method of Embodiment F1, wherein the neurodegenerative condition is schizophrenia.

Embodiment F31. The method of Embodiment F1, further comprising, based on the level of the protein in the subject, administering to the subject a therapeutically-effective amount of the Nurr1 agonist.

Embodiment F32. The method of Embodiment F31, wherein the therapeutically-effective amount is about 5 mg to about 100 mg.

Embodiment F33. The method of Embodiment F31, wherein the administering is oral.

Embodiment F34. The method of Embodiment F31, wherein the administering is oral by a solid dosage form.

Embodiment F35. The method of Embodiment F31, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment F36. The method of Embodiment F1, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment F37. The method of Embodiment F1, wherein the subject is human.

Embodiment F38. The method of Embodiment F1, wherein the Nurr1 agonist is a compound of Formula (I),

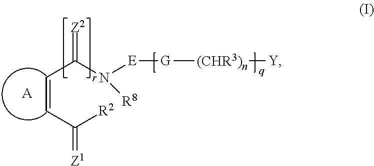

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment F39. The method of Embodiment F38, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment F40. The method of Embodiment F38, wherein the compound is of Formula (II):

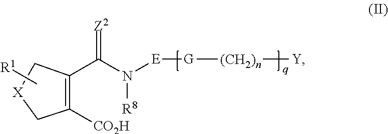

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment F41. The method of Embodiment F38, wherein the compound is of Formula (III):

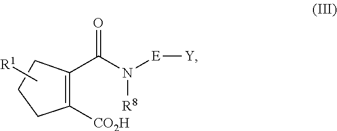

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment F42. The method of Embodiment F38, wherein the compound is of Formula (IV):

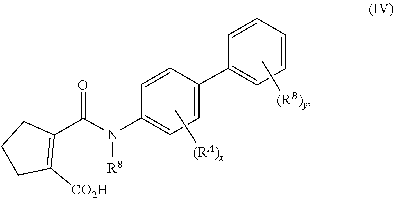

wherein: each R^{A} and R^{B} is independently —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment F43. The method of Embodiment F38, wherein the compound is

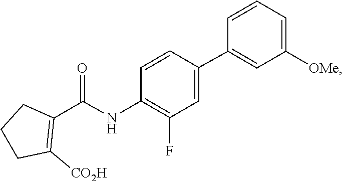 [SMILES: COc1cccc(-c2ccc(NC(=O)C3=C(C(=O)O)CCC3)c(F)c2)c1]

or a pharmaceutically-acceptable salt or solvate thereof.

Embodiment F44. The method of Embodiment F38, wherein the compound is

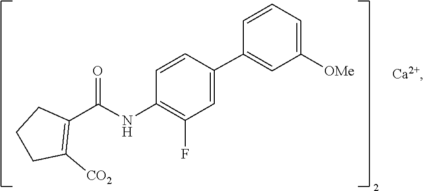

or a solvate thereof.

Embodiment F45. The method of Embodiment F44, wherein the compound is the solvate, wherein the solvate is a dihydrate.

Embodiment F46. The method of Embodiment F38, wherein the compound of Formula (I) is the compound of Formula (VI) or a pharmaceutically acceptable salt or solvate thereof:

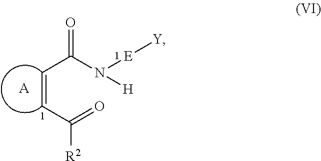

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

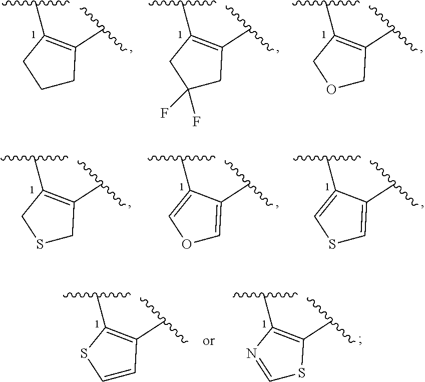

E is

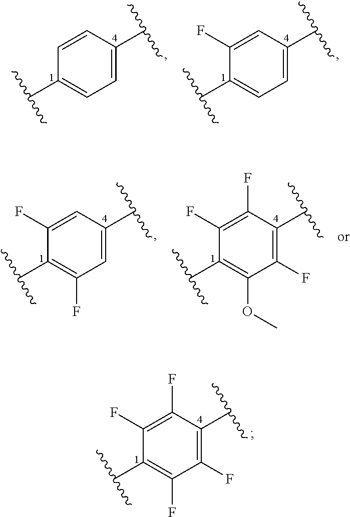 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

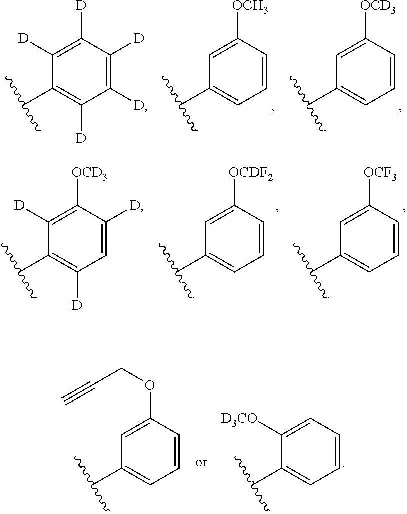

Embodiment G1. A method comprising: a) obtaining a level of a protein in a subject who is undergoing a therapy for a neurodegenerative condition, wherein the protein is downstream of Nurr1 in a biological pathway in the subject, wherein the therapy is Nurr1 agonism; and b) based on the level of the protein in the subject, determining whether to continue the therapy for the neurodegenerative condition.

Embodiment G2. The method of Embodiment G1, wherein the obtaining the level of the protein in the subject comprises obtaining a result of an assay of an ex vivo biological sample of the subject, wherein the assay determines a level of the protein in the ex vivo biological sample of the subject.

Embodiment G3. The method of Embodiment G1, wherein the obtaining the level of the protein in the subject comprises performing an assay of an ex vivo biological sample of the subject, wherein the assay determines a level of the protein in the ex vivo biological sample of the subject.

Embodiment G4. The method of Embodiment G3, wherein the assay determines the level of the protein in the ex vivo biological sample of the subject by determining a concentration of the protein in the ex vivo biological sample of the subject.

Embodiment G5. The method of Embodiment G3, wherein the assay determines the level of the protein in the ex vivo biological sample of the subject by determining a level of activity of the protein in the ex vivo biological sample of the subject.

Embodiment G6. The method of Embodiment G3, wherein the assay is an ELISA assay, a multiplex nucleic acid hybridization assay, a quantitative real time (qRT)-PCR assay, or a RNA sequencing (RNAseq) assay.

Embodiment G7. The method of Embodiment G1, wherein the level of the protein in the subject is higher than the level was prior to the therapy.

Embodiment G8. The method of Embodiment G1, wherein the level of the protein in the subject is lower than the level was prior to the therapy.

Embodiment G9. The method of Embodiment G3, wherein the ex vivo biological sample is a blood sample.

Embodiment G10. The method of Embodiment G1, wherein the protein is Pitx3.

Embodiment G11. The method of Embodiment G1, wherein the protein is Pitx3, and further comprising determining that the level of the protein in the subject is higher than the level was prior to the therapy.

Embodiment G12. The method of Embodiment G1, wherein the protein is TH.

Embodiment G13. The method of Embodiment G1, wherein the protein is TH, and further comprising determining that the level of the protein in the subject is higher than the level was prior to the therapy.

Embodiment G14. The method of Embodiment G1, wherein the protein is VMAT2.

Embodiment G15. The method of Embodiment G1, wherein the protein is VMAT2, and further comprising determining that the level of the protein in the subject is higher than the level was prior to the therapy.

Embodiment G16. The method of Embodiment G1, wherein the protein is dopa decarboxylase (DDC).

Embodiment G17. The method of Embodiment G1, wherein the protein is dopa decarboxylase (DDC), and further comprising determining that the level of the protein in the subject is higher than the level was prior to the therapy.

Embodiment G18. The method of Embodiment G1, wherein the protein is dopamine transporter (DAT).

Embodiment G19. The method of Embodiment G1, wherein the protein is dopamine transporter (DAT), and further comprising determining that the level of the protein in the subject is higher than the level was prior to the therapy.

Embodiment G20. The method of Embodiment G1, wherein the protein is BDNF.

Embodiment G21. The method of Embodiment G1, wherein the protein is BDNF, and further comprising determining that the level of the protein in the subject is higher than the level was prior to the therapy.

Embodiment G22. The method of Embodiment G1, wherein the protein is NGF.

Embodiment G23. The method of Embodiment G1, wherein the protein is NGF, and further comprising determining that the level of the protein in the subject is higher than the level was prior to the therapy.

Embodiment G24. The method of Embodiment G1, wherein the protein is GDNF receptor c-Ret.

Embodiment G25. The method of Embodiment G1, wherein the protein is GDNF receptor c-Ret, and further comprising determining that the level of the protein in the subject is higher than the level was prior to the therapy.

Embodiment G26. The method of Embodiment G1, wherein the protein is GFAP.

Embodiment G27. The method of Embodiment G1, wherein the protein is GFAP, and further comprising determining that the level of the protein in the subject is higher than the level was prior to the therapy.

Embodiment G28. The method of Embodiment G1, wherein the protein is SOD1.

Embodiment G29. The method of Embodiment G1, wherein the protein is SOD1, and further comprising determining that the level of the protein in the subject is higher than the level was prior to the therapy.

Embodiment G30. The method of Embodiment G1, wherein the protein is TNFα.

Embodiment G31. The method of Embodiment G1, wherein the protein is TNFα, and further comprising determining that the level of the protein in the subject is lower than the level was prior to the therapy.

Embodiment G32. The method of Embodiment G1, wherein the protein is iNOS.

Embodiment G33. The method of Embodiment G1, wherein the protein is iNOS, and further comprising determining that the level of the protein in the subject is lower than the level was prior to the therapy.

Embodiment G34. The method of Embodiment G1, wherein the protein is IL-1β.

Embodiment G35. The method of Embodiment G1, wherein the protein is IL-1β, and further comprising determining that the level of the protein in the subject is lower than the level was prior to the therapy.

Embodiment G36. The method of Embodiment G1, wherein the neurodegenerative condition is an inflammatory condition.

Embodiment G37. The method of Embodiment G1, wherein the neurodegenerative condition is a mental condition.

Embodiment G38. The method of Embodiment G1, wherein the neurodegenerative condition is a central nervous system condition.

Embodiment G39. The method of Embodiment G1, wherein the neurodegenerative condition is Parkinson's Disease.

Embodiment G40. The method of Embodiment G1, wherein the neurodegenerative condition is Alzheimer's Disease.

Embodiment G41. The method of Embodiment G1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment G41a. The method of Embodiment G1, wherein the neurodegenerative condition is active SPMS.

Embodiment G41b. The method of Embodiment G1, wherein the neurodegenerative condition is PPMS.

Embodiment G41c. The method of Embodiment G1, wherein the neurodegenerative condition is non-active SPMS.

Embodiment G41d. The method of Embodiment G1, wherein the neurodegenerative condition is PIRA.

Embodiment G41e. The method of Embodiment G1, wherein the neurodegenerative condition is clinically isolated syndrome (CIS).

Embodiment G41f. The method of Embodiment G1, wherein the neurodegenerative condition is transitioning MS.

Embodiment G42. The method of Embodiment G1, wherein the neurodegenerative condition is amyotrophic lateral sclerosis.

Embodiment G43. The method of Embodiment G1, wherein the neurodegenerative condition is schizophrenia.

Embodiment G44. The method of Embodiment G1, wherein the therapy for the neurodegenerative condition comprises administering to the subject a therapeutically-effective amount of a therapeutic agent for the neurodegenerative condition.

Embodiment G45. The method of Embodiment G44, wherein the therapeutically-effective amount is about 5 mg to about 100 mg.

Embodiment G46. The method of Embodiment G44, wherein the administering is oral.

Embodiment G47. The method of Embodiment G44, wherein the administering is oral by a solid dosage form.

Embodiment G48. The method of Embodiment G44, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment G49. The method of Embodiment G44, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment G50. The method of Embodiment G1, wherein the subject is human.

Embodiment G51. The method of Embodiment G44, wherein the therapeutic agent is a compound of Formula (I),

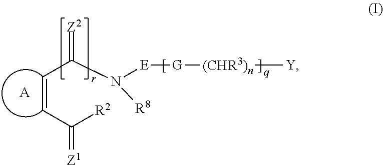

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment G52. The method of Embodiment G51, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment G53. The method of Embodiment G51, wherein the compound is of Formula (II):

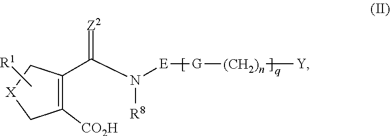

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment G54. The method of Embodiment G51, wherein the compound is of Formula

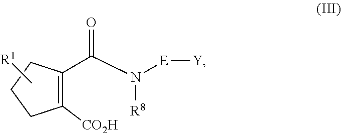

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment G55. The method of Embodiment G51, wherein the compound is of Formula (IV):

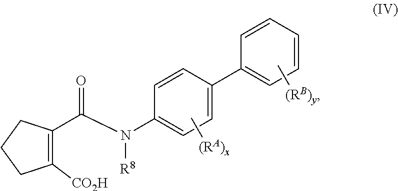

wherein: each R^{A} and R^{B} is independently —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment G56. The method of Embodiment G51, wherein the compound is

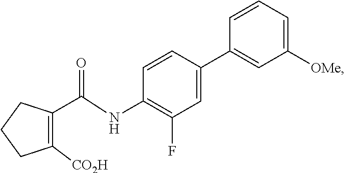 [SMILES: COc1cccc(-c2ccc(NC(=O)C3=C(C(=O)O)CCC3)c(F)c2)c1]

or a pharmaceutically-acceptable salt or solvate thereof.

Embodiment G57. The method of Embodiment G51, wherein the compound is

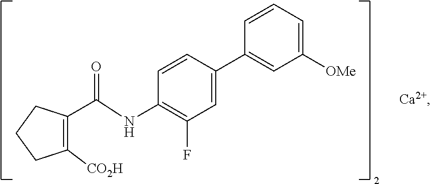

or a solvate thereof.

Embodiment G58. The method of Embodiment G57, wherein the compound is the solvate, wherein the solvate is a dihydrate.

Embodiment G59. The method of Embodiment G51, wherein the compound of Formula (I) is the compound of Formula (VI) or a pharmaceutically acceptable salt or solvate thereof:

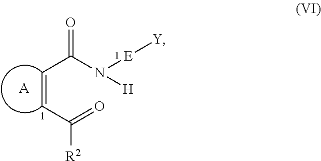

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

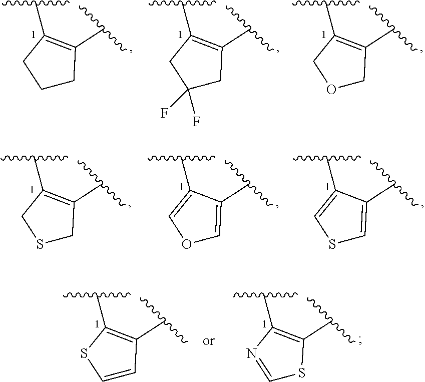

E is

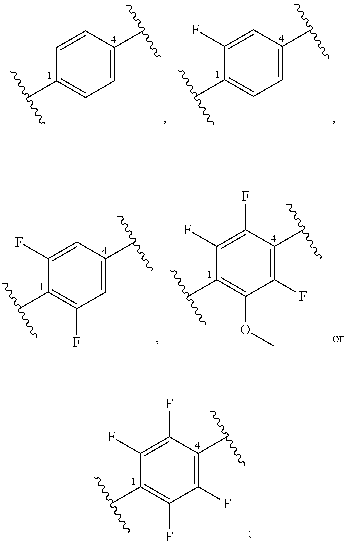 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

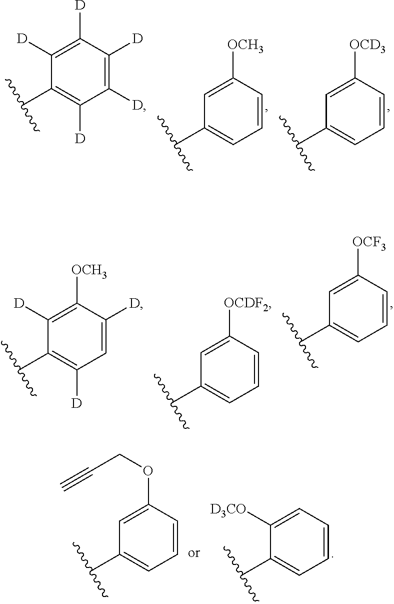

Embodiment H1. A method comprising: a) obtaining a level of activity of a gene in a subject, wherein the gene is downstream of Nurr1 in a biological pathway in the subject; and b) based on the level of activity of the gene in the subject, determining whether to administer to the subject a Nurr1 agonist for a neurodegenerative condition.

Embodiment H2. The method of Embodiment H1, wherein the obtaining the level of activity of the gene in the subject comprises obtaining a result of an assay of an ex vivo biological sample of the subject, wherein the assay determines a level of activity of the gene in the ex vivo biological sample of the subject.

Embodiment H3. The method of Embodiment H1, wherein the obtaining the level of activity of the gene in the subject comprises performing an assay of an ex vivo biological sample of the subject, wherein the assay determines a level of activity of the gene in the ex vivo biological sample of the subject.

Embodiment H4. The method of Embodiment H3, wherein the assay is a multiplex nucleic acid hybridization assay, a quantitative real time (qRT)-PCR assay, or a RNA sequencing (RNAseq) assay.

Embodiment H5. The method of Embodiment H1, wherein the level of activity of the gene in the subject indicates that the gene is upregulated.

Embodiment H6. The method of Embodiment H3, wherein the ex vivo biological sample is a blood sample.

Embodiment H7. The method of Embodiment H1, wherein the gene is NCAM.

Embodiment H8. The method of Embodiment H1, wherein the gene is CFB.

Embodiment H9. The method of Embodiment H1, wherein the gene is LTA.

Embodiment H10. The method of Embodiment H1, wherein the gene is A2M.

Embodiment H11. The method of Embodiment H1, wherein the gene is HSD11B1.

Embodiment H12. The method of Embodiment H1, wherein the gene is BHLHE41.

Embodiment H13. The method of Embodiment H1, wherein the gene is MARCO.

Embodiment H14. The method of Embodiment H1, wherein the neurodegenerative condition is an inflammatory condition.

Embodiment H15. The method of Embodiment H1, wherein the neurodegenerative condition is a mental condition.

Embodiment H16. The method of Embodiment H1, wherein the neurodegenerative condition is a central nervous system condition.

Embodiment H17. The method of Embodiment H1, wherein the neurodegenerative condition is Parkinson's Disease.

Embodiment H18. The method of Embodiment H1, wherein the neurodegenerative condition is Alzheimer's Disease.

Embodiment H19. The method of Embodiment H1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment H19a. The method of Embodiment H1, wherein the neurodegenerative condition is active SPMS.

Embodiment H19b. The method of Embodiment H1, wherein the neurodegenerative condition is PPMS.

Embodiment H19c. The method of Embodiment H1, wherein the neurodegenerative condition is non-active SPMS.

Embodiment H19d. The method of Embodiment H1, wherein the neurodegenerative condition is PIRA.

Embodiment H19e. The method of Embodiment H1, wherein the neurodegenerative condition is clinically isolated syndrome (CIS).

Embodiment H19f. The method of Embodiment H1, wherein the neurodegenerative condition is transitioning MS.

Embodiment H20. The method of Embodiment H1, wherein the neurodegenerative condition is amyotrophic lateral sclerosis.

Embodiment H21. The method of Embodiment H1, wherein the neurodegenerative condition is schizophrenia.

Embodiment H22. The method of Embodiment H1, further comprising, based on the level of activity of the gene in the subject, administering to the subject a therapeutically-effective amount of the Nurr1 agonist.

Embodiment H23. The method of Embodiment H22, wherein the therapeutically-effective amount is about 5 mg to about 100 mg.

Embodiment H24. The method of Embodiment H22, wherein the administering is oral.

Embodiment H25. The method of Embodiment H22, wherein the administering is oral by a solid dosage form.

Embodiment H26. The method of Embodiment H22, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment H27. The method of Embodiment H22, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment H28. The method of Embodiment H1, wherein the subject is human.

Embodiment H29. The method of Embodiment H1, wherein the Nurr1 agonist is a compound of Formula (I),

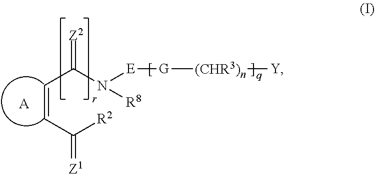

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment H30. The method of Embodiment H29, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment H31. The method of Embodiment H29, wherein the compound is of Formula (II):

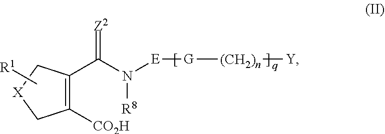

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment H32. The method of Embodiment H29, wherein the compound is of Formula (III):

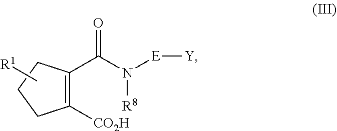

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment H33. The method of Embodiment H29, wherein the compound is of Formula (IV):

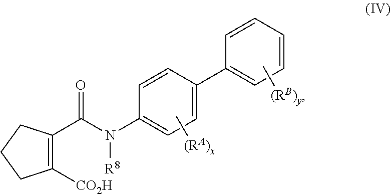

wherein: each R^{A} and R^{B} is independently —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment H34. The method of Embodiment H29, wherein the compound is

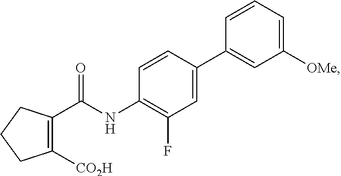 [SMILES: COc1cccc(-c2ccc(NC(=O)C3=C(C(=O)O)CCC3)c(F)c2)c1]

or a pharmaceutically-acceptable salt or solvate thereof.

Embodiment H35. The method of Embodiment H29, wherein the compound is

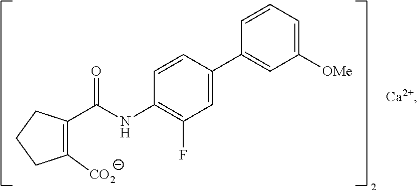

or a solvate thereof.

Embodiment H36. The method of Embodiment H35, wherein the compound is the solvate, wherein the solvate is a dihydrate.

Embodiment H37. The method of Embodiment H29, wherein the compound of Formula (I) is the compound of Formula (VI) or a pharmaceutically acceptable salt or solvate thereof:

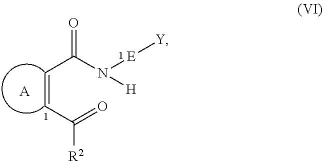

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

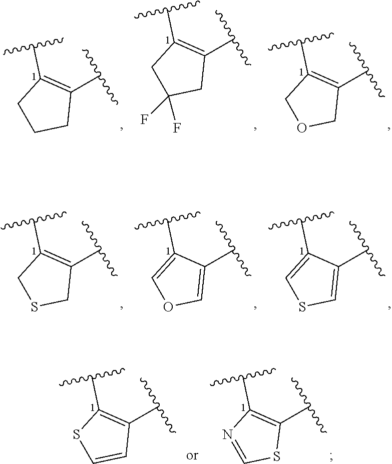

E is

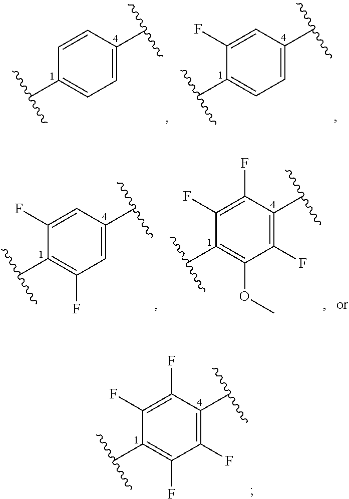 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

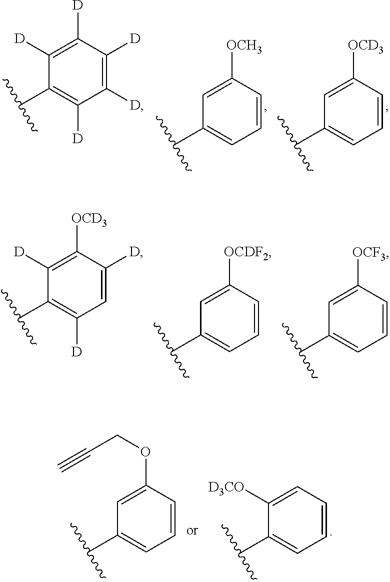

Embodiment I1. A method comprising: a) obtaining a level of activity of a gene in a subject who is undergoing a therapy for a neurodegenerative condition, wherein the gene is downstream of Nurr1 in a biological pathway in the subject, wherein the therapy is Nurr1 agonism; and b) based on the level of activity of the gene in the subject, determining whether to continue the therapy for the neurodegenerative condition.

Embodiment I2. The method of Embodiment I1, wherein the obtaining the level of activity of the gene in the subject comprises obtaining a result of an assay of an ex vivo biological sample of the subject, wherein the assay determines a level of activity of the gene in the ex vivo biological sample of the subject.

Embodiment I3. The method of Embodiment I1, wherein the obtaining the level of activity of the gene in the subject comprises performing an assay of an ex vivo biological sample of the subject, wherein the assay determines a level of activity of the gene in the ex vivo biological sample of the subject.

Embodiment I4. The method of Embodiment I3, wherein the assay is a multiplex nucleic acid hybridization assay, a quantitative real time (qRT)-PCR assay, or a RNA sequencing (RNAseq) assay.

Embodiment I5. The method of Embodiment I1, wherein the level of activity of the gene in the subject is higher than the level was prior to the therapy.

Embodiment I6. The method of Embodiment I3, wherein the ex vivo biological sample is a blood sample.

Embodiment I7. The method of Embodiment I1, wherein the gene is NCAM.

Embodiment I8. The method of Embodiment I1, wherein the gene is CFB.

Embodiment I9. The method of Embodiment I1, wherein the gene is LTA.

Embodiment I10. The method of Embodiment I1, wherein the gene is A2M.

Embodiment I11. The method of Embodiment I1, wherein the gene is HSD11B1.

Embodiment I12. The method of Embodiment I1, wherein the gene is BHLHE41.

Embodiment I13. The method of Embodiment I1, wherein the gene is MARCO.

Embodiment I14. The method of Embodiment I1, wherein the neurodegenerative condition is an inflammatory condition.

Embodiment I15. The method of Embodiment I1, wherein the neurodegenerative condition is a mental condition.

Embodiment I16. The method of Embodiment I1, wherein the neurodegenerative condition is a central nervous system condition.

Embodiment I17. The method of Embodiment I1, wherein the neurodegenerative condition is Parkinson's Disease.

Embodiment I18. The method of Embodiment I1, wherein the neurodegenerative condition is Alzheimer's Disease.

Embodiment I19. The method of Embodiment I1, wherein the neurodegenerative condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment I19a. The method of Embodiment I1, wherein the neurodegenerative condition is active SPMS.

Embodiment I19b. The method of Embodiment I1, wherein the neurodegenerative condition is PPMS.

Embodiment I19c. The method of Embodiment I1, wherein the neurodegenerative condition is non-active SPMS.

Embodiment I19d. The method of Embodiment I1, wherein the neurodegenerative condition is PIRA.

Embodiment I19e. The method of Embodiment I1, wherein the neurodegenerative condition is clinically isolated syndrome (CIS).

Embodiment I19f. The method of Embodiment I1, wherein the neurodegenerative condition is transitioning MS.

Embodiment I20. The method of Embodiment I1, wherein the neurodegenerative condition is amyotrophic lateral sclerosis.

Embodiment I21. The method of Embodiment I1, wherein the neurodegenerative condition is schizophrenia.

Embodiment I22. The method of Embodiment I1, wherein the therapy for the neurodegenerative condition comprises administering to the subject a therapeutically-effective amount of a therapeutic agent for the neurodegenerative condition.

Embodiment I23. The method of Embodiment I22, wherein the therapeutically-effective amount is about 5 mg to about 100 mg.

Embodiment I24. The method of Embodiment I22, wherein the administering is oral.

Embodiment I25. The method of Embodiment I22, wherein the administering is oral by a solid dosage form.

Embodiment I26. The method of Embodiment I22, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment I27. The method of Embodiment I22, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment I28. The method of Embodiment I1, wherein the subject is human.

Embodiment I29. The method of Embodiment I22, wherein the therapeutic agent is a compound of Formula (I),

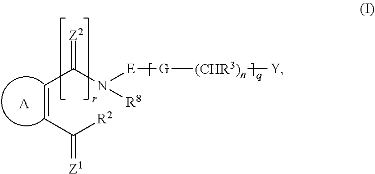

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment I30. The method of Embodiment I29, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment I31. The method of Embodiment I29, wherein the compound is of Formula (II):

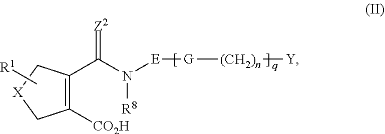

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment I32. The method of Embodiment I29, wherein the compound is of Formula (III):

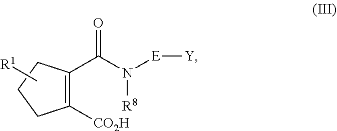

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment I33. The method of Embodiment I29, wherein the compound is of Formula (IV):

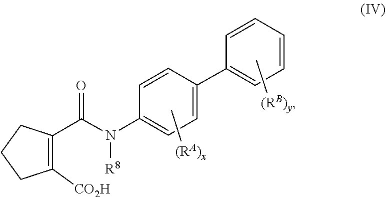

wherein: each R^{A} and R^{B} is independently —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment I34. The method of Embodiment I29, wherein the compound is

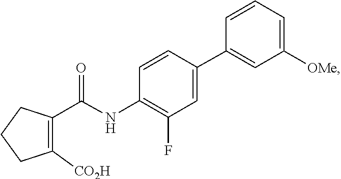 [SMILES: COc1cccc(-c2ccc(NC(=O)C3=C(C(=O)O)CCC3)c(F)c2)c1]

or a pharmaceutically-acceptable salt or solvate thereof.

Embodiment I35. The method of Embodiment I29, wherein the compound is

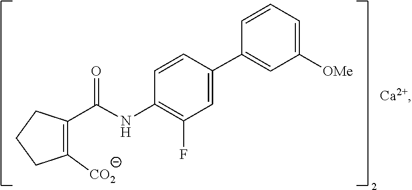

or a solvate thereof.

Embodiment I36. The method of Embodiment I35, wherein the compound is the solvate, wherein the solvate is a dihydrate.

Embodiment I37. The method of Embodiment I29, wherein the compound of Formula (I) is the compound of Formula (VI) or a pharmaceutically acceptable salt or solvate thereof:

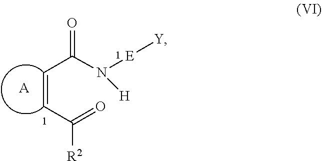

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

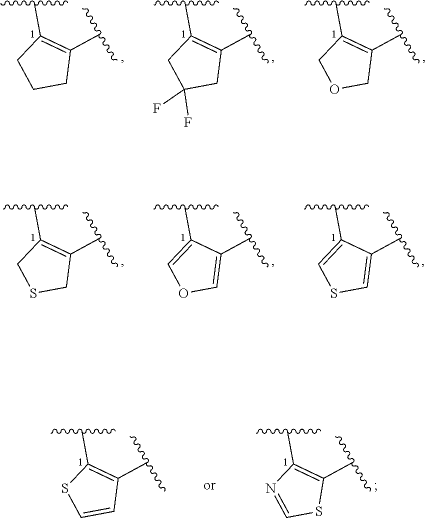

E is

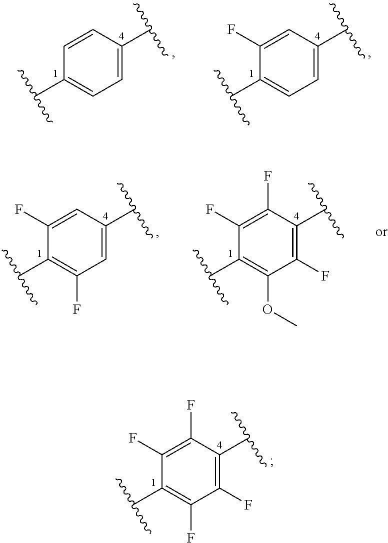 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

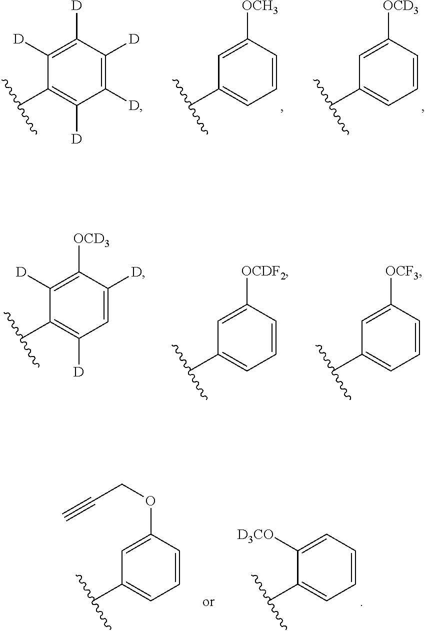

Embodiment J1. A method comprising: a) determining that a subject exhibits downregulated Nurr1; b) determining that the subject exhibits upregulated miR-132; and c) based on the determining that the subject exhibits downregulated Nurr1 and the determining that the subject exhibits upregulated miR-132, identifying the subject as being at risk for a condition.

Embodiment J2. The method of Embodiment J1, wherein the determining that the subject exhibits downregulated Nurr1 comprises obtaining a result of a first assay of an ex vivo biological sample of the subject, wherein the first assay determines a level of Nurr1 in the ex vivo biological sample of the subject; and the determining that the subject exhibits upregulated miR-132 comprises obtaining a result of a second assay of the ex vivo biological sample of the subject, wherein the second assay determines a level of miR-132 in the ex vivo biological sample of the subject.

Embodiment J3. The method of Embodiment J1, wherein the determining that the subject exhibits downregulated Nurr1 comprises performing a first assay of an ex vivo biological sample of the subject, wherein the first assay determines a level of Nurr1 in the ex vivo biological sample of the subject; and the determining that the subject exhibits upregulated miR-132 comprises performing a second assay of the ex vivo biological sample of the subject, wherein the second assay determines a level of miR-132 in the ex vivo biological sample of the subject.

Embodiment J4. The method of Embodiment J3, wherein the first assay determines the level of Nurr1 in the ex vivo biological sample of the subject by determining a concentration of Nurr1 in the ex vivo biological sample of the subject.

Embodiment J5. The method of Embodiment J3, wherein the first assay determines the level of Nurr1 in the ex vivo biological sample of the subject by determining a level of activity of Nurr1 in the ex vivo biological sample of the subject.

Embodiment J6. The method of Embodiment J3, wherein the first assay is an ELISA assay, a multiplex nucleic acid hybridization assay, a quantitative real time (qRT)-PCR assay, or a RNA sequencing (RNAseq) assay.

Embodiment J7. The method of Embodiment J3, wherein the second assay determines the level of miR-132 in the ex vivo biological sample of the subject by determining a concentration of miR-132 in the ex vivo biological sample of the subject.

Embodiment J8. The method of Embodiment J3, wherein the second assay determines the level of miR-132 in the ex vivo biological sample of the subject by determining a level of activity of miR-132 in the ex vivo biological sample of the subject.

Embodiment J9. The method of Embodiment J3, wherein the second assay is a multiplex nucleic acid hybridization assay, a quantitative real time (qRT)-PCR assay, or a RNA sequencing (RNAseq) assay.

Embodiment J10. The method of Embodiment J3, wherein the ex vivo biological sample is a blood sample.

Embodiment J11. The method of Embodiment J1, wherein the condition is a neurodegenerative condition.

Embodiment J12. The method of Embodiment J1, wherein the condition is an inflammatory condition.

Embodiment J13. The method of Embodiment J1, wherein the condition is a mental condition.

Embodiment J14. The method of Embodiment J1, wherein the condition is a central nervous system condition.

Embodiment J15. The method of Embodiment J1, wherein the condition is Parkinson's Disease.

Embodiment J16. The method of Embodiment J1, wherein the condition is Alzheimer's Disease.

Embodiment J17. The method of Embodiment J1, wherein the condition is Multiple Sclerosis, in particular relapsing form of multiple sclerosis (RMS), such as relapsing-remitting multiple sclerosis (RRMS) or active secondary progressive multiple sclerosis (active SPMS), or progressive form of multiple sclerosis, such as primary progressive multiple sclerosis (PPMS) or non-active secondary progressive multiple sclerosis (non-active SPMS).

Embodiment J17a. The method of Embodiment J1, wherein the condition is active SPMS.

Embodiment J17b. The method of Embodiment J1, wherein the condition is PPMS.

Embodiment J17c. The method of Embodiment J1, wherein the condition is non-active SPMS.

Embodiment J17d. The method of Embodiment J1, wherein the condition is PIRA.

Embodiment J17e. The method of Embodiment J1, wherein the condition is clinically isolated syndrome (CIS).

Embodiment J17f. The method of Embodiment J1, wherein the condition is transitioning MS.

Embodiment J18. The method of Embodiment J1, wherein the condition is amyotrophic lateral sclerosis.

Embodiment J19. The method of Embodiment J1, wherein the condition is schizophrenia.

Embodiment J20. The method of Embodiment J1, wherein the condition is drug addiction.

Embodiment J21. The method of Embodiment J1, further comprising, based on the determining that the subject exhibits downregulated Nurr1 and the determining that the subject exhibits upregulated miR-132, identifying the subject as a candidate for therapy for a condition.

Embodiment J22. The method of Embodiment J1, further comprising, based on the determining that the subject exhibits downregulated Nurr1 and the determining that the subject exhibits upregulated miR-132, administering to the subject a therapeutically-effective amount of a compound that modulates Nurr1.

Embodiment J23. The method of Embodiment J22, wherein the compound that modulates Nurr1 agonizes Nurr1.

Embodiment J24. The method of Embodiment J22, wherein the therapeutically-effective amount is about 5 mg to about 100 mg.

Embodiment J25. The method of Embodiment J22, wherein the administering is oral.

Embodiment J26. The method of Embodiment J22, wherein the administering is oral by a solid dosage form.

Embodiment J27. The method of Embodiment J22, wherein the administering is over a first period and a second period, wherein: a) the first period comprises at least five consecutive days of once-daily dosing of a first period amount of the compound, wherein the first period amount of the compound is about 5 mg to about 50 mg; b) the second period follows the first period; and c) the second period comprises once-daily dosing of a second period amount of the compound, wherein the second period amount is greater than the first period amount is.

Embodiment J28. The method of Embodiment J22, wherein the administering is over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 40 mg to about 50 mg.

Embodiment J29. The method of Embodiment J1, wherein the subject is human.

Embodiment J30. The method of Embodiment J22, wherein the compound that modulates Nurr1 is a compound of Formula (I),

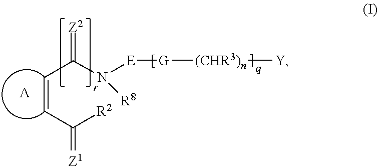

wherein: A is a ring that is unsubstituted or substituted; Z¹ and Z² are each independently O, S, or NR⁹; E is a linker or is absent; G is a linker or is absent; Y is a ring; R² is H, OR¹¹, NR¹¹OR¹¹, NR¹¹SO₂R¹¹, or NR¹¹R¹²; R³ is H, halo, —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted; each R⁸, R⁹, R¹¹, R¹², R¹³, and R¹⁴ is independently H, alkyl, alkenyl, alkynyl, aryl, cycloalkyl, heteroaryl, or heterocyclyl, each of which is independently substituted or unsubstituted; n is 0, 1, 2, 3, 4, or 5; q is 0, 1, 2, 3, 4, 5, 6, 7, 8, 9, or 10; and r is 0 or 1, or a pharmaceutically-acceptable salt thereof.

Embodiment J31. The method of Embodiment J30, wherein: Ring A is a 5- or 6-membered carbocyclic or heterocyclic ring, each of which is substituted or unsubstituted; Z¹ and Z² are each independently O, S, or NR⁹; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R² is H, OR¹¹, or NR¹¹R¹²; R³ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; q is 0, 1, 2, 3, 4, 5, 6, 7, 8, 9, or 10; and r is 0 or 1.

Embodiment J32. The method of Embodiment J30, wherein the compound is of Formula (II):

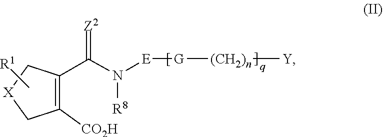

wherein: X is O, S, NR⁹, SO, or SO₂; Z² is O, S, or NR¹²; E is alkylene, alkenylene, alkynylene, arylene, heteroarylene, or cycloalkylene, each of which is independently substituted or unsubstituted; G is O, S, SO₂, NR¹⁰, or CH₂; Y is aryl, heteroaryl, heterocyclyl, or cycloalkyl, each of which is independently substituted or unsubstituted; R¹ is —OR¹¹, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each R⁸, R⁹, R¹⁰, R¹¹, R¹², R¹³, and R¹⁴ is independently alkyl, alkenyl, alkynyl, aryl, cycloalkyl, heteroaryl, or heterocyclyl, each of which is independently substituted or unsubstituted, or hydrogen; n is 0 or 1; and q is 0, 1, 2, 3, 4, 5, 6, 7, 8, 9, or 10.

Embodiment J33. The method of Embodiment J30, wherein the compound is of Formula (III):

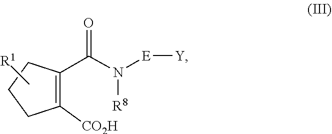

wherein: E is alkylene, alkenylene, alkynylene, arylene, heteroarylene, or cycloalkylene, each of which is independently substituted or unsubstituted; Y is aryl, heteroaryl, heterocyclyl, or cycloalkyl, each of which is independently substituted or unsubstituted; R¹ is —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment J33. The method of Embodiment J30, wherein the compound is of Formula (IV):

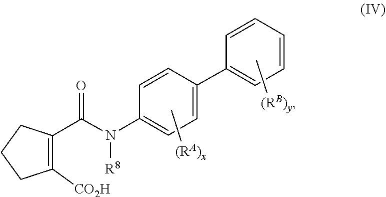

wherein: each R^{A} and R^{B} is independently —OR¹³, —SR¹³, —NR¹³R¹⁴, alkyl, alkenyl, alkynyl, alkoxy, aryl, cycloalkyl, heteroaryl, or heterocyclyl, each of which is independently substituted or unsubstituted, or hydrogen or halo; each x and y is independently 0, 1, 2, 3, or 4; and each R⁸, R¹³, and R¹⁴ is independently alkyl, alkenyl, alkynyl, aryl, cycloalkyl, heteroaryl, or heterocyclyl, each of which is independently substituted or unsubstituted, or hydrogen.

Embodiment J35. The method of Embodiment J30, wherein the compound is

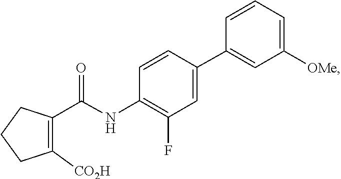 [SMILES: COc1cccc(-c2ccc(NC(=O)C3=C(C(=O)O)CCC3)c(F)c2)c1]

or a pharmaceutically-acceptable salt or solvate thereof.

Embodiment J36. The method of Embodiment J30, wherein the compound is

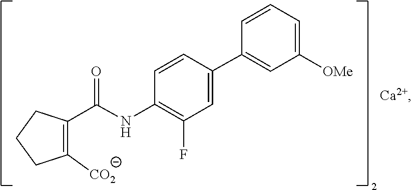

or a solvate thereof.

Embodiment J37. The method of Embodiment J36, wherein the compound is the solvate, wherein the solvate is a dihydrate.

Embodiment J38. The method of Embodiment J30, wherein the compound of Formula (I) is the compound of Formula (VI) or a pharmaceutically acceptable salt or solvate thereof:

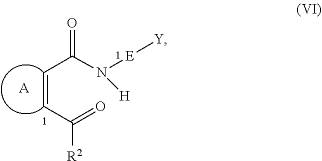

wherein:
   R² is NR¹¹R¹²;
   R¹¹ is selected from H, OH, optionally substituted alkyl, optionally substituted cycloalkyl, having one or more hydrogen atoms in the alkyl or cycloalkyl group optionally replaced by deuterium;
   R¹² is selected from H or optionally substituted alkyl, having one or more hydrogen atoms in the alkyl group optionally replaced by deuterium;
   Ring A is

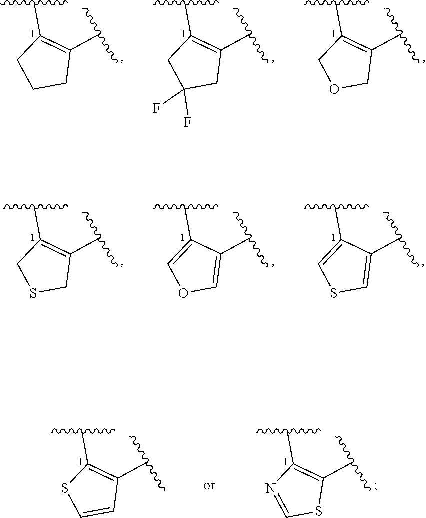

E is

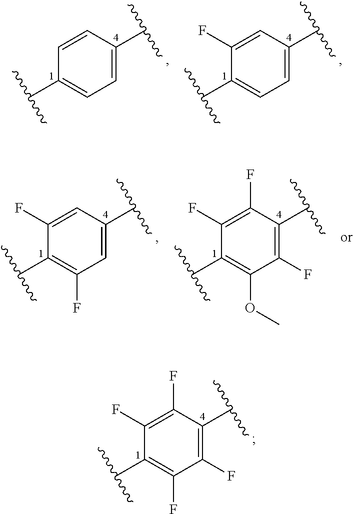 [SMILES: *c1ccc(*)cc1 ; *c1ccc(*)c(F)c1 ; *c1cc(F)c(*)c(F)c1 ; *c1c(F)c(F)c(*)c(OC)c1F ; *c1c(F)c(F)c(*)c(F)c1F]

and
Y is

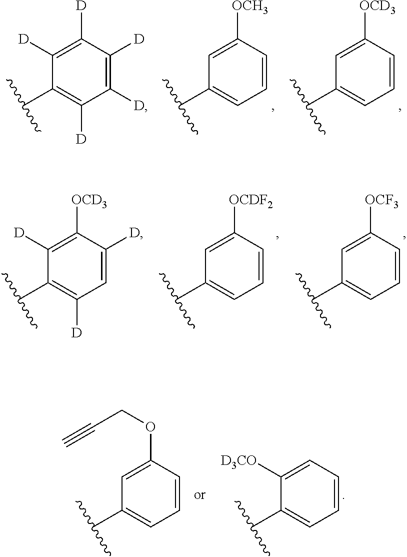

Embodiment K1. A method of treating a neurogenerative condition and/or a method of neuroprotection wherein the method comprises: a) determining a level or activity of Nurr1 in an ex vivo biological sample of a subject with an assay selected from (a) a real-time PCR assay of Nurr1 gene expression against relevant housekeeping genes/internal controls (e.g. GAPDH), (b) an immunoassay (such as ELISA) with the suitable antibodies for Nurr1 protein, and (c) a Western blot for Nurr1 protein from the biological sample selected from peripheral blood, peripheral blood lymphocytes, serum, plasma, CSF and, peripheral blood mononuclear cells; and b) if the level or activity of Nurr1 is no greater than about 90% of the level in a healthy subject of same age, gender and/or BMI, administering to the patient a therapeutically-effective amount of a compound herein.

Embodiment K2. The method of embodiment K1, wherein the neurodegenerative condition is primary progressive multiple sclerosis.

Embodiment K3. The method of embodiment K1, wherein the neurodegenerative condition is relapsing remitting multiple sclerosis.

Embodiment K4. The method of embodiment K1, wherein the neurodegenerative condition is multiple sclerosis, wherein the multiple sclerosis is acquired through relapse-associated worsening (RAW).

Embodiment K5. The method of embodiment K1, wherein the neurodegenerative condition is multiple sclerosis, wherein the multiple sclerosis is acquired through progression independent of relapse activity (PIRA).

Embodiment K6. The method of embodiment K1, wherein the neurodegenerative condition is brain atrophy induced by multiple sclerosis.

Embodiment K7. The method of embodiment K1, wherein the neurodegenerative condition is non-inflammatory multiple sclerosis worsening.

Embodiment K8. The method of embodiment K1, wherein the neuroprotection is slowing or reducing a likelihood of loss of neurons induced by multiple sclerosis.

Embodiment K9. The method of embodiment K1, wherein the neuroprotection is slowing or reducing a likelihood of loss of dopaminergic neurons induced by multiple sclerosis.

Embodiment K10. The method of embodiment K1, wherein the neurodegenerative condition is PIRA.

Embodiment K11. The method of embodiment K1, wherein the neurodegenerative condition is active secondary progressive multiple sclerosis (active SPMS).

Embodiment K12. The method of embodiment K1, wherein the neurodegenerative condition is non-active secondary progressive multiple sclerosis (non-active SPMS).

Embodiment K13. The method of embodiment K1, wherein the neurodegenerative condition is clinically isolated syndrome (CIS).

Embodiment K13. The method of embodiment K1, wherein the neurodegenerative condition is transitioning MS.

Embodiment L1. In a first embodiment, provided herein is a method of treating PIRA in a subject in need thereof, comprising administering to the subject vidofludimus, or an isotopic variant, pharmaceutically acceptable salt or solvate thereof.

Embodiment L2. The method of Embodiment L1, administering to the subject about 45 mg vidofludimus in the maintenance phase, or an equivalent amount of an isotopic variant, pharmaceutically acceptable salt or solvate thereof.

Embodiment L2a. The method of Embodiment L1, administering to the subject about 30 mg vidofludimus in the maintenance phase, or an equivalent amount of an isotopic variant, pharmaceutically acceptable salt or solvate thereof.

Embodiment L3. The method of Embodiment L1, administering to the subject in the maintenance phase about 45 mg vidofludimus calcium salt dihydrate as “Polymorph A”.

Embodiment L4. The method of Embodiment L1, administering to the subject in the maintenance phase about 30 mg vidofludimus calcium salt dihydrate as “Polymorph A”.

Embodiment L5. The method of any one of Embodiments L1 to L4, further comprising evaluating disability progression in the subject.

Embodiment L6. The method of Embodiment L5, wherein disability progression is evaluated using the Expanded Disability Status Scale (EDSS), the 9-Hole Peg Test (9-HPT), or the Timed 25-Foot Walk Test (T25FWT), or any combinations thereof.

Embodiment L7. The method of Embodiment L5 or L6, comprising evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT, and wherein the progression event is confirmed at least 12 weeks after the initial progression.

Embodiment L7a. A method of reducing the risk of experiencing eCDP12 in a subject with PIRA, comprising administering to the subject about 45 mg vidofludimus calcium salt dihydrate as “Polymorph A” once daily in the maintenance phase.

Embodiment L7b. A method of reducing the risk of experiencing eCDP12 in a subject with PIRA, comprising administering to the subject about 30 mg vidofludimus calcium salt dihydrate as “Polymorph A” once daily in the maintenance phase.

Embodiment L7c. The method of Embodiment L7a or L7b, wherein cCDP12 comprises the first occurrence of a progression event in the subject after beginning of administration of vidofludimus calcium salt dihydrate, wherein the progression event is confirmed at least 12 weeks after the initial disability progression.

Embodiment L7d. A method of reducing time to onset of cCDP12 in a subject with PIRA, comprising administering to the subject about 45 mg vidofludimus calcium salt dihydrate as “Polymorph A” once daily in the maintenance phase, wherein time to onset of cCDP12 comprises the period from before beginning administration of vidofludimus calcium salt dihydrate to the first occurrence of a progression event, wherein the progression event is confirmed at least 12 weeks after the initial disability progression.

Embodiment L7e. A method of reducing time to onset of cCDP12 in a subject with PIRA, comprising administering to the subject about 30 mg vidofludimus calcium salt dihydrate as “Polymorph A” once daily in the maintenance phase, wherein time to onset of cCDP12 comprises the period from before beginning administration of vidofludimus calcium salt dihydrate to the first occurrence of a progression event, wherein the progression event is confirmed at least 12 weeks after the initial disability progression.

Embodiment L7f. The method of any one of Embodiments L7c, L7d or L7e, wherein the progression event is one of:
(a) an increase from baseline in Expanded Disability Status Scale (EDSS) score of ≥1.0 point in a subject with a baseline EDSS score of ≤5.5 or an increase of ≥0.5 points in a subject with a baseline EDSS score of ≥5.5 (confirmed disability progression [CDP]);
(b) ≥20% increase from baseline in the Timed 25-Foot Walk Test (T25FWT); or
(c) ≥2.0% increase from baseline in time to complete the 9-Hole Peg Test (9-HPT).

Embodiment L8. The method of any one of Embodiments L5 to L7f, comprising evaluating the onset of 12-week confirmed disability progression (CDP12) in the subject, wherein the onset of CDP12 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 12 weeks after the initial progression.

Embodiment L9. The method of any one of Embodiments L5 to L8, comprising evaluating the onset of composite 24-week confirmed disability progression (cCDP24), wherein the onset of eCDP24 comprises at least one progression event selected from the group consisting of:
(a) an increase from baseline in EDSS score of at least 1.0 point in a subject, with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment L10. The method of any one of Embodiments L5 to L9, comprising evaluating the onset of 24-week confirmed disability progression (CDP24) in the subject, wherein the onset of CDP24 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 24 weeks after the initial progression.

Embodiment L11. The method of any one of Embodiments L1 to L10, wherein time to a progression event in the subject is increased, wherein the progression event is:
(a) an increase from baseline in EDSS score of at least 1.0 point in a subject with a baseline EDSS score of less than or equal to 5.5 points; or
(b) an increase from baseline in EDSS score of at least 0.5 point in a subject with a baseline EDSS score of greater than 5.5 points.

Embodiment L12. The method of any one of Embodiments L1 to L11, wherein time to a progression event in the subject is increased, wherein the progression event is an increase from baseline of at least 20% in time to complete the 9-HPT.

Embodiment L13. The method of any one of Embodiments L1 to L12, wherein time to a progression event in the subject is increased, wherein the progression event is an increase from baseline of at least 20% in T25FWT.

Embodiment L14. The method of any one of Embodiments L1 to L13, wherein time to onset of CDP12 is increased.

Embodiment L15. The method of any one of Embodiments L1 to L14, wherein time to onset of cCDP12 is increased.

Embodiment L16. The method of any one of Embodiments L1 to L15, wherein time to onset of CDP24 is increased.

Embodiment L17. The method of any one of Embodiments L1 to L16, wherein time to onset of cCDP24 is increased.

Embodiment L18. The method of any one of Embodiments L11 to L17, wherein the increase is in comparison to a subject with PIRA that is not administered vidofludimus, or an isotopic variant, pharmaceutically acceptable salt or solvate thereof.

Embodiment L19. The method of any one of Embodiments L11 to L18, wherein the increase is in comparison to a subject with PIRA who is administered an anti-CD20 antibody.

Embodiment L20. The method of any one of Embodiments L11 to L19, wherein the increase is at least 5%, at least 10%, at least 15%, at least 20%, at least 25%, at least 30%, or at least 35%.

Embodiment L21. The method of any one of Embodiments L1 to L20, wherein the progression of PIRA in the subject is slowed.

Embodiment L22. The method of any one of Embodiments L1 to L21, wherein the onset of at least one progression event in the subject is delayed.

Embodiment L23. The method of any one of Embodiments L1 to L22, wherein the risk of the subject having at least one progression event is reduced.

Embodiment L23a. The method of any one of Embodiments L7a, L7b, L7c, L7f to L10, or L23, wherein the risk is reduced by at least 5%, at least 10%, at least 15%, at least 20%, at least 25%, at least 30%, or at least 35%.

Embodiment L23b. The method of any one of Embodiments L7a, L7b, L7c, L7f to L10, L23, or L23a, wherein the risk reduction is in comparison to a subject with PIRA that is not administered vidofludimus, or an isotopic variant, pharmaceutically acceptable salt or solvate thereof.

Embodiment L23c. The method of any one of Embodiments L7a, L7b, L7c, L7f to L10, or L23 to L23b, wherein the risk reduction is in comparison to a subject with PIRA who is administered an anti-CD20 antibody.

Embodiment L24. A method of slowing the progression of PIRA in a subject in need thereof, comprising administering to the subject about 45 mg vidofludimus calcium salt dihydrate as “Polymorph A” once daily in the maintenance phase.

Embodiment L24a. A method of slowing the progression of PIRA in a subject in need thereof, comprising administering to the subject about 30 mg vidofludimus calcium salt dihydrate as “Polymorph A” once daily in the maintenance phase.

Embodiment L25. A method of delaying the onset of at least one progression event in a subject with PIRA, the method comprising administering to the subject about 45 mg vidofludimus calcium salt dihydrate as “Polymorph A” once daily in the maintenance phase.

Embodiment L25a. A method of delaying the onset of at least one progression event in a subject with PIRA, the method comprising administering to the subject about 30 mg vidofludimus calcium salt dihydrate as “Polymorph A” once daily in the maintenance phase.

Embodiment L26. A method of reducing the risk of a subject with PIRA having at least one progression event, the method comprising administering to the subject about 45 mg vidofludimus calcium salt dihydrate as “Polymorph A” once daily in the maintenance phase.

Embodiment L26a. A method of reducing the risk of a subject with PIRA having at least one progression event, the method comprising administering to the subject about 30 mg vidofludimus calcium salt dihydrate as “Polymorph A” once daily in the maintenance phase.

Embodiment L27. The method of any one of Embodiments L21 to L26a, wherein the progression of PIRA is evaluated using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level.

Embodiment L27a. The method of any one of Embodiments L21 to L26a, wherein the progression of PIRA is evaluated using BDNF, GDNF, C-RET, GFAP, DAT, Pitx3, TH, VMAT2, SOD1, AADC, TNFα, iNOS, YKL-40 or IL-1β.

Embodiment L27b. The method of any one of Embodiments L21 to L26a, wherein the progression of PIRA is evaluated using EDSS.

Embodiment L27c. The method of any one of Embodiments L21 to L26a, wherein the progression of PIRA is evaluated using MRI.

Embodiment L27d. The method of any one of Embodiments L21 to L26a, wherein the progression of PIRA is evaluated using NFL.

Embodiment L27e. The method of any one of Embodiments L21 to L26a, wherein the progression of PIRA is evaluated using Nurr1.

Embodiment L27f. The method of any one of Embodiments L21 to L26a, wherein the progression of PIRA is evaluated using GFAP.

Embodiment L27g. The method of any one of Embodiments L21 to L26a, wherein the progression of PIRA is evaluated using BDNF.

Embodiment L28. The method of any one of Embodiments L21 to L27g, wherein the progression of PIRA is evaluated using the CDP12, the cCDP12, the CDP2.4, or the cCDP24.

Embodiment L29. The method of Embodiment L22 or L28, wherein the progression of PIRA comprises the subject experiencing at least one progression event.

Embodiment L30. The method of any one of Embodiments L22 to L29, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

Embodiment L31. The method of any one of Embodiments L22, L23, or L25 to L30, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

Embodiment L32. The method of any one of Embodiments L22, L23, or L25 to L31, wherein the progression event is confirmed at least 12 weeks after the initial progression.

Embodiment L33. The method of any one of Embodiments L22, L23, or L25 to L31, wherein the progression event is confirmed at least 24 weeks after the initial progression.

Embodiment L34. The method of any one of Embodiments L21 to L33, wherein the progression is slowed, or the onset is delayed, or the risk is decreased, in comparison to a subject with PIRA that is not administered vidofludimus, or an isotopic variant, pharmaceutically acceptable salt or solvate thereof.

Embodiment L35. The method of any one of Embodiments L21 to L34, wherein the progression is slowed, or the onset is delayed, or the risk is decreased in comparison to a subject that is administered an anti-CD20 antibody.

Embodiment L36. The method of any one of Embodiments L1 to L35, wherein progression of PIRA in the subject is slowed, or the onset of at least one progression event in the subject is delayed, or the risk of having at least one progression event in the subject is decreased by at least 5%, at least 10%, at least 15%, at least 20%, at least 25%, at least 30%, or at least 35%.

Embodiment L37. A method of reducing disability in a subject with PIRA, the method comprising administering to the subject in the maintenance phase about 45 mg vidofludimus, or an equivalent amount of an isotopic variant, pharmaceutically acceptable salt or solvate thereof.

Embodiment L37a. A method of reducing disability in a subject with PIRA, the method comprising administering to the subject in the maintenance phase about 30 mg vidofludimus, or an equivalent amount of an isotopic variant, pharmaceutically acceptable salt or solvate thereof.

Embodiment L37b. A method of reducing disability in a subject with PIRA, the method comprising administering to the subject about 45 mg vidofludimus calcium salt dihydrate as “Polymorph A” once daily in the maintenance phase.

Embodiment L37c. A method of reducing disability in a subject with PIRA, the method comprising administering to the subject in the maintenance phase about 30 mg vidofludimus calcium salt dihydrate as “Polymorph A” once daily in the maintenance phase.

Embodiment L38. The method of any one of Embodiments L37 to L37c, wherein reducing disability comprises:
reducing the psychological impact of MS;
increasing upper limb function;
increasing walking ability;
decreasing fatigue;
improving work status; or
decreasing global impression of MS severity;
or any combinations thereof.

Embodiment L39. The method of any one of Embodiments L1 to L38, wherein the subject has a reduction in one or more symptoms of PIRA after beginning treatment with vidofludimus, or an isotopic variant, pharmaceutically acceptable salt or solvate thereof.

Embodiment L40. The method of any one of Embodiments L1 to L39, wherein one or more physical impacts of multiple sclerosis on the subject is decreased.

Embodiment L41. A method of slowing the progression of PIRA in a subject in need thereof, comprising administering to the subject about 45 mg vidofludimus calcium salt dihydrate as “Polymorph A” once daily in the maintenance phase; wherein progression of PIRA comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment L41a. A method of slowing the progression of PIRA in a subject in need thereof, comprising administering to the subject about 30 mg vidofludimus calcium salt dihydrate as “Polymorph A” once daily in the maintenance phase; wherein progression of PIRA comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment L42. The method of Embodiment L41 or L41a, wherein the progression event is confirmed at least 12 weeks after the initial progression.

Embodiment L43. The method of any one of Embodiments L41, L41a or L42, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

Embodiment L44. The method of any one of Embodiments L41 to L43, wherein the progression is slowed in comparison to a subject with PIRA that is not administered vidofludimus, or an isotopic variant, pharmaceutically acceptable salt or solvate thereof.

Embodiment L45. The method of any one of Embodiments L41 to L44, wherein the progression is slowed in comparison to a subject that is administered an anti-CD20 antibody.

Embodiment L46. The method of any one of Embodiments L41 to L45, wherein the progression is slowed by at least 5%, at least 10%, at least 15%, at least 20%, at least 25%, at least 30%, or at least 35%.

Embodiment L47. The method of any one of Embodiments L1 to L46, wherein the method further comprises the step of measuring one or more clinical or laboratory endpoints in the subject in order to evaluate the efficacy of treating PIRA.

Embodiment L48. The method of Embodiment L47, wherein the one or more clinical or laboratory endpoints are selected from the group consisting of the subject's MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level.

Embodiment L48a. The method of Embodiment L47, wherein the one or more clinical or laboratory endpoints are selected from the group consisting of BDNF, GDNF, C-RET, GFAP, DAT, Pitx3, TH, VMAT2, SOD1, AADC, TNFα, iNOS, YKL-40 or IL-1β.

Embodiment L48b. The method of Embodiment L47, wherein the one clinical or laboratory endpoints is EDSS.

Embodiment L48c. The method of Embodiment L47, wherein the one clinical or laboratory endpoints is MRI.

Embodiment L48d. The method of Embodiment L47, wherein the one clinical or laboratory endpoints is NFL.

Embodiment L48e. The method of Embodiment L47, wherein the one clinical or laboratory endpoints is Nurr1.

Embodiment L48f. The method of Embodiment L47, wherein the one clinical or laboratory endpoints is GFAP.

Embodiment L48g. The method of Embodiment L47, wherein the one clinical or laboratory endpoints is BDNF.

Embodiment L49. The method of Embodiment L47 or L48g, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of vidofludimus, or an isotopic variant, pharmaceutically acceptable salt or solvate thereof.

Embodiment L49a. The method of Embodiment L47 or L48g, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of vidofludimus calcium salt dihydrate as “Polymorph A”.

Embodiment L50. The method of any one of Embodiments L47 to L49a, wherein the clinical or laboratory endpoint is measured 120 weeks after beginning administration of vidofludimus, or an isotopic variant, pharmaceutically acceptable salt or solvate thereof.

Embodiment L50a. The method of any one of Embodiments L47 to L49a, wherein the clinical or laboratory endpoint is measured 120 weeks after beginning administration of vidofludimus calcium salt dihydrate as “Polymorph A”.

Embodiment L51. The method of any one of Embodiments L1 to L50a, wherein the development of one or more new MS-related brain lesion types in the subject is evaluated after the subject begins administration of vidofludimus, or an isotopic variant, pharmaceutically acceptable salt or solvate thereof, wherein the one or more lesion types is selected from the group consisting of new gadolinium-enhancing lesions on a T1-weighted MRI (T1Gd+), new/enlarging T2-weighted lesions detected by MRI, or new T1-hypointense lesions detected by MRI.

Embodiment L52. The method of Embodiment E51, wherein the development of one or more new lesions in the subject, is reduced as compared to a subject with PIRA that is not administered vidofludimus, or an isotopic variant, pharmaceutically acceptable salt or solvate thereof, or a subject that is administered an anti-CD20 antibody, or a combination thereof.

Embodiment L53. The method of any one of Embodiments L5 to L50a, wherein the period of evaluation is 120 weeks after beginning administration of vidofludimus, or an isotopic variant, pharmaceutically acceptable salt or solvate thereof.

Embodiment L54. The method of any one of Embodiments L1 to L53, wherein vidofludimus, or an isotopic variant, pharmaceutically acceptable salt or solvate thereof is administered orally.

Embodiment L54a. The method of any one of Embodiments L1 to L53, wherein vidofludimus calcium salt dihydrate as “Polymorph A” is administered orally.

Embodiment L55. The method of any one of Embodiments L1 to L54a, wherein vidofludimus, or an isotopic variant, pharmaceutically acceptable salt or solvate thereof is administered in the form of one or more tablets or capsules.

Embodiment L55a. The method of any one of Embodiments L1 to L54a, wherein vidofludimus calcium salt dihydrate as “Polymorph A” is administered in the form of one or more tablets or capsules.

Embodiment L56. The method of any one of Embodiments L1 to L55a, wherein vidofludimus, or an isotopic variant, pharmaceutically acceptable salt or solvate thereof is administered in the form of one tablet once daily, the tablet comprising in the maintenance phase about 45 mg vidofludimus, or an equivalent amount of an isotopic variant, pharmaceutically acceptable salt or solvate thereof.

Embodiment L56a. The method of any one of Embodiments L1 to L55a, wherein vidofludimus, or an isotopic variant, pharmaceutically acceptable salt or solvate thereof is administered in the form of one tablet once daily, the tablet comprising in the maintenance phase about 30 mg vidofludimus, or an equivalent amount of an isotopic variant, pharmaceutically acceptable salt or solvate thereof.

Embodiment L56b. The method of any one of Embodiments L1 to L55a, wherein vidofludimus calcium salt dihydrate as “Polymorph A” is administered in the form of one tablet once daily, the tablet comprising in the maintenance phase about 45 mg vidofludimus calcium salt dihydrate.

Embodiment L56c. The method of any one of Embodiments L1 to L55a, wherein vidofludimus calcium salt dihydrate as “Polymorph A” is administered in the form of one tablet once daily, the tablet comprising in the maintenance phase about 30 mg vidofludimus calcium salt dihydrate.

Embodiment L57. The method of any one of Embodiments L1 to L56c, wherein the tablet is administered in the morning.

Embodiment L57a. The method of any one of Embodiments L1 to L56c, wherein the tablet is administered in the evening.

Embodiment L58. The method of any one of Embodiments L1 to L57a, wherein PIRA is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof.

Embodiment L58a. The method of any one of Embodiments L1 to L57a, wherein PIRA is the subgroup of active secondary progressive multiple sclerosis (aSPMS).

Embodiment L58b. The method of any one of Embodiments L1 to L57a, wherein PIRA is the subgroup of non-active secondary progressive multiple sclerosis (n-aSPMS).

Embodiment L58c. The method of any one of Embodiments L1 to L57a, wherein PIRA is the subgroup of secondary progressive multiple sclerosis (SPMS).

Embodiment L58d. The method of any one of Embodiments L1 to L57a, wherein PIRA is the subgroup of primary progressive multiple sclerosis (PPMS).

Embodiment L58e. The method of any one of Embodiments L1 to L57a, wherein PIRA is the subgroup of progressive multiple sclerosis (PMS).

Embodiment L59. The method of any one of Embodiments L1 to L57a, wherein subject has paediatric-onset multiple sclerosis (POMS).

Embodiment L59a. The method of any one of Embodiments L1 to L57a, wherein subject has late-onset multiple sclerosis (LOMS).

Embodiment L59b. The method of any one of Embodiments L1 to L57a, wherein subject has adult-onset MS (AOMS).

Embodiment L59c. The method of any one of Embodiments L1 to L57a, wherein subject has clinically isolated syndrome (CIS).

Embodiment L59d. The method of any one of Embodiments L1 to L57a, wherein subject has transitioning MS.

Embodiment L60. The method of any one of Embodiments L1 to L59d, wherein subject has a gender selected from male, female or diverse.

Embodiment L60a. The method of any one of Embodiments L1 to L59d, wherein subject is female.

Embodiment L60b. The method of any one of Embodiments L1 to L59d, wherein subject is male.

Embodiment M1. A compound for use in a method of treating PIRA in a subject in need thereof, wherein the compound is vidofludimus, or an isotopic variant, pharmaceutically acceptable salt or solvate thereof.

Embodiment M1a. A compound for use in a method of treating PIRA in a subject in need thereof, wherein the compound is vidofludimus, or an equivalent amount of an isotopic variant, pharmaceutically acceptable salt or solvate thereof, and wherein the method comprises administering to the subject in the maintenance phase about 45 mg vidofludimus once daily, or an equivalent amount of an isotopic variant, pharmaceutically acceptable salt or solvate thereof.

Embodiment M1b. A compound for use in a method of treating PIRA in a subject in need thereof, wherein the compound is vidofludimus, or an equivalent amount of an isotopic variant, pharmaceutically acceptable salt or solvate thereof, and wherein the method comprises administering to the subject in the maintenance phase about 30 mg vidofludimus once daily, or an equivalent amount of an isotopic variant, pharmaceutically acceptable salt or solvate thereof.

Embodiment M1c. A compound for use in a method of treating PIRA in a subject in need thereof, wherein the compound is vidofludimus calcium salt dihydrate as “Polymorph A”, and wherein the method comprises administering to the subject in the maintenance phase about 45 mg vidofludimus calcium salt dihydrate once daily.

Embodiment M1d. A compound for use in a method of treating PIRA in a subject in need thereof, wherein the compound is vidofludimus calcium salt dihydrate as “Polymorph A”, and wherein the method comprises administering to the subject in the maintenance phase about 30 mg vidofludimus calcium salt dihydrate once daily.

Embodiment M2. The compound for use of any one of Embodiments M1 to M1d, wherein disability progression in the subject is evaluated.

Embodiment M3. The compound for use of Embodiment M2, wherein disability progression is evaluated using the Expanded Disability Status Scale (EDSS), the 9-Hole Peg Test (9-HPT), or the Timed 25-Foot Walk Test (T25FWT), or any combinations thereof.

Embodiment M4. The compound for use of Embodiment M2 or M3, wherein the onset of composite 12-week confirmed disability progression (cCDP12) is evaluated, wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT, and wherein the progression event is confirmed at least 12 weeks after the initial progression.

Embodiment M4a. A compound for use in reducing the risk of experiencing cCDP12 in a subject with PIRA, comprising administering to the subject in the maintenance phase about 45 mg vidofludimus, or an equivalent amount of an isotopic variant, pharmaceutically acceptable salt or solvate thereof.

Embodiment M4b. A compound for use in reducing the risk of experiencing cCDP12 in a subject with PIRA, comprising administering to the subject in the maintenance phase about 30 mg vidofludimus, or an equivalent amount of an isotopic variant, pharmaceutically acceptable salt or solvate thereof.

Embodiment M4c. A compound for use in reducing the risk of experiencing cCDP12 in a subject with PIRA, comprising administering to the subject in the maintenance phase about 45 mg vidofludimus calcium salt dihydrate as “Polymorph A”.

Embodiment M4d. A compound for use in reducing the risk of experiencing cCDP12 in a subject with PIRA, comprising administering to the subject in the maintenance phase about 30 mg vidofludimus calcium salt dihydrate as “Polymorph A”.

Embodiment M4e. The compound for use of any one of Embodiments M4a to M4d, wherein cCDP12 comprises the first occurrence of a progression event in the subject after beginning of administration of vidofludimus, or an isotopic variant, pharmaceutically acceptable salt or solvate thereof, wherein the progression event is confirmed at least 12 weeks after the initial disability progression.

Embodiment M4f. A compound for use in reducing time to onset of cCDP12 in a subject with PIRA, comprising administering to the subject about 45 mg vidofludimus calcium salt dihydrate as “Polymorph A” once daily in the maintenance phase, wherein time to onset of cCDP12 comprises the period from before beginning administration of vidofludimus calcium salt dihydrate to the first occurrence of a progression event, wherein the progression event is confirmed at least 12 weeks after the initial disability progression.

Embodiment M4g. A compound for use in reducing time to onset of cCDP12 in a subject with PIRA, comprising administering to the subject about 30 mg vidofludimus calcium salt dihydrate as “Polymorph A” once daily in the maintenance phase, wherein time to onset of cCDP12 comprises the period from before beginning administration of vidofludimus calcium salt dihydrate to the first occurrence of a progression event, wherein the progression event is confirmed at least 12 weeks after the initial disability progression.

Embodiment M4h. The compound for use of any one of Embodiments M4a or M4e, wherein the progression event is one of:
(a) an increase from baseline in Expanded Disability Status Scale (EDSS) score of ≥1.0 point in a subject with a baseline EDSS score of ≤5.5 or an increase of 0.5 points in a subject with a baseline EDSS score of ≥5.5 (confirmed disability progression [CDP]);
(b) ≥20% increase from baseline in the Timed 25-Foot Walk Test (T25FWT); or
(c) ≥2.0% increase from baseline in time to complete the 9-Hole Peg Test (9-HPT).

Embodiment M5. The compound for use of any one of Embodiments M2 to M4h, wherein the method further comprises evaluating the onset of 12-week confirmed disability progression (CDP12) in the subject, wherein the onset of CDP12 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 12 weeks after the initial progression.

Embodiment M6. The compound for use of any one of Embodiments M2 to M5, wherein the method further comprises evaluating the onset of composite 24-week confirmed disability progression (eCDP24), wherein the onset of cCDP24 comprises at least one progression event selected from the group consisting of:
(a) an increase from baseline in EDSS score of at least 1.0 point in a subject, with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment M7. The compound for use of any one of Embodiments M2 to M6, wherein the method further comprises evaluating the onset of 24-week confirmed disability progression (CDP24) in the subject, wherein the onset of CDP24 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 24 weeks after the initial progression.

Embodiment M8. The compound for use of any one of Embodiments M1 to M7, wherein time to a progression event is increased, wherein the progression event is:
(a) an increase from baseline in EDSS score of at least 1.0 point in a subject with a baseline EDSS score of less than or equal to 5.5 points; or
(b) an increase from baseline in EDSS score of at least 0.5 point in a subject with a baseline EDSS score of greater than 5.5 points.

Embodiment M9. The compound for use of any one of Embodiments M1 to M8, wherein time to a progression event in the subject is increased, wherein the progression event is an increase from baseline of at least 20% in time to complete the 9-HPT.

Embodiment M10. The compound for use of any one of Embodiments M1 to M9, wherein time to a progression event in the subject is increased, wherein the progression event is an increase from baseline of at least 20% in T25FWT.

Embodiment M11. The compound for use of any one of Embodiments M1 to M10, wherein time to onset of CDP12 is increased.

Embodiment M12. The compound for use of any one of Embodiments M1 to M11, wherein time to onset of cCDP12 is increased.

Embodiment M13. The compound for use of any one of Embodiments M1 to M12, wherein time to onset of CDP24 is increased.

Embodiment M14. The compound for use of any one of Embodiments M1 to M13, wherein time to onset of cCDP24 is increased.

Embodiment M15. The compound for use of any one of Embodiments M8 to M14, wherein the increase is in comparison to a subject with PIRA who is not administered vidofludimus, or an isotopic variant, pharmaceutically acceptable salt or solvate thereof.

Embodiment M16. The compound for use of any one of Embodiments M8 to M15, wherein the increase is in comparison to a subject with PIRA who is administered an anti-CD2Q antibody.

Embodiment M17. The compound for use of any one of Embodiments M8 to M16, wherein the increase is at least 5%, at least 10%, at least 15%, at least 20%, at least 25%, at least 30%, or at least 35%.

Embodiment M18 The compound for use of any one of Embodiments M1 to M17, wherein the progression of PIRA in the subject is slowed.

Embodiment M19. The compound for use of any one of Embodiments M1 to M18, wherein the onset of at least one progression event is delayed.

Embodiment M20. The compound for use of any one of Embodiments M1 to M19, wherein the risk of the subject having at least one progression event is reduced.

Embodiment M20a. The compound for use of any one of Embodiments M4a to M4e, M4h to M7, or M20, wherein the risk is reduced by at least 5%, at least 10%, at least 15%, at least 20%, at least 25%, at least 30%, or at least 35%.

Embodiment M20b. The compound for use of any one of Embodiments M4a to M4e, M4h to M7, M20, or M20a, wherein the risk reduction is in comparison to a subject with PIRA that is not administered vidofludimus, or an isotopic variant, pharmaceutically acceptable salt or solvate thereof.

Embodiment M20c. The compound for use of any one of Embodiments M4a to M4e, M4h to M7, or M20 to M20b, wherein the risk reduction is in comparison to a subject with PIRA who is administered an anti-CD20 antibody.

Embodiment M21. A compound for use in a method of slowing the progression of PIRA in a subject in need thereof, wherein the compound is vidofludimus, or an isotopic variant, pharmaceutically acceptable salt or solvate thereof, and wherein the method comprises administering to the subject about 45 mg vidofludimus, or an equivalent amount of an isotopic variant, pharmaceutically acceptable salt or solvate thereof once daily in the maintenance phase.

Embodiment M21a. A compound for use in a method of slowing the progression of PIRA in a subject in need thereof, wherein the compound is vidofludimus, or an isotopic variant, pharmaceutically acceptable salt or solvate thereof, and wherein the method comprises administering to the subject about 30 mg vidofludimus, or an equivalent amount of an isotopic variant, pharmaceutically acceptable salt or solvate thereof once daily in the maintenance phase.

Embodiment M21b. A compound for use in a method of slowing the progression of PIRA in a subject in need thereof, wherein the compound is vidofludimus calcium salt dihydrate as “Polymorph A”, and wherein the method comprises administering to the subject about 45 mg vidofludimus calcium salt dihydrate once daily in the maintenance phase.

Embodiment M21c. A compound for use in a method of slowing the progression of PIRA in a subject in need thereof, wherein the compound is vidofludimus calcium salt dihydrate as “Polymorph A”, and wherein the method comprises administering to the subject about 30 mg vidofludimus calcium salt dihydrate once daily in the maintenance phase.

Embodiment M22. A compound for use in a method of delaying the onset of at least one progression event in a subject with PIRA, wherein the compound is vidofludimus, or an isotopic variant, pharmaceutically acceptable salt or solvate thereof, and wherein the method comprises administering to the subject about 45 mg vidofludimus, or an equivalent amount of an isotopic variant, pharmaceutically acceptable salt or solvate thereof once daily in the maintenance phase.

Embodiment M22a. A compound for use in a method of delaying the onset of at least one progression event in a subject with PIRA, wherein the compound is vidofludimus, or an isotopic variant, pharmaceutically acceptable salt or solvate thereof, and wherein the method comprises administering to the subject about 30 mg vidofludimus, or an equivalent amount of an isotopic variant, pharmaceutically acceptable salt or solvate thereof once daily in the maintenance phase.

Embodiment M22b. A compound for use in a method of delaying the onset of at least one progression event in a subject with PIRA, wherein the compound is vidofludimus calcium salt dihydrate, and wherein the method comprises administering to the subject about 45 mg vidofludimus calcium salt dihydrate once daily in the maintenance phase.

Embodiment M22c. A compound for use in a method of delaying the onset of at least one progression event in a subject with PIRA, wherein the compound is vidofludimus calcium salt dihydrate, and wherein the method comprises administering to the subject about 30 mg vidofludimus calcium salt dihydrate once daily in the maintenance phase.

Embodiment M23. A compound for use in a method of reducing the risk of a subject with PIRA having at least one progression event, wherein the compound is vidofludimus, or an isotopic variant, pharmaceutically acceptable salt or solvate thereof, and wherein the method comprises administering to the subject about 45 mg vidofludimus, or an equivalent amount of an isotopic variant, pharmaceutically acceptable salt or solvate thereof once daily in the maintenance phase.

Embodiment M23a. A compound for use in a method of reducing the risk of a subject with PIRA having at least one progression event, wherein the compound is vidofludimus, or an isotopic variant, pharmaceutically acceptable salt or solvate thereof, and wherein the method comprises administering to the subject about 30 mg vidofludimus, or an equivalent amount of an isotopic variant, pharmaceutically acceptable salt or solvate thereof once daily in the maintenance phase.

Embodiment M23b. A compound for use in a method of reducing the risk of a subject with PIRA having at least one progression event, wherein the compound is vidofludimus calcium salt dihydrate, and wherein the method comprises administering to the subject about 45 mg vidofludimus calcium salt dihydrate once daily in the maintenance phase.

Embodiment M23c. A compound for use in a method of reducing the risk of a subject with PIRA having at least one progression event, wherein the compound is vidofludimus calcium salt dihydrate, and wherein the method comprises administering to the subject about 30 mg vidofludimus calcium salt dihydrate once daily in the maintenance phase.

Embodiment M24. The compound for use of any one of Embodiments M18 to M23c, wherein the progression of PIRA is evaluated using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level.

Embodiment M24a. The compound for use of any one of Embodiments M18 to M23c, wherein the progression of PIRA is evaluated using BDNF, GDNF, C-RET, GFAP, DAT, Pitx3, TH, VMAT2, SOD1, AADC, TNFα, iNOS, YKL-40 or IL-1β.

Embodiment M25. The compound for use of any one of Embodiments M18 to M24a, wherein the progression of PIRA is evaluated using the CDP12, the cCDP12, the CDP24, or the cCDP24.

Embodiment M26. The compound for rise of M25, wherein the progression of PIRA comprises at least one progression event.

Embodiment M27. The compound for use of any one of Embodiments M19 to M26, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

Embodiment M28. The compound for use of any one of Embodiments M19 to M27, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

Embodiment M29. The compound for use of any one of Embodiments M22 to M28, wherein the progression event is confirmed at least 12 weeks after the initial progression.

Embodiment M30. The compound for use of any one of Embodiments M22 to M28, wherein the progression event is confirmed at least 24 weeks after the initial progression.

Embodiment M31. The compound for use of any one of Embodiments M22 to M30, wherein the progression is slowed, or the onset is delayed, or the risk is decreased, in comparison to a subject with PIRA that is not administered vidofludimus, or an isotopic variant, pharmaceutically acceptable salt or solvate thereof.

Embodiment M32. The compound for use of any one of Embodiments M22 to M31, wherein the progression is slowed, or the onset is delayed, or the risk is decreased in comparison to a subject that is administered an anti-CD20 antibody.

Embodiment M33. The compound for use of any one of Embodiments M1 to M32, wherein the progression of PIRA in the subject is slowed, or the onset of at least one progression event in the subject is delayed, or the risk of having at least one progression event in the subject is decreased by at least 5%, at least 10%, at least 15%, at least 20%, at least 25%, at least 30%, or at least 35%.

Embodiment M34. A compound for use in a method of reducing disability in a subject with PIRA, wherein the compound is vidofludimus, or an isotopic variant, pharmaceutically acceptable salt or solvate thereof, and wherein the method comprises administering to the subject about 45 mg vidofludimus, or an equivalent amount of an isotopic variant, pharmaceutically acceptable salt or solvate thereof once daily in the maintenance phase.

Embodiment M34a. A compound for use in a method of reducing disability in a subject with PIRA, wherein the compound is vidofludimus, or an isotopic variant, pharmaceutically acceptable salt or solvate thereof, and wherein the method comprises administering to the subject about 30 mg vidofludimus, or an equivalent amount of an isotopic variant, pharmaceutically acceptable salt or solvate thereof once daily in the maintenance phase.

Embodiment M34b. A compound for use in a method of reducing disability in a subject with PIRA, wherein the compound is vidofludimus calcium salt dihydrate, and wherein the method comprises administering to the subject about 45 mg vidofludimus calcium salt dihydrate once daily in the maintenance phase.

Embodiment M34c. A compound for use in a method of reducing disability in a subject with PIRA, wherein the compound is vidofludimus calcium salt dihydrate, and wherein the method comprises administering to the subject about 30 mg vidofludimus calcium salt dihydrate once daily in the maintenance phase.

Embodiment M35. The compound for use of any one of Embodiments M34 to M34c, wherein reducing disability comprises:
reducing the psychological impact of MS;
increasing upper limb function;
increasing walking ability;
decreasing fatigue;
improving work status; or
decreasing global impression of MS severity;
or any combinations thereof.

Embodiment M36. The compound for use of any one of Embodiments M1 to M35, wherein the subject has a reduction in one or more symptoms of IRA after beginning treatment with vidofludimus, or an isotopic variant, pharmaceutically acceptable salt or solvate thereof.

Embodiment M37. The compound for use of any one of Embodiments M1 to M36, wherein one or more physical impacts of multiple sclerosis on the subject is decreased.

Embodiment M38. A compound for use in a method of slowing the progression of PIRA in a subject in need thereof, wherein the compound is vidofludimus calcium salt dihydrate as “Polymorph A”, and wherein the method comprises administering to the subject about 45 mg vidofludimus calcium salt dihydrate once daily in the maintenance phase; wherein progression of PIRA comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment M38a. A compound for use in a method of slowing the progression of PIRA in a subject in need thereof, wherein the compound is vidofludimus calcium salt dihydrate as “Polymorph A”, and wherein the method comprises administering to the subject about 30 mg vidofludimus calcium salt dihydrate once daily in the maintenance phase; wherein progression of PIRA comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT;
and wherein the progression event is confirmed at least 24 weeks after the initial progression.

Embodiment M39. The compound for use of Embodiment M38 or M38a, wherein the progression event is confirmed at least 12 weeks after the initial progression.

Embodiment M40. The compound for use of any one of Embodiments M38 to M39, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

Embodiment M41. The compound for use of any one of Embodiments M38 to M40, wherein the progression is slowed in comparison to a subject with PIRA that is not administered vidofludimus, or an isotopic variant, pharmaceutically acceptable salt or solvate thereof.

Embodiment M42. The compound for use of any one of Embodiments M38 to M41, wherein the progression is slowed in comparison to a subject that is administered an anti-CD20 antibody.

Embodiment M43. The compound for use of any one of Embodiments M38 to M42, wherein the progression is slowed by at least 5%, at least 10%, at least 15%, at least 2.0%, at least 25%, at least 30%, or at least 35%.

Embodiment M44. The compound for use of any one of Embodiments M1 to M43, wherein the method further comprises the step of measuring one or more clinical or laboratory endpoints in the subject in order to evaluate the efficacy of treating PIRA.

Embodiment M45. The compound for use of Embodiment M44, wherein the one or more clinical or laboratory endpoints are selected from the group consisting of the subject's MSIS-29, Neuro-QoL Upper Extremity, PROMIS-FatigueMS, MSWS-12, PGI-S, WPAI:MS, PGI-C, EQ-5D-5L, C-SSRS, 9-HPT, T25EWT, EDSS, SDMT, MRI, NFL level, GFAP level, BDNF level or Nurr1 expression level.

Embodiment M45a. The compound for use of Embodiment M44, wherein the one or more clinical or laboratory endpoints are selected from the group consisting of BDNF, GDNF, C-RET, GFAP, DAT, Pitx3, TH, VMAT2, SOD1, AADC, TNFα, iNOS, YKL-40 or IL-1β.

Embodiment M46. The compound tor use of Embodiment M44 or M45a, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of vidofludimus, or an isotopic variant, pharmaceutically acceptable salt or solvate thereof.

Embodiment M46a. The compound tor use of Embodiment M44 or M45a, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of vidofludimus calcium salt dihydrate as “Polymorph A”.

Embodiment M47. The compound for use of any one of Embodiments M44 to M46a, wherein the clinical or laboratory endpoint is measured 120 weeks after beginning administration of vidofludimus, or an isotopic variant, pharmaceutically acceptable salt or solvate thereof.

Embodiment M47a. The compound for use of any one of Embodiments M44 to M46a, wherein the clinical or laboratory endpoint is measured 120 weeks after beginning administration of vidofludimus calcium salt dihydrate as “Polymorph A”.

Embodiment M48. The compound tor use of any one of Embodiments M1 to M47a, wherein the development of one or more new MS-related brain lesion types in the subject is evaluated after the subject begins administration of vidofludimus, or an isotopic variant, pharmaceutically acceptable salt or solvate thereof, wherein the one or more lesion types is selected from the group consisting of new gadolinium-enhancing lesions on a T1-weighted MRI (T1Gd+), new/enlarging T2-weighted lesions detected by MRI, or new T1-hypoin tense lesions detected by MRI.

Embodiment M49. The compound for use of Embodiment M48, wherein the development of one or more new lesions in the subject is reduced as compared to a subject with PIRA that is not administered vidofludimus, or an isotopic variant, pharmaceutically acceptable salt or solvate thereof, or a subject that is administered an anti-CD20 antibody, or a combination thereof.

Embodiment M50. The compound for use of any one of Embodiments M2 to M47a, wherein the period of evaluation is 120 weeks after beginning administration of vidofludimus, or an isotopic variant, pharmaceutically acceptable salt or solvate thereof.

Embodiment M51. The compound for use of any one of Embodiments M1 to M50, wherein vidofludimus, or an isotopic variant, pharmaceutically acceptable salt or solvate thereof is administered orally.

Embodiment M51a. The compound for use of any one of Embodiments M1 to M50, wherein vidofludimus calcium salt dihydrate as “Polymorph A” is administered orally.

Embodiment M52. The compound for use of any one of Embodiments M1 to M51a, wherein vidofludimus, or an isotopic variant, pharmaceutically acceptable salt or solvate thereof is administered in the form of one or more tablets or capsules.

Embodiment M52a. The compound for use of any one of Embodiments M1 to M51a, wherein vidofludimus calcium salt dihydrate as “Polymorph A” is administered in the form of one or more tablets or capsules.

Embodiment M53. The compound for use of any one of Embodiments M1 to M52a, wherein vidofludimus, or an isotopic variant, pharmaceutically acceptable salt or solvate thereof is administered in the form of one tablet once daily, the tablet comprising in the maintenance phase about 45 mg vidofludimus, or an equivalent amount of an isotopic variant, pharmaceutically acceptable salt or solvate thereof.

Embodiment M53a. The compound for use of any one of Embodiments M1 to M52a, wherein vidofludimus, or an isotopic variant, pharmaceutically acceptable salt or solvate thereof is administered in the form of one tablet once daily, the tablet comprising in the maintenance phase about 30 mg vidofludimus, or an equivalent amount of an isotopic variant, pharmaceutically acceptable salt or solvate thereof.

Embodiment M53b. The compound for use of any one of Embodiments M1 to M52a, wherein vidofludimus calcium salt dihydrate as “Polymorph A” is administered in the form of one tablet once daily, the tablet comprising in the maintenance phase about 45 mg vidofludimus calcium salt dihydrate.

Embodiment M53c. The compound for use of any one of Embodiments M1 to M52a, wherein vidofludimus calcium salt dihydrate as “Polymorph A” is administered in the form of one tablet once daily, the tablet comprising in the maintenance phase about 30 mg vidofludimus calcium salt dihydrate.

Embodiment M54. The compound tor use of any one of Embodiments M1 to M53c, wherein the tablet is administered in the morning.

Embodiment M54a. The compound tor use of any one of Embodiments M1 to M53c, wherein the tablet is administered in the evening.

Embodiment M55. The compound tor use of any one of Embodiments M1 to M54a, wherein PIRA is selected from the subgroup consisting of: (a) active secondary progressive multiple sclerosis (aSPMS); (b) non-active secondary progressive multiple sclerosis (n-aSPMS); (c) secondary progressive multiple sclerosis (SPMS); (d) primary progressive multiple sclerosis (PPMS); (e) progressive multiple sclerosis (PMS); (f) early PIRA; (g) late PIRA; (h) active PIRA; (i) nonactive PIRA; (j) clinically isolated syndrome (CIS); (k) transitioning MS; or a combination thereof.

Embodiment M55a. The compound tor use of any one of Embodiments M1 to M54a, wherein PIRA is the subgroup of active secondary progressive multiple sclerosis (aSPMS).

Embodiment M55b. The compound tor use of any one of Embodiments M1 to M54a, wherein PIRA is the subgroup of non-active secondary progressive multiple sclerosis (n-aSPMS).

Embodiment M55c. The compound tor use of any one of Embodiments M1 to M54a, wherein PIRA is the subgroup of secondary progressive multiple sclerosis (SPMS).

Embodiment M55d. The compound tor use of any one of Embodiments M1 to M54a, wherein PIRA is the subgroup of primary progressive multiple sclerosis (PPMS).

Embodiment M55e. The compound tor use of any one of Embodiments M1 to M54a, wherein PIRA is the subgroup of progressive multiple sclerosis (PMS).

Embodiment M56. The compound tor use of any one of Embodiments M1 to M55e, wherein subject has paediatric-onset multiple sclerosis (POMS).

Embodiment M56a. The compound tor use of any one of Embodiments M1 to M55e, wherein subject has late-onset multiple sclerosis (LOMS).

Embodiment M56b. The compound tor use of any one of Embodiments M1 to M55e, wherein subject has adult-onset MS (AOMS).

Embodiment M56c. The method of any one of Embodiments L1 to M55e, wherein subject has clinically isolated syndrome (CIS).

Embodiment M56d. The method of any one of Embodiments L1 to M55e, wherein subject has transitioning MS.

Embodiment M57. The compound tor use of any one of Embodiments M1 to M56d, wherein subject has a gender selected from male, female or diverse.

Embodiment M57a. The compound tor use of any one of Embodiments M1 to M56d, wherein subject is female.

Embodiment M57b. The compound tor use of any one of Embodiments M1 to M56d, wherein subject is male.

Embodiment N1. Further provided herein is a compound for use in the manufacture of a medicament for any of the methods of Embodiments L1 to L60b, wherein the compound is vidofludimus, or an isotopic variant, pharmaceutically acceptable salt or solvate thereof.

Embodiment N1a. Further provided herein is a compound for use in the manufacture of a medicament for any of the methods of Embodiments L1 to L60b, wherein the compound is vidofludimus calcium salt dihydrate as “Polymorph A”.

Embodiment N2. The method of any one of Embodiments L1 to L60b, or the compound for use of any one of Embodiments M1 to M57b, wherein the subject with PIRA has had progressive disease from the onset and has been in a progressive stage for at least 12 months prior to beginning administration of vidofludimus, or an isotopic variant, pharmaceutically acceptable salt or solvate thereof.

Embodiment N2a. The method of any one of Embodiments L1 to L60b, or the compound for use of any one of Embodiments M1 to M57b, wherein the subject with PIRA has had progressive disease from the onset and has been in a progressive stage for at least 12 months prior to beginning administration of vidofludimus calcium salt dihydrate as “Polymorph A”.

Embodiment N3. The method of any one of Embodiments L1 to L60b or N2 to N2a, or the compound tor use of any one of Embodiments M1 to M57b or N2 to N2a, wherein prior to beginning administration of vidofludimus, or an isotopic variant, pharmaceutically acceptable salt or solvate thereof, the subject has at least two of:
(a) one or more T2-hyperintense lesions characteristic of MS in one or more of the periventricular, cortical or juxtacortical, or infratentorial the following brain regions;
(b) two or more T2-hyperintense lesions in the spinal cord; and
(c) the presence of cerebrospinal fluid-specific oligoclonal bands.

Embodiment N3a. The method of any one of Embodiments L1 to L60b or N2 to N2a, or the compound tor use of any one of Embodiments M1 to M57b or N2 to N2a, wherein prior to beginning administration of vidofludimus calcium salt dihydrate as “Polymorph A”, the subject has at least two of:
(a) one or more T2-hyperintense lesions characteristic of MS in one or more of the periventricular, cortical or juxtacortical, or infratentorial the following brain regions;
(b) two or more T2-hyperintense lesions in the spinal cord; and
(c) the presence of cerebrospinal fluid-specific oligoclonal bands.

Embodiment N4. The method of any one of Embodiments L1 to L60b or N2 to N3a, or the compound for use of any one of Embodiments M1 to M57b or N2 to N3, wherein the subject has an EDSS score from 3.0 to 6.5 prior to beginning administration of vidofludimus, or an isotopic variant, pharmaceutically acceptable salt or solvate thereof.

Embodiment N4a. The method of any one of Embodiments L1 to L60b or N2 to N3a, or the compound for use of any one of Embodiments M1 to M57b or N2 to N3, wherein the subject has an EDSS score from 3.0 to 6.5 prior to beginning administration of vidofludimus calcium salt dihydrate as “Polymorph A”.

### EXAMPLES

The present disclosure is further illustrated by the following non-limiting examples.

### Example 1. Preparation of Compounds

The carboxylic acid containing intermediates of the present invention can be prepared. By using appropriate deuterated building blocks or via hydrogen-deuterium exchange, the deuterated intermediates can be prepared. The carboxylic acid containing intermediates of the present invention can be prepared as outlined in WO 2003/006425 and WO 2004/056797 (and references cited therein). By using appropriate deuterated building blocks or via hydrogen-deuterium exchange (e.g. Synthesis 2019; 51:1319 or Angew. Chem. Int. Ed. 2018; 57:3022) the deuterated intermediates can be prepared as recently described in WO 2022/214691. Replacing the carboxylic acid by a bioisosteric moiety has been described in WO 2023/118576.

### A. Prepararative Example P1

### Step 1: 1-Bromo-3-(2-bromoethoxy)benzene (3a)

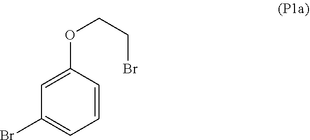 [SMILES: BrCCOc1cccc(Br)c1]

To a solution of 3-bromophenol (1.0 g) in ACN (50 mL) was added 1,2-dibromoethane (2.2 g), K₂CO₃ (2.4 g) and KI (100 mg) at rt. The mixture was stirred at 65° C. overnight, cooled to rt, poured into water (100 mL) and extracted with EA (3×100 mL). The combined organic layer was dried over Na₂SO₄, filtered, concentrated and purified by FCC (PE:EA=20:1) to afford compound P1a as a colorless oil. ¹H-NMR (400 MHz, DMSO-d₆) δ 7.28 (t, J=10.4 Hz, 1H), 7.21-7.17 (m, 2H), 7.03-6.99 (m, 1H), 4.37 (t, J=7.2 Hz, 2H), 3.82 (t, J=7.2 Hz, 2H).

### Step 2: 1-(2-Azidoethoxy)-3-bromobenzene (P1)

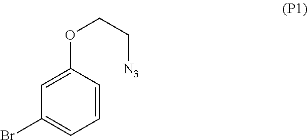 [SMILES: [N-]=[N+]=NCCOc1cccc(Br)c1]

To a solution of compound P1a (238 mg) in DMF (10 mL) was added NaN₃ (111 mg) at rt. The mixture was stirred at 50° C. for 3 h, cooled to rt, poured into water (50 mL) and extracted with EA (3×20 mL). The combined organic layer was dried over Na₂SO₄, filtered, concentrated and purified by FCC (PE:EA=10:1) to afford compound P1 as a colorless oil.

### 2-((3-Fluoro-3′-methoxy-[1,1′-biphenyl]-4-yl)carbamoyl)cyclopent-1-ene-1-carboxylic acid (1)

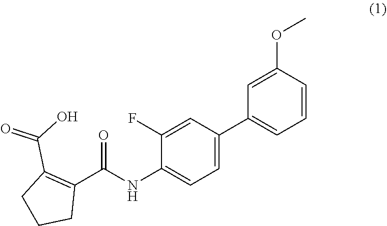 [SMILES: COc1cccc(-c2ccc(NC(=O)C3=C(C(=O)O)CCC3)c(F)c2)c1]

Compound (1) can be obtained from e.g., Medchemexpress (Catalogue Nr. HY-14908)

### Example 2. Compounds 2/1 to 2/31

The following compounds were prepared as described in WO 2022/214691 or in W 2023/118576.

| Compound # | Structure |
|---|---|
| 2/1 | 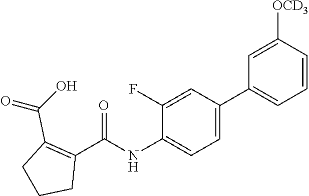 |
| 2/2 | 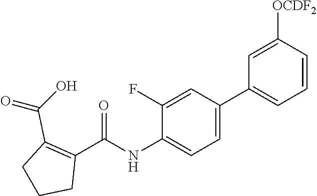 |
| 2/3 | 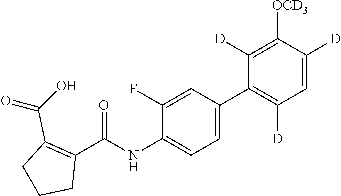 |
| 2/4 | 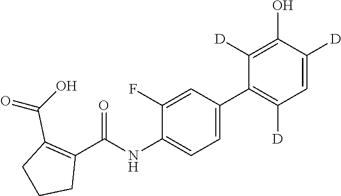 [SMILES: [2H]c1cc([2H])c(-c2ccc(NC(=O)C3=C(C(=O)O)CCC3)c(F)c2)c([2H])c1O] |
| 2/5 | 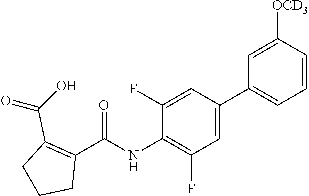 |
| 2/6 | 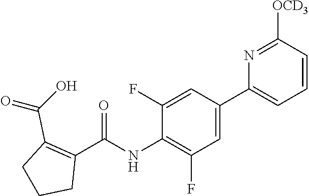 |
| 2/7 | 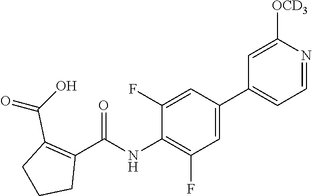 |
| 2/8 | 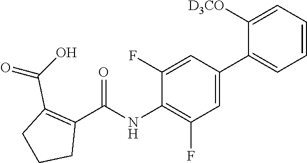 |
| 2/9 | 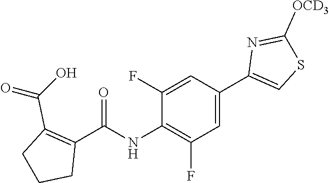 |
| 2/10 | 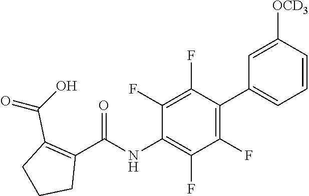 |
| 2/11 | 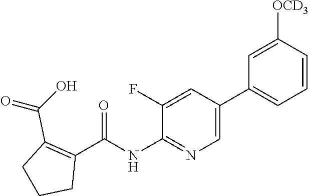 |
| 2/12 | 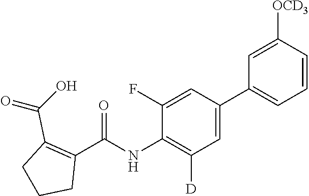 |
| 2/13 | 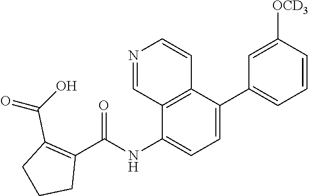 |
| 2/14 | 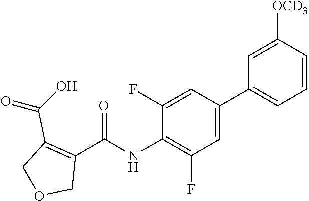 |
| 2/15 | 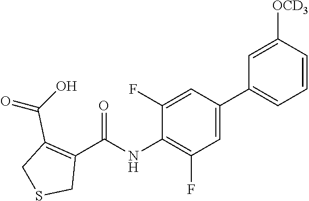 |
| 2/16 | 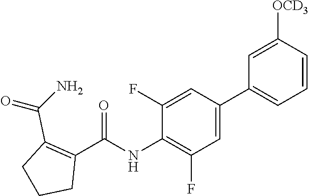 |
| 2/17 | 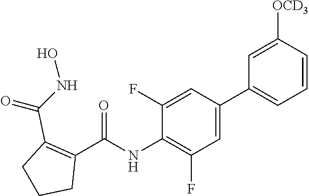 |
| 2/18 | 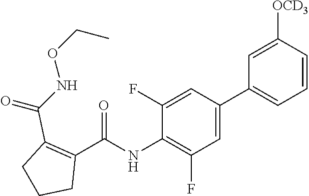 |
| 2/19 | 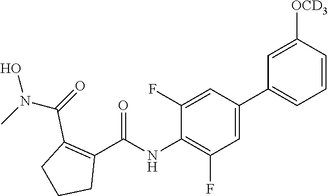 |
| 2/20 | 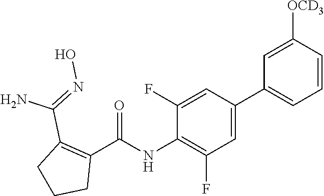 |
| 2/21 | 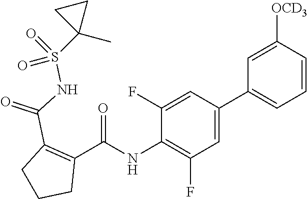 |
| 2/22 | 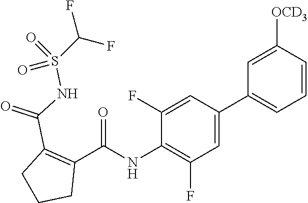 |
| 2/23 | 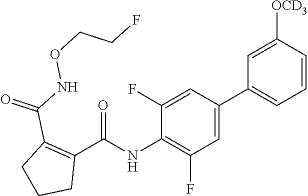 |
| 2/24 | 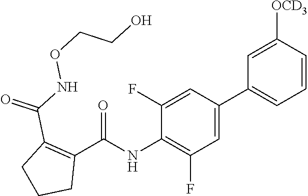 |
| 2/25 | 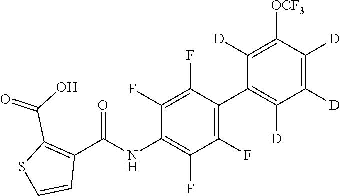 [SMILES: [2H]c1c([2H])c(OC(F)(F)F)c([2H])c(-c2c(F)c(F)c(NC(=O)c3ccsc3C(=O)O)c(F)c2F)c1[2H]] |
| 2/26 | 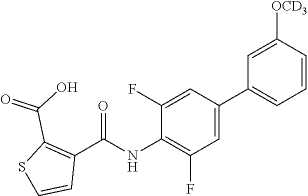 |
| 2/27 | 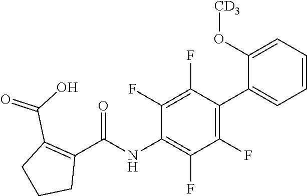 [SMILES: [2H]C([2H])([2H])Oc1ccccc1-c1c(F)c(F)c(NC(=O)C2=C(C(=O)O)CCC2)c(F)c1F] |
| 2/28 | 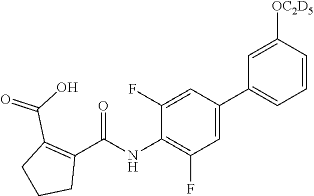 |
| 2/29 | 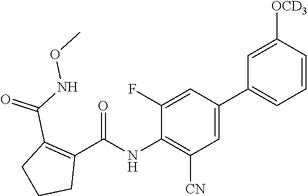 |
| 2/30 | 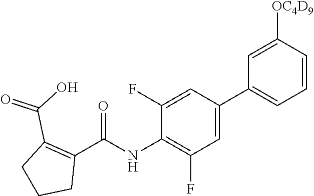 |
| 2/31 | 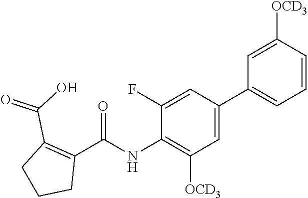 |

### Example 3. Compound 3a

This Example describes Compound 3a.

### Step 1: Prop-2-yn-1-yl 2-((3-fluoro-3′-(prop-2-yn-1-yloxy)-[1,1′-biphenyl]-4-yl)carbamoyl)cyclopent-1-ene-1-carboxylate (3a)

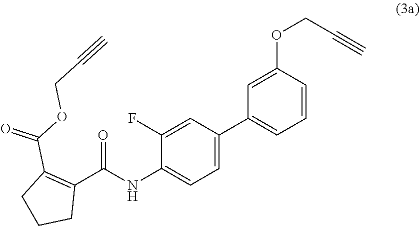 [SMILES: C#CCOC(=O)C1=C(C(=O)Nc2ccc(-c3cccc(OCC#C)c3)cc2F)CCC1]

To a solution of 2-((3-fluoro-3′-hydroxy-[1,1′-biphenyl]-4-yl)carbamoyl)cyclopent-1-ene-1-carboxylic acid (500 mg) in DMF (10 mL) was added 3-bromoprop-1-yne (346 mg) and K₂CO₃ (607 mg) and then the mixture was heated at 60° C. for 16 hours, cooled to rt, diluted with water and extracted with EtOAc (3 ×). The combined organic layer was washed with brine, dried over Na₂SO₄, concentrated and purified by FCC (PE:EA=2:1) to afford compound 3a as a yellow solid. LCMS (ESI): m/z 418.2 (M+H)⁺.

### Step 2: 2-((3-Fluoro-3′-(prop-2-yn-1-yloxy)-[1,1′-biphenyl]-4-yl)carbamoyl)cyclopent-1-ene-1-carboxylic acid (3)

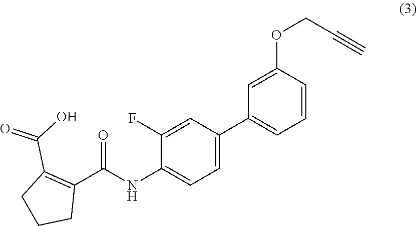 [SMILES: C#CCOc1cccc(-c2ccc(NC(=O)C3=C(C(=O)O)CCC3)c(F)c2)c1]

To a solution of compound 3a (450 mg) in MeOH (1 mL) and THF (3 mL) was added 2N NaOH (3 mL) at 0° C. The mixture was stirred at 0° C. for 3 h, diluted with 2N HCl to adjust the pH to 5-6, concentrated, and purified by reversed-phase flash chromatography (C18) (0.1% TFA in water, 10 to 100% MeCN) to give compound 3 as a yellow solid. ¹H-NMR (400 MHz, MeOD-d₄) δ 8.12 (t, J=8.2 Hz, 1H), 7.46-7.43 (m, 2H), 7.37 (t, J=8.0 Hz, 1H), 7.25-7.22 (m, 2H), 7.00-6.97 (m, 1H), 4.80 (d, J=2.4 Hz, 2H), 2.97-2.83 (m, 5H), 2.02-1.93 (m, 2H). LCMS (ESI): m/z 380.2 (M+H)⁺.

### Example 4. Compounds 4a and 4/1 to 4/3

### A. Compound 4a

This Example describes Compound 4a.

### Step 1: 3,5-Difluoro-3′-methoxy-[1,1′-biphenyl]-4-amine (4a)

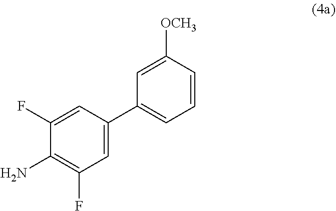 [SMILES: COc1cccc(-c2cc(F)c(N)c(F)c2)c1]

To a solution of 1-bromo-3-methoxybenzene (300 mg) in 1,4-dioxane (5 mL) and H₂O (0.5 mL) was added 2,6-difluoro-4-(4,4,5,5-tetramethyl-1,3,2-dioxaborolan-2-yl)aniline (411 mg), Cs₂CO₃ (786 mg) and Pd(PPh₃)₄ (30 mg). The mixture was heated at 90° C. for 3 h, cooled to rt, diluted with water, and extracted with EtOAc (3 ×). The combined organic layer was washed with brine, dried of Na₂SO₄, concentrated and purified by FCC (PE:EA=5:1) to give compound 4a as a white solid. LCMS (ESI): m/z 235.9 (M+H)+.

### Step 2: 2-((3,5-Difluoro-3′-methoxy-[1,1′-biphenyl]-4-yl)carbamoyl)cyclopent-1-ene-1-carboxylic acid (4)

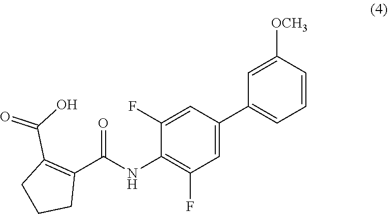 [SMILES: COc1cccc(-c2cc(F)c(NC(=O)C3=C(C(=O)O)CCC3)c(F)c2)c1]

A solution of compound 4a (100 mg) in CH₂C₁₂ (5 mL) was added 5,6-dihydro-1H-cyclopenta[c]furan-1,3(4H)-dione (70 mg). The mixture was stirred at rt for 2 h. The reaction mixture was filtered, and the filter cake washed with MeCN. The solid was dried in vacuum to afford compound 4 as a white solid. ¹H-NMR (400 MHz, DMSO-d6) δ 12.94 (br s, 1H), δ 10.13 (s, 1H), 7.56 (d, J=9.2 Hz, 2H), 7.40 (t, J=7.8 Hz, 1H), 7.32-7.28 (m, 2H), 7.00-6.98 (m, 1H), 3.84 (s, 3H), 2.80 (t, J=7.4 Hz, 2H), 2.70-2.67 (m, 2H), 1.97-1.89 (m, 2H). LCMS (ESI): m/z 374.1 (M+H)⁺.

### B. Compounds 4/1 to 4/3

The following compounds were prepared as described in for Example 4 using the appropriate building blocks.

| # | Building Block | Compound | Analytical Data |
|---|---|---|---|
| 4/1 | 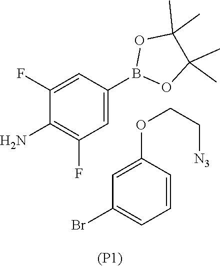 | 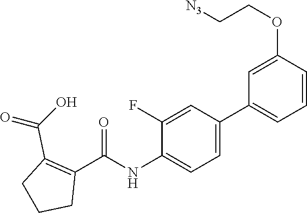 [SMILES: [N-]=[N+]=NCCOc1cccc(-c2ccc(NC(=O)C3=C(C(=O)O)CCC3)c(F)c2)c1] | ¹H-NMR (400 MHz, DMSO-d6) δ 13.02 (br s, 1H), δ 10.94 (br s, 1H), 8.09 (t, J = 8.4 Hz, 1H), 7.63 (dd, J = 1.8, 12.2 Hz, 1H), 7.53 (dd, J = 1.8, 8.6 Hz, 1H), 7.39 (t, J = 7.8 Hz, 1H), 7.30 (d, J = 7.6 Hz, 1H); 7.26 (t, J = 2.0 Hz, 1H), 6.97 (dd, J = 1.8, 8.2 Hz, 1H), 4.28 (t, J = 4.8 Hz, 2H), 3.68 (t, J = 4.8 Hz, 2H), 2.80 (t, J = 7.4 Hz, 2H), 2.70 (t, J = 7.2 Hz, 2H), 1.87 (q, J = 7.6 Hz, 2H). LCMS (ESI): m/z 411.0 (M + H)⁺ |
| 4/2 | 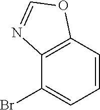 | 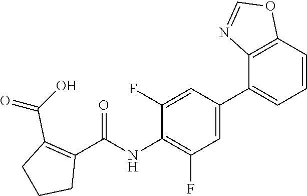 [SMILES: O=C(O)C1=C(C(=O)Nc2c(F)cc(-c3cccc4ocnc34)cc2F)CCC1] | ¹H-NMR (500 MHz, MeOD-d4) δ 8.51 (s, 1H), 7.80 (d, J = 9.0 Hz, 2H), 7.71-7.68 (m, 2H), 7.53 (t, J = 8.0 Hz, 1H), 2.92-2.86 (m, 4H), 1.94 (q, J = 7.5 Hz, 2H). LCMS (ESI): m/z 385.2 (M + H)⁺ |
| 4/3 | 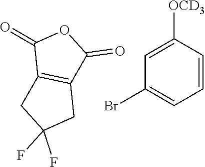 | 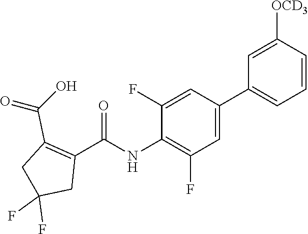 | ¹H NMR (400 MHz, MeOD-d4) δ 7.39-7.33 (m, 3H), 7.20 (d, J = 8.0 Hz, 1H), 7.16 (t, J = 2.0 Hz, 1H), 6.98-6.95 (m, 1H), 3.45-3.28 (m, 4H). LCMS (ESI): m/z 413.0 (M + H)⁺ |

### Example 5. Compound 5a

### Step 1: 3′-(Methoxy-d₃)-[1,1′-biphenyl]-4-amine (5a)

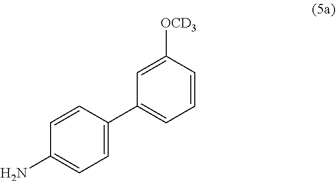

To a solution of 4-bromoaniline (150 mg) in 1,4-dioxane (5 mL) and H₂O (0.5 mL) were added (3-(methoxy-d3)phenyl)boronic acid (164 mg), Cs₂CO₃ (860 mg) and Pd(PPh₃)₄ (20 mg) and then the mixture was heated at 90° C. for 3 h, allowed to reach rt, diluted with water, and extracted with EtOAc (3 ×). The combined organic layer was washed with brine, dried of Na₂SO₄, concentrated and purified by FCC (PE:EA=8:1) to give compound 5a as a yellow solid. LCMS (ESI): m/z 203.2 (M+H)⁺.

### Step 2: 2-((3′-(Methoxy-d₃)-[1,1′-biphenyl]-4-yl)carbamoyl)cyclopent-1-ene-1-carboxylic acid (5)

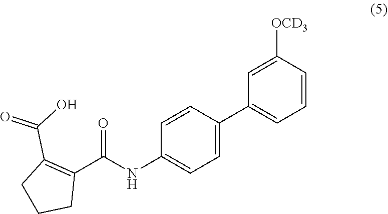

To a solution of compound 5a (100 mg) in CH₂C₁₂ (5 mL) was added 5,6-dihydro-1H-cyclopenta[c]furan-1,3(4H)-dione (116 mg). The mixture was stirred at room temperature for 5 h, concentrated and purified by preparative HPLC to afford compound 5 as a white solid. ¹H-NMR (400 MHz, DMSO-d6) δ 12.75 (br s, 1H), δ 10.34 (s, 1H), 7.72 (d, J=8.8 Hz, 2H), 7.40 (t, J=8.4 Hz, 2H), 7.35 (t, J=8.0 Hz, 1H), 7.21 (d, J=8.0 Hz, 1H), 7.17 (t, J=2.0 Hz, 1H), 6.90 (dd, J=2.0, 8.0 Hz, 1H), 2.79 (t, J=7.6 Hz, 2H), 2.67 (t, J=7.2 Hz, 2H), 1.93 (q, J=7.6 Hz, 2H). LCMS (ESI): m/z 341.2 (M+H)⁺.

### Example 6. Compounds 6/1 to 6/61

The following Examples were prepared similar as described in WO 2022/214691, WO 2023/118576 or EP23182205 (filed 28 Jun. 2023) by using the appropriate building blocks.

| # | Compound | Analytical Data |
|---|---|---|
| 6/1 | 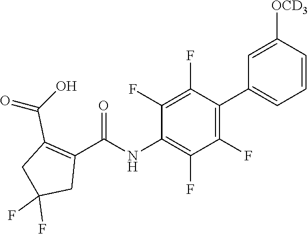 | ¹H-NMR (400 MHz, DMSO-d6) δ 13.26 (br s, 1H), 10.89 (s, 1H), 7.47 (t, J = 8.0 Hz, 1H), 7.13-7.08 (m, 3H), 3.40 (t, J = 14.8 Hz, 2H), 3.26 (t, J = 15.2 Hz, 2H). LCMS (ESI): m/z 449.0 (M + H)⁺ |
| 6/2 | 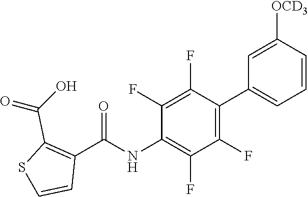 | ¹H-NMR (400 MHz, DMSO-d6) δ 11.42 (br s, 1H), 7.93 (d, J = 5.2 Hz, 1H), 7.50-7.44 (m, 2H), 7.14-7.08 (m, 3H). LCMS (ESI): m/z 429.1 (M + H)⁺ |
| 6/3 | 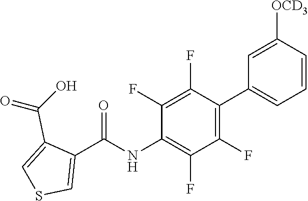 | ¹H-NMR (400 MHz, DMSO-d6) δ 13.18 (br s, 1H), 11.08 (s, 1H), 8.35 (d, J = 3.2 Hz, 1H), 8.15 (d, J = 2.8 Hz, 1H), 7.48 (t, J = 7.8 Hz, 1H), 7.14-7.08 (m, 3H). LCMS (ESI): m/z 429.1 (M + H)⁺ |
| 6/4 | 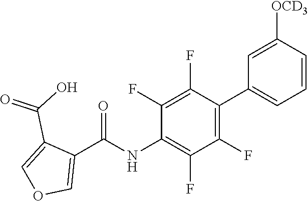 | ¹H-NMR (500 MHz, MeOD-d4) δ 8.24 (d, J = 1.5 Hz, 1H), 8.11 (d, J = 1.5 Hz, 1H), 7.42 (dd, J = 7.5, 9.0 Hz, 1H), 7.06-7.03 (m, 3H). LCMS (ESI): m/z 413.2 (M + H)⁺ |
| 6/5 | 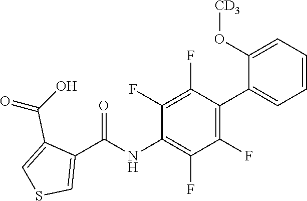 [SMILES: [2H]C([2H])([2H])Oc1ccccc1-c1c(F)c(F)c(NC(=O)c2cscc2C(=O)O)c(F)c1F] | ¹H-NMR (400 MHz, MeOD-d4) δ 8.37 (d, J = 3.6 Hz, 1H), 8.39 (d, J = 3.6 Hz, 1H), 7.50-7.46 (m, 1H), 7.29 (d, J = 7.6 Hz, 1H), 7.15 (d, J = 7.2 Hz, 1H), 7.07 (t, J = 7.4 Hz, 1H). LCMS (ESI): m/z 429.2 (M + H)⁺ |
| 6/6 | 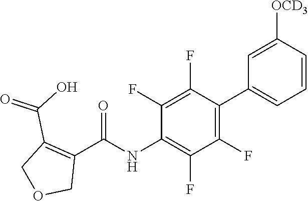 | ¹H-NMR (400 MHz, DMSO-d6) δ 11.26 (s, 1H), 7.47 (t, J = 7.8 Hz, 1H), 7.12-7.08 (m, 3H), 4.98 (t, J = 5.4 Hz, 2H), 4.89 (t, J = 4.8 Hz, 2H). LCMS (ESI): m/z 514.2 (M + H)⁺ |
| 6/7 | 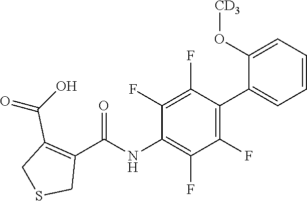 [SMILES: [2H]C([2H])([2H])Oc1ccccc1-c1c(F)c(F)c(NC(=O)C2=C(C(=O)O)CSC2)c(F)c1F] | ¹H-NMR (400 MHz, MeOD-d4) δ 7.50-7.46 (m, 1H), 7.27 (d, J = 7.2 Hz, 1H), 7.14 (d, J = 8.4 Hz, 1H), 7.07 (t, J = 7.6 Hz, 1H), 4.22 (t, J = 5.0 Hz, 2H), 4.11 (t, J = 4.8 Hz, 2H). LCMS (ESI): m/z 431.0 (M + H)⁺ |
| 6/8 | 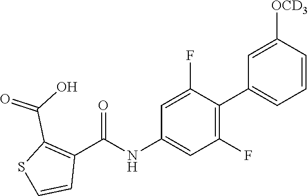 | ¹H-NMR (400 MHz, DMSO-d6) δ 11.64 (br s, 1H), 7.88 (s, 1H), 7.52- 7.40 (m, 4H), 6.99 (s, 3H). LCMS (ESI): m/z 393.0 (M + H)⁺ |
| 6/9 | 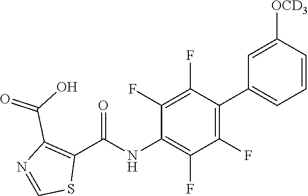 | ¹H-NMR (400 MHz, MeOD-d4) δ 8.94 (s, 1H), 7.45-7.41 (m, 1H), 7.07-7.03 (m, 3H). LCMS (ESI): m/z 430.1 (M + H)⁺ |
| 6/10 | 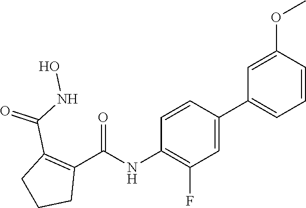 [SMILES: COc1cccc(-c2ccc(NC(=O)C3=C(C(=O)NO)CCC3)c(F)c2)c1] | ¹H-NMR (400 MHz, MeOD-d4) δ 8.19 (t, 1H), 7.46-7.41 (m, 2H), 7.34 (t, J = 7.8 Hz, 1H), 7.19 (d, J = 7.4 Hz, 1H), 7.14 (t, J = 1.8 Hz, 1H), 6.91 (dd, J = 2.4, 7.8 Hz, 1H), 3.85 (s, 3H), 2.90-2.80 (m, 4H), 1.97-1.89 (m, 2H). LCMS (ESI): m/z 371.1 (M + H)⁺ |
| 6/11 | 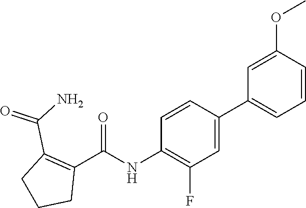 [SMILES: COc1cccc(-c2ccc(NC(=O)C3=C(C(N)=O)CCC3)c(F)c2)c1] | ¹H-NMR (400 MHz, MeOD-d4) δ 8.21 (t, J = 8.4 Hz, 1H), 7.45-7.41 (m, 2H), 7.34 (t, J = 8.2 Hz, 1H), 7.19-7.13 (m, 2H), 6.93-6.90 (m, 1H), 3.85 (s, 3H), 2.92-2.87 (m, 4H), 2.00-1.92 (m, 2H). LCMS (ESI): m/z 355.2 (M + H)⁺ |
| 6/12 | 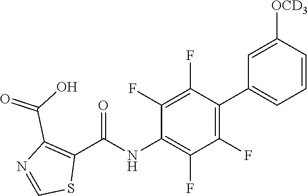 | ¹H-NMR (400 MHz, MeOD-d4) δ 9.03 (s, 1H), 7.45-7.41 (m, 1H), 7.06-7.04 (m, 3H). LCMS (ESI): m/z 430.2 (M + H)⁺ |
| 6/13 | 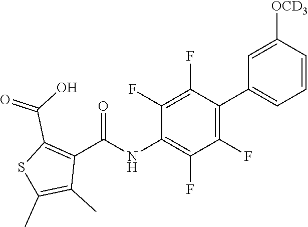 | ¹H-NMR (400 MHz, MeOD-d4) δ 7.44-7.40 (m, 1H), 7.06-7.03 (m, 3H), 2.35 (s, 3H), 2.29 (m, 3H). LCMS (ESI): m/z 457.2 (M + H)⁺ |
| 6/14 | 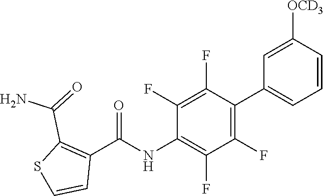 | ¹H-NMR (400 MHz, MeOD-d4) δ 7.81 (d, J = 5.6 Hz, 1H), 7.62 (d, J = 5.2 Hz, 1H), 7.43 (t, J = 8.4 Hz, 1H), 7.07-7.04 (m, 3H). LCMS (ESI): m/z 428.1 (M + H)⁺ |
| 6/15 | 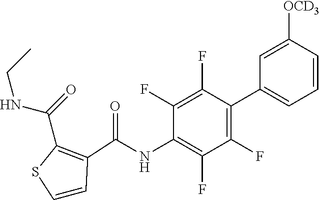 | ¹H-NMR (500 MHz, MeOD-d4) δ 7.73 (d, J = 5.5 Hz, 1H), 7.66 (d, J = 5.5 Hz, 1H), 7.45-7.42 (m, 1H), 7.07-7.05 (m, 3H), 3.41 (q, J = 7.2 Hz, 2H), 1.13 (t, J = 7.5 Hz, 3H). LCMS (ESI) m/z 456.2 (M + H)⁺ |
| 6/16 | 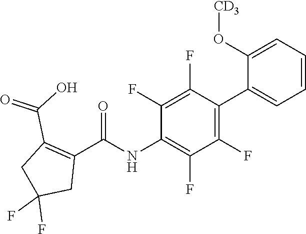 [SMILES: [2H]C([2H])([2H])Oc1ccccc1-c1c(F)c(F)c(NC(=O)C2=C(C(=O)O)CC(F)(F)C2)c(F)c1F] | ¹H-NMR (400 MHz, MeOD-d4) δ 7.51-7.46 (m, 1H), 7.27 (d, J = 6.4 Hz, 1H), 7.15 (d, J = 8.0 Hz, 1H), 7.07 (dd, J = 7.0, 7.8 Hz, 1H), 3.45- 3.30 (m, 4H). LCMS (ESI): m/z 449.2 (M + H)⁺ |
| 6/17 | 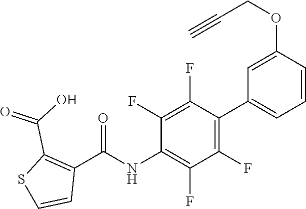 [SMILES: C#CCOc1cccc(-c2c(F)c(F)c(NC(=O)c3ccsc3C(=O)O)c(F)c2F)c1] | ¹H-NMR (400 MHz, MeOD-d4) δ 7.82 (d, J = 5.2 Hz, 1H), 7.68 (d, J = 4.8 Hz, 1H), 7.48-7.42 (m, 1H), 7.14-7.10 (m, 3H), 4.79 (d, J = 2.0 Hz, 2H), 2.97 (t, J = 2.4 Hz, 1H). LCMS (ESI): m/z 450.1 (M + H)⁺ |
| 6/18 | 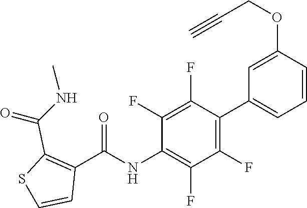 [SMILES: C#CCOc1cccc(-c2c(F)c(F)c(NC(=O)c3ccsc3C(=O)NC)c(F)c2F)c1] | ¹H-NMR (400 MHz, MeOD-d4) δ 7.72 (d, J = 5.2 Hz, 1H), 7.66 (d, J = 5.2 Hz, 1H), 7.48-7.43 (m, 1H), 7.14-7.10 (m, 3H), 4.79 (d, J = 2.4 Hz, 2H), 2.98 (t, J = 2.6 Hz, 1H), 2.93 (s, 3H). LCMS (ESI): m/z 463.1 (M + H)⁺ |
| 6/19 | 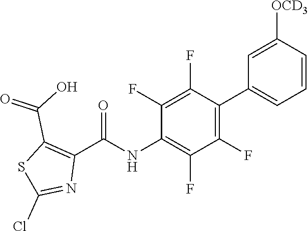 | ¹H-NMR (400 MHz, MeOD-d4) δ 7.64 (dd, J = 7.6, 9.0 Hz, 1H), 7.06- 7.04 (m, 3H). LCMS (ESI): m/z 464.0 (M + H)⁺ |
| 6/20 | 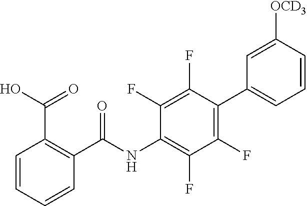 | ¹H-NMR (400 MHz, DMSO-d6) δ 13.21 (br s, 1H), 10.70 (s, 1H), 7.91 (d, J = 7.6 Hz, 1H), 7.73 (m, 3H), 7.48 (t, J = 8.0 Hz, 1H), 7.15- 7.08 (m, 3H). LCMS (ESI): m/z 421.1 (M + H)⁺ |
| 6/21 | 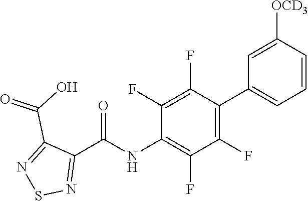 | ¹H-NMR (500 MHz, MeOD-d4) δ 7.20 (dd, J = 7.5, 8.5 Hz, 1H), 7.07- 7.05 (m, 3H). LCMS (ESI): m/z 431.1 (M + H)⁺ |
| 6/22 | 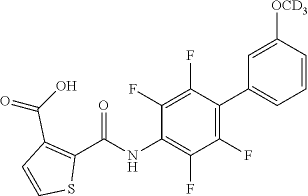 | (ESI): m/z 427.3 (M + H)⁺ |
| 6/23 | 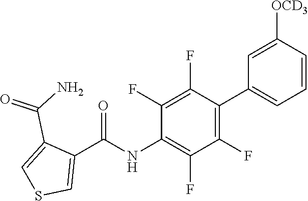 | ¹H-NMR (400 MHz, DMSO-d6) δ 12.22 (s, 1H), 8.38 (d, J = 3.2 Hz, 2H), 8.26 (d, J = 3.6 Hz, 1H), 7.86 (s, 1H), 7.48 (t, H = 7.8 Hz, 1H), 7.13-7.08 (m, 3H). LCMS (ESI): m/z 428.0 (M + H)⁺ |
| 6/24 | 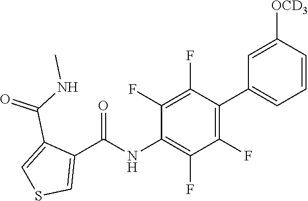 | (ESI): m/z 426.1 (M + H)⁺ |
| 6/25 | 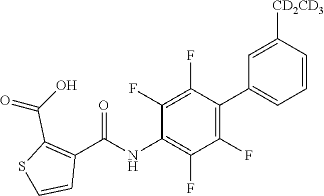 | ¹H-NMR (500 MHz, MeOD-d4) δ 7.70 (d, J = 5.5 Hz, 1H), 7.44 (d, J = 5.5 Hz, 1H), 7.41 (d, J = 8.0 Hz, 1H), 7.33-7.29 (m, 3H). LCMS (ESI): m/z 429.0 (M + H)⁺ |
| 6/26 | 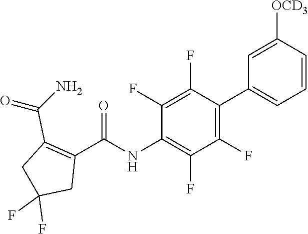 | (ESI): m/z 446.4 (M − H)⁻ |
| 6/27 | 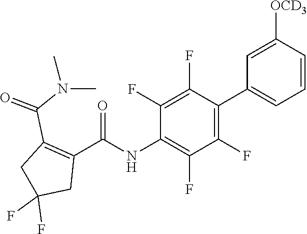 | (ESI): m/z 476.4 (M + H)⁺ |
| 6/28 | 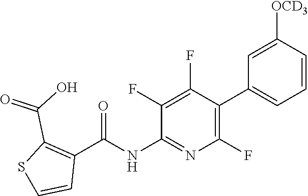 | ¹H-NMR (400 MHz, MeOD-d4) δ 7.73 (d, J = 5.6 Hz, 1H), 7.49 (d, J = 5.2 Hz, 1H), 7.41 (t, J = 8.2 Hz, 1H), 7.07-7.02 (m, 3H). LCMS (ESI): m/z 412.2 (M + H)⁺ |
| 6/29 | 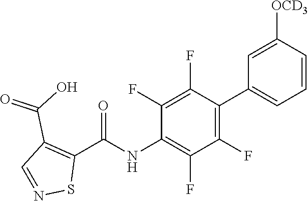 | ¹H-NMR (400 MHz, MeOD-d4) δ 9.01 (s, 1H), 7.43 (dd, J = 7.6, 8.8 Hz, 1H), 7.07-7.05 (m, 3H). LCMS (ESI): m/z 430.2 (M + H)⁺ |
| 6/30 | 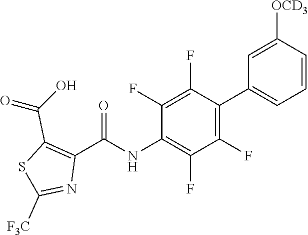 | ¹H-NMR (400 MHz, MeOD-d4) δ 7.45-7.41 (m, 1H), 7.07-7.04 (m, 3H). LCMS (ESI): m/z 498.2 (M + H)⁺ |
| 6/31 | 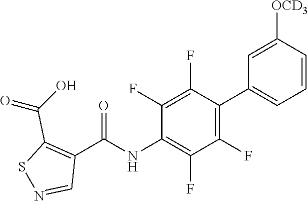 | ¹H-NMR (400 MHz, MeOD-d4) δ 9.00 (s, 1H), 7.43 (dd, J = 8.0, 8.8 Hz, 1H), 7.07-7.04 (m, 3H). LCMS (ESI): m/z 430.1 (M + H)⁺ |
| 6/32 | 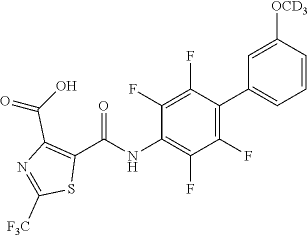 | ¹H-NMR (500 MHz, MeOD-d4) δ 7.43 (dd, J = 7.5, 9.0 Hz, 1H), 7.06- 7.04 (m, 3H). LCMS (ESI): m/z 498.0 (M + H)⁺ |
| 6/33 | 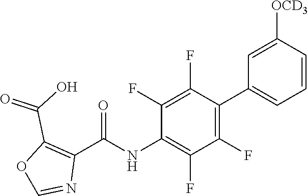 | ¹H-NMR (400 MHz, MeOD-d4) δ 8.34 (s, 1H), 7.43 (dd, J = 7.8, 8.6 Hz, 1H), 7.06-7.04 (m, 3H). LCMS (ESI): m/z 414.1 (M + H)⁺ |
| 6/34 | 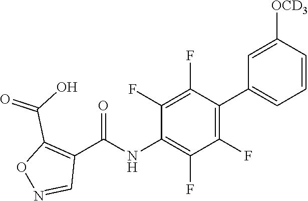 | ¹H-NMR (400 MHz, MeOD-d4) δ 8.98 (s, 1H), 7.43 (dd, J = 7.2, 8.4 Hz, 1H), 7.07-7.04 (m, 3H). LCMS (ESI): m/z 414.1 (M + H)⁺ |
| 6/35 | 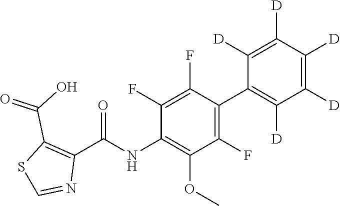 [SMILES: [2H]c1c([2H])c([2H])c(-c2c(F)c(F)c(NC(=O)c3ncsc3C(=O)O)c(OC)c2F)c([2H])c1[2H]] | ¹H-NMR (400 MHz, MeOD-d4) δ 8.92 (s, 1H), 3.94 (s, 3H). LCMS (ESI): m/z 414.0 (M + H)⁺ |
| 6/36 | 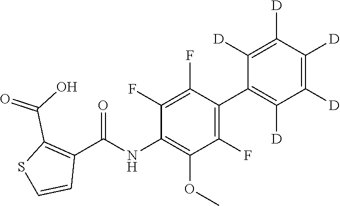 [SMILES: [2H]c1c([2H])c([2H])c(-c2c(F)c(F)c(NC(=O)c3ccsc3C(=O)O)c(OC)c2F)c([2H])c1[2H]] | ¹H-NMR (500 MHz, MeOD-d4) δ 7.71 (s, 2H), 3.94 (s, 3H). LCMS (ESI): m/z 413.1 (M + H)⁺ |
| 6/37 | 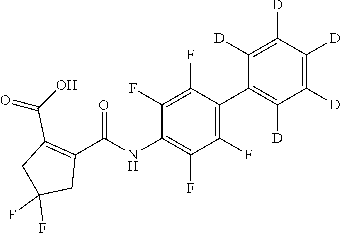 [SMILES: [2H]c1c([2H])c([2H])c(-c2c(F)c(F)c(NC(=O)C3=C(C(=O)O)CC(F)(F)C3)c(F)c2F)c([2H])c1[2H]] | ¹H-NMR (40 0MHz, MeOD-d4) δ 3.45-3.30 (m, 4H). LCMS (ESI): m/z 421.1 (M + H)⁺ |
| 6/38 | 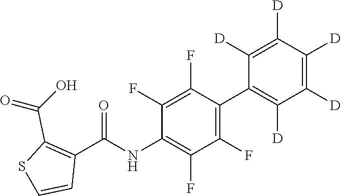 [SMILES: [2H]c1c([2H])c([2H])c(-c2c(F)c(F)c(NC(=O)c3ccsc3C(=O)O)c(F)c2F)c([2H])c1[2H]] | ¹H-NMR (400 MHz, MeOD-d4) δ 7.70 (d, J = 5.2 Hz, 1H), 7.45 (d, J = 5.2 Hz, 1H). LCMS (ESI): m/z 401.1 (M + H)⁺ |
| 6/39 | 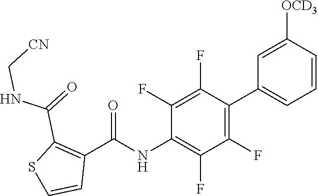 | ¹H-NMR (500 MHz, MeOD-d4) δ 7.83 (d, J = 5.0 Hz, 1H), 7.71 (d, J = 5.5 Hz, 1H), 7.44 (t, J = 7.8 Hz, 1H), 7.08-7.05 (m, 3H), 4.36 (s, 2H). LCMS (ESI): m/z 467.1 (M + H)⁺ |
| 6/40 | 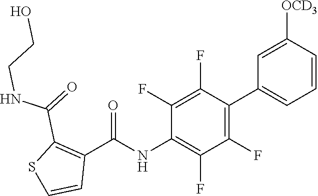 | ¹H-NMR (500 MHz, DMSO-d6) δ 11.23 (s, 1H), 9.52 (s, 1H), 7.90 (d, J = 5.5 Hz, 1H), 7.68 (d, J = 5.5 Hz, 1H), 7.48 (t, J = 8.3 Hz, 1H), 7.16-7.09 (m, 3H), 4.75 (br s, 1H), 3.51-3.47 (m, 2H), 3.37-3.29 (m, 2H). LCMS (ESI): m/z 472.0 (M + H)⁺ |
| 6/41 | 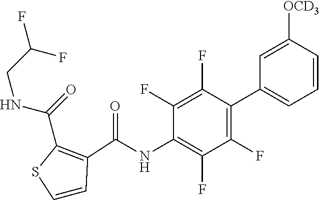 | ¹H-NMR (500 MHz, MeOD-d4) δ 7.80 (d, J = 5.0 Hz, 1H), 7.69 (d, J = 5.0 Hz, 1H), 7.45-7.42 (m, 1H), 7.07-7.05 (m, 3H), 6.12-5.90 (m, 1H), 3.76 (dt, J = 4.3, 15.0 Hz, 2H). LCMS (ESI): m/z 492.2 (M + H)⁺ |
| 6/42 | 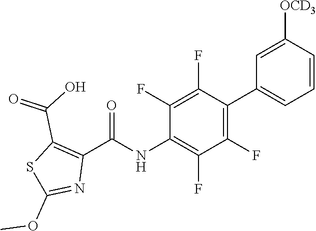 | ¹H-NMR (500 MHz, MeOD-d4) δ 7.45 (dd, J = 7.5, 8.5 Hz, 1H), 7.08- 7.06 (m, 3H), 4.26 (s, 3H). LCMS (ESI): m/z 459.9 (M + H)⁺ |
| 6/43 | 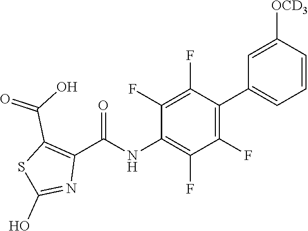 | ¹H-NMR (500 MHz, MeOD-d4) δ 7.45 (t, J = 8.3 Hz, 1H), 7.05-7.03 (m, 3H). LCMS (ESI): m/z 445.9 (M + H)⁺ |
| 6/44 | 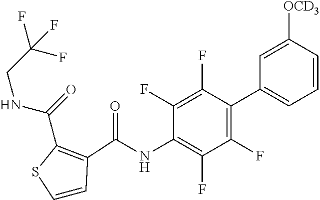 | ¹H -NMR (500 MHz, DMSO-d6) δ 11.07 (s, 1H), 10.05 (t, J = 6.0 Hz, 1H), 8.00 (d, J = 5.5 Hz, 1H), 7.73 (d, J = 5.5 Hz, 1H), 7.48 (t, J = 8.0 Hz, 1H), 7.18-7.08 (m, 3H), 4.20-4.13 (m, 2H). LCMS (ESI): m/z 510.1 (M + H)⁺ |
| 6/45 | 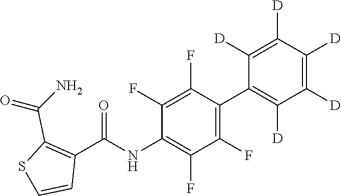 [SMILES: [2H]c1c([2H])c([2H])c(-c2c(F)c(F)c(NC(=O)c3ccsc3C(N)=O)c(F)c2F)c([2H])c1[2H]] | (ESI): m/z 398.2 (M + H)⁺ |
| 6/46 | 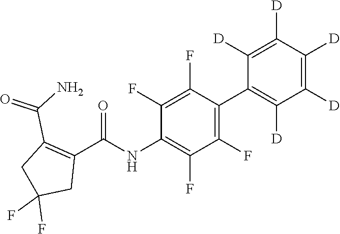 [SMILES: [2H]c1c([2H])c([2H])c(-c2c(F)c(F)c(NC(=O)C3=C(C(N)=O)CC(F)(F)C3)c(F)c2F)c([2H])c1[2H]] | (ESI): m/z 418.2 (M + H)⁺ |
| 6/47 | 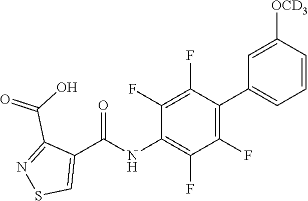 | ¹H-NMR (400 MHz, MeOD-d4) δ 9.52 (s, 1H), 7.43 (dd, J = 7.6, 8.4 Hz, 1H), 7.07-7.04 (m, 3H). LCMS (ESI): m/z 430.0 (M + H)⁺ |
| 6/48 | 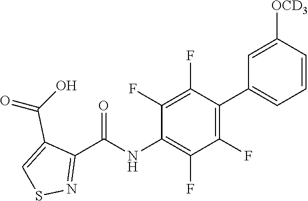 | ¹H-NMR (400 MHz, MeOD-d4) δ 9.65 (s, 1H), 7.45 (dd, J = 7.6, 8.4 Hz, 1H), 7.08-7.06 (m, 3H). LCMS (ESI): m/z 430.0 (M + H)⁺ |
| 6/49 | 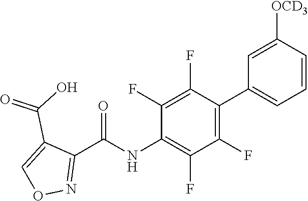 | ¹H-NMR (400 MHz, MeOD-d4) δ 9.49 (s, 1H), 7.45 (dd, J = 7.2, 9.2 Hz, 1H), 7.08-7.05 (m, 3H). LCMS (ESI): m/z 414.1 (M + H)⁺ |
| 6/50 | 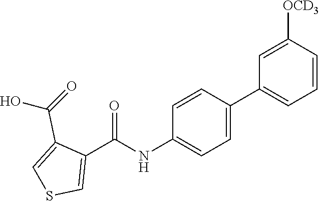 | ¹H-NMR (400 MHz, MeOD-d4) δ 8.43 (d, J = 3.6 Hz, 1H), 8.22 (d, J = 3.6 Hz, 1H), 7.77 (d, J = 8.8 Hz, 2H), 7.62 (d, J = 8.4 Hz, 2H), 7.33 (t, J = 8.0 Hz, 1H), 7.18 (d, J = 7.6 Hz, 1H), 7.14 (s, 1H), 6.89 (dd, J = 2.2, 8.2 Hz, 1H). LCMS (ESI): m/z 357.1 (M + H)⁺ |
| 6/51 | 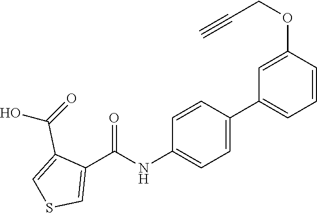 [SMILES: C#CCOc1cccc(-c2ccc(NC(=O)c3cscc3C(=O)O)cc2)c1] | ¹H-NMR (500 MHz, MeOD-d4) δ 8.43 (d, J = 3.5 Hz, 1H), 8.22 (d, J = 3.5 Hz, 1H), 7.77 (dd, J = 2.0, 6.5 Hz, 2H), 7.62 (dd, J = 2.0, 6.5 Hz, 2H), 7.35 (t, J = 7.8 Hz, 1H), 7.25-7.22 (m, 2H), 6.97-6.94 (m, 1H), 4.79 (d, J = 2.0 Hz, 2H), 2.96 (t, J = 2.5 Hz, 1H). LCMS (ESI): m/z 378.1 (M + H)⁺ |
| 6/52 | 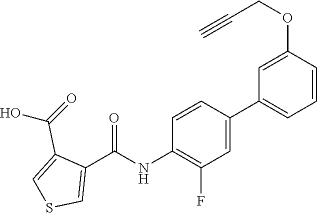 [SMILES: C#CCOc1cccc(-c2ccc(NC(=O)c3cscc3C(=O)O)c(F)c2)c1] | ¹H-NMR (500 MHz, DMSO-d6) δ 13.45 (br s, 1H), 11.33 (s, 1H), 8.42 (d, J = 3.0 Hz, 1H), 8.26 (d, J = 3.5 Hz, 1H), 8.19 (t, J = 8.3 Hz, 1H), 7.65 (dd, J = 2.0, 7.5 Hz, 2H), 7.57 (dd, J = 1.5, 8.5 Hz, 2H), 7.40 (t, J = 7.8 Hz, 1H), 7.34-7.31 (m, 2H), 7.01-6.99 (m, 1H), 4.90 (d, J = 2.0 Hz, 2H), 3.59 (t, J = 2.5 Hz, 1H). LCMS (ESI): m/z 395.9 (M + H)⁺ |
| 6/53 | 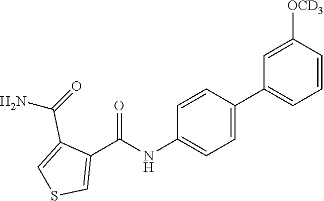 | ¹H-NMR (400 MHz, MeOD-d4) δ 8.24 (d, J = 2.8 Hz, 1H), 8.14 (d, J = 3.6 Hz, 1H), 7.77 (d, J = 8.4 Hz, 2H), 7.61 (d, J = 8.4 Hz, 2H), 7.33 (t, J = 8.0 Hz, 1H), 7.20-7.14 (m, 2H), 6.90-6.87 (m, 1H). LCMS (ESI): m/z 356.1 (M + H)⁺ |
| 6/54 | 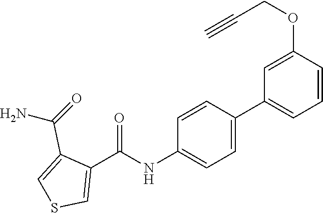 [SMILES: C#CCOc1cccc(-c2ccc(NC(=O)c3cscc3C(N)=O)cc2)c1] | ¹H-NMR (400 MHz, MeOD-d4) δ 8.23 (d, J = 3.6 Hz, 1H), 8.14 (d, J = 3.2 Hz, 1H), 7.77 (d, J = 8.8 Hz, 2H), 7.61 (d, J = 8.8 Hz, 2H), 7.35 (t, J = 8.0 Hz, 1H), 7.25-7.22 (m, 2H), 6.97-6.94 (m, 1H), 4.79 (d, J = 2.0 Hz, 2H), 2.96 (t, J = 2.4 Hz, 1H). LCMS (ESI): m/z 377.1 (M + H)⁺ |
| 6/55 | 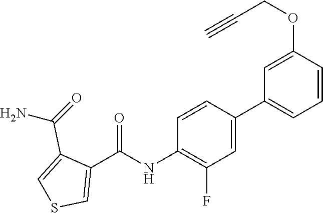 [SMILES: C#CCOc1cccc(-c2ccc(NC(=O)c3cscc3C(N)=O)c(F)c2)c1] | ¹H-NMR (500 MHz, MeOD-d4) δ 8.33 (d, J = 3.0 Hz, 1H), 8.22 (t, J = 8.0 Hz, 1H), 8.16 (d, J = 3.5 Hz, 1H), 7.48-7.45 (m, 2H), 7.37 (t, J = 7.8 Hz, 1H), 7.26-7.23 (m, 2H), 7.00-6.97 (m, 1H), 4.80 (d, J = 2.0 Hz, 2H), 2.97 (t, J = 2.3 Hz, 1H). LCMS (ESI): m/z 395.1 (M + H)⁺ |
| 6/56 | 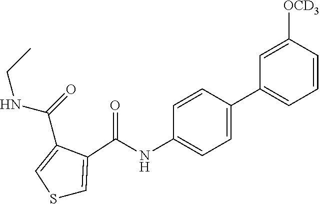 | ¹H-NMR (400 MHz, MeOD-d4) δ 8.22 (d, J = 3.2 Hz, 1H), 8.01 (d, J = 3.2 Hz, 1H), 7.76 (d, J = 8.4 Hz, 2H), 7.61 (d, J = 8.8 Hz, 2H), 7.33 (t, J = 8.0 Hz, 1H), 7.18 (d, J = 8.0 Hz, 1H), 7.14 (t, J = 2.0 Hz, 1H), 6.88 (dd, J = 2.0, 8.4 Hz, 1H), 3.42 (q, J = 7.2 Hz, 2H), 1.24 (t, J = 7.2 Hz, 3H). LCMS (ESI): m/z 384.1 (M + H)⁺ |
| 6/57 | 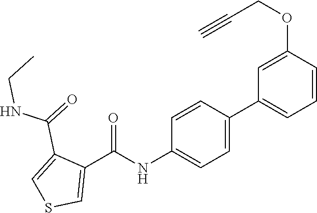 [SMILES: C#CCOc1cccc(-c2ccc(NC(=O)c3cscc3C(=O)NCC)cc2)c1] | ¹H-NMR (500 MHz, MeOD-d4) δ 8.22 (d, J = 3.0 Hz, 1H), 8.02 (d, J = 3.5 Hz, 1H), 7.77 (d, J = 8.5 Hz, 2H), 7.62 (d, J = 9.0 Hz, 2H), 7.35 (t, J = 7.8 Hz, 1H), 7.24-7.22 (m, 2H), 6.95 (dd, J = 2.3, 7.8 Hz, 1H), 4.79 (d, J = 2.0 Hz, 2H), 3.42 (q, J = 7.3 Hz, 2H), 2.96 (t, J = 2.5 Hz, 1H), 1.24 (t, J = 7.3 Hz, 3H). LCMS (ESI): m/z 405.1 (M + H)⁺ |
| 6/58 | 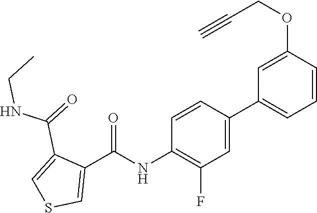 [SMILES: C#CCOc1cccc(-c2ccc(NC(=O)c3cscc3C(=O)NCC)c(F)c2)c1] | ¹H-NMR (500 MHz, MeOD-d4) δ 8.30 (d, J = 3.5 Hz, 1H), 8.19 (t, J = 8.0 Hz, 1H), 8.02 (d, J = 3.5 Hz, 1H), 7.48-7.45 (m, 2H), 7.37 (t, J = 8.0 Hz, 1H), 7.26-7.24 (m, 2H), 7.00-6.98 (m, 1H), 4.80 (d, J = 2.0 Hz, 2H), 3.42 (q, J = 7.5 Hz, 2H), 2.97 (t, J = 2.5 Hz, 1H), 1.24 (t, J = 7.3 Hz, 3H). LCMS (ESI): m/z 423.1 (M + H)⁺ |
| 6/59 | 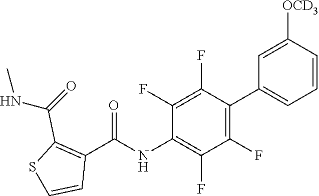 | ¹H-NMR (400 MHz, DMSO-d6) δ 11.39 (s, 1H), 9.27 (br s, 1H), 7.88 (d, J = 5.2 Hz, 1H), 7.66 (d, J = 5.2 Hz, 1H), 7.48 (t, J = 8.0 Hz, 1H), 7.14-7.09 (m, 3H), 2.80 (d, J = 4.8 Hz, 2H). LCMS (ESI): m/z 442.1 (M + H)⁺ |
| 6/60 | 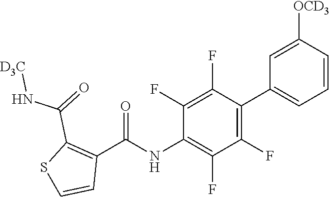 | ¹H-NMR (400 MHz, DMSO-d6) δ 11.39 (s, 1H), 9.25 (s, 1H), 7.88 (d, J = 5.6 Hz, 1H), 7.66 (d, J = 5.2 Hz, 1H), 7.48 (t, J = 8.0 Hz, 1H), 7.15-7.09 (m, 3H). LCMS (ESI): m/z 445.1 (M + H)⁺ |
| 6/61 | 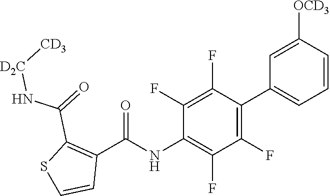 | ¹H-NMR (400 MHz, DMSO-d6) δ 11.32 (br s, 1H), 9.44 (br s, 1H), 7.87 (d, J = 5.2 Hz, 1H), 7.66 (d, J = 5.2 Hz, 1H), 7.48 (t, J = 7.8 Hz, 1H), 7.15-7.09 (m, 3H). LCMS (ESI): m/z 461.2 (M + H)⁺ |

### Example 100: Isothermal Titration Calorimetry (ITC)

ITC experiments were conducted on an Affinity ITC instrument (TA Instruments, New Castle, DE) at 25° C. with a stirring rate of 75 rpm. Nurr1 LBD protein (10 or 30 μM) in buffer (20 mM Tris pH 7.5, 100 mM NaCl, 5% glycerol) containing 3% or 5% DMSO was titrated with the test compounds (100 μM in the same buffer containing 3% or 5% DMSO) in 25 injections (1×1 μL and 24×3 μL or 1×1 μL and 24×4 μL) with an injection interval of 180 s or 300 s. As control experiments, the test compounds were titrated to the buffer, and the buffer was titrated to the Nurr1 LBD protein under otherwise identical conditions. The heat rates of the compound-Nurr1 LBD titrations were analyzed using NanoAnalyze software (TA Instruments, New Castle, DE) with an independent binding model.

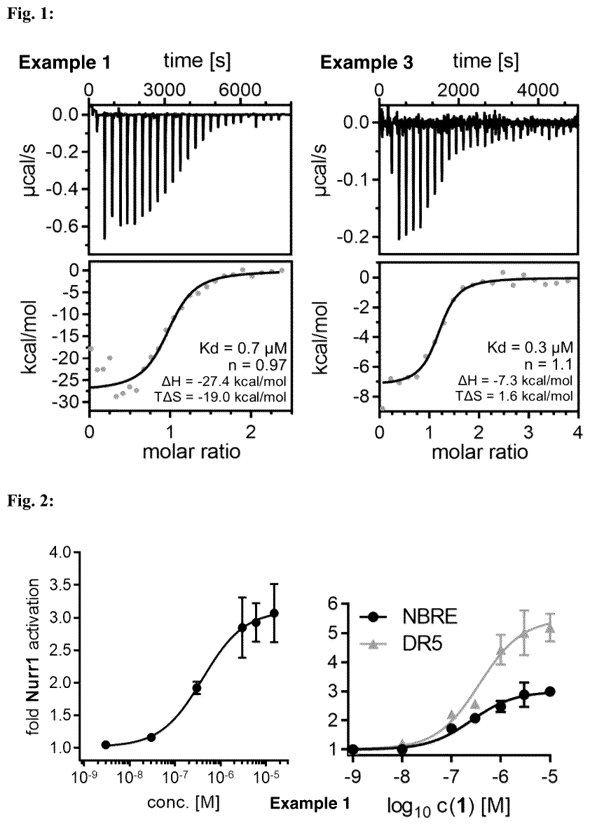

FIG. 1 depicts the binding of Example 1 (vidofludimus) and Example 3 to transcription factor Nurr1 (NR⁴A2). The data shows a K_{d} of 0.7 μM and 0.3 μM, respectively.

### Example 101-A: Hybrid Gal4-Nurr1 Reporter Gene Assays to Assess Nurr1 Agonism

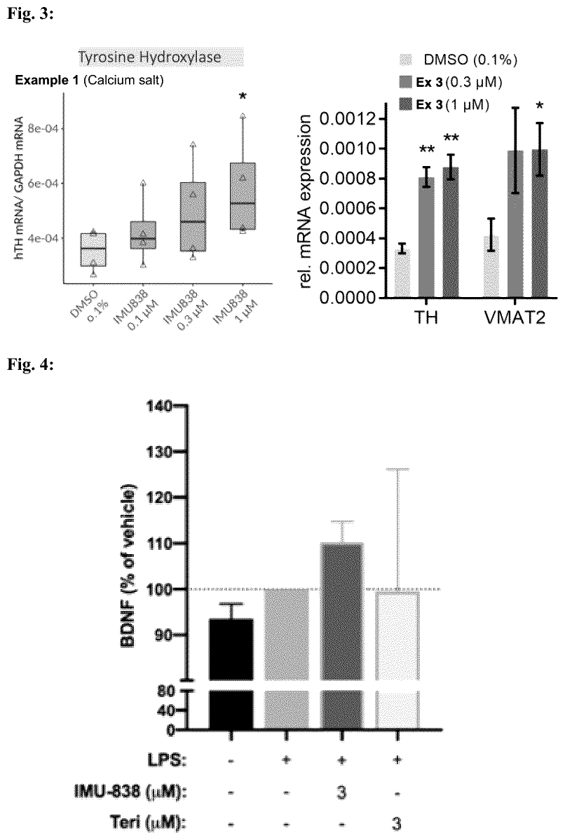

Nurr1 modulation was determined in a Ga14 hybrid reporter gene assays in HEK293T cells (German Collection of Microorganisms and Cell Culture GmbH, DSMZ) using pFR-Luc (Stratagene, La Jolla, CA, USA; reporter), pRL-SV40 (Promega, Madison, WI, USA; internal control) and pFA-CMV-hNurr1-LBD, coding for the hinge region and ligand binding domain of the canonical isoform of human Nurr1. Cells were cultured in Dulbecco's modified Eagle's medium (DMEM), high glucose supplemented with 10% fetal calf serum (FCS), sodium pyruvate (1 mM), penicillin (100 U/mL), and streptomycin (100 μg/mL) at 37° C. and 5% CO₂ and seeded in 96-well plates (3×10⁴ cells/well). After 24 h, medium was changed to Opti-MEM without supplements and cells were transiently transfected using Lipofectamine LTX reagent (Invitrogen) according to the manufacturer's protocol. Five hours after transfection, cells were incubated with the test compounds in Opti-MEM supplemented with penicillin (100 U/mL), streptomycin (100 μg/mL) and 0.1% DMSO for 16 h before luciferase activity was measured using the Dual-Glo Luciferase Assay System (Promega) according to the manufacturer's protocol on a Tecan Spark luminometer (Tecan Deutschland GmbH, Germany). Firefly luminescence was divided by Renilla luminescence and multiplied by 1000 resulting in relative light units (RLU) to normalize for transfection efficiency and cell growth. Fold activation was obtained by dividing the mean RLU of test compound by the mean RLU of the untreated control. All samples were tested in at least three biologically independent experiments in duplicates. For dose-response curve fitting and calculation of EC₅₀ values, the equation “[Agonist] versus response (three parameters)” was used in GraphPad Prism (version 7.00, GraphPad Software, La Jolla, CA, USA). FIG. 2 (left) depicts a representative example of the Nurr1-activation with compound of Example 1. The DHODH inhibition was measured as shown in Example 102.

| | | | Nurr1 agonism | | DHODH |
|---|---|---|---|---|---|
| | Nurr1-Gal4 | max. fold | | max. fold | inhibition |
| Example# | EC₅₀ range | activation | EC₅₀ [μM] | activation | IC₅₀ [μM] |
| 1 | | | 0.40 ± 0.20 | 3.1 ± 0.4 | 0.61 ± 0.07 |
| 2/1 | | | 0.079 ± 0.001 | 2.61 ± 0.07 | 0.40 ± 0.10 |
| 2/2 | ++ | +++ | | | + |
| 2/3 | ++ | +++ | | | |
| 2/4 | ++ | + | | | |
| 2/5 | | | 0.20 ± 0.01 | 1.6 ± 0.1 | 0.064 ± 0.009 |
| 2/6 | | | 1.07 ± 0.04 | 2.56 ± 0.07 | 0.60 ± 0.08 |
| 2/7 | | | 9.9 ± 0.3 | 3.5 ± 0.1 | 0.64 ± 0.03 |
| 2/8 | | | 0.33 ± 0.04 | 2.04 ± 0.08 | 0.20 ± 0.06 |
| 2/9 | | | 6.8 ± 0.2 | 3.18 ± 0.08 | 10.2 ± 4.7 |
| 2/10 | +++ | + | | | |
| 2/11 | | | 25 ± 1 | 4.2 ± 0.1 | 1.98 ± 0.02 |
| 2/12 | ++ | +++ | | | |
| 2/13 | + | +++ | | | |
| 2/14 | | | 1.0 ± 0.3 | 1.45 ± 0.06 | 0.72 ± 0.04 |
| 2/15 | | | 0.43 ± 0.02 | 2.25 ± 0.05 | 0.022 ± 0.009 |
| 2/16 | ++ | + | | | |
| 2/17 | ++ | ++ | | | |
| 2/18 | + | ++ | | | |
| 2/19 | | | 5.8 ± 0.2 | 2.46 ± 0.05 | inactive (>30) |
| 2/20 | ++ | ++ | | | |
| 2/21 | + | ++ | | | |
| 2/22 | + | ++ | | | |
| 2/23 | ++ | + | | | |
| 2/24 | + | + | | | |
| 2/31 | ++ | + | | | |
| 3 | | | 0.23 ± 0.03 | 5.7 ± 0.5 | 1.80 ± 0.43 |
| 4 | ++ | + | | | |
| 4/1 | +++ | +++ | | | |
| 4/2 | | | 1.9 ± 0.2 | 2.2 ± 0.1 | 0.27 ± 0.07 |
| 4/3 | | | 0.30 ± 0.20 | 3.7 ± 0.8 | 0.013 ± 0.001 |
| 5 | | | 0.10 ± 0.03 | 3.9 ± 0.3 | 0.89 ± 0.24 |
| 6/4 | +++ | ++ | | | +++ |
| 6/5 | +++ | + | | | ++++ |
| 6/11 | ++ | + | | | 0 |
| 6/12 | +++ | + | | | ++++ |
| 6/13 | +++ | ++ | | | +++ |
| 6/14 | ++ | + | | | + |
| 6/15 | ++ | + | | | 0 |
| 6/20 | +++ | ++ | | | +++ |
| 6/23 | +++ | ++ | | | ++ |
| 6/24 | ++ | + | | | 0 |
| 6/26 | +++ | + | | | + |
| 6/27 | + | + | | | + |
| 6/39 | + | ++ | | | ++ |
| 6/40 | + | + | | | 0 |
| 6/45 | ++ | + | | | + |
| 6/46 | ++ | + | | | + |
| 6/50 | +++ | ++ | | | ++ |
| 6/51 | ++ | +++ | | | + |
| 6/52 | +++ | ++ | | | ++ |
| 6/53 | ++ | + | | | 0 |
| 6/54 | + | + | | | 0 |
| 6/55 | +++ | ++ | | | 0 |
| 6/56 | + | +++ | | | 0 |
| 6/57 | + | + | | | 0 |
| 6/58 | + | + | | | 0 |

EC₅₀ ranges for the Nurr1-Gal4 assay as described herein: +++: <100 nM; ++: 100 nM to <1 μM; +: 1 μM to <10 μM; 0: ≥10 μM. Maximal fold activation ranges for the Nurr1-Gal4 assay as described herein: +++: ≥3-fold; ++: ≥2-fold to <3-fold; +: ≥1.3-fold to <2-fold.
IC₅₀ ranges for the DHODH assay as described herein: ++++: <10 nM; +++: <100 nM; ++: 100 nM to <1 μM; +: 1 μM to <10 μM; 0: ≥10 μM (inactive).

### Example 101-B: Reporter Gene Assays for Full-Length Human Nurr1 to Confirm Nurr1 Agonism

Activation of full length human Nurr1 was studied in transiently transfected HEK293T cells using the reporter plasmids pFR-Luc-NBRE or pFR-Luc-DR5, each containing one copy of the respective human Nurr1 response element NBRE N13 (SEQ ID NO: 1: TGA TAT CGA AAA CAA AAG GTC A) or DR5 (SEQ ID NO: 2: TGA TAG GTT CAC CGA AAG GTC A). The full length human nuclear receptor Nurr1 (pcDNA3.1-hNurr1-NE; Addgene plasmid #102363) and, for DR5, RXRα (pSG5-hRXR) were overexpressed. pFL-SV40 (Promega) was used for normalization of transfection efficacy and to observe test compound toxicity. Cells were cultured in Dulbecco's modified Eagle's medium (DMEM), high glucose supplemented with 10% fetal calf serum (FCS), sodium pyruvate (1 mM), penicillin (100 U/mL), and streptomycin (100 μg/mL) at 37° C. and 5% CO₂ and seeded in 96-well plates (3×10⁴ cells/well). After 24 h, medium was changed to Opti-MEM without supplements and cells were transiently transfected using Lipofectamine LTX reagent (Invitrogen) according to the manufacturer's protocol. Five hours after transfection, cells were incubated with the test compounds in Opti-MEM supplemented with penicillin (100 U/mL), streptomycin (100 μg/mL) and 0.1% DMSO for 16 h before luciferase activity was measured using the Dual-Glo Luciferase Assay System (Promega) according to the manufacturer's protocol on a Tecan Spark luminometer (Tecan Deutschland GmbH, Germany). Firefly luminescence was divided by Renilla luminescence and multiplied by 1000 resulting in relative light units (RLU) to normalize for transfection efficiency and cell growth. Fold activation was obtained by dividing the mean RLU of test compound by the mean RLU of the untreated control. All samples were tested in at least three biologically independent experiments in duplicates. For dose-response curve fitting and calculation of EC₅₀ values, the equation “[Agonist] versus response (three parameters)” was used in GraphPad Prism (version 7.00, GraphPad Software, La Jolla, CA, USA).

The compound of Example 1 activated full-length human Nurr1 on the response elements for the Nurr1 monomer (NBRE, EC₅₀=0.3±0.1 μM) and the RXR:Nurr1 heterodimer (DR5, EC₅₀=0.4±0.2 μM). Data are the mean±SD, n≥3 (FIG. 2 (right)).

### Example 101-C: Expression Levels of Nurr1 in the Hybrid Gal4-Nurr1 Reporter Gene Assay at One Concentration

Similarly as described in Part A, the Nurr1 modulation was determined in a Gal4 hybrid reporter gene assay at a fixed concentration of 3 μM. The following data was obtained:

| | Fold Activation | Fold Activation |
|---|---|---|
| Example# | @ 3 μM | @ 0.3 μM |
| 2/25 | ++ | +++ |
| 2/26 | + | |
| 2/27 | +++ | |
| 2/28 | ++ | |
| 2/29 | + | |
| 2/30 | +++ | |
| 6/1 | ++ | |
| 6/2 | ++ | |
| 6/3 | ++ | |
| 6/4 | +++ | |
| 6/6 | + | |
| 6/7 | ++ | |
| 6/8 | +++ | |
| 6/9 | ++ | |
| 6/10 | ++ | |
| 6/17 | +++ | |
| 6/18 | + | |
| 6/41 | | + |
| 6/44 | + | |

Maximal fold activation ranges:
+++: ≥3-fold;
++: ≥2-fold to <3-fold;
+: ≥1.3-fold to <2-fold

### Example 102: Structure-Activity-Relationship for DHODH Inhibition Versus Nurr Agonism

The in vitro inhibition of hDHODH was measured using an N-terminally truncated recombinant hDHODH enzyme. Briefly, the hDHODH concentration was adjusted in a way that an average slope of approximately 0.2 AU/min served as the positive control (e.g., without inhibitor). The assay mixture contained 60 μM 2,6-dichloroindophenol, 50 μM decylubiquinone and 100 μM dihydroorotate. The hDHODH enzyme with or without at least six different concentrations of the compounds was added and measurements were performed in 50 μM TrisHCl, 150 mM KCl and 0.1% Triton X-100 at pH 8.0 and at 30° C. The reaction was started by adding dihydroorotate and measuring the absorption at 600 nm for 2 min. For the determination of the IC₅₀ values, each data point was recorded in triplicate.

| | DHODH inhibition | |
|---|---|---|
| Comparative Compound | IC50 | Nurr1 activation EC50 |
| 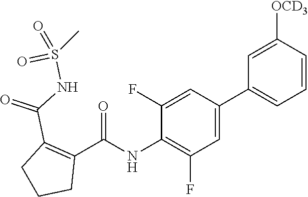 | 0.045 ± 0.020 μM | inactive (>10 μM) |
| 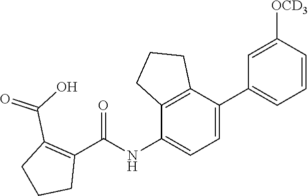 | 0.053 ± 0.002 μM | inactive (>10 μM) |
| 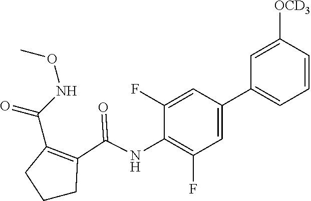 | 0.15 ± 0.03 μM | inactive (>10 μM) |
| 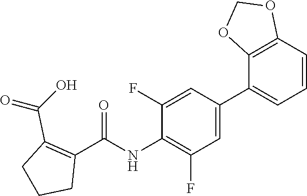 [SMILES: O=C(O)C1=C(C(=O)Nc2c(F)cc(-c3cccc4c3OCO4)cc2F)CCC1] | 0.30 ± 0.02 μM | inactive (>10 μM) |
| 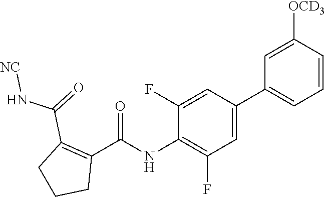 | 0.31 ± 0.00 μM | inactive (>10 μM) |
| 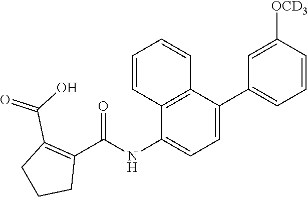 | 5.22 ± 1.39 μM | inactive (>10 μM) |
| 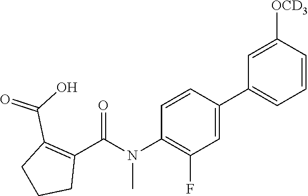 | inactive (>50 μM) | inactive (>10 μM) |
| 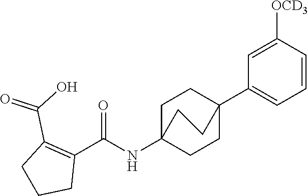 | inactive (>50 μM) | inactive (>10 μM) |

As shown with the comparison above, the structure-activity-relationship for DHODH inhibition is different compared to Nurr1 agonism. Although no activity towards Nurr1 was observed for the comparative compounds, DHODH inhibition was observed.

### Example 103: Evaluation of Nurr1 Regulated Gene Expression of Tyrosine Hydroxylase (TH) and Vesicular Amino Acid Transporter 2 (VMAT2)

T98G (ATCC CRL-1690™) were grown in DMEM, high glucose supplemented with 10% FCS, sodium pyruvate (1 mM), penicillin (100 U/mL), and streptomycin (100 μg/mL) at 37° C. and 5% CO₂ and seeded at a density of 250,000 cells per well in a 12 well plate. After 24 h, medium was changed to DMEM, high glucose supplemented with 0.2% fetal calf serum (FCS), penicillin (100 U/mL) and streptomycin (100 μg/mL) and the cells were incubated for another 24 h before stimulation with compound of Example 3 (0.3 μM and 1 μM). After 16 h of incubation the medium was removed, cells were washed with phosphate buffered saline (PBS) and after full aspiration of residual liquids immediately frozen at −80° C. until further procession. Total RNA was isolated using E.Z.N.A.® Total RNA Kit I (Omega Bio-tek, Norcross, USA) following the manufacturer's instructions. RNA concentration and purity was assessed using a NanoDrop™ One UV/VIS spectrophotometer (Thermo Fisher Scientific, Waltham, USA) at 260/280 nm. Right before reverse transcription (RT), RNA was linearized at a concentration of 133 ng/μL at 65° C. for 10 min and then immediately incubated on ice for at least 1 min. Reverse transcription was performed using 2 μg total RNA, 20 U Recombinant RNasin® Ribonuclease Inhibitor (Promega, Mannheim, Germany), 100 U SuperScript® IV Reverse Transcriptase including 5× First Strand Buffer and 0.1 M dithiothreitol (Thermo Fisher Scientific, Waltham, USA), 3.75 ng linear acrylamide, 625 ng random hexamere primers (#11277081001, Merck, Darmstadt, Germany) and 11.25 nmol deoxynucleoside triphosphate mix (2.8 nmol each ATP, TTP, CTP, GTP; #R0186, Thermo Fisher Scientific, Waltham, USA) at a volume of 22.45 μL at 50° C. for 10 min and 80° C. for 10 min using a Thermal cycler XT⁹⁶ (VWR International, Darmstadt, Germany). Quantitative polymerase chain reaction (qPCR) was conducted using an Applied Biosystems™ QuantStudio 1 (Waltham, USA) and a SYBR green based detection method. 0.2 μL of prepared cDNA were added to 6 μmol of forward and reverse primer, respectively, 0.8 U Taq DNA Polymerase (#M0267, New England Biolabs, Ipswich, USA), 40 ppm SYBR® Green I (#S9430, Sigma Aldrich, St. Louis, USA), 15 nmol deoxynucleoside triphosphate mix (as indicated above), 60 nmol MgCl₂, 4 μg bovine serum albumin (#B14, Thermo Fisher Scientific, Waltham, USA), 20% BioStab PCR Optimizer II (#53833, Merck, Darmstadt, Germany) and 10% Taq buffer without detergents (#B55, Thermo Fisher Scientific, Waltham, USA) topped up at a final volume of 20 μL with ddH₂O. Samples underwent 40 cycles of 15 s denaturation at 95° C., 15 s of primer annealing at 62.4° C., and 20 s of elongation at 68° C. PCR product specificity was evaluated using a melting curve analysis ranging from 65 to 95° C. TH and VMAT2 mRNA expression was normalized to GAPDH mRNA expression per each sample using the ΔCt-method. The following primers were used. VMAT2 (SLC18A2): 5′-GCT ATG CCT TCC TGC TGA TTG C-3′ (fw) (SEQ ID NO: 3) and 5′-CCA AGG CGA TTC CCA TGA CGT T-3′ (rev) (SEQ I NO: 4); TH: 5′-GCT GGA CAA GTG TCA TCA CCT G-3′ (fw) (SEQ ID NO: 5) and 5′-CCT GTA CTG GAA GGC GAT CTC A-3′ (rev) (SEQ ID NO: 6); GAPDH: 5′-AGG TCG GAG TCA ACG GAT TT-3′ (fw) (SEQ ID NO: 7) and 5′-TTC CCG TTC TCA GCC TTG AC-3′ (rev) (SEQ ID NO: 8).

As shown in FIG. 3 as a representative example, the compounds of Example 1 and Example 3 exhibit a dose-dependent gene expression on tyrosine hydroxylase (TH) and vesicular amino acid transporter 2 (VMAT2).

### Example 104-A: PBMC Isolation

RNA is isolated from either patients' whole blood or isolated peripheral blood mononuclear cells (PBMCs).

Whole blood is collected in BD Vacutainer. 20-25 mL of whole blood is transferred into an accuspin tube and centrifuged at 2200 rpm for 10 min at RT. The plasma supernatant is carefully aspirated. Lymphocyte ring between plasma supernatant and frit is transferred to a new tube, and then filled up and mixed with PBS+1% FCS. Upon centrifugation (1200 rpm, RT, 10 min) and washing the cell pellet with 25 mL PBS+1% FCS, cells are resuspended in 25 mL of erythrocyte lysis buffer (8.29 g ammonium chloride, 1.0 g potassium hydrogen carbonate, 0.073 g EDTA, add 1 L H₂O dest.) and incubated at RT for 10 min. After centrifugation (1200 rpm, RT, 10 min), supernatant is aspirated and cells resuspended in 10-20 mL PBMC medium (500 mL RPMI 1640, 50 mL heat-inactivated FCS, 5 mL 100× Pen/Strep, 5 mL 100× L-glutamine).

Whole blood expression profiling can be performed using PAXgene (Qiagen) or Tempus (Life Technologies) tubes which allow preservation of RNA. RNA is isolated according to respective manufacturer's protocol. Downstream applications include e.g., quantitative real time (qRT)-PCR or genome-wide approaches like microarray, RNA sequencing (RNAseq) and nanoString assays for quantification of target transcripts.

PBMC samples can be stored in RLT buffer containing β-mercaptoethanol (DNeasy Blood & Tissue Mini Kit, Qiagen) at −80° C.

### Example 104-B: Total RNA Isolation

Total RNA is isolated using silica membrane-based purification columns, e.g., included in the RNeasy Mini Kit (Qiagen) and procedure is performed according to manufacturer's instructions.

RNA can also be extracted using TRIZOL reagent (Thermo Fisher Scientific) following standard procedures. Shortly, for one sample: 200 μL blood is incubated with 800 μL TRIZOL reagent for 5 min at room temperature (RT), followed by supplementation with 200 μL chloroform. After vigorous mixing, the sample is centrifuged at 12,000×g for 15 min at 4° C. Upon transferring the upper layer to a new tube and addition of 500 μL of isopropanol, the resulting mixture is incubated for 10 min at RT. Subsequently, RNA is pelleted by centrifugation at 12,000×g for 15 min at 4° C., washed with 1 mL 75% ethanol and air-dried for 5 min. RNA is dissolved in approximately 20 μL RNase-free water.

### Example 104-C: Determination of RNA Concentration and RNA Integrity Control

RNA concentration and purity is determined on a NanoDrop spectral photometer. To evaluate RNA integrity, the RNA integrity number (RIN) is assessed. The RIN scale ranges from 10 (excellent RNA quality) to 1 (massive degradation). In general, RNA samples with RIN values ≥7.0 are suitable for next generation sequencing (NGS) library preparation.

### Example 104-D: RNA Seq Analysis

Libraries are prepared using NEBNext® Poly(A) Ultra™ II Directional RNA library prep technology according to manufacturer's instructions. Libraries are quality-controlled with high sensitivity DNA assays, e.g., DNA 1000 LabChips on 2100 Bioanalyzer (Agilent Technologies). Samples are quantified using the Qubit® dsDNA HS Assay (Thermo Fisher Scientific). Afterwards, all single libraries are pooled into a sequencing library pool and cluster generation as well as RNA sequencing is performed on the Illumina NovaSeq® 6000 according to the standard workflow.

Primary image processing is performed on the NovaSeq® 6000 instrument using Real-Time Analysis software (RTA). Primary data analysis is performed using the bcl2fastq software. The Illumina Sequence Analysis Viewer (SAV) is used for imaging and evaluation of technical quality parameters. High-quality sequenced reads are imported into CLC Genomics Workbench (Qiagen) and aligned to human reference genome (GRCh38.p7, NCBI).

Expression values are processed to RPKM (reads per kilobase of exon model per million mapped reads) values as normalized measures of relative abundance of transcripts.

Small RNA sequencing is used to isolate, sequence, and examine the differential expression of small RNA species with less than 200 nt in length. Small RNAs are often not translated into proteins and can be categorized into microRNA (miRNA), small interfering RNA (siRNA) and piwi-interacting RNA (piRNA). Small RNA sequencing is performed and analyzed according to guidelines and state of the art protocols.

### Example 104-E: qPCR Analysis

The expression of target genes and at least one housekeeping gene can also be measured by reverse-transcribing the RNA (High-Capacity cDNA Reverse Transcription Kit, Thermo Fisher Scientific) and followed by real-time PCR analysis using TaqMan™ Universal PCR Mastermix and TaqMan® Gene Expression Assays (Thermo Fisher Scientific) as described in manufacturer's protocol. Assays are run in on optical 96-well plates (4titude) using a LightCycler® 96 System (Roche) with following thermal cycling parameters: 2 min at 50° C., 10 min at 95° C., 40 cycles of 15 s at 95° C. and 1 min at 60° C. For each target gene, expression is normalized to a housekeeping gene and expressed as fold induction relative to the vehicle-treated control condition.

### Example 104-F: Microarray

Transcriptome-wide gene expression analysis via microarray technology can be conducted using GeneChip™ Human Gene 1.0/2.0 ST Array (ThermoFisher Scientific). The GeneChip whole transcript sense targeting labeling assay generates amplified and biotinylated sense-strand DNA targets from the entire expressed genome. Assay is performed according to manufacturer's instructions.

### Example 104-G: nanoSTRING

To measure nucleic acid content by counting target molecules directly without amplification, NanoString nCounter Analysis System (NanoString Technologies) is utilized. The system employs fluorescent barcodes for the direct, digital detection of target molecules. Samples are hybridized and loaded onto the Nanostring Chipset according to the manufacturer's instructions. Shortly, isolated RNA is mixed with a Master Mix (reporter code set plus hybridization buffer). Out of the NanoString panel, the appropriate capture probe sets are selected and added. After heating at 65° C. for 16 h, samples are transferred to the preparation station with prepared nCounter master kit and cartridge. After the preparation station run, cartridges are transferred to the Digital Analyzer and scanned.

### Example 105: Induction of BDNF, a Nurr1 Target Gene

In a representative experiment similar to that described above, human PBMCs were stimulated with LPS and either treated with IMU-838 (3 μM) or teriflunomide (3 μM). Supernatant of these cells was collected 14 h after stimulation and centrifuged to remove remaining cells or cell debris. Supernatant was stored and used in a BDNF ELISA. LPS is used to stimulate the monocytes and this test has been established as a surrogate test for microglia.

As shown in FIG. 4 as a representative example, BDNF is produced by immune cells/monocytes when treated with IMU-838 (3 μM). BDNF production was not observed in cells treated with comparative DHODH inhibitor teriflunomide at the same concentration. Since monocytes express Nurr1, activation of Nurr1 by IMU-838 but not by teriflunomide is consistent with the observations.

### Example 106: Additional Target Genes Upregulated by IMU-838 but not with Teriflunomide

In a representative experiment stimulated human PBMCs were either treated with IMU-838 or teriflunomide. Human PBMC isolation was performed similarly as described above. Five million isolated human PBMCs were stimulated with anti-CD3 (eBioscience #16-0038-81, 10 ng/mL) and anti-CD28 antibody (eBioscience #16-0289-81, mit 100 ng/mL) for 20 h. In parallel, cells were treated with 30 μM vidofludimus or DMSO for 20 h. Four biological replicates were used per condition. Cells were detached from the cell culture plates by using MACS buffer, were washed with PBS and then resuspended in RLT buffer for subsequent RNA isolation with RNAeasy Mini Kit (Quiagen). 100 ng total RNA per sample were introduced into an RT-IVT reaction. Prior to RT-IVT, the total RNA samples were spiked with in-vitro synthesized polyadenylated transcripts (One-Color RNA Spike-In Mix, Agilent Technologies), which serves as an internal labeling control for linearity, sensitivity and accuracy. The spiked total RNA was reverse transcribed into cDNA and then converted into labeled cRNA by in vitro transcription (Low Input Quick-Amp Labeling Kit One-Color, Agilent Technologies) incorporating Cyanine-3-CTP. All steps were carried out according to the manufacturer's instructions. Following cRNA clean-up and quantification (NanoDrop ND-1000), 600 ng of each cyanine-3-labeled cRNA sample was fragmented and prepared for One-Color based hybridization (Gene Expression Hybridization Kit, Agilent Technologies). cRNA samples were hybridized at 65° C. for 17 h on separate SurePrint G3 Human GE Microarrays (8×60K format). Afterwards, microarrays were washed with increasing stringency using Gene Expression Wash Buffers (Agilent Technologies) followed by drying with acetonitrile (SIGMA). Fluorescent signal intensities were detected with Scan Control A.8.4.1 Software (Agilent Technologies) on the Agilent DNA Microarray Scanner and extracted from the images using Feature Extraction 10.7.3.1 Software (Agilent Technologies). Differential gene expression was identified by applying appropriate biostatistics to the data set. GeneSpring GX11 analysis software was used to normalize and analyze the raw data. Different filtering approaches were applied to identify significantly up- and down-regulated genes in the comparisons of interest. The induced and repressed genes were further characterized for involvement in functional categories (biological processes, molecular functions, cellular components, protein classes and pathways) based on the PANTHER database.

The following Nurr1 target genes (Phelan et al. 2021) were upregulated by IMU-838 but not by teriflunomide:

| Gene | Function | Upregulation |
|---|---|---|
| NCAM | Cell adhesion, synaptic plasticity, | 1.6 |
| | learning and memory | |
| CFB | Core binding factor | 2.4 |
| LTA | Lymphotaxin alpha, involved in cell survival, | 2.0 |
| | proliferation, differentiation and apoptosis | |
| A2M | Antiproteinase alpha2, protection against | 1.6 |
| | invading pathogens | |
| HSD11B1 | Hydroxysteroid dehydrogenase | 2.5 |
| BHLHE41 | Basic helix loop helix, macroglia | 1.5 |
| | associated marker | |
| MARCO | Macrophage receptor with collagenous | 2.6 |
| | structure | |

Comparison with teriflunomide as another DHODH inhibitor not affecting Nurr1 activation corroborates the Nurr1-IMU-838 axis.

### Example 107: Sustaining Survival in an Apoptosis Assay in a Human SH-SY5Y and Murine N2A Cell Line

In representative experiments, the human neuronal-like cell line SH-SY5Y and the murine neuronal-like cell line N2A were cultured in the presence or absence of Example 3 for 1 h after which apoptosis was induced by TNFα (50 ng/mL) and cycloheximide (CHX, 20 μg/mL) treatment for 6 hours. Cell viability was assessed by flow cytometry after staining the cells with Annexin V and a live/dead dye. Alive cells are defined as live/dead dye AnnexinV⁻ cells.

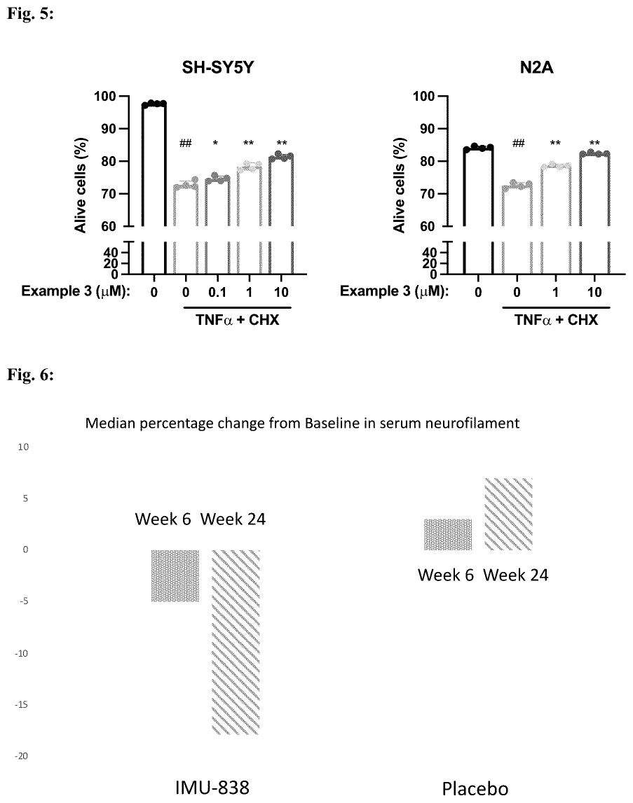

As shown in FIG. 5 as a representative example, treatment with TNFα and CHX reduced the percentage of alive cells in both SH-SY5Y and N2A cells. Moreover, the induction of apoptosis was reduced when cells were pretreated with Example 3 at the different concentrations.

### Example 200: Change from Baseline in Serum Neurofilament by Number of Relapses During Main Treatment Period

Study Objective: Quantification of neurofilament light polypeptide (NFL) by an electrochemiluminescent immunoassay (ECLIA) in serum samples from a Phase 2 Study following administration of vidofludimus calcium (IMU-838) in patients with relapsing-remitting multiple sclerosis. The study was carried out in accordance with current ICH E6 (Good Clinical Practice) guidelines and the EMA Reflection paper for laboratories that perform the analysis or evaluation of clinical trial samples (EMA/INS/GCP/532137/2010, 28 Feb. 2012). The sample analysis was performed on a Meso QuickPlex SQ 120 analyzer by an ECLIA technique using a validated method.

In order to confirm an effect of IMU-838 on the plasma levels of NFL the subgroup of patients with no relapses was investigated

Results: The mean change of NFL was a 18% reduction after 24 weeks in the treatment group whereas the mean NFL level in placebo patients increased by 7% at the same time (see FIG. 6). Patients under treatment with IMU-838 that did not have relapses exhibited reduced NFL levels compared with patients treated with placebo.

Conclusion: In relapse free patients, vidofludimus calcium (IMU-838) can protect relapse free patients from NFL secretion and thus has shown a neuroprotective effect. Or in other words: patients under treatment with IMU-838 which don't have relapses show reduced NFL levels compared with patients treated with placebo.

### Example 201: Neurofilament Measurement

Study Objective: High sensitivity quantification of neurofilament light polypeptide (NFL) can be performed using the Simoa® technology in serum, plasma and CSF samples from patients to either determine baseline levels compared to age and gender matched healthy volunteers or analysis the change from baseline following treatment. The sample analysis is performed on a HD-X™ Automated Immunoassay Analyzer, SR-X™ Biomarker Detection System or similar analyzer.

### Example 202: GFAP Measurement

Study Objective: High sensitivity quantification of GFAP can be performed using the Simoa® technology or using a high sensitivity Electrochemiluminescence assay in serum, plasma and CSF samples from patients to either determine baseline levels compared to age and gender matched healthy volunteers or analysis the change from baseline following treatment. The sample analysis is performed on a HD-X™ Automated Immunoassay Analyzer, SR-X™ Biomarker Detection System or similar analyzer for the Simoa® technology or a Meso QuickPlex SQ 120 analyzer or similar for the (ECLIA).

### Example 203: Study to Evaluate Efficacy, Safety, and Tolerability of IMU-838 in Patients with Progressive Multiple Sclerosis (CALLIPER)

Study Objective: CALLIPER enrolment was completed in August 2023 with 467 patients randomized 1:1 to 45 mg of vidofludimus calcium or placebo. The neurofilament light chain (NFL) analysis on week 24 of treatment was performed in an interims analysis. Safety analysis was performed by an Independent Data Monitoring Committee.

The primary endpoint of the trial is brain volume change, meaning brain atrophy. The key secondary endpoint is confirmed disability worsening. The study population includes about 35% of patients with Primary Progressive Multiple Sclerosis (PPMS) and about 60% of patients with non-active Secondary Progressive Multiple Sclerosis (non-active SPMS), considered the most difficult to treat patients in MS and for which no relevant treatment is currently approved in the US. Progressive MS (in particular non-active progressive MS populations) have shown to have little to no effect in response to anti-inflammatory drugs that are commonly used in relapsing MS.

Patient inclusion criteria were:
Adult patients, age 18 to 65 years (inclusive).
Male or female.
EDSS score at screening between 3.0 to 6.5 (both inclusive)
No evidence of relapse in the last 24 months before randomization, AND Patients diagnosed according to 2017 revised McDonald Criteria 1 and the 2013 revised classification of disease courses 2 as either 1. SPMS inpatients showing evidence of Gd+MRI lesions (active SPMS) or without Gd+MRI lesions (non-active SPMS) in the last 12 months, OR 2. PPMS
Willingness and ability to comply with the protocol.
Written informed consent given by the patient before the beginning of any study-related procedure.
Documented evidence of disability progression not temporarily related to a relapse in the last 24 months before randomization, adjudicated by a central independent reviewer.

While the patients who met the inclusion criteria had no evidence of relapse in the 24 months prior to enrollment, the inclusion criteria did not allow prediction of patients whose disease would progress without relapse during the course of the study and patients who would experience relapse during the study. Notably, the inclusion criteria do not select for only patients with PIRA.

Neurofilament is a neuronal biomarker originating from breakdown of proteins that occur when nerve cells are injured or are destroyed. Neurofilament initially is released into cerebrospinal fluid in the brain but can also be measured in blood. This biomarker is used in a range of neurodegenerative diseases. NFL is well characterized in multiple sclerosis and is found to be correlated with brain tissue loss. Bar-Or et al. (eBioMedicine 2023; 93:104662) summarizes a recent analysis of neurofilament data in the Phase 3 trial of ocrelizumab (called ORATORIO) that led to its approval in primary progressive MS. The analysis only included patients that were on active treatment and followed them for 8 years on treatment. Since PPMS patients can have some level of MRI or relapse activity initially, the neurofilament values in these patients were re-baselined after 48 weeks. This ensured a progressive MS population free of focal inflammatory disease and any future disability is not related to focal inflammatory disease. At the re-baselining after 48 weeks, patients were then divided into patients with low NFL and with high NFL. The analysis has shown that progressive MS patients with low NFL (in the absence of focal inflammatory disease) experience substantially less disability worsening events over the next 8 years. So, lower NFL correlated with lower risk for disability in PPMS patients. Furthermore, at week 24 a difference of 12.4% between the active and placebo groups was observed.

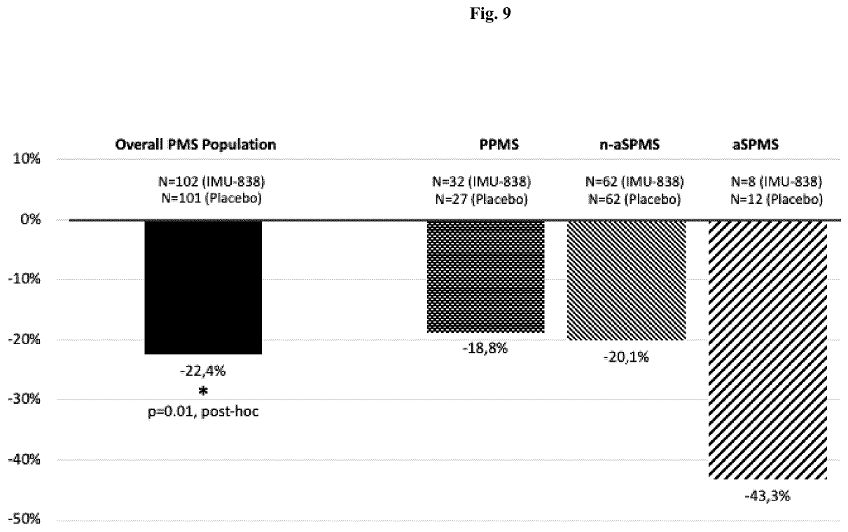

In the present clinical study, vidofludimus calcium consistently demonstrated positive responses and statistically significant reduction of neurodegenerative disease biomarker NFL in blood serum across all subpopulations and stages of progressive MS with or without MRI activity (See FIG. 9).

FIG. 9 shows the standard deviation for change from baseline in % of baseline: CALLIPER week 24: IMU-838 35.7%, PPMS: IMU-838 7.1%, n-aSPMS: IMU-838 14.7%, aSPMS: IMU-838 10.3%, 95% Hodges-Lehmann confidence bound EMPhASIS week 24 for 45 mg IMU-838: lower boundary −41.0%, upper boundary −12.0%, includes all randomized patients with available neurofilament data at interim analysis, arithmetic mean value for group averages; aSPMS and n-aSPMS designation as per diagnosis by clinical investigator at study entry. Abbreviations: RRMS: relapsing-remitting multiple sclerosis; PPMS: primary progressive multiple sclerosis; SPMS: secondary progressive multiple sclerosis; n-a: non-active; a: active.

At week 24 in the overall PMS population the value of NFL in blood was statistically significantly reduced from baseline by 22.4% compared to Placebo (p=0.001, post-hoc statistical analysis). In particular, a −18.8% reduction for vidofludimus calcium versus placebo in PPMS and a −20% reduction in non-active SPMS was observed. Active SPMS patients treated with vidofludimus calcium showed an even better response in neurofilament with a 43% reduction.

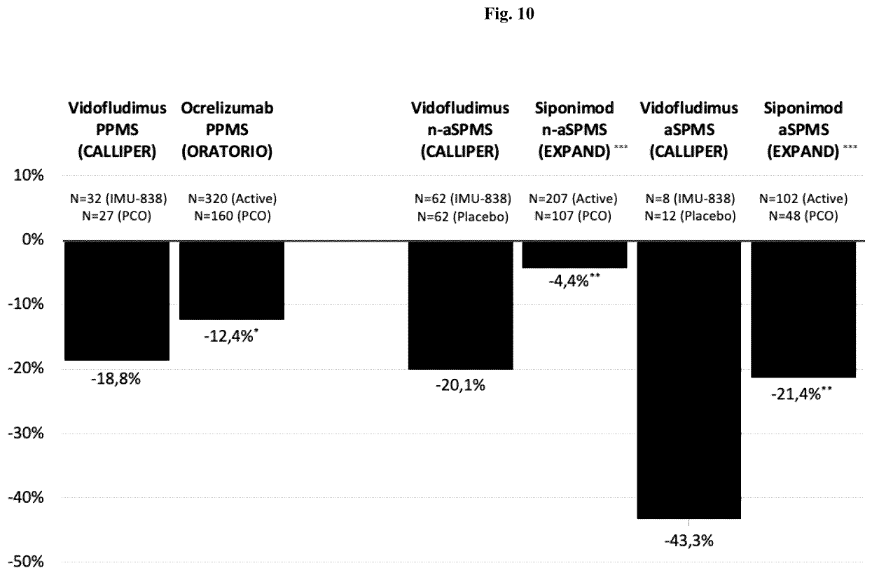

In FIG. 10 the comparison of CALLIPER interim data on week 24 with the best available historical studies ORATORIO (ocrelizumab) and EXPAND (siponimod) in the same progressive MS subtypes is shown. N=Number of patients in the 45 mg IMU-838 groups, only patients with both baseline and week 24 values considered for change from baseline analysis, arithmetic mean value for group averages; includes all randomized patients with available NFL data at interim analysis. Standard deviation for change from baseline in % of baseline: CALLIPER week 24: IMU-838 35.7%; 95% Hodges-Lehmann confidence bound EMPhASIS week 24 for 45 mg IMU-838: lower boundary −41.0%, upper boundary −12.0%. ORATORIO: Bar-Or A. et al., eBioMedicine 2023; 93:104662; EXPAND: Leppert D. et al., Neurology 2022; 98: e2120.*plasma NFL levels; ** 12-month data; *** Displayed are data for subpopulation without relapses (n-aSPMS) and with relapses (aSPMS); PCO: placebo; PPMS: primary progressive multiple sclerosis; SPMS: secondary progressive multiple sclerosis; n-a: non-active; a: active.

For the PPMS group at ORATORIO approval study ocrelizumab showed a difference of NFL values between active and placebo at weeks 24 of 12.4%. In the CALLIPER trial, a 18.8% improvement of active over placebo at week 24 was observed.

In regards of SPMS, the Phase 3 EXPAND trial of siponimod led to approval in active SPMS based on clinical outcomes, however, it was negative for the non-active SPMS group. The NFL reduction in EXPAND study in the earlier available 12-month point was almost exclusively observed in the active SPMS population, where focal inflammatory processes characterized by MRI lesions and relapses are still present. For non-active SPMS without such focal inflammatory processes ongoing, siponimod essentially had little or no effect on neurofilament.

In contrast, vidofludimus with its combination of anti-inflammatory, anti-viral and neuroprotective mechanisms was able to show a substantial reduction in neurofilament in both the active and non-active SPMS populations.

In summary, in the overall progressive MS population at week 24, neurofilament showed an improvement for vidofludimus calcium over placebo of more than 22%. A similar amplitude of effect was consistently shown across all subtype and subgroup analyses. The NFL effect shown especially in the non-active SPMS subtype reinforces the neuroprotective potential of vidofludimus calcium in the absence of focal inflammatory disease. The data from this CALLIPER interim analysis are comparable or even numerically superior to the best historical data available in the same patient population.

### EQUIVALENTS AND SCOPE

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of treating multiple sclerosis (MS) in a patient diagnosed with progression independent of relapse (PIRA), comprising administering the subject a compound of Formula (V), wherein the compound is:

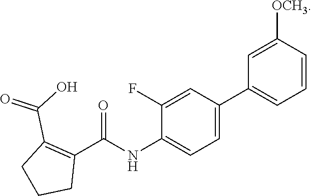 [SMILES: COc1cccc(-c2ccc(NC(=O)C3=C(C(=O)O)CCC3)c(F)c2)c1]

2. A method of treating multiple sclerosis (MS) in a subject, comprising administering to the subject a compound of Formula (V), wherein:
the compound is 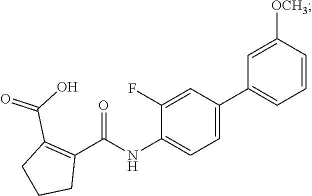 [SMILES: COc1cccc(-c2ccc(NC(=O)C3=C(C(=O)O)CCC3)c(F)c2)c1]
and
the MS is characterized by progression independent of relapse (PIRA).

3. A method of treating progression independent of relapse (PIRA) in a subject suffering from multiple sclerosis (MS), comprising administering to the subject a compound of Formula (V), wherein the compound is

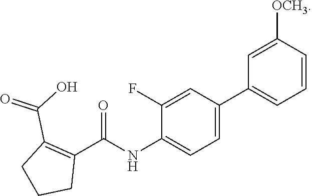 [SMILES: COc1cccc(-c2ccc(NC(=O)C3=C(C(=O)O)CCC3)c(F)c2)c1]

4. A method of treating multiple sclerosis (MS) in a subject, comprising administering to the subject a compound of Formula (V), wherein:
the compound is 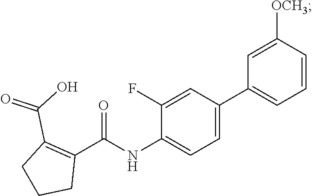 [SMILES: COc1cccc(-c2ccc(NC(=O)C3=C(C(=O)O)CCC3)c(F)c2)c1]
and
the subject has MS characterized by progression independent of relapse (PIRA).

5. The method of claim 1, wherein the patient has no evidence of relapse for 24 months.

6. The method of claim 1, wherein the MS is progressive MS (PMS).

7. The method of claim 6, wherein the PMS is characterized by few or no active lesions.

8. The method of claim 6, wherein the MS is primary progressive MS (PPMS).

9. The method of claim 6, wherein the MS is non-active secondary progressive MS (n-aSPMS).

10. The method of claim 9, wherein the n-aSPMS is characterized by no lesions for 12 months.

11. The method of claim 1, wherein the MS is active secondary progressive MS (a-SPMS).

12. The method of claim 1, wherein the method reduces serum NFL levels in the subject, as compared to a control.

13. The method of claim 1, wherein the method reduces serum GFAP levels in the subject, as compared to a control.

14. The method of claim 1, wherein the method reduces the rate of percent brain volume change (PBVC) in the subject, as compared to a control.

15. The method of claim 1, wherein the method reduces the rate of change in brain parenchymal fraction (BPF) in the subject, as compared to a control.

16. The method of claim 1, wherein the method increases the time to confirmed disability worsening, e.g, based on an expanded disability status scale (EDSS), in the subject as compared to a control.

17. The method of claim 1, wherein the method prevents and/or slows disease progression and secondary injuries by halting or at least slowing (mitigate) the loss of neurons.

18. The method of claim 1, wherein the compound is administered in a therapeutically-effective amount of about 5 mg to about 100 mg.

19. The method of claim 1, wherein the compound is administered orally by a solid dosage form.

20. The method of claim 1, wherein the compound is administered over a first period and a second period, wherein: a) the first period is five to ten consecutive days of once-daily dosing of a first period amount of the compound, wherein the first period amount of the compound is about 15 mg to about 25 mg; b) the second period follows the first period; and c) the second period comprises once-daily dosing of a second period amount of the compound, wherein the second period amount is about 30 mg to about 50 mg.
